(19) United States
(12) Patent Application Publication
(10) Pub. No.: US 2026/0199447 A1
(43) Pub. Date: Jul. 16, 2026

(54) NEUTRALIZING MONOCLONAL ANTIBODIES AGAINST WESTERN, EASTERN, AND VENEZUELAN EQUINE ENCEPHALITIS VIRUS (EEV)

(71) Applicants: The U.S. of America, as Represented by the Secretary , Department of Health & Human Services, Bethesda, MD (US); The Trustees of Columbia University in the City of New York, New York, NY (US)

(72) Inventors: Mario Roederer, Bethesda, MD (US); Sungyoul Ko, Bucheon-si (KR); Matthew Scott Sutton, Arlington, VA (US); Peter D. Kwong, Washington, DC (US); Raffaello Verardi, Washington, DC (US); Reda Rawi, North Bethesda, MD (US); Chen-Hsiang Shen, North Bethesda, MD (US); Julie Fox, Bethesda, MD (US); Victoria Callahan, Gainesville, VA (US); Gabriele Cerutti, New York, NY (US); Ryan Gavin Casner, New York, NY (US); Lawrence Shapiro, New York, NY (US); Yaroslav Tsybovsky, Brunswick, MD (US); Sergei Pletnev, Bethesda, MD (US)

(21) Appl. No.: 19/136,772

(22) PCT Filed: Dec. 7, 2023
(86) PCT No.: PCT/US2023/082822

§ 371 Date: Jun. 6, 2025

Related U.S. Application Data
(60) Provisional application No. 63/386,488, filed on Dec. 7, 2022.

(51) Int. Cl. A61K 39/12; A61K 9/00; A61K 38/00; A61K 39/00; A61P 31/14; C07K 16/116

(52) U.S. Cl. CPC A61K 39/12; A61K 9/0019; A61K 38/00; A61P 31/14; C07K 16/116; A61K 2039/505; A61K 2039/5258; A61K 2039/543; A61K 2039/70; C07K 2317/33; C07K 2317/35; C07K 2317/565; C07K 2317/76

(57) ABSTRACT

Disclosed herein are isolated antibodies that bind one or more species of alphaviruses. In certain aspects, the disclosed antibodies bind and neutralize three or more species of alphaviruses. Also disclosed are methods of using the disclosed antibodies to prevent infection of a cell with an alpha virus, to protect an individual from alphavirus infection, and to treat an individual for an alphavirus infection.

### FIELD OF THE DISCLOSURE

The field of the disclosure relates generally to antibodies that neutralize one or more viruses of the genus Alphaviruses.

### BACKGROUND OF THE DISCLOSURE

Alphaviruses are enveloped, positive-sense, single-stranded RNA viruses in the family Togaviridae with substantial impact on animal and human health. Transmitted by mosquitos, alphavirus infection in humans results in a spectrum of disease that can be categorized as either arthritogenic or encephalitic. Arthritogenic alphaviruses, such as chikungunya virus (CHIKV), Ross River virus (RRV), Mayaro virus (MAYV), or o'nyong'nyong virus (ONNV), most often cause acute and chronic musculoskeletal disease. While rarely fatal, symptoms include fever, rash, and myalgia; incapacitating arthralgia and myalgia can persist for months to years after infection. In contrast, infection with encephalitic alphaviruses, including western, eastern, or Venezuelan equine encephalitis virus (W/E/V/EEV), can vary from asymptomatic or acute febrile illness to severe neurological impairment and death. Eastern EEV (EEEV) is the most virulent, with a human case fatality rate estimated at 30-75% in symptomatic individuals with neuro-invasive disease. While Venezuelan EEV (VEEV) is the least lethal, with death occurring in <1% of cases, it may be the most important in terms of morbidity. Unlike WEEV and EEEV, for which humans represent dead-end hosts, outbreaks of VEEV can become self-sustaining as virus can replicate to levels sufficient for domestic/urban transmission between humans. Moreover, most infections with VEEV are apparent and present abruptly with debilitating symptoms like those caused by arthritogenic alphaviruses. Though outbreaks of alphavirus infection are often geographically isolated by vector and host reservoir availability, social and environmental factors leading to the spread of mosquito vectors into new geographic areas is cause for concern.

The alphavirus genome encodes four non-structural proteins (nsP1, nsP2, nsP3, and nsP4) and five structural proteins (capsid, E3, E2, 6K, and E1). On the surface of a mature virion, heterodimers of the envelope proteins, E1 and E2, assemble into 80 trimeric spikes that are arranged with T=4 icosahedral symmetry. At the apex of the trimer, the E2 protein engages host entry factors and receptors, while the membrane-proximal E1 protein mediates viral entry via the hydrophobic fusion loop. While the fusion loop is theoretically an appealing target for broadly reactive anti-EEV monoclonal antibodies (mAbs), it is occluded by the E2 protein and only accessible following a conformation change that occurs when exposed to low pH in solution or within endosomes. Several studies highlight the importance of antibody-mediated protection against alphavirus infection, although not until recently have antibodies been identified with a wide breadth against all three EEVs, as well as against multiple arthritogenic alphaviruses. These pan-alphavirus antibodies target a conserved, cryptic E1 epitope proximal to and within the fusion peptide and were shown to protect mice against multiple arthritogenic and encephalitic alphaviruses despite poor neutralization activity. While these antibodies may prove important as prophylactic or post-exposure therapies, a vaccine candidate that elicits pan-alphavirus antibodies remains to be demonstrated.

Multiple vaccine and therapeutic candidates have been explored to combat alphavirus infections, yet none have been approved for public use in the United States. All three EEVs, as well as CHIKV, are also transmissible via aerosol exposure. Accordingly, the EEVs and CHIKV are considered NIAID Category B priority pathogens and USDA/CDC Select Agents due to their potential as bioterrorism agents. Investigational vaccines against each EEV have been used by the US military since the 1970s for at-risk laboratory and military personnel, but have resulted in poor immunogenicity, limited longevity, and substantial side effects. Accordingly, there is a need for new therapeutics that may be used to treat infection by not only by individual alphaviruses, but also by multiple alphaviruses. The present disclosure provides such novel therapeutics and provides other benefits as well.

### BRIEF DESCRIPTION OF THE DISCLOSURE

The present disclosure relates to isolated antibodies that binds one or more species of virus from the genus alphavirus. In certain aspects, an isolated antibody may bind at least three different species of virus from the genus alphavirus, which may be selected from the group consisting of Western equine encephalitis virus (WEEV), Eastern equine encephalitis virus (EEEV), Venezuelan equine encephalitis virus (VEEV), Chikungunya virus (CHIKV), Ross River Virus (RRV), Mayaro Virus (MAYV) and O‘nyong’nyong virus (ONNV). In certain aspects, an isolated antibody may neutralize at least three, or at least four, species of alphavirus.

In certain aspects, specificity determining residues in the isolated antibody may contact at least 15 amino acid residues in an epitope selected from the group comprising, consisting of, or consisting essentially of: an epitope comprising Y89 of the E1 protein of EEEV, G90 of the E1 protein of EEEV, D3 of the E2 protein of EEEV, F6 of the E2 protein of EEEV, T7 of the E2 protein of EEEV, Y9 of the E2 protein of EEEV, K10 of the E2 protein of EEEV, L11 of the E2 protein of EEEV, R13 of the E2 protein of EEEV, T57 of the E2 protein of EEEV, D58 of the E2 protein of EEEV, G59 of the E2 protein of EEEV, M68 of the E2 protein of EEEV, N69 of the E2 protein of EEEV, G70 of the E2 protein of EEEV, K71 of the E2 protein of EEEV, T72 of the E2 protein of EEEV, Q73 of the E2 protein of EEEV, K74 of the E2 protein of EEEV, S75 of the E2 protein of EEEV5, V172, S176 of the E2 protein of EEEV, K186 of the E2 protein of EEEV, T188 of the E2 protein of EEEV, V189 of the E2 protein of EEEV, P190 of the E2 protein of EEEV, S191 of the E2 protein of EEEV, G192 of the E2 protein of EEEV, A193 of the E2 protein of EEEV, Q194 of the E2 protein of EEEV, T209 of the E2 protein of EEEV, S210 of the E2 protein of EEEV, S211 of the E2 protein of EEEV, L227 of the E2 protein of EEEV, I228 of the E2 protein of EEEV, D229 of the E2 protein of EEEV, N230 of the E2 protein of EEEV, K231 of the E2 protein of EEEV, and K232 of the E2 protein of EEEV; an epitope comprising, consisting of, or consisting essentially of G64 of the E1 protein of VEEV, S65 of the E1 protein of VEEV, Q66 of the E1 protein of VEEV, E67 of the E1 protein of VEEV, C68 of the E1 protein of VEEV, T69 of the E1 protein of VEEV, P70 of the E1 protein of VEEV, Y72 of the E1 protein of VEEV, D75 of the E1 protein of VEEV, E76 of the E1 protein of VEEV, Q77 of the E1 protein of VEEV, C78 of the E1 protein of VEEV, K79 of the E1 protein of VEEV, V80 of the E1 protein of VEEV, F81 of the E1 protein of VEEV, T82 of the E1 protein of VEEV, T98 of the E1 protein of VEEV, E99 of the E1 protein of VEEV, Y214 of the E1 protein of VEEV, A215 of the E1 protein of VEEV, N216 of the E1 protein of VEEV, N218 of the E1 protein of VEEV, L219 of the E1 protein of VEEV, V220 of the E1 protein of VEEV, R223 of the E1 protein of VEEV, S141 of the E1 protein of VEEV, V143 of the E1 protein of VEEV, D311 of the E1 protein of VEEV, K340 of the E1 protein of VEEV, T389 of the E1 protein of VEEV, H390 of the E1 protein of VEEV, and Y393 of the E1 protein of VEEV; an epitope comprising, consisting of, or consisting essentially of F87 of the E1 protein of VEEV, M88 of the E1 protein of VEEV, W89 of the E1 protein of VEEV, G90 of the E1 protein of VEEV, G91 of the E1 protein of VEEV, Y93 of the E1 protein of VEEV, F95 of the E1 protein of VEEV, C96 of the E1 protein of VEEV, D97 of the E1 protein of VEEV, E99 of the E1 protein of VEEV, A226 of the E1 protein of VEEV, G227 of the E1 protein of VEEV, H28 of the E2 protein of VEEV, L55 of the E2 protein of VEEV, G63 of the E2 protein of VEEV, R64 of the E2 protein of VEEV, D94 of the E2 protein of VEEV, G95 of the E2 protein of VEEV, H96 of the E2 protein of VEEV, H156 of the E2 protein of VEEV, D157 of the E2 protein of VEEV, A158 of the E2 protein of VEEV, and Q159 of the E2 protein of VEEV; an epitope comprising, consisting of, or consisting essentially of E348 of the E1 protein of VEEV, Q349 of the E1 protein of VEEV, K319 of the E1 protein of VEEV, S325 of the E1 protein of VEEV, T347 of the E1 protein of VEEV, E348 of the E1 protein of VEEV, Q349 of the E1 protein of VEEV, A298 of the E1 protein of VEEV, T317 of the E1 protein of VEEV, K319 of the E1 protein of VEEV, S323 of the E1 protein of VEEV, K324 of the E1 protein of VEEV, S325 of the E1 protein of VEEV, G326 of the E1 protein of VEEV, E345 of the E1 protein of VEEV, T347 of the E1 protein of VEEV, E348 of the E1 protein of VEEV, Q349 of the E1 protein of VEEV, G350 of the E1 protein of VEEV, and S351 of the E1 protein of VEEV; an epitope comprising, consisting of, or consisting essentially of S180 of the E2 protein of VEEV, L181 of the E2 protein of VEEV, S182 of the E2 protein of VEEV, G183 of the E2 protein of VEEV, S185 of the E2 protein of VEEV, T187 of the E2 protein of VEEV, T189 of the E2 protein of VEEV, P190 of the E2 protein of VEEV, P191 of the E2 protein of VEEV, D192 of the E2 protein of VEEV, G193 of the E2 protein of VEEV, E209 of the E2 protein of VEEV, T210 of the E2 protein of VEEV, 1211 of the E2 protein of VEEV, N212 of the E2 protein of VEEV, K213 of the E2 protein of VEEV, T214 of the E2 protein of VEEV, K215 of the E2 protein of VEEV, Q216 of the E2 protein of VEEV, and S218 of the E2 protein of VEEV; an epitope comprising, consisting of, or consisting essentially of C63 of the E1 protein of WEEV, S65 of the E1 protein of WEEV, L66 of the E1 protein of WEEV, E67 of the E1 protein of WEEV, C68 of the E1 protein of WEEV, K69 of the E1 protein of WEEV, A70 of the E1 protein of WEEV, S71 of the E1 protein of WEEV, D75 of the E1 protein of WEEV, Y76 of the E1 protein of WEEV, T77 of the E1 protein of WEEV, C78 of the E1 protein of WEEV, R79 of the E1 protein of WEEV, V80 of the E1 protein of WEEV, F81 of the E1 protein of WEEV, G82 of the E1 protein of WEEV, G83 of the E1 protein of WEEV, S98 of the E1 protein of WEEV, E99 of the E1 protein of WEEV, N100 of the E1 protein of WEEV, T101 of the E1 protein of WEEV, M195 of the E1 protein of WEEV, A215 of the E1 protein of WEEV, R216 of the E1 protein of WEEV, D218 of the E1 protein of WEEV, I219 of the E1 protein of WEEV, L221 of the E1 protein of WEEV, and K223 of the E1 protein of WEEV; an epitope comprising, consisting of, or consisting essentially of N57 of the E2 protein of WEEV, Q58 of the E2 protein of WEEV, A59 of the E2 protein of WEEV, T61 of the E2 protein of WEEV, R173 of the E2 protein of WEEV, G197 of the E2 protein of WEEV, K198 of the E2 protein of WEEV, K233 of the E2 protein of WEEV, D235 of the E2 protein of WEEV, Q236 of the E2 protein of WEEV, T237 of the E2 protein of WEEV, W239 of the E2 protein of WEEV, D251 of the E2 protein of WEEV, S253 of the E2 protein of WEEV, V254 of the E2 protein of WEEV, Q255 of the E2 protein of WEEV, G256 of the E2 protein of WEEV, and K257 of the E2 protein of WEEV; an epitope comprising, consisting of, or consisting essentially of K61 of the E1 protein of WEEV, C63 of the E1 protein of WEEV, G64 of the E1 protein of WEEV, S65 of the E1 protein of WEEV, F95 of the E1 protein of WEEV, N199 of the E2 protein of WEEV, T201 of the E2 protein of WEEV, Y202 of the E2 protein of WEEV, K205 of the E2 protein of WEEV, D208 of the E2 protein of WEEV, Y209 of the E2 protein of WEEV, S210 of the E2 protein of WEEV, T211 of the E2 protein of WEEV, G212 of the E2 protein of WEEV, I213 of the E2 protein of WEEV, V214 of the E2 protein of WEEV, S215 of the E2 protein of WEEV, T216 of the E2 protein of WEEV, T218 of the E2 protein of WEEV, K219 of the E2 protein of WEEV, M220 of the E2 protein of WEEV, and N221 of the E2 protein of WEEV; and, an epitope comprising, consisting of, or consisting essentially of Q59 of the E1 protein of WEEV, V60 of the E1 protein of WEEV, K61 of the E1 protein of WEEV, C62 of the E1 protein of WEEV, Q93 of the E1 protein of WEEV, C94 of the E1 protein of WEEV, F95 of the E1 protein of WEEV, R173 of the E2 protein of WEEV, P176 of the E2 protein of WEEV, A178 of the E2 protein of WEEV, T201 of the E2 protein of WEEV, Y202 of the E2 protein of WEEV, E203 of the E2 protein of WEEV, K205 of the E2 protein of WEEV, G207 of the E2 protein of WEEV, D208 of the E2 protein of WEEV, Y209 of the E2 protein of WEEV, S210 of the E2 protein of WEEV, T211 of the E2 protein of WEEV, G212 of the E2 protein of WEEV, I213 of the E2 protein of WEEV, N221 of the E2 protein of WEEV, 1230 of the E2 protein of WEEV, Y232 of the E2 protein of WEEV, S234 of the E2 protein of WEEV, Q236 of the E2 protein of WEEV, 1247 of the E2 protein of WEEV, R248 of the E2 protein of WEEV, H249 of the E2 protein of WEEV, T250 of the E2 protein of WEEV, D251 of the E2 protein of WEEV, S253 of the E2 protein of WEEV, and Q255 of the E2 protein of WEEV.

In certain aspects, the isolated antibody may be selected from the group consisting of: an isolated antibody in which the specificity determining residues of the isolated antibody interact with at least 15, optionally at least 20, optionally at least 22, optionally at least 24, optionally at least 26, optionally at least 28, optionally at least 30, optionally at least 32, optionally at least 34, optionally at least 36, optionally at least 37, or all, contact residues selected from the group consisting of Y89 of the E1 protein of EEEV, G90 of the E1 protein of EEEV, D3 of the E2 protein of EEEV, F6 of the E2 protein of EEEV, T7 of the E2 protein of EEEV, Y9 of the E2 protein of EEEV, K10 of the E2 protein of EEEV, L11 of the E2 protein of EEEV, R13 of the E2 protein of EEEV, T57 of the E2 protein of EEEV, D58 of the E2 protein of EEEV, G59 of the E2 protein of EEEV, M68 of the E2 protein of EEEV, N69 of the E2 protein of EEEV, G70 of the E2 protein of EEEV, K71 of the E2 protein of EEEV, T72 of the E2 protein of EEEV, Q73 of the E2 protein of EEEV, K74 of the E2 protein of EEEV, S75 of the E2 protein of EEEV5, V172, S176 of the E2 protein of EEEV, K186 of the E2 protein of EEEV, T188 of the E2 protein of EEEV, V189 of the E2 protein of EEEV, P190 of the E2 protein of EEEV, S191 of the E2 protein of EEEV, G192 of the E2 protein of EEEV, A193 of the E2 protein of EEEV, Q194 of the E2 protein of EEEV, T209 of the E2 protein of EEEV, S210 of the E2 protein of EEEV, S211 of the E2 protein of EEEV, L227 of the E2 protein of EEEV, I228 of the E2 protein of EEEV, D229 of the E2 protein of EEEV, N230 of the E2 protein of EEEV, K231 of the E2 protein of EEEV, and K232 of the E2 protein of EEEV; an isolated antibody in which the specificity determining residues of the isolated antibody interact with at least 15, optionally at least 20, optionally at least 22, optionally at least 24, optionally at least 26, optionally at least 28, optionally at least 30, optionally at least 31, optionally at least 32, or all, contact residues selected from the group consisting of G64 of the E1 protein of VEEV, S65 of the E1 protein of VEEV, Q66 of the E1 protein of VEEV, E67 of the E1 protein of VEEV, C68 of the E1 protein of VEEV, T69 of the E1 protein of VEEV, P70 of the E1 protein of VEEV, Y72 of the E1 protein of VEEV, D75 of the E1 protein of VEEV, E76 of the E1 protein of VEEV, Q77 of the E1 protein of VEEV, C78 of the E1 protein of VEEV, K79 of the E1 protein of VEEV, V80 of the E1 protein of VEEV, F81 of the E1 protein of VEEV, T82 of the E1 protein of VEEV, T98 of the E1 protein of VEEV, E99 of the E1 protein of VEEV, Y214 of the E1 protein of VEEV, A215 of the E1 protein of VEEV, N216 of the E1 protein of VEEV, N218 of the E1 protein of VEEV, L219 of the E1 protein of VEEV, V220 of the E1 protein of VEEV, R223 of the E1 protein of VEEV, S141 of the E1 protein of VEEV, V143 of the E1 protein of VEEV, D311 of the E1 protein of VEEV, K340 of the E1 protein of VEEV, T389 of the E1 protein of VEEV, H390 of the E1 protein of VEEV, and Y393 of the E1 protein of VEEV; an isolated antibody in which the specificity determining residues of the isolated antibody interact with at least 15, optionally at least 18, optionally at least 20, optionally at least 21, optionally at least 22, or all, contact residues selected from the group consisting of F87 of the E1 protein of VEEV, M88 of the E1 protein of VEEV, W89 of the E1 protein of VEEV, G90 of the E1 protein of VEEV, G91 of the E1 protein of VEEV, Y93 of the E1 protein of VEEV, F95 of the E1 protein of VEEV, C96 of the E1 protein of VEEV, D97 of the E1 protein of VEEV, E99 of the E1 protein of VEEV, A226 of the E1 protein of VEEV, G227 of the E1 protein of VEEV, H28 of the E2 protein of VEEV, L55 of the E2 protein of VEEV, G63 of the E2 protein of VEEV, R64 of the E2 protein of VEEV, D94 of the E2 protein of VEEV, G95 of the E2 protein of VEEV, H96 of the E2 protein of VEEV, H156 of the E2 protein of VEEV, D157 of the E2 protein of VEEV, A158 of the E2 protein of VEEV, and Q159 of the E2 protein of VEEV; an isolated antibody in which the specificity determining residues of the isolated antibody interact with at least 15, optionally at least 16, optionally at least 17, optionally at least 18, optionally at least 19, or all, contact residues selected from the group consisting of E348 of the E1 protein of VEEV, Q349 of the E1 protein of VEEV, K319 of the E1 protein of VEEV, S325 of the E1 protein of VEEV, T347 of the E1 protein of VEEV, E348 of the E1 protein of VEEV, Q349 of the E1 protein of VEEV, A298 of the E1 protein of VEEV, T317 of the E1 protein of VEEV, K319 of the E1 protein of VEEV, S323 of the E1 protein of VEEV, K324 of the E1 protein of VEEV, S325 of the E1 protein of VEEV, G326 of the E1 protein of VEEV, E345 of the E1 protein of VEEV, T347 of the E1 protein of VEEV, E348 of the E1 protein of VEEV, Q349 of the E1 protein of VEEV, G350 of the E1 protein of VEEV, and S351 of the E1 protein of VEEV; an isolated antibody in which the specificity determining residues of the isolated antibody interact with at least 15, optionally at least 16, optionally at least 17, optionally at least 18, optionally at least 19, or all, contact residues selected from the group consisting of S180 of the E2 protein of VEEV, L181 of the E2 protein of VEEV, S182 of the E2 protein of VEEV, G183 of the E2 protein of VEEV, S185 of the E2 protein of VEEV, T187 of the E2 protein of VEEV, T189 of the E2 protein of VEEV, P190 of the E2 protein of VEEV, P191 of the E2 protein of VEEV, D192 of the E2 protein of VEEV, G193 of the E2 protein of VEEV, E209 of the E2 protein of VEEV, T210 of the E2 protein of VEEV, 1211 of the E2 protein of VEEV, N212 of the E2 protein of VEEV, K213 of the E2 protein of VEEV, T214 of the E2 protein of VEEV, K215 of the E2 protein of VEEV, Q216 of the E2 protein of VEEV, and S218 of the E2 protein of VEEV; an isolated antibody in which the specificity determining residues of the isolated antibody interact with at least 15, optionally at least 20, optionally at least 22, optionally at least 24, optionally at least 26, or all, contact residues selected from the group consisting of C63 of the E1 protein of WEEV, S65 of the E1 protein of WEEV, L66 of the E1 protein of WEEV, E67 of the E1 protein of WEEV, C68 of the E1 protein of WEEV, K69 of the E1 protein of WEEV, A70 of the E1 protein of WEEV, S71 of the E1 protein of WEEV, D75 of the E1 protein of WEEV, Y76 of the E1 protein of WEEV, T77 of the E1 protein of WEEV, C78 of the E1 protein of WEEV, R79 of the E1 protein of WEEV, V80 of the E1 protein of WEEV, F81 of the E1 protein of WEEV, G82 of the E1 protein of WEEV, G83 of the E1 protein of WEEV, S98 of the E1 protein of WEEV, E99 of the E1 protein of WEEV, N100 of the E1 protein of WEEV, T101 of the E1 protein of WEEV, M195 of the E1 protein of WEEV, A215 of the E1 protein of WEEV, R216 of the E1 protein of WEEV, D218 of the E1 protein of WEEV, I219 of the E1 protein of WEEV, L221 of the E1 protein of WEEV, and K223 of the E1 protein of WEEV; an isolated antibody in which the specificity determining residues of the isolated antibody interact with at least 15, optionally at least 16, optionally at least 17, or all, contact residues selected from the group consisting of N57 of the E2 protein of WEEV, Q58 of the E2 protein of WEEV, A59 of the E2 protein of WEEV, T61 of the E2 protein of WEEV, R173 of the E2 protein of WEEV, G197 of the E2 protein of WEEV, K198 of the E2 protein of WEEV, K233 of the E2 protein of WEEV, D235 of the E2 protein of WEEV, Q236 of the E2 protein of WEEV, T237 of the E2 protein of WEEV, W239 of the E2 protein of WEEV, D251 of the E2 protein of WEEV, S253 of the E2 protein of WEEV, V254 of the E2 protein of WEEV, Q255 of the E2 protein of WEEV, G256 of the E2 protein of WEEV, and K257 of the E2 protein of WEEV; an isolated a antibody in which the specificity determining residues of the isolated antibody interact with at least 15, optionally at least 20, or all, contact residues selected from the group consisting of K61 of the E1 protein of WEEV, C63 of the E1 protein of WEEV, G64 of the E1 protein of WEEV, S65 of the E1 protein of WEEV, F95 of the E1 protein of WEEV, N199 of the E2 protein of WEEV, T201 of the E2 protein of WEEV, Y202 of the E2 protein of WEEV, K205 of the E2 protein of WEEV, D208 of the E2 protein of WEEV, Y209 of the E2 protein of WEEV, S210 of the E2 protein of WEEV, T211 of the E2 protein of WEEV, G212 of the E2 protein of WEEV, I213 of the E2 protein of WEEV, V214 of the E2 protein of WEEV, S215 of the E2 protein of WEEV, T216 of the E2 protein of WEEV, T218 of the E2 protein of WEEV, K219 of the E2 protein of WEEV, M220 of the E2 protein of WEEV, and N221 of the E2 protein of WEEV; and, an isolated antibody in which the specificity determining residues of the isolated antibody interact with at least 15, optionally at least 20, optionally at least 22, optionally at least 24, optionally at least 26, optionally at least 28, optionally at least 30, or all, contact residues selected from the group consisting of Q59 of the E1 protein of WEEV, V60 of the E1 protein of WEEV, K61 of the E1 protein of WEEV, C62 of the E1 protein of WEEV, Q93 of the E1 protein of WEEV, C94 of the E1 protein of WEEV, F95 of the E1 protein of WEEV, R173 of the E2 protein of WEEV, P176 of the E2 protein of WEEV, A178 of the E2 protein of WEEV, T201 of the E2 protein of WEEV, Y202 of the E2 protein of WEEV, E203 of the E2 protein of WEEV, K205 of the E2 protein of WEEV, G207 of the E2 protein of WEEV, D208 of the E2 protein of WEEV, Y209 of the E2 protein of WEEV, S210 of the E2 protein of WEEV, T211 of the E2 protein of WEEV, G212 of the E2 protein of WEEV, I213 of the E2 protein of WEEV, N221 of the E2 protein of WEEV, 1230 of the E2 protein of WEEV, Y232 of the E2 protein of WEEV, S234 of the E2 protein of WEEV, Q236 of the E2 protein of WEEV, 1247 of the E2 protein of WEEV, R248 of the E2 protein of WEEV, H249 of the E2 protein of WEEV, T250 of the E2 protein of WEEV, D251 of the E2 protein of WEEV, S253 of the E2 protein of WEEV, and Q255 of the E2 protein of WEEV. The isolated antibody may be SKW10, SKW11, SKW14, SKW15, SKW16, SKW17, SKW18, SKW19, SKW24, SKW29, SKW30, SKE25, SKE26, SKE27, SKE33, SKV09, SKV11, SKV16, SKV18, SKV20, SKT01, SKT02, SKT05, SKT05.hu13, SKT05.hu21, SKT05.hu25, SKT05.hu29, SKT05.hu37, SKT05.hu41, SKT05.hu47, SKT05.hu52, SKT05.hu56, SKT05.hu60, SKT14, or SKT20, the sequences of which are disclosed herein. The isolated antibody may be from a non-human primate (NHP) and may be humanized.

One aspect is a method of preventing infection of a cell by an alphavirus, comprising administering to the individual the isolated antibody of the disclosure.

One aspect is a method of protecting an individual against infection by an alphavirus, comprising contacting the cell with the isolated antibody of the disclosure.

One aspect is a method of treating an individual for an alphavirus infection, comprising administering to the individual the isolated antibody of the disclosure.

In certain aspects, the alphavirus may be EEV, VEEV, WEEV, HKV, or Ross-River virus.

### BRIEF DESCRIPTION OF THE DRAWINGS

The embodiments described herein may be better understood by referring to the following description in conjunction with the accompanying drawings.

FIGS. 1A & 1B show that extended, trivalent VLP immunization elicits single-specific and triple-specific a-EEV mAbs in NHP. FIG. 1A shows the immunization scheme. Briefly, Cynomolgus macaques (n=16) were immunized intramuscularly or intranasally, with or without α-GalCer as an adjuvant, five times over 52 weeks. At weeks 0, 4, and 21, animals received 20 μg of each monovalent WEE, EEE, and VEE VLP, and at weeks 30 and 52 they received 60 μg of each VLP. Animals in the adjuvant groups received 8 μg of α-GalCer for the first three immunizations and 24 μg for the remaining two immunizations. FIG. 1B Multiple lineages of a-EEV mAbs of varying specificities were sorted from the vaccinated animal with the greatest response. Single B cells positive for binding only a single or all three VLPs were index sorted into wells; mAbs from each cell were sequenced and synthesized. Lineages were defined as having a unique V-gene allele, CDR3 sequence, and CDR3 length. FIG. 1D shows ELISA binding curves of single-specific and triple-specific α-EEV mAbs to WEEV VLPs (top), EEEV VLPs (middle), and VEEV VLPs (bottom). Data are representative of two or three independent experiments.

FIGS. 2A-2D show heat maps identifying distinct binding groups of single-specific and triple-specific α-EEV mAbs. FIG. 2A shows results from binding ELISAs identifying overlap in VLP surface binding areas within α-WEVEEV mAbs using VEEV VLP. FIG. 2B shows results from binding ELISAs identifying overlap in VLP surface binding areas within α-WEVEEV mAbs using WEEV VLP. FIG. 2C shows results from binding ELISAs identifying overlap in VLP surface binding areas within single-specific mAbs as well as previously published broadly reactive α-EEV mAbs (DC2.112, D2.315, EEEV-138, EEEV-179, and EEEV-346) using WEEV VLP (left), EEEV VLP (middle), or VEEV VLP (right). Heat maps display percent inhibition ranging from light (minimal competition) to grey (moderate competition) to dark (maximal competition). Negative control mAbs include either the human-HIV mAb VRC01 or the NHP-SIV mAb ITS103.01.

FIGS. 3A-3D show that vaccine elicited α-EEV mAbs bind, neutralize, and protect against encephalitic alphavirus challenge in vivo. FIG. 3A shows neutralization IC₅₀ values for single- and triple-specific α-EEV mAbs against WEEV, EEEV, and VEEV Env-pseudotyped lentiviral reporter viruses. Fractions above the X-axis indicate the number of neutralizing mAbs out of the total tested. FIGS. 3B & 3C shows a VEEV (TC-83) challenge outcome in mice that received SKT05, SKT20, or an NHP α-SIV mAb as a control 1 day prior to inoculation. Survival rate analysis (FIG. 3B) and change in relative weight (FIG. 3C), shown as mean±SD, in mice 14 days after inoculation. Viral load was determined in spleen 5 days after inoculation (FIG. 3D). Statistical significance related to viral RNA was determined by Kruskal-Wallis test (**p<0.01, ***<0.001, ****p<0.0001). The dotted line indicates the limit of detection for viral analysis.

FIGS. 4A-4E illustrate that antibody SKT05 broadly recognizes and protects against arthritogenic alphaviruses. FIG. 4A shows the ability of representative single-specific and triple-specific mAbs to bind Chikungunya VLP. FIG. 4B shows that SKT05 binds cells infected with multiple arthritogenic alphaviruses. Vero cells were infected with CHIKV, MAYV, ONNV, or RRV, or left uninfected for 16 hr followed by staining with SKT05, CHK-265, or α-RRV ascites fluid. Cells were analyzed by flow cytometry and negative gates were set based on an isotype control mAb. FIGS. 4C-4F show arthritogenic alphavirus challenge outcome in mice that received SKT05 or the NHP-SIV mAb ITS103.01 as a control I day prior to inoculation. Viral load was determined in the indicated tissues 3 days following inoculation with CHIKV (FIG. 4C & 4D) or RRV (FIG. 4F & 4G).

FIGS. 5A & 5B show that humanized SKT05 variants display binding and neutralization profiles similar to SKT05. FIG. 5A shows the binding affinity of SKT05 variants for WEEV VLP, EEEV VLP, and VEEV VLP. FIG. 5B shows the neutralization titers of 14 humanized SKT05 variants for WEEV VLP, EEEV VLP, and VEEV VLP.

FIGS. 6A & 6B show the ability of ten humanized SKT05 variants to bind live cells infected with CHIKV (FIG. 6D, left), MAYV (FIG. 6A, right), ONNV (FIG. 6B, left), or RRV (FIG. 6B, right). Vero cells were infected with CHIKV, MAYV, ONNV, or RVV, or left uninfected, for 19 hours following by staining with humanized SKT05 variant, CHK-265, or RRV immune ascites fluid. Cells were analyzed by flow cytometry, and negative gates were set based on control mAb.

FIG. 7 shows that SKT05 protects non-human primates against EEEV. The graph shows a survival rate analysis following aerosol EEEV (FL93-939) challenge in NHP (n=3/group) that received SKT05, SKT20, SKE26, or an NHP anti-SIV mAb as a control 5 days prior to challenge. * p<0.05. ns: not significant

### DETAILED DESCRIPTION OF THE DISCLOSURE

The present disclosure relates to therapeutic agents capable of neutralizing at least one, and preferably more than one, species of virus from the genus alphavirus. More specifically, the present disclosure provides isolated antibodies that are capable of binding and neutralizing one or more species of virus from the genus alphavirus. Preferably, an isolated antibody of the disclosure neutralizes at least two, and preferably, at least three species of virus from the genus alphavirus. Thus, the teachings of the disclosure may generally be practiced by producing an antibody that binds to at least one, preferably at least two, preferably at least three, species of virus from the genus alphavirus.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the invention will be limited only by the claims.

It must be noted that as used herein and in the appended claims, the singular forms “a,” “an,” and “the” include plural referents unless the context clearly dictates otherwise. For example, a nucleic acid molecule refers to one or more nucleic acid molecules. As such, the terms “a”, “an”, “one or more” and “at least one” can be used interchangeably.

Similarly, the terms “comprising”, “including” and “having” can be used interchangeably. As used herein, the term “comprising” may be replaced with “consisting” or with “consisting essentially of” in particular aspect, as desired.

It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed. In addition, all sub-combinations are also specifically embraced by the present invention and arm disclosed herein just as if each and every such sub-combination was individually and explicitly disclosed herein.

Publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that vary depending upon the desired properties sought to be obtained by a particular embodiment. In some embodiments, the numerical parameters are be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

All compositions and/or methods disclosed and claimed herein may be made and/or executed without undue experimentation in light of the present disclosure. While the compositions and methods of this disclosure have been described in terms of the embodiments included herein, it will be apparent to those of ordinary skill in the art that variations may be applied to the compositions and/or methods and in the steps or in the sequence of steps of the method described herein without departing from the concept, spirit, and scope of the disclosure. All such similar substitutes and modifications apparent to those skilled in the art are deemed to be within the spirit, scope, and concept of the disclosure as defined by the appended claims.

All numerical designations, e.g., pH, temperature, time, concentration and molecular weight, including ranges, are approximations which are varied (+) or (−) by increments of 1.0 or 0.1, as appropriate, or alternatively by a variation of +/−15%, or alternatively 10%, or alternatively 5% or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein. Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

As used herein, the term “antibody” refers to a polypeptide ligand comprising at least a light chain or heavy chain complementarity determining region (CDR) [immunoglobulin variable region] that specifically recognizes and binds an epitope of an antigen. More specifically, “antibody” refers to immunoglobulins or immunoglobulin-like molecules including by way of example and without limitation, IgA, IgD, IgE, IgG and IgM, combinations thereof, portions thereof, and similar molecules produced during an immune response in an individual. The term “antibody” includes whole immunoglobulins/whole antibodies and fragments thereof (a.k.a., “antibody fragments” or “antigen binding fragments”) that specifically bind to an antigen of interest or to a group of highly similar antigens of interest. As such, the term “antibody” encompasses whole immunoglobulins, digestion fragments of whole immunoglobulins, specified portions of immunoglobulins, derivatives and variants thereof, including antibody mimetics or portions of antibodies that mimic the structure and/or function of an antibody or specified fragment or portion thereof, including single chain antibodies and fragments thereof. Examples of types of antibodies useful for practicing the present disclosure include, but are not limited to, Fab fragments. F(ab′)₂ fragments, Fab′ fragments, Fab′-SH fragments, a dAB fragment, sFᵥ fragments, dsFᵥ fragments, bispecific scFᵥ fragments, bispecific dsFᵥ fragments, single chain Fv proteins (“scFᵥ”), sc(Fᵥ)₂ fragments, disulfide stabilized Fᵥ proteins (“dsFᵥ”), minibodies, diabodies, triabodies (as are known in the art), DART antibodies, BiKE antibodies, TriKE antibodies, camelid antibodies (see, for example, U.S. Pat. No. 6,015,695), shark antibodies. VnH domain antibodies, V-NAR domain antibodies and variations thereon. The term “antibody” also includes genetically engineered forms of antibodies such as chimeric antibodies (e.g., humanized murine antibodies) and heteroconjugate antibodies, such as, bispecific antibodies.

Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody.

Typically, a whole immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991). The Kabat database is now maintained online. The sequences of the framework regions of different light or heavy chains are relatively conserved within a species. The framework region of an antibody, that is the combined framework regions of the constituent light and heavy chains, largely adopt β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. Although it is the CDRs that vary from antibody to antibody, only a limited number of amino acid positions within the CDRs are directly involved in antigen binding. These positions within the CDRs are called specificity determining residues (SDRs). SDRs interact with amin acid residues (called contact residues) in an antigen, resulting in binding of the antibody to the antigen.

As used herein, an “epitope” or “antigenic determinant” refers to particular chemical groups or contiguous or non-contiguous peptide sequences on a molecule that are antigenic, i.e., that elicit a specific immune response. An antibody binds a particular antigenic epitope. Epitopes usually consist of chemically active surface groupings of molecules such as amino acids or sugar side chains and may, but need not, have specific three-dimensional structural characteristics, as well as specific charge characteristics. Conformational and non-conformational epitopes are distinguished in that the binding to the former but not the latter is lost in the presence of denaturing solvents.

As used herein, the term “antibody derivative” is intended to encompass molecules that bind an epitope as defined herein and which are modifications or derivatives of an isolated antibody of this disclosure. Derivatives include, but are not limited to, for example, bispecific, heterospecific, trispecific, tetraspecific, multispecific antibodies, diabodies, chimeric, recombinant, and humanized. As used herein, the term “bispecific molecule” an antibody that has two different binding specificities. As used herein, the term “multispecific molecule” or “heterospecific molecule” is intended to include any antibody that has more than two different binding specificities.

As used herein, the term “antigen” refers to a compound or complex (e.g., protein complex) that elicits an immune response and is specifically bound by the products of the immune response, such as an antibody molecule or a T-cell receptor. Antigens can comprise any type of molecule including, for example, haptens, sugars (e.g., oligosaccharides), lipids, as well as macromolecules such as complex carbohydrates (e.g., polysaccharides), phospholipids, and proteins.

As used herein, “specifically binds”, “specific binding”, and the like, means that an antibody binds an antigen of interest to the substantial exclusion of binding to other molecules (for example, antibodies and antibody fragments that have a binding constant for the molecule of interest that is at least 10³ M⁻¹ greater, at least 10⁴ M⁻¹ greater, at least 10⁵ M⁻¹, or at least 10⁶ M⁻¹ greater than their binding constant for other molecules in a biological sample). As used herein, “binding affinity” refers to the strength of binding (typically non-covalent) between one molecule and a second molecule, such as the strength of binding between an antibody and an antigen. A binding affinity can be measured as a binding constant, which binding affinity for a specific binding pair (such as an antibody/antigen pair) may be at least 1×10⁻⁵ M, at least 1×10⁻⁶ M, at least 1×10⁻⁷ M, at least 1×10⁻⁸ M, at least 1×10⁻⁹ M, at least 1×10⁻¹⁰ M, at least 1×10⁻¹¹ M or at least 1×10⁻¹²M. Methods of determining binding affinity are known to those skilled in the art and include, but are not limited to, the Scatchard method described by Frankel et al., Mol. Immunol., 16:101-106, 1979, determining antigen/antibody dissociation rate, and competition radioimmunoassay.

As used herein, the term “alphavirus” is used as commonly understood by those in the art and refers to RNA viruses of the Togavirus family. While any alphavirus may be bound and neutralized by an antibody of the disclosure, exemplary species of virus include, but are not limited to, WEEV, EEEV, VEEV, CHKV, RRV, MAYV, ONNV, Barmah Forest virus (BFV), Middleburg virus (MV), Ndumu virus (NV), Semliki Forest virus (SFV), Tontate virus (TV), and Una virus (UV), Aura virus (AV), Bebaru virus (BV) and salmonid alphavirus (SV).

In aspects in which the molecule of interest is a protein, “highly similar” means the proteins have at least 80%, at least 85%, at least 89%, at least 95%, at least 97%, or at least 99% sequence homology or identity across the entire length of the proteins being compared, for example, in mammals such as humans, goats, rabbits and mice, as well as non-mammalian species, such as shark immunoglobulins.

One aspect of the disclosure is an isolated antibody that specifically binds at least one species of virus from the genus alphavirus. In certain aspects, the isolated antibody specifically binds at least two species of virus from the genus alphavirus. In certain aspects, the isolated antibody specifically binds at least three species of virus from the genus alphavirus. In certain aspects, the isolated antibody specifically binds at least four species of virus from the genus alphavirus. In certain aspects, the isolated antibody neutralizes the at least one species of virus from the genus alphavirus. As used herein, the term “neutralizes” means the antibody blocks or reduces the infectivity of a virus. “Infectivity” refers to the ability of a virus to enter a host cell, replicate and spread to another host cell. Thus, an antibody of the disclosure may neutralize a virus by interfering with any step of the replicative cycle. For example, a neutralizing antibody may inhibit binding of the virus to the cell, entry of the virus into the cell, uncoating of the virus, escape of the virus from an endocytic vesicle, replication of the viral genome, formation of new virus particles, and/or release of the virus from the infected cell. In certain aspects, neutralization of infectivity reduces the amount of viral replication by at least 25%, at least 50%, at least 75% or at least 100%. In certain aspects, neutralization of infectivity reduces the amount of viral replication by at least 0.5×10⁻¹, at least 10⁻², at least 10⁻³, at least 10⁻⁴, at least 10⁻⁵, or at least 10⁻⁶.

In certain aspects, the isolated antibody neutralizes the at least two species of virus from the genus alphavirus. In certain aspects, the isolated antibody neutralizes the at least three species of virus from the genus alphavirus. In certain aspects, the isolated antibody neutralizes the at least four species of virus from the genus alphavirus. In certain aspects, the at least one, at least two, at least three, or at least four species of virus are selected from the group consisting of WEEV, EEEV, VEEV, CHKV, RRV, MAYV, ONNV, BFV, MV, NV, SFV, TV, UV, AV, BV and SV. In certain aspects, the at least one, at least two, at least three, or at least four species of virus are selected from the group consisting of WEEV, EEEV, VEEV, CHKV, RRV, MAYV, ONNV. In certain aspects, the at least one, at least two, or at least three species of virus are selected from the group consisting of WEEV, EEEV, and VEEV.

In certain aspects, an isolated antibody of the disclosure inhibits binding of a first antibody to WEEV or EEEV, wherein the first antibody is selected from the group consisting of:
a. a first antibody (e.g., SKE26) in which the specificity determining residues of the first antibody interact with at least 15, optionally at least 20, optionally at least 22, optionally at least 24, optionally at least 26, optionally at least 28, optionally at least 30, optionally at least 32, optionally at least 34, optionally at least 36, optionally at least 37, optionally at least 38, or all, contact residues selected from the group consisting of Y89 of the E1 protein of EEEV, G90 of the E1 protein of EEEV, D3 of the E2 protein of EEEV, F6 of the E2 protein of EEEV, T7 of the E2 protein of EEEV, Y9 of the E2 protein of EEEV, K10 of the E2 protein of EEEV, L11 of the E2 protein of EEEV, R13 of the E2 protein of EEEV, T57 of the E2 protein of EEEV, D58 of the E2 protein of EEEV, G59 of the E2 protein of EEEV, M68 of the E2 protein of EEEV, N69 of the E2 protein of EEEV, G70 of the E2 protein of EEEV, K71 of the E2 protein of EEEV, T72 of the E2 protein of EEEV, Q73 of the E2 protein of EEEV, K74 of the E2 protein of EEEV, S75 of the E2 protein of EEEV, V172 of the E2 protein of EEEV, S176 of the E2 protein of EEEV, K186 of the E2 protein of EEEV, T188 of the E2 protein of EEEV, V189 of the E2 protein of EEEV, P190 of the E2 protein of EEEV, S191 of the E2 protein of EEEV, G192 of the E2 protein of EEEV, A193 of the E2 protein of EEEV, Q194 of the E2 protein of EEEV, T209 of the E2 protein of EEEV, S210 of the E2 protein of EEEV, S211 of the E2 protein of EEEV, L227 of the E2 protein of EEEV, I228 of the E2 protein of EEEV, D229 of the E2 protein of EEEV, N230 of the E2 protein of EEEV, K231 of the E2 protein of EEEV, and K232 of the E2 protein of EEEV;
b. a first antibody (e.g., SKT05) in which the specificity determining residues of the first antibody interact with at least 15, optionally at least 20, optionally at least 22, optionally at least 24, optionally at least 26, optionally at least 28, optionally at least 30, optionally at least 31, or all, contact residues selected from the group consisting of G64 of the E1 protein of VEEV, S65 of the E1 protein of VEEV, Q66 of the E1 protein of VEEV, E67 of the E1 protein of VEEV, C68 of the E1 protein of VEEV, T69 of the E1 protein of VEEV, P70 of the E1 protein of VEEV, Y72 of the E1 protein of VEEV, D75 of the E1 protein of VEEV, E76 of the E1 protein of VEEV, Q77 of the E1 protein of VEEV, C78 of the E1 protein of VEEV, K79 of the E1 protein of VEEV, V80 of the E1 protein of VEEV, F81 of the E1 protein of VEEV, T82 of the E1 protein of VEEV, T98 of the E1 protein of VEEV, E99 of the E1 protein of VEEV, Y214 of the E1 protein of VEEV, A215 of the E1 protein of VEEV, N216 of the E1 protein of VEEV, N218 of the E1 protein of VEEV, L219 of the E1 protein of VEEV, V220 of the E1 protein of VEEV, R223 of the E1 protein of VEEV, S141 of the E1 protein of VEEV, V143 of the E1 protein of VEEV, D311 of the E1 protein of VEEV, K340 of the E1 protein of VEEV, T389 of the E1 protein of VEEV, H390 of the E1 protein of VEEV, and Y393 of the E1 protein of VEEV;
c. a first antibody (e.g., SKT20) in which the specificity determining residues of the first antibody interact with at least 15, optionally at least 18, optionally at least 20, optionally at least 21, optionally at least 22, or all, contact residues selected from the group consisting of F87 of the E1 protein of VEEV, M88 of the E1 protein of VEEV, W89 of the E1 protein of VEEV, G90 of the E1 protein of VEEV, G91 of the E1 protein of VEEV, Y93 of the E1 protein of VEEV, F95 of the E1 protein of VEEV, C96 of the E1 protein of VEEV, D97 of the E1 protein of VEEV, E99 of the E1 protein of VEEV, A226 of the E1 protein of VEEV, G227 of the E1 protein of VEEV, H28 of the E2 protein of VEEV, L55 of the E2 protein of VEEV, G63 of the E2 protein of VEEV, R64 of the E2 protein of VEEV, D94 of the E2 protein of VEEV, G95 of the E2 protein of VEEV, H96 of the E2 protein of VEEV, H156 of the E2 protein of VEEV, D157 of the E2 protein of VEEV, A158 of the E2 protein of VEEV, and Q159 of the E2 protein of VEEV;
d. a first antibody (e.g., SKV09) in which the specificity determining residues of the a first antibody in which the specificity determining residues of the first antibody interact with at least 15, optionally at least 16, optionally at least 17, optionally at least 18, optionally at least 19, or all, contact residues selected from the group consisting of antibody contact residues selected from the group consisting of E348 of the E1 protein of VEEV, Q349 of the E1 protein of VEEV, K319 of the E1 protein of VEEV, S325 of the E1 protein of VEEV, T347 of the E1 protein of VEEV, E348 of the E1 protein of VEEV, Q349 of the E1 protein of VEEV, A298 of the E1 protein of VEEV, T317 of the E1 protein of VEEV, K319 of the E1 protein of VEEV, S323 of the E1 protein of VEEV, K324 of the E1 protein of VEEV, S325 of the E1 protein of VEEV, G326 of the E1 protein of VEEV, E345 of the E1 protein of VEEV, T347 of the E1 protein of VEEV, E348 of the E1 protein of VEEV, Q349 of the E1 protein of VEEV, G350 of the E1 protein of VEEV, and S351 of the E1 protein of VEEV;
e. (SKV16) a first antibody (e.g., SKV16) in which the specificity determining residues of the first antibody interact with at least 15, optionally at least 16, optionally at least 17, optionally at least 18, optionally at least 19, or all, contact residues selected from the group consisting of S180 of the E2 protein of VEEV, L181 of the E2 protein of VEEV, S182 of the E2 protein of VEEV, G183 of the E2 protein of VEEV, S185 of the E2 protein of VEEV, T187 of the E2 protein of VEEV, T189 of the E2 protein of VEEV, P190 of the E2 protein of VEEV, P191 of the E2 protein of VEEV, D192 of the E2 protein of VEEV, G193 of the E2 protein of VEEV, E209 of the E2 protein of VEEV, T210 of the E2 protein of VEEV, 1211 of the E2 protein of VEEV, N212 of the E2 protein of VEEV, K213 of the E2 protein of VEEV, T214 of the E2 protein of VEEV, K215 of the E2 protein of VEEV, Q216 of the E2 protein of VEEV, and S218 of the E2 protein of VEEV;
f. a first antibody (e.g., SKT5) in which the specificity determining residues of the first antibody interact with at least 15, optionally at least 20, optionally at least 22, optionally at least 24, optionally at least 26, optionally at least 27, or all, contact residues selected from the group consisting of C63 of the E1 protein of WEEV, S65 of the E1 protein of WEEV, L66 of the E1 protein of WEEV, E67 of the E1 protein of WEEV, C68 of the E1 protein of WEEV, K69 of the E1 protein of WEEV, A70 of the E1 protein of WEEV, S71 of the E1 protein of WEEV, D75 of the E1 protein of WEEV, Y76 of the E1 protein of WEEV, T77 of the E1 protein of WEEV, C78 of the E1 protein of WEEV, R79 of the E1 protein of WEEV, V80 of the E1 protein of WEEV, F81 of the E1 protein of WEEV, G82 of the E1 protein of WEEV, G83 of the E1 protein of WEEV, S98 of the E1 protein of WEEV, E99 of the E1 protein of WEEV, N100 of the E1 protein of WEEV, T101 of the E1 protein of WEEV, M195 of the E1 protein of WEEV, A215 of the E1 protein of WEEV, R216 of the E1 protein of WEEV, D218 of the E1 protein of WEEV, I219 of the E1 protein of WEEV, L221 of the E1 protein of WEEV, and K223 of the E1 protein of WEEV;
g. a first antibody (e.g., SKW11) in which the specificity determining residues of the first antibody interact with at least 15, optionally at least 16, optionally at least 17, or all, contact residues selected from the group consisting of N57 of the E2 protein of WEEV, Q58 of the E2 protein of WEEV, A59 of the E2 protein of WEEV, T61 of the E2 protein of WEEV, R173 of the E2 protein of WEEV, G197 of the E2 protein of WEEV, K198 of the E2 protein of WEEV, K233 of the E2 protein of WEEV, D235 of the E2 protein of WEEV, Q236 of the E2 protein of WEEV, T237 of the E2 protein of WEEV, W239 of the E2 protein of WEEV, D251 of the E2 protein of WEEV, S253 of the E2 protein of WEEV, V254 of the E2 protein of WEEV, Q255 of the E2 protein of WEEV, G256 of the E2 protein of WEEV, and K257 of the E2 protein of WEEV;
h. (SKW19) a first antibody (e.g., SKW19) in which the specificity determining residues of the first antibody interact with at least 15, optionally at least 16, optionally at least 18, optionally at least 20, optionally at least 21, or all, contact residues selected from the group consisting of K61 of the E1 protein of WEEV, C63 of the E1 protein of WEEV, G64 of the E1 protein of WEEV, S65 of the E1 protein of WEEV, F95 of the E1 protein of WEEV, N199 of the E2 protein of WEEV, T201 of the E2 protein of WEEV, Y202 of the E2 protein of WEEV, K205 of the E2 protein of WEEV, D208 of the E2 protein of WEEV, Y209 of the E2 protein of WEEV, S210 of the E2 protein of WEEV, T211 of the E2 protein of WEEV, G212 of the E2 protein of WEEV, I213 of the E2 protein of WEEV, V214 of the E2 protein of WEEV, S215 of the E2 protein of WEEV, T216 of the E2 protein of WEEV, T218 of the E2 protein of WEEV, K219 of the E2 protein of WEEV, M220 of the E2 protein of WEEV, and N221 of the E2 protein of WEEV; and,
i. (SKW24) a first antibody in which the specificity determining residues of the antibody interact with at least 15, optionally at least 20, optionally at least 22, optionally at least 24, optionally at least 26, optionally at least 28, optionally at least 30, optionally at least 31, optionally at least 32, or all, contact residues selected from the group consisting of Q59 of the E1 protein of WEEV, V60 of the E1 protein of WEEV, K61 of the E1 protein of WEEV, C62 of the E1 protein of WEEV, Q93 of the E1 protein of WEEV, C94 of the E1 protein of WEEV, F95 of the E1 protein of WEEV, R173 of the E2 protein of WEEV, P176 of the E2 protein of WEEV, A178 of the E2 protein of WEEV, T201 of the E2 protein of WEEV, Y202 of the E2 protein of WEEV, E203 of the E2 protein of WEEV, K205 of the E2 protein of WEEV, G207 of the E2 protein of WEEV, D208 of the E2 protein of WEEV, Y209 of the E2 protein of WEEV, S210 of the E2 protein of WEEV, T211 of the E2 protein of WEEV, G212 of the E2 protein of WEEV, I213 of the E2 protein of WEEV, N221 of the E2 protein of WEEV, 1230 of the E2 protein of WEEV, Y232 of the E2 protein of WEEV, S234 of the E2 protein of WEEV, Q236 of the E2 protein of WEEV, 1247 of the E2 protein of WEEV, R248 of the E2 protein of WEEV, H249 of the E2 protein of WEEV, T250 of the E2 protein of WEEV, D251 of the E2 protein of WEEV, S253 of the E2 protein of WEEV, and Q255 of the E2 protein of WEEV.

In certain aspects, the first antibody interacts with the recited contact residues in the E1 and/or E2 protein when the E1 and E2 proteins are present in the intact viral particle. In certain aspects, the first antibody is selected from the group consisting of SKW10 (comprising a heavy chain comprising or consisting of SEQ ID NO:289 and a light chain comprising or consisting of SEQ ID NO:290), SKW11 (comprising a heavy chain comprising or consisting of SEQ ID NO:291 and a light chain comprising or consisting of SEQ ID N0292:), SKW14 (comprising a heavy chain comprising or consisting of SEQ ID NO:293 and a light chain comprising or consisting of SEQ ID NO:294), SKW15 (comprising a heavy chain comprising or consisting of SEQ ID NO:295 and a light chain comprising or consisting of SEQ ID NO:296), SKW16 (comprising a heavy chain comprising or consisting of SEQ ID NO:297 and a light chain comprising or consisting of SEQ ID NO:298), SKW17 (comprising a heavy chain comprising or consisting of SEQ ID NO:299 and a light chain comprising or consisting of SEQ ID NO:300), SKW18 (comprising a heavy chain comprising or consisting of SEQ ID NO:301 and a light chain comprising or consisting of SEQ ID NO:302), SKW19 (comprising a heavy chain comprising or consisting of SEQ ID NO:303 and a light chain comprising or consisting of SEQ ID NO:304), SKW24 (comprising a heavy chain comprising or consisting of SEQ ID NO:305 and a light chain comprising or consisting of SEQ ID NO:306), SKW29 (comprising a heavy chain comprising or consisting of SEQ ID NO:307 and a light chain comprising or consisting of SEQ ID NO:308), SKW30 (comprising a heavy chain comprising or consisting of SEQ ID NO:309 and a light chain comprising or consisting of SEQ ID NO:310), SKE25 (comprising a heavy chain comprising or consisting of SEQ ID NO:311 and a light chain comprising or consisting of SEQ ID NO:312), SKE26 (comprising a heavy chain comprising or consisting of SEQ ID NO:313 and a light chain comprising or consisting of SEQ ID NO:314), SKE27 (comprising a heavy chain comprising or consisting of SEQ ID NO:315 and a light chain comprising or consisting of SEQ ID NO:316), SKE33 (comprising a heavy chain comprising or consisting of SEQ ID NO:317 and a light chain comprising or consisting of SEQ ID NO:318), SKV09 (comprising a heavy chain comprising or consisting of SEQ ID NO:319 and a light chain comprising or consisting of SEQ ID NO:320), SKV11 (comprising a heavy chain comprising or consisting of SEQ ID NO:321 and a light chain comprising or consisting of SEQ ID NO:322), SKV16 (comprising a heavy chain comprising or consisting of SEQ ID NO:323 and a light chain comprising or consisting of SEQ ID NO:324), SKV18 (comprising a heavy chain comprising or consisting of SEQ ID NO:325 and a light chain comprising or consisting of SEQ ID NO:326), SKV20 (comprising a heavy chain comprising or consisting of SEQ ID NO:327 and a light chain comprising or consisting of SEQ ID NO:328), SKT01 (comprising a heavy chain comprising or consisting of SEQ ID NO:329 and a light chain comprising or consisting of SEQ ID NO:330), SKT02 (comprising a heavy chain comprising or consisting of SEQ ID NO:331 and a light chain comprising or consisting of SEQ ID NO:332), SKT05 (comprising a heavy chain comprising or consisting of SEQ ID NO:333 and a light chain comprising or consisting of SEQ ID NO:334), SKT14 (comprising a heavy chain comprising or consisting of SEQ ID NO:335 and a light chain comprising or consisting of SEQ ID NO:336), SKT19 (comprising a heavy chain comprising or consisting of SEQ ID NO:337 and a light chain comprising or consisting of SEQ ID NO:338), SKT20 (comprising a heavy chain comprising or consisting of SEQ ID NO:339 and a light chain comprising or consisting of SEQ ID NO:340), SKT05.hu13 (comprising a heavy chain comprising or consisting of SEQ ID NO:341 and a light chain comprising or consisting of SEQ ID NO:342), SKT05.hu21 (comprising a heavy chain comprising or consisting of SEQ ID NO:343 and a light chain comprising or consisting of SEQ ID NO:344), SKT05.hu25 (comprising a heavy chain comprising or consisting of SEQ ID NO:345 and a light chain comprising or consisting of SEQ ID NO:346), SKT05.hu29 (comprising a heavy chain comprising or consisting of SEQ ID NO:347 and a light chain comprising or consisting of SEQ ID NO:348), SKT05.hu37 (comprising a heavy chain comprising or consisting of SEQ ID NO:349 and a light chain comprising or consisting of SEQ ID NO:350), SKT05.hu41 (comprising a heavy chain comprising or consisting of SEQ ID NO:351 and a light chain comprising or consisting of SEQ ID NO:352), SKT05.hu47 (comprising a heavy chain comprising or consisting of SEQ ID NO:353 and a light chain comprising or consisting of SEQ ID NO:354), SKT05.hu52 (comprising a heavy chain comprising or consisting of SEQ ID NO:355 and a light chain comprising or consisting of SEQ ID NO:356), SKT05.hu56 (comprising a heavy chain comprising or consisting of SEQ ID NO:357 and a light chain comprising or consisting of SEQ ID NO:358), and SKT05.hu60 (comprising a heavy chain comprising or consisting of SEQ ID NO:359 and a light chain comprising or consisting of SEQ ID NO:360). In certain aspects, the first antibody is STK05 or STK20. In certain aspects, the first antibody is STK05 or variants thereof, such as SKT05.hu13, SKT05.hu21, SKT05.hu25, SKT05.hu29, SKT05.hu37, SKT05.hu41, SKT05.hu47, SKT05.hu52, SKT05.hu56, and SKT05.hu60.

As used herein, “inhibits binding” with regard to the first antibody means that the isolated antibody reduces the detectable amount of the first antibody bound to the WEEV E1 protein by at least 25%, at least 30%, at last 40%, at least 50%, at last 75% or at least 90%. In certain aspects, the isolated antibody completely blocks binding of the first antibody to the WEEV E1 protein. Any method of measuring binding of an antibody to an antigen may be used to determine inhibition. Such methods are known to those skilled in the art.

The instant inventors have discovered that certain antibodies that are able to neutralize two or more species of alphavirus have at least one specificity determining residue that interacts with a backbone atom of the WEEV E1 protein. Without being bound by theory, the inventors believe that such interaction contributes to antibodies cross-neutralizing activity. Thus, in certain aspects, the isolated antibody inhibits binding of a first antibody to Western Equine Encephalitis virus (WEEV) E1 protein, wherein the specificity determining residues of the first antibody interact with at least 15, optionally at least 20, optionally at least 22, optionally at least 24, optionally at least 26, or all, contact residues selected from the group consisting of C63 of the E1 protein of WEEV, S65 of the E1 protein of WEEV, L66 of the E1 protein of WEEV, E67 of the E1 protein of WEEV, C68 of the E1 protein of WEEV, K69 of the E1 protein of WEEV, A70 of the E1 protein of WEEV, S71 of the E1 protein of WEEV, D75 of the E1 protein of WEEV, Y76 of the E1 protein of WEEV, T77 of the E1 protein of WEEV, C78 of the E1 protein of WEEV, R79 of the E1 protein of WEEV, V80 of the E1 protein of WEEV, F81 of the E1 protein of WEEV, G82 of the E1 protein of WEEV, G83 of the E1 protein of WEEV, S98 of the E1 protein of WEEV, E99 of the E1 protein of WEEV, N100 of the E1 protein of WEEV, T101 of the E1 protein of WEEV, M195 of the E1 protein of WEEV, A215 of the E1 protein of WEEV, R216 of the E1 protein of WEEV, D218 of the E1 protein of WEEV, I219 of the E1 protein of WEEV, L221 of the E1 protein of WEEV, and K223 of the E1 protein of WEEV, and wherein at least one specificity determining reside in the first antibody contacts at least one backbone atom in the WEEV E1 protein. In certain aspects, the first antibody is selected from the group consisting of SKW10, SKW11, SKW14, SKW15, SKW16, SKW17, SKW18, SKW19, SKW24, SKW29, SKW30, SKE25, SKE26, SKE27, SKE33, SKV09, SKV11, SKV16, SKV18, SKV20, SKT01, SKT02, SKT05, SKT14, SKT19, and SKT20. In certain aspects, the first antibody is STK05 or STK20.

In one aspect, the isolated antibody is selected from the group consisting of:
a. an isolated antibody (e.g., SKE26) in which the specificity determining residues of the antibody interact with at least 15, optionally at least 20, optionally at least 22, optionally at least 24, optionally at least 26, optionally at least 28, optionally at least 30, optionally at least 32, optionally at least 34, optionally at least 36, optionally at least 37, optionally at least 38, or all, contact residues selected from the group consisting of Y89 of the E1 protein of EEEV, G90 of the E1 protein of EEEV, D3 of the E2 protein of EEEV, F6 of the E2 protein of EEEV, T7 of the E2 protein of EEEV, Y9 of the E2 protein of EEEV, K10 of the E2 protein of EEEV, L11 of the E2 protein of EEEV, R13 of the E2 protein of EEEV, T57 of the E2 protein of EEEV, D58 of the E2 protein of EEEV, G59 of the E2 protein of EEEV, M68 of the E2 protein of EEEV, N69 of the E2 protein of EEEV, G70 of the E2 protein of EEEV, K71 of the E2 protein of EEEV, T72 of the E2 protein of EEEV, Q73 of the E2 protein of EEEV, K74 of the E2 protein of EEEV, S75 of the E2 protein of EEEV, V172 of the E2 protein of EEEV, S176 of the E2 protein of EEEV, K186 of the E2 protein of EEEV, T188 of the E2 protein of EEEV, V189 of the E2 protein of EEEV, P190 of the E2 protein of EEEV, S191 of the E2 protein of EEEV, G192 of the E2 protein of EEEV, A193 of the E2 protein of EEEV, Q194 of the E2 protein of EEEV, T209 of the E2 protein of EEEV, S210 of the E2 protein of EEEV, S211 of the E2 protein of EEEV, L227 of the E2 protein of EEEV, I228 of the E2 protein of EEEV, D229 of the E2 protein of EEEV, N230 of the E2 protein of EEEV, K231 of the E2 protein of EEEV, and K232 of the E2 protein of EEEV;
b. an isolated antibody (e.g., SKT05) in which the specificity determining residues of the antibody interact with at least 15, optionally at least 20, optionally at least 22, optionally at least 24, optionally at least 26, optionally at least 28, optionally at least 30, optionally at least 31, or all, contact residues selected from the group consisting of G64 of the E1 protein of VEEV, S65 of the E1 protein of VEEV, Q66 of the E1 protein of VEEV, E67 of the E1 protein of VEEV, C68 of the E1 protein of VEEV, T69 of the E1 protein of VEEV, P70 of the E1 protein of VEEV, Y72 of the E1 protein of VEEV, D75 of the E1 protein of VEEV, E76 of the E1 protein of VEEV, Q77 of the E1 protein of VEEV, C78 of the E1 protein of VEEV, K79 of the E1 protein of VEEV, V80 of the E1 protein of VEEV, F81 of the E1 protein of VEEV, T82 of the E1 protein of VEEV, T98 of the E1 protein of VEEV, E99 of the E1 protein of VEEV, Y214 of the E1 protein of VEEV, A215 of the E1 protein of VEEV, N216 of the E1 protein of VEEV, N218 of the E1 protein of VEEV, L219 of the E1 protein of VEEV, V220 of the E1 protein of VEEV, R223 of the E1 protein of VEEV, S141 of the E1 protein of VEEV, V143 of the E1 protein of VEEV, D311 of the E1 protein of VEEV, K340 of the E1 protein of VEEV, T389 of the E1 protein of VEEV, H390 of the E1 protein of VEEV, and Y393 of the E1 protein of VEEV;
c. an isolated antibody (e.g., SKT20) in which the specificity determining residues of the antibody interact with at least 15, optionally at least 18, optionally at least 20, optionally at least 21, optionally at least 22, or all, contact residues selected from the group consisting of F87 of the E1 protein of VEEV, M88 of the E1 protein of VEEV, W89 of the E1 protein of VEEV, G90 of the E1 protein of VEEV, G91 of the E1 protein of VEEV, Y93 of the E1 protein of VEEV, F95 of the E1 protein of VEEV, C96 of the E1 protein of VEEV, D97 of the E1 protein of VEEV, E99 of the E1 protein of VEEV, A226 of the E1 protein of VEEV, G227 of the E1 protein of VEEV, H28 of the E2 protein of VEEV, L55 of the E2 protein of VEEV, G63 of the E2 protein of VEEV, R64 of the E2 protein of VEEV, D94 of the E2 protein of VEEV, G95 of the E2 protein of VEEV, H96 of the E2 protein of VEEV, H156 of the E2 protein of VEEV, D157 of the E2 protein of VEEV, A158 of the E2 protein of VEEV, and Q159 of the E2 protein of VEEV;
d. an isolated antibody (e.g., SKV09) in which the specificity determining residues of the antibody interact with at least 15, optionally at least 16, optionally at least 17, optionally at least 18, optionally at least 19, or all, contact residues selected from the group consisting of E348 of the E1 protein of VEEV, Q349 of the E1 protein of VEEV, K319 of the E1 protein of VEEV, S325 of the E1 protein of VEEV, T347 of the E1 protein of VEEV, E348 of the E1 protein of VEEV, Q349 of the E1 protein of VEEV, A298 of the E1 protein of VEEV, T317 of the E1 protein of VEEV, K319 of the E1 protein of VEEV, S323 of the E1 protein of VEEV, K324 of the E1 protein of VEEV, S325 of the E1 protein of VEEV, G326 of the E1 protein of VEEV, E345 of the E1 protein of VEEV, T347 of the E1 protein of VEEV, E348 of the E1 protein of VEEV, Q349 of the E1 protein of VEEV, G350 of the E1 protein of VEEV, S351 of the E1 protein of VEEV;
e. an isolated antibody (e.g., SKV16) in which the specificity determining residues of the antibody interact with at least 15, optionally at least 16, optionally at least 17, optionally at least 18, optionally at least 19, or all, contact residues selected from the group consisting of S180 of the E2 protein of VEEV, L181 of the E2 protein of VEEV, S182 of the E2 protein of VEEV, G183 of the E2 protein of VEEV, S185 of the E2 protein of VEEV, T187 of the E2 protein of VEEV, T189 of the E2 protein of VEEV, P190 of the E2 protein of VEEV, P191 of the E2 protein of VEEV, D192 of the E2 protein of VEEV, G193 of the E2 protein of VEEV, E209 of the E2 protein of VEEV, T210 of the E2 protein of VEEV, 1211 of the E2 protein of VEEV, N212 of the E2 protein of VEEV, K213 of the E2 protein of VEEV, T214 of the E2 protein of VEEV, K215 of the E2 protein of VEEV, Q216 of the E2 protein of VEEV, and S218 of the E2 protein of VEEV;
f. an isolated antibody (e.g., SKT5) in which the specificity determining residues of the antibody interact with at least 15, optionally at least 20, optionally at least 22, optionally at least 24, optionally at least 26, optionally at least 27, or all, contact residues selected from the group consisting of C63 of the E1 protein of WEEV, S65 of the E1 protein of WEEV, L66 of the E1 protein of WEEV, E67 of the E1 protein of WEEV, C68 of the E1 protein of WEEV, K69 of the E1 protein of WEEV, A70 of the E1 protein of WEEV, S71 of the E1 protein of WEEV, D75 of the E1 protein of WEEV, Y76 of the E1 protein of WEEV, T77 of the E1 protein of WEEV, C78 of the E1 protein of WEEV, R79 of the E1 protein of WEEV, V80 of the E1 protein of WEEV, F81 of the E1 protein of WEEV, G82 of the E1 protein of WEEV, G83 of the E1 protein of WEEV, S98 of the E1 protein of WEEV, E99 of the E1 protein of WEEV, N100 of the E1 protein of WEEV, T101 of the E1 protein of WEEV, M195 of the E1 protein of WEEV, A215 of the E1 protein of WEEV, R216 of the E1 protein of WEEV, D218 of the E1 protein of WEEV, I219 of the E1 protein of WEEV, L221 of the E1 protein of WEEV, and K223 of the E1 protein of WEEV;
g. an isolated antibody (e.g., SKW11) in which the specificity determining residues of the antibody interact with at least 15, optionally at least 16, optionally at least 17, or all, contact residues selected from the group consisting of N57 of the E2 protein of WEEV, Q58 of the E2 protein of WEEV, A59 of the E2 protein of WEEV, T61 of the E2 protein of WEEV, R173 of the E2 protein of WEEV, G197 of the E2 protein of WEEV, K198 of the E2 protein of WEEV, K233 of the E2 protein of WEEV, D235 of the E2 protein of WEEV, Q236 of the E2 protein of WEEV, T237 of the E2 protein of WEEV, W239 of the E2 protein of WEEV, D251 of the E2 protein of WEEV, S253 of the E2 protein of WEEV, V254 of the E2 protein of WEEV, Q255 of the E2 protein of WEEV, G256 of the E2 protein of WEEV, and K257 of the E2 protein of WEEV;
h. an isolated antibody (e.g., SKW19) in which the specificity determining residues of the antibody interact with at least 15, optionally at least 16, optionally at least 18, optionally at least 20, optionally at least 21, or all, contact residues selected from the group consisting of K61 of the E1 protein of WEEV, C63 of the E1 protein of WEEV, G64 of the E1 protein of WEEV, S65 of the E1 protein of WEEV, F95 of the E1 protein of WEEV, N199 of the E2 protein of WEEV, T201 of the E2 protein of WEEV, Y202 of the E2 protein of WEEV, K205 of the E2 protein of WEEV, D208 of the E2 protein of WEEV, Y209 of the E2 protein of WEEV, S210 of the E2 protein of WEEV, T211 of the E2 protein of WEEV, G212 of the E2 protein of WEEV, I213 of the E2 protein of WEEV, V214 of the E2 protein of WEEV, S215 of the E2 protein of WEEV, T216 of the E2 protein of WEEV, T218 of the E2 protein of WEEV, K219 of the E2 protein of WEEV, M220 of the E2 protein of WEEV, and N221 of the E2 protein of WEEV; and,
i. an isolated antibody (e.g., SKW24) in which the specificity determining residues of the antibody interact with at least 15, optionally at least 20, optionally at least 22, optionally at least 24, optionally at least 26, optionally at least 28, optionally at least 30, optionally at least 31, optionally at least 32, or all, contact residues selected from the group consisting of Q59 of the E1 protein of WEEV, V60 of the E1 protein of WEEV, K61 of the E1 protein of WEEV, C62 of the E1 protein of WEEV, Q93 of the E1 protein of WEEV, C94 of the E1 protein of WEEV, F95 of the E1 protein of WEEV, R173 of the E2 protein of WEEV, P176 of the E2 protein of WEEV, A178 of the E2 protein of WEEV, T201 of the E2 protein of WEEV, Y202 of the E2 protein of WEEV, E203 of the E2 protein of WEEV, K205 of the E2 protein of WEEV, G207 of the E2 protein of WEEV, D208 of the E2 protein of WEEV, Y209 of the E2 protein of WEEV, S210 of the E2 protein of WEEV, T211 of the E2 protein of WEEV, G212 of the E2 protein of WEEV, I213 of the E2 protein of WEEV, N221 of the E2 protein of WEEV, 1230 of the E2 protein of WEEV, Y232 of the E2 protein of WEEV, S234 of the E2 protein of WEEV, Q236 of the E2 protein of WEEV, 1247 of the E2 protein of WEEV, R248 of the E2 protein of WEEV, H249 of the E2 protein of WEEV, T250 of the E2 protein of WEEV, D251 of the E2 protein of WEEV, S253 of the E2 protein of WEEV, and Q255 of the E2 protein of WEEV.

In certain aspects, the isolated antibody of the disclosure is an antibody (e.g., SKT5) in which the specificity determining residues of the antibody interact with at least 15, optionally at least 20, optionally at least 22, optionally at least 24, optionally at least 26, or all, contact residues selected from the group consisting of C63 of the E1 protein of WEEV, S65 of the E1 protein of WEEV, L66 of the E1 protein of WEEV, E67 of the E1 protein of WEEV, C68 of the E1 protein of WEEV, K69 of the E1 protein of WEEV, A70 of the E1 protein of WEEV, S71 of the E1 protein of WEEV, D75 of the E1 protein of WEEV, Y76 of the E1 protein of WEEV, T77 of the E1 protein of WEEV, C78 of the E1 protein of WEEV, R79 of the E1 protein of WEEV, V80 of the E1 protein of WEEV, F81 of the E1 protein of WEEV, G82 of the E1 protein of WEEV, G83 of the E1 protein of WEEV, S98 of the E1 protein of WEEV, E99 of the E1 protein of WEEV, N100 of the E1 protein of WEEV, T101 of the E1 protein of WEEV, M195 of the E1 protein of WEEV, A215 of the E1 protein of WEEV, R216 of the E1 protein of WEEV, D218 of the E1 protein of WEEV, I219 of the E1 protein of WEEV, L221 of the E1 protein of WEEV, and K223 of the E1 protein of WEEV, wherein at least one specificity determining reside in the antibody contacts at least one backbone atom in the WEEV E1 protein. In certain aspects, the isolated antibody is selected from the group consisting of SKW10, SKW11, SKW14, SKW15, SKW16, SKW17, SKW18, SKW19, SKW24, SKW29, SKW30, SKE25, SKE26, SKE27, SKE33, SKV09, SKV11, SKV16, SKV18, SKV20, SKT01, SKT02, SKT05, SKT14, and SKT20. In certain aspects, the isolated antibody is STK05, STK20, SKT05.hu13, SKT05.hu21, SKT05.hu25, SKT05.hu29, SKT05.hu37, SKT05.hu41, SKT05.hu47, SKT05.hu52, SKT05.hu56, or SKT05.hu60. In certain aspects, at least one specificity determining reside in an isolated antibody of the disclosure may contact at least one backbone atom in an E1 and/or E2 protein, wherein the E1 or E2 protein may be from WEEV, EEEV, or VEEV.

In one aspect, the isolated antibody comprises a V_{L} CDR3 selected from the group consisting of: a V_{L} CDR3 comprising SEQ ID NO:3, a V_{L} CDR3 comprising SEQ ID NO: 11, a V_{L} CDR3 comprising SEQ ID NO:19, a V_{L} CDR3 comprising SEQ ID NO:27, a V_{L} CDR3 comprising SEQ ID NO:35, a V_{L} CDR3 comprising SEQ ID NO:43, a V_{L} CDR3 comprising SEQ ID NO:51, a V_{L} CDR3 comprising SEQ ID NO:59, a V_{L} CDR3 comprising SEQ ID NO:67, a V_{L} CDR3 comprising SEQ ID NO:83, a V_{L} CDR3 comprising SEQ ID NO:91, a V_{L} CDR3 comprising SEQ ID NO:99, a V_{L} CDR3 comprising SEQ ID NO:107, a V_{L} CDR3 comprising SEQ ID NO:115, a V_{L} CDR3 comprising SEQ ID NO:123, a V_{L} CDR3 comprising SEQ ID NO:131, a V_{L} CDR3 comprising SEQ ID NO:139, a V_{L} CDR3 comprising SEQ ID NO:147, a V_{L} CDR3 comprising SEQ ID NO:155, a V_{L} CDR3 comprising SEQ ID NO:163, a V_{L} CDR3 comprising SEQ ID NO:171, a V_{L} CDR3 comprising SEQ ID NO:179, a V_{L} CDR3 comprising SEQ ID NO:187, a V_{L} CDR3 comprising SEQ ID NO:195, a V_{L} CDR3 comprising SEQ ID NO:203, a V_{L} CDR3 comprising SEQ ID NO:211, a V_{L} CDR3 comprising SEQ ID NO:219, a V_{L} CDR3 comprising SEQ ID NO:227, a V_{L} CDR3 comprising SEQ ID NO:235, a V_{L} CDR3 comprising SEQ ID NO:243, a V_{L} CDR3 comprising SEQ ID NO:251, a V_{L} CDR3 comprising SEQ ID NO:259, a V_{L} CDR3 comprising SEQ ID NO:267, a V_{L} CDR3 comprising SEQ ID NO:275, and a V_{L} CDR3 comprising SEQ ID NO:283.

In one aspect, the isolated antibody comprises a V_{H} CDR3 selected from the group consisting of: a V_{H} CDR3 comprising SEQ ID NO:7, a V_{H} CDR3 comprising SEQ ID NO:15, a V_{H} CDR3 comprising SEQ ID NO:23, a V_{H} CDR3 comprising SEQ ID NO:31, a VR CDR3 comprising SEQ ID NO:39, a V_{H} CDR3 comprising SEQ ID NO:47, a V_{H} CDR3 comprising SEQ ID NO:55, a V_{H} CDR3 comprising SEQ ID NO:63, a V_{H} CDR3 comprising SEQ ID NO:71, a V_{H} CDR3 comprising SEQ ID NO:79, a V_{H} CDR3 comprising SEQ ID NO:87, a V_{H} CDR3 comprising SEQ ID NO:95, a V_{H} CDR3 comprising SEQ ID NO:103, a V_{H} CDR3 comprising SEQ ID NO: 11, a V_{H} CDR3 comprising SEQ ID NO:119, a V_{H} CDR3 comprising SEQ ID NO:127, a V_{H} CDR3 comprising SEQ ID NO:135, a V_{H} CDR3 comprising SEQ ID NO:143, a V_{H} CDR3 comprising SEQ ID NO:151, a V_{H} CDR3 comprising SEQ ID NO:159, a V_{H} CDR3 comprising SEQ ID NO:167, a V_{H} CDR3 comprising SEQ ID NO:175, a V_{H} CDR3 comprising SEQ ID NO:183, a V_{H} CDR3 comprising SEQ ID NO:191, a V_{H} CDR3 comprising SEQ ID NO:199, a V_{H} CDR3 comprising SEQ ID NO:207, a V_{H} CDR3 comprising SEQ ID NO:215, a V_{H} CDR3 comprising SEQ ID NO:223, a V_{H} CDR3 comprising SEQ ID NO:231, a V_{H} CDR3 comprising SEQ ID NO:239, a V_{H} CDR3 comprising SEQ ID NO:247, a V_{H} CDR3 comprising SEQ ID NO:255, a V_{H} CDR3 comprising SEQ ID NO:263, a V_{H} CDR3 comprising SEQ ID NO:271, a V_{H} CDR3 comprising SEQ ID NO:279, and a V_{H} CDR3 comprising SEQ ID NO:287.

In one aspect, the isolated antibody comprises: i) a V_{L} CDR3 selected from the group consisting of a V_{L} CDR3 comprising SEQ ID NO:3, a V_{L} CDR3 comprising SEQ ID NO:11, a V_{L} CDR3 comprising SEQ ID NO:19, a V_{L} CDR3 comprising SEQ ID NO:27, a V_{L} CDR3 comprising SEQ ID NO:35, a V_{L} CDR3 comprising SEQ ID NO:43, a V_{L} CDR3 comprising SEQ ID NO:51, a V_{L} CDR3 comprising SEQ ID NO:59, a V_{L} CDR3 comprising SEQ ID NO:67, a V_{L} CDR3 comprising SEQ ID NO:83, a V_{L} CDR3 comprising SEQ ID NO:91, a V_{L} CDR3 comprising SEQ ID NO:99, a V_{L} CDR3 comprising SEQ ID NO:107, a V_{L} CDR3 comprising SEQ ID NO:115, a V_{L} CDR3 comprising SEQ ID NO:123, a V_{L} CDR3 comprising SEQ ID NO:131, a V_{L} CDR3 comprising SEQ ID NO:139, a V_{L} CDR3 comprising SEQ ID NO:147, a V_{L} CDR3 comprising SEQ ID NO:155, a V_{L} CDR3 comprising SEQ ID NO:163, and a V_{L} CDR3 comprising SEQ ID NO:171, a V_{L} CDR3 comprising SEQ ID NO:179, a V_{L} CDR3 comprising SEQ ID NO:187, a V_{L} CDR3 comprising SEQ ID NO:195, and a V_{L} CDR3 comprising SEQ ID NO:203, a V_{L} CDR3 comprising SEQ ID NO:211, a V_{L} CDR3 comprising SEQ ID NO:219, a V_{L} CDR3 comprising SEQ ID NO:227, a V_{L} CDR3 comprising SEQ ID NO:235, a V_{L} CDR3 comprising SEQ ID NO:243, a V_{L} CDR3 comprising SEQ ID NO:251, a V_{L} CDR3 comprising SEQ ID NO:259, a V_{L} CDR3 comprising SEQ ID NO:267, a V_{L} CDR3 comprising SEQ ID NO:275, and a V_{L} CDR3 comprising SEQ ID NO:283; and, ii) a heavy chain variable region comprising a V_{H} CDR3 selected from the group consisting of: a V_{H} CDR3 comprising SEQ ID NO:7, a V_{H} CDR3 comprising SEQ ID NO:15, a V_{H} CDR3 comprising SEQ ID NO:23, a V_{H} CDR3 comprising SEQ ID NO:31, a V_{H} CDR3 comprising SEQ ID NO:39, a V_{H} CDR3 comprising SEQ ID NO:47, a V_{H} CDR3 comprising SEQ ID NO:55, a V_{H} CDR3 comprising SEQ ID NO:63, a V_{H} CDR3 comprising SEQ ID NO:71, a V_{H} CDR3 comprising SEQ ID NO:79, a V_{H} CDR3 comprising SEQ ID NO:87, a V_{H} CDR3 comprising SEQ ID NO:95, a V_{H} CDR3 comprising SEQ ID NO:103, a V_{H} CDR3 comprising SEQ ID NO: 11, a V_{H} CDR3 comprising SEQ ID NO:119, a V_{H} CDR3 comprising SEQ ID NO:127, a V_{H} CDR3 comprising SEQ ID NO:135, a V_{H} CDR3 comprising SEQ ID NO:143, a V_{H} CDR3 comprising SEQ ID NO:151, a V_{H} CDR3 comprising SEQ ID NO:159, a V_{H} CDR3 comprising SEQ ID NO:167, a V_{H} CDR3 comprising SEQ ID NO:175, a V_{H} CDR3 comprising SEQ ID NO:183, a V_{H} CDR3 comprising SEQ ID NO:191, a V_{H} CDR3 comprising SEQ ID NO:199, and a V_{H} CDR3 comprising SEQ ID NO:207, a V_{H} CDR3 comprising SEQ ID NO:215, a V_{H} CDR3 comprising SEQ ID NO:223, a V_{H} CDR3 comprising SEQ ID NO:231, a V_{H} CDR3 comprising SEQ ID NO:239, a V_{H} CDR3 comprising SEQ ID NO:247, a V_{H} CDR3 comprising SEQ ID NO:255, a V_{H} CDR3 comprising SEQ ID NO:263, a V_{H} CDR3 comprising SEQ ID NO:271, a V_{H} CDR3 comprising SEQ ID NO:279, and a V_{H} CDR3 comprising SEQ ID NO:287.

In one aspect, the isolated antibody comprises a V_{L} CDR3 and a V_{H} CDR3. In certain aspects, the V_{L} CDR3 may comprise or consist of SEQ ID NO:3 and the V_{H} CDR3 may comprise or consist of SEQ ID NO:7. In certain aspects, the V_{L} CDR3 may comprise or consist of SEQ ID NO:11 and the V_{H} CDR3 may comprise or consist of SEQ ID NO: 15. In certain aspects, the V_{L} CDR3 may comprise or consist of SEQ ID NO:19 and the V_{H} CDR3 may comprise or consist of SEQ ID NO:23. In certain aspects, the V_{L} CDR3 may comprise or consist of SEQ ID NO:27 and the V_{H} CDR3 may comprise or consist of SEQ ID NO:31. In certain aspects, the V_{L} CDR3 may comprise or consist of SEQ ID NO:35 and the V_{H} CDR3 may comprise or consist of SEQ ID NO:39. In certain aspects, the V_{L} CDR3 may comprise or consist of SEQ ID NO:43 and the V_{H} CDR3 may comprise or consist of SEQ ID NO:47. In certain aspects, the V_{L} CDR3 may comprise or consist of SEQ ID NO:51 and the V_{H} CDR3 may comprise or consist of SEQ ID NO:55. In certain aspects, the V_{L} CDR3 may comprise or consist of SEQ ID NO:59 and the V_{H} CDR3 may comprise or consist of SEQ ID NO:63. In certain aspects, the V_{L} CDR3 may comprise or consist of SEQ ID NO:67 and the V_{H} CDR3 may comprise or consist of SEQ ID NO:71. In certain aspects, the V_{L} CDR3 may comprise or consist of SEQ ID NO:75 and the V_{H} CDR3 may comprise or consist of SEQ ID NO:79. In certain aspects, the V_{L} CDR3 may comprise or consist of SEQ ID NO:83 and the V_{H} CDR3 may comprise or consist of SEQ ID NO:87. In certain aspects, the V_{L} CDR3 may comprise or consist of SEQ ID NO:91 and the V_{H} CDR3 may comprise or consist of SEQ ID NO:95. In certain aspects, the V_{L} CDR3 may comprise or consist of SEQ ID NO:99 and the V_{H} CDR3 may comprise or consist of SEQ ID NO:103. In certain aspects, the V_{L} CDR3 may comprise or consist of SEQ ID NO:107 and the V_{H} CDR3 may comprise or consist of SEQ ID NO:111. In certain aspects, the V_{L} CDR3 may comprise or consist of SEQ ID NO:115 and the V_{H} CDR3 may comprise or consist of SEQ ID NO: 119. In certain aspects, the V_{L} CDR3 may comprise or consist of SEQ ID NO:123 and the V_{H} CDR3 may comprise or consist of SEQ ID NO: 127. In certain aspects, the V_{L} CDR3 may comprise or consist of SEQ ID NO:131 and the V_{H} CDR3 may comprise or consist of SEQ ID NO:135. In certain aspects, the V_{L} CDR3 may comprise or consist of SEQ ID NO:139 and the V_{H} CDR3 may comprise or consist of SEQ ID NO: 143. In certain aspects, the V_{L} CDR3 may comprise or consist of SEQ ID NO:147 and the V_{H} CDR3 may comprise or consist of SEQ ID NO:151. In certain aspects, the V_{L} CDR3 may comprise or consist of SEQ ID NO:155 and the V_{H} CDR3 may comprise or consist of SEQ ID NO:159. In certain aspects, the V_{L} CDR3 may comprise or consist of SEQ ID NO:163 and the V_{H} CDR3 may comprise or consist of SEQ ID NO: 167. In certain aspects, the V_{L} CDR3 may comprise or consist of SEQ ID NO:171 and the V_{H} CDR3 may comprise or consist of SEQ ID NO:175. In certain aspects, the V_{L} CDR3 may comprise or consist of SEQ ID NO:179 and the V_{H} CDR3 may comprise or consist of SEQ ID NO:183. In certain aspects, the V_{L} CDR3 may comprise or consist of SEQ ID NO: 187 and the V_{H} CDR3 may comprise or consist of SEQ ID NO:191. In certain aspects, the V_{L} CDR3 may comprise or consist of SEQ ID NO:195 and the V_{H} CDR3 may comprise or consist of SEQ ID NO:199. In certain aspects, the V_{L} CDR3 may comprise or consist of SEQ ID NO:203 and the V_{H} CDR3 may comprise or consist of SEQ ID NO:207. In certain aspects, the V_{L} CDR3 may comprise or consist of SEQ ID NO 211; and the V_{H} CDR3 may comprise or consist of SEQ ID NO:215. In certain aspects, the V_{L} CDR3 may comprise or consist of SEQ ID NO 219; and the V_{H} CDR3 may comprise or consist of SEQ ID NO:223. In certain aspects, the V_{L} CDR3 may comprise or consist of SEQ ID NO 227; and the V_{H} CDR3 may comprise or consist of SEQ ID NO:231. In certain aspects, the V_{L} CDR3 may comprise or consist of SEQ ID NO:235 and the V_{H} CDR3 may comprise or consist of SEQ ID NO:239. In certain aspects, the V_{L} CDR3 may comprise or consist of SEQ ID NO:243 and the V_{H} CDR3 may comprise or consist of SEQ ID NO:247. In certain aspects, the V_{L} CDR3 may comprise or consist of SEQ ID NO:251 and the V_{H} CDR3 may comprise or consist of SEQ ID NO:255. In certain aspects, the V_{L} CDR3 may comprise or consist of SEQ ID NO 259; and the V_{H} CDR3 may comprise or consist of SEQ ID NO:263. In certain aspects, the V_{L} CDR3 may comprise or consist of SEQ ID NO:267 and the V_{H} CDR3 may comprise or consist of SEQ ID NO:271. In certain aspects, the V_{L} CDR3 may comprise or consist of SEQ ID NO:275 and the V_{H} CDR3 may comprise or consist of SEQ ID NO:279. In certain aspects, the V_{L} CDR3 may comprise or consist of SEQ ID NO:283 and the V_{H} CDR3 may comprise or consist of SEQ ID NO:287.

In one aspect, the isolated antibody comprises a V_{L} CDR1, a V_{L} CDR2, and a V_{L} CDR3. In certain aspects, the V_{L} CDR1 may comprise or consist of SEQ ID NO:1, the V_{L} CDR2 may comprise or consist of the amino acid sequence QAS (SEQ ID NO:2), and the V_{L} CDR3 may comprise or consist of SEQ ID NO:3. In certain aspects, the V_{L} CDR1 may comprise or consist of SEQ ID NO:9, the V_{L} CDR2 may comprise or consist of the amino acid sequence QAS (SEQ ID NO:10), and V_{L} CDR3 may comprise or consist of SEQ ID NO:11. In certain aspects, the V_{L} CDR1 may comprise or consist of SEQ ID NO:17, the V_{L} CDR2 may comprise or consist of the amino acid sequence EVS (SEQ ID NO:18), and the V_{L} CDR3 may comprise or consist of SEQ ID NO: 19. In certain aspects, the V_{L} CDR1 may comprise or consist of SEQ ID NO:25, the V_{L} CDR2 may comprise or consist of the amino acid sequence EVS (SEQ ID NO:26), and the V_{L} CDR3 may comprise or consist of SEQ ID NO:27. In certain aspects, the V_{L} CDR1 may comprise or consist of SEQ ID NO:33, the V_{L} CDR2 may comprise or consist of the amino acid sequence WAS (SEQ ID NO:34), and the V_{L} CDR3 may comprise or consist of SEQ ID NO:35. In certain aspects, the V_{L} CDR1 may comprise or consist of SEQ ID NO:41, the V_{L} CDR2 may comprise or consist of the amino acid sequence WAS (SEQ ID NO:42), and the V_{L} CDR3 may comprise or consist of SEQ ID NO:43. In certain aspects, the V_{L} CDR1 may comprise or consist of SEQ ID NO:49, the V_{L} CDR2 may comprise or consist of the amino acid sequence WAS (SEQ ID NO:50), and the V_{L} CDR3 may comprise or consist of SEQ ID NO:51. In certain aspects, the V_{L} CDR1 may comprise or consist of SEQ ID NO:57, the V_{L} CDR2 may comprise or consist of the amino acid sequence QVS (SEQ ID NO:58), and the V_{L} CDR3 may comprise or consist of SEQ ID NO:59. In certain aspects, the V_{L} CDR1 may comprise or consist of SEQ ID NO:65, the V_{L} CDR2 may comprise or consist of the amino acid sequence ANG (SEQ ID NO:66), and the V_{L} CDR3 may comprise or consist of SEQ ID NO:67. In certain aspects, the V_{L} CDR1 may comprise or consist of SEQ ID NO:73, the V_{L} CDR2 may comprise or consist of the amino acid sequence EVS (SEQ ID NO:74), and the V_{L} CDR3 may comprise or consist of SEQ ID NO:75. In certain aspects, the V_{L} CDR1 may comprise or consist of SEQ ID NO:81, the V_{L} CDR2 may comprise or consist of the amino acid sequence EVS (SEQ ID NO:82), and the V_{L} CDR3 may comprise or consist of SEQ ID NO:83. In certain aspects, the V_{L} CDR1 may comprise or consist of SEQ ID NO:89, the V_{L} CDR2 may comprise or consist of the amino acid sequence GGS (SEQ ID NO:90), and the V_{L} CDR3 may comprise or consist of SEQ ID NO:91. In certain aspects, the V_{L} CDR1 may comprise or consist of SEQ ID NO:97, the V_{L} CDR2 may comprise or consist of the amino acid sequence GGS (SEQ ID NO:98), and the V_{L} CDR3 may comprise or consist of SEQ ID NO:99. In certain aspects, the V_{L} CDR1 may comprise or consist of SEQ ID NO:105, the V_{L} CDR2 may comprise or consist of the amino acid sequence GGS (SEQ ID NO: 106), and the V_{L} CDR3 may comprise or consist of SEQ ID NO:107. In certain aspects, the V_{L} CDR1 may comprise or consist of SEQ ID NO: 113, the V_{L} CDR2 may comprise or consist of the amino acid sequence YGE (SEQ ID NO: 114), and the V_{L} CDR3 may comprise or consist of SEQ ID NO:115. In certain aspects, the V_{L} CDR1 may comprise or consist of SEQ ID NO:121, the V_{L} CDR2 may comprise or consist of the amino acid sequence KAS (SEQ ID NO:122), and the V_{L} CDR3 may comprise or consist of SEQ ID NO:123. In certain aspects, the V_{L} CDR1 may comprise or consist of SEQ ID NO: 129, the V_{L} CDR2 may comprise or consist of the amino acid sequence AAS (SEQ ID NO: 130), and the V_{L} CDR3 may comprise or consist of SEQ ID NO:131. In certain aspects, the V_{L} CDR1 may comprise or consist of SEQ ID NO:137, the V_{L} CDR2 may comprise or consist of the amino acid sequence DDR (SEQ ID NO:138), and the V_{L} CDR3 may comprise or consist of SEQ ID NO:139. In certain aspects, the V_{L} CDR1 may comprise or consist of SEQ ID NO:145, the V_{L} CDR2 may comprise or consist of the amino acid sequence LAS (SEQ ID NO:146), and the V_{L} CDR3 may comprise or consist of SEQ ID NO:147. In certain aspects, the V_{L} CDR1 may comprise or consist of SEQ ID NO:153, the V_{L} CDR2 may comprise or consist of the amino acid sequence ENN (SEQ ID NO:154), and the V_{L} CDR3 may comprise or consist of SEQ ID NO:155. In certain aspects, the V_{L} CDR1 may comprise or consist of SEQ ID NO:161, the V_{L} CDR2 may comprise or consist of the amino acid sequence DAS (SEQ ID NO:162), and the V_{L} CDR3 may comprise or consist of SEQ ID NO:163. In certain aspects, the V_{L} CDR1 may comprise or consist of SEQ ID NO:169, the V_{L} CDR2 may comprise or consist of the amino acid sequence DAS (SEQ ID NO:170), and the V_{L} CDR3 may comprise or consist of SEQ ID NO:171. In certain aspects, the V_{L} CDR1 may comprise or consist of SEQ ID NO:177, the V_{L} CDR2 may comprise or consist of the amino acid sequence DAS (SEQ ID NO:178), and the V_{L} CDR3 may comprise or consist of SEQ ID NO:179. In certain aspects, the V_{L} CDR1 may comprise or consist of SEQ ID NO:185, the V_{L} CDR2 may comprise or consist of the amino acid sequence ADS (SEQ ID NO:186), and the V_{L} CDR3 may comprise or consist of SEQ ID NO:187. In certain aspects, the V_{L} CDR1 may comprise or consist of SEQ ID NO: 193, the V_{L} CDR2 may comprise or consist of the amino acid sequence SVS (SEQ ID NO:194), and the V_{L} CDR3 may comprise or consist of SEQ ID NO:195. In certain aspects, the V_{L} CDR1 may comprise or consist of SEQ ID NO:201, the V_{L} CDR2 may comprise or consist of the amino acid sequence GGS (SEQ ID NO:202), and the V_{L} CDR3 may comprise or consist of SEQ ID NO:203. In certain aspects, the V_{L} CDR1 may comprise or consist of SEQ ID NO:209, the V_{L} CDR2 may comprise or consist of the amino acid sequence DAS (SEQ ID NO:210), and the V_{L} CDR3 may comprise or consist of SEQ ID NO:211. In certain aspects, the V_{L} CDR1 may comprise or consist of SEQ ID NO:217, the V_{L} CDR2 may comprise or consist of the amino acid sequence DAS (SEQ ID NO:218), and the V_{L} CDR3 may comprise or consist of SEQ ID NO:219. In certain aspects, the V_{L} CDR1 may comprise or consist of SEQ ID NO:225, the V_{L} CDR2 may comprise or consist of the amino acid sequence DAS (SEQ ID NO:226), and the V_{L} CDR3 may comprise or consist of SEQ ID NO:227. In certain aspects, the V_{L} CDR1 may comprise or consist of SEQ ID NO:233, the V_{L} CDR2 may comprise or consist of the amino acid sequence DAS (SEQ ID NO:234), and the V_{L} CDR3 may comprise or consist of SEQ ID NO:235. In certain aspects, the V_{L} CDR1 may comprise or consist of SEQ ID NO:241, the V_{L} CDR2 may comprise or consist of the amino acid sequence DAS (SEQ ID NO:242), and the V_{L} CDR3 may comprise or consist of SEQ ID NO:243. In certain aspects, the V_{L} CDR1 may comprise or consist of SEQ ID NO:249, the V_{L} CDR2 may comprise or consist of the amino acid sequence DAS (SEQ ID NO:250), and the V_{L} CDR3 may comprise or consist of SEQ ID NO:251. In certain aspects, the V_{L} CDR1 may comprise or consist of SEQ ID NO:257, the V_{L} CDR2 may comprise or consist of the amino acid sequence DAS (SEQ ID NO:258), and the V_{L} CDR3 may comprise or consist of SEQ ID NO:259. In certain aspects, the V_{L} CDR1 may comprise or consist of SEQ ID NO:265, the V_{L} CDR2 may comprise or consist of the amino acid sequence DAS (SEQ ID NO:266), and the V_{L} CDR3 may comprise or consist of SEQ ID NO:267. In certain aspects, the V_{L} CDR1 may comprise or consist of SEQ ID NO:273, the V_{L} CDR2 may comprise or consist of the amino acid sequence DAS (SEQ ID NO:274), and the V_{L} CDR3 may comprise or consist of SEQ ID NO:275. In certain aspects, the V_{L} CDR1 may comprise or consist of SEQ ID NO:281, the V_{L} CDR2 may comprise or consist of the amino acid sequence DAS (SEQ ID NO:282), and the V_{L} CDR3 may comprise or consist of SEQ ID NO:283.

In one aspect, the isolated antibody may comprise a V_{H} CDR1, a V_{H} CDR2, and a V_{H} CDR3. In certain aspects, the V_{H} CDR1 may comprise or consist of SEQ ID NO:5, the V_{H} CDR2 may comprise or consist of SEQ ID NO:6, and the V_{H} CDR3 may comprise or consist of SEQ ID NO:7. In certain aspects, the V_{H} CDR1 may comprise or consist of SEQ ID NO:13, the V_{H} CDR2 may comprise or consist of SEQ ID NO:14, and the V_{H} CDR3 may comprise or consist of SEQ ID NO:15. In certain aspects, the V_{H} CDR1 may comprise or consist of SEQ ID NO:21, the V_{H} CDR2 may comprise or consist of SEQ ID NO:22, and the V_{H} CDR3 may comprise or consist of SEQ ID NO:23. In certain aspects, the V_{H} CDR1 may comprise or consist of SEQ ID NO:29, the V_{H} CDR2 may comprise or consist of SEQ ID NO:30, and the V_{H} CDR3 may comprise or consist of SEQ ID NO:31. In certain aspects, the V_{H} CDR1 may comprise or consist of SEQ ID NO:37, the V_{H} CDR2 may comprise or consist of SEQ ID NO:38, and the V_{H} CDR3 may comprise or consist of SEQ ID NO:39. In certain aspects, the V_{H} CDR1 may comprise or consist of SEQ ID NO:45, the V_{H} CDR2 may comprise or consist of SEQ ID NO:46, and the V_{H} CDR3 may comprise or consist of SEQ ID NO:47. In certain aspects, the V_{H} CDR1 may comprise or consist of SEQ ID NO:53, the V_{H} CDR2 may comprise or consist of SEQ ID NO:54, and the V_{H} CDR3 may comprise or consist of SEQ ID NO:55. In certain aspects, the V_{H} CDR1 may comprise or consist of SEQ ID NO:61, the V_{H} CDR2 may comprise or consist of SEQ ID NO:62, and the V_{H} CDR3 may comprise or consist of SEQ ID NO:63. In certain aspects, the V_{H} CDR1 may comprise or consist of SEQ ID NO:69, the V_{H} may comprise or consist of SEQ ID NO:70, and the V_{H} CDR3 may comprise or consist of SEQ ID NO:71. In certain aspects, the V_{H} CDR1 may comprise or consist of SEQ ID NO:77, the V_{H} CDR2 may comprise or consist of SEQ ID NO:78, and the V_{H} CDR3 may comprise or consist of SEQ ID NO:79. In certain aspects, the V_{H} CDR1 may comprise or consist of SEQ ID NO:85, the V_{H} CDR2 may comprise or consist of SEQ ID NO:86, and the V_{H} CDR3 may comprise or consist of SEQ ID NO:87. In certain aspects, the V_{H} CDR1 may comprise or consist of SEQ ID NO:93, the V_{H} CDR2 may comprise or consist of SEQ ID NO:94, and the V_{H} CDR3 may comprise or consist of SEQ ID NO:95. In certain aspects, the V_{H} CDR1 may comprise or consist of SEQ ID NO:101, the V_{H} CDR2 may comprise or consist of SEQ ID NO:102, and the V_{H} CDR3 may comprise or consist of SEQ ID NO:103. In certain aspects, the V_{H} CDR1 may comprise or consist of SEQ ID NO:109, the V_{H} CDR2 may comprise or consist of SEQ ID NO:110, and the V_{H} CDR3 may comprise or consist of SEQ ID NO:111. In certain aspects, the V_{H} CDR1 may comprise or consist of SEQ ID NO:117, the V_{H} CDR2 may comprise or consist of SEQ ID NO:118, and the V_{H} CDR3 may comprise or consist of SEQ ID NO:119. In certain aspects, the V_{H} CDR1 may comprise or consist of SEQ ID NO:125, the V_{H} CDR2 may comprise or consist of SEQ ID NO:126, and the V_{H} CDR3 may comprise or consist of SEQ ID NO: 127. In certain aspects, the V_{H} CDR1 may comprise or consist of SEQ ID NO:133, the V_{H} CDR2 may comprise or consist of SEQ ID NO:134, and the V_{H} CDR3 may comprise or consist of SEQ ID NO:135. In certain aspects, the V_{H} CDR1 may comprise or consist of SEQ ID NO:141, the V_{H} CDR2 may comprise or consist of SEQ ID NO:142, and the V_{H} CDR3 may comprise or consist of SEQ ID NO:143. In certain aspects, the V_{H} CDR1 may comprise or consist of SEQ ID NO:149, the V_{H} CDR2 may comprise or consist of SEQ ID NO: 150, and the V_{H} CDR3 may comprise or consist of SEQ ID NO:151. In certain aspects, the V_{H} CDR1 may comprise or consist of SEQ ID NO: 157, the V_{H} CDR2 may comprise or consist of SEQ ID NO:158, and the V_{H} CDR3 may comprise or consist of SEQ ID NO:159. In certain aspects, the V_{H} CDR1 may comprise or consist of SEQ ID NO:165, the V_{H} CDR2 may comprise or consist of SEQ ID NO:166, and the V_{H} CDR3 may comprise or consist of SEQ ID NO:167. In certain aspects, the V_{H} CDR1 may comprise or consist of SEQ ID NO:173, the V_{H} CDR2 may comprise or consist of SEQ ID NO: 174, and the V_{H} CDR3 may comprise or consist of SEQ ID NO: 175. In certain aspects, the V_{H} CDR1 may comprise or consist of SEQ ID NO:181, the V_{H} CDR2 may comprise or consist of SEQ ID NO:182, and the V_{H} CDR3 may comprise or consist of SEQ ID NO:183. In certain aspects, the V_{H} CDR1 may comprise or consist of SEQ ID NO:189, the V_{H} CDR2 may comprise or consist of SEQ ID NO:190, and the V_{H} CDR3 may comprise or consist of SEQ ID NO:191. In certain aspects, the V_{H} CDR1 may comprise or consist of SEQ ID NO:205, the V_{H} CDR2 may comprise or consist of SEQ ID NO:206, and the V_{H} CDR3 may comprise or consist of SEQ ID NO:207. In certain aspects, the V_{H} CDR1 may comprise or consist of SEQ ID NO:213, the V_{H} CDR2 may comprise or consist of SEQ ID NO:214, and the V_{H} CDR3 may comprise or consist of SEQ ID NO:215. In certain aspects, the V_{H} CDR1 may comprise or consist of SEQ ID NO:221, the V_{H} CDR2 may comprise or consist of SEQ ID NO:222, and the V_{H} CDR3 may comprise or consist of SEQ ID NO:223. In certain aspects, the V_{H} CDR1 may comprise or consist of SEQ ID NO:229, the V_{L} CDR2 may comprise or consist of SEQ ID NO:230, and the V_{H} CDR3 may comprise or consist of SEQ ID NO:231. In certain aspects, the V_{H} CDR1 may comprise or consist of SEQ ID NO:237, the V_{H} CDR2 may comprise or consist of SEQ ID NO:238, and the V_{H} CDR3 may comprise or consist of SEQ ID NO:239. In certain aspects, the V_{H} CDR1 may comprise or consist of SEQ ID NO:245, the V_{H} CDR2 may comprise or consist of SEQ ID NO:246, and the V_{H} CDR3 may comprise or consist of SEQ ID NO:247. In certain aspects, the V_{H} CDR1 may comprise or consist of SEQ ID NO:253, the V_{H} CDR2 may comprise or consist of SEQ ID NO:254, and the V_{H} CDR3 may comprise or consist of SEQ ID NO:255. In certain aspects, the V_{H} CDR1 may comprise or consist of SEQ ID NO:261, the V_{H} CDR2 may comprise or consist of SEQ ID NO:262, and the V_{H} CDR3 may comprise or consist of SEQ ID NO:263. In certain aspects, the V_{H} CDR1 may comprise or consist of SEQ ID NO:269, the V_{H} CDR2 may comprise or consist of SEQ ID NO:270, and the V_{H} CDR3 may comprise or consist of SEQ ID NO:271. In certain aspects, the V_{H} CDR1 may comprise or consist of SEQ ID NO:277, the V_{H} CDR2 may comprise or consist of SEQ ID NO:278, and the V_{H} CDR3 may comprise or consist of SEQ ID NO:279. In certain aspects, the V_{H} CDR1 may comprise or consist of SEQ ID NO:285, the V_{H} CDR2 may comprise or consist of SEQ ID NO:286, and the V_{H} CDR3 may comprise or consist of SEQ ID NO:287.

In one aspect the isolated antibody comprises a V_{L} CDR1, a V_{L} CDR2, V_{L} CDR3, a V_{H} CDR1, a V_{H} CDR2, and a V_{H} CDR3. In certain aspects, the V_{L} CDR1 may comprise or consist of SEQ ID NO:1, the V_{L} CDR2 may comprise or consist of SEQ ID NO:2, and the V_{L} CDR3 may comprise or consist of SEQ ID NO:3. In certain aspects, the V_{L} CDR1 may comprise or consist of SEQ ID NO:9, the V_{L} CDR2 may comprise or consist of SEQ ID NO:10, and the V_{L} CDR3 may comprise or consist of SEQ ID NO:11. In certain aspects, the V_{L} CDR1 may comprise or consist of SEQ ID NO:17, the V_{L} CDR2 may comprise or consist of SEQ ID NO:18, and the V_{L} CDR3 may comprise or consist of SEQ ID NO:19. In certain aspects, the V_{L} CDR1 may comprise or consist of SEQ ID NO:25, the V_{L} CDR2 may comprise or consist of SEQ ID NO:26, and the V_{L} CDR3 may comprise or consist of SEQ ID NO:27. In certain aspects, the V_{L} CDR1 may comprise or consist of SEQ ID NO:33, the V_{L} CDR2 may comprise or consist of SEQ ID NO:34, and the V_{L} CDR3 may comprise or consist of SEQ ID NO:35. In certain aspects, the V_{L} CDR1 may comprise or consist of SEQ ID NO:41, the V_{L} CDR2 may comprise or consist of SEQ ID NO:42, and the V_{L} CDR3 may comprise or consist of SEQ ID NO:43. In certain aspects, the V_{L} CDR1 may comprise or consist of SEQ ID NO:49, the V_{L} CDR2 may comprise or consist of SEQ ID NO:50, and the V_{L} CDR3 may comprise or consist of SEQ ID NO:51. In certain aspects, the V_{L} CDR1 may comprise or consist of SEQ ID NO:57, the V_{L} CDR2 may comprise or consist of SEQ ID NO:58, and the V_{L} CDR3 may comprise or consist of SEQ ID NO:59. In certain aspects, the V_{L} CDR1 may comprise or consist of SEQ ID NO:65, the V_{L} CDR2 may comprise or consist of SEQ ID NO:66, and the V_{L} CDR3 may comprise or consist of SEQ ID NO:67. In certain aspects, the V_{L} CDR1 may comprise or consist of SEQ ID NO:73, the V_{L} CDR2 may comprise or consist of SEQ ID NO:74, and the V_{L} CDR3 may comprise or consist of SEQ ID NO:75. In certain aspects, the V_{L} CDR1 may comprise or consist of SEQ ID NO:81, the V_{L} CDR2 may comprise or consist of SEQ ID NO:82, and the V_{L} CDR3 may comprise or consist of SEQ ID NO:83. In certain aspects, the V_{L} CDR1 may comprise or consist of SEQ ID NO:89, the V_{L} CDR2 may comprise or consist of SEQ ID NO:90, and the V_{L} CDR3 may comprise or consist of SEQ ID NO:91. In certain aspects, the V_{L} CDR1 may comprise or consist of SEQ ID NO:97, the V_{L} CDR2 may comprise or consist of EQ ID NO:98, and the V_{L} CDR3 may comprise or consist of SEQ ID NO:99. In certain aspects, the V_{L} CDR1 may comprise or consist of SEQ ID NO:105, the V_{L} CDR2 may comprise or consist of SEQ ID NO:106, and the V_{L} CDR3 may comprise or consist of SEQ ID NO:107. In certain aspects, the V_{L} CDR1 may comprise or consist of SEQ ID NO:113, the V_{L} CDR2 may comprise or consist of SEQ ID NO:114, and the V_{L} CDR3 may comprise or consist of SEQ ID NO: 115. In certain aspects, the V_{L} CDR1 may comprise or consist of SEQ ID NO:121, the V_{L} CDR2 may comprise or consist of SEQ ID NO: 122, and the V_{L} CDR3 may comprise or consist of SEQ ID NO:123. In certain aspects, the V_{L} CDR1 may comprise or consist of SEQ ID NO:129, the V_{L} CDR2 may comprise or consist of SEQ ID NO: 130, and the V_{L} CDR3 may comprise or consist of SEQ ID NO:131. In certain aspects, the V_{L} CDR1 may comprise or consist of SEQ ID NO:137, the V_{L} CDR2 may comprise or consist of SEQ ID NO:138, and the V_{L} CDR3 may comprise or consist of SEQ ID NO:139. In certain aspects, the V_{L} CDR1 may comprise or consist of SEQ ID NO:145, the V_{L} CDR2 may comprise or consist of SEQ ID NO:146, and the V_{L} CDR3 may comprise or consist of SEQ ID NO:147. In certain aspects, the V_{L} CDR1 may comprise or consist of SEQ ID NO:153, the V_{L} CDR2 may comprise or consist of SEQ ID NO:154, and the V_{L} CDR3 may comprise or consist of SEQ ID NO:155. In certain aspects, the V_{L} CDR1 may comprise or consist of SEQ ID NO:161, the V_{L} CDR2 may comprise or consist of SEQ ID NO:162, and the V_{L} CDR3 may comprise or consist of SEQ ID NO:163. In certain aspects, the V_{L} CDR1 may comprise or consist of SEQ ID NO:169, the V_{L} CDR2 may comprise or consist of SEQ ID NO:170, and the V_{L} CDR3 may comprise or consist of SEQ ID NO:171. In certain aspects, the V_{L} CDR1 may comprise or consist of SEQ ID NO:177, the V_{L} CDR2 may comprise or consist of SEQ ID NO:178, and the V_{L} CDR3 may comprise or consist of SEQ ID NO:179. In certain aspects, the V_{L} CDR1 may comprise or consist of SEQ ID NO:185, the V_{L} CDR2 may comprise or consist of SEQ ID NO:186, and the V_{L} CDR3 may comprise or consist of SEQ ID NO:187. In certain aspects, the V_{L} CDR1 may comprise or consist of SEQ ID NO:193, the V_{L} CDR2 may comprise or consist of SEQ ID NO:194, and the V_{L} CDR3 may comprise or consist of SEQ ID NO:195. In certain aspects, the V_{L} CDR1 may comprise or consist of SEQ ID NO:201, the V_{L} CDR2 may comprise or consist of SEQ ID NO:202, and the V_{L} CDR3 may comprise or consist of SEQ ID NO:203. In certain aspects, the V_{L} CDR1 may comprise or consist of SEQ ID NO:209, the V_{L} CDR2 may comprise or consist of SEQ ID NO:210, and the V_{L} CDR3 may comprise or consist of SEQ ID NO:211. In certain aspects, the V_{L} CDR1 may comprise or consist of SEQ ID NO:217, the V_{L} CDR2 may comprise or consist of SEQ ID NO:218, and the V_{L} CDR3 may comprise or consist of SEQ ID NO:219. In certain aspects, the V_{L} CDR1 may comprise or consist of SEQ ID NO:225, the V_{L} CDR2 may comprise or consist of SEQ ID NO:226, and the V_{L} CDR3 may comprise or consist of SEQ ID NO:227. In certain aspects, the V_{L} CDR1 may comprise or consist of SEQ ID NO:233, the V_{L} CDR2 may comprise or consist of SEQ ID NO:234, and the V_{L} CDR3 may comprise or consist of SEQ ID NO:235. In certain aspects, the V_{L} CDR1 may comprise or consist of SEQ ID NO:241, the V_{L} CDR2 may comprise or consist of SEQ ID NO:242, and the V_{L} CDR3 may comprise or consist of SEQ ID NO:243. In certain aspects, the V_{L} CDR1 may comprise or consist of SEQ ID NO:249, the V_{L} CDR2 may comprise or consist of SEQ ID NO:250, and the V_{L} CDR3 may comprise or consist of SEQ ID NO:251. In certain aspects, the V_{L} CDR1 may comprise or consist of SEQ ID NO:257, the V_{L} CDR2 may comprise or consist of SEQ ID NO:258, and the V_{L} CDR3 may comprise or consist of SEQ ID NO:259. In certain aspects, the V_{L} CDR1 may comprise or consist of SEQ ID NO:265, the V_{L} CDR2 may comprise or consist of SEQ ID NO:266, and the V_{L} CDR3 may comprise or consist of SEQ ID NO:267. In certain aspects, the V_{L} CDR1 may comprise or consist of SEQ ID NO:273, the V_{L} CDR2 may comprise or consist of SEQ ID NO:274, and the V_{L} CDR3 may comprise or consist of SEQ ID NO:275. In certain aspects, the V_{L} CDR1 may comprise or consist of SEQ ID NO:281, the V_{L} CDR2 may comprise or consist of SEQ ID NO:282, and the V_{H} CDR3 may comprise or consist of SEQ ID NO:283. In certain aspects, the V_{H} CDR1 may comprise or consist of SEQ ID NO:5, the V_{H} CDR2 may comprise or consist of SEQ ID NO:6, and the V_{H} CDR3 may comprise or consist of SEQ ID NO:7. In certain aspects, the V_{H} CDR1 may comprise or consist of SEQ ID NO:13, the V_{H} CDR2 may comprise or consist of SEQ ID NO:14, and the V_{H} CDR3 may comprise or consist of SEQ ID NO: 15. In certain aspects, the V_{H} CDR1 may comprise or consist of SEQ ID NO:21, the V_{H} CDR2 may comprise or consist of SEQ ID NO:22, and the V_{H} CDR3 may comprise or consist of SEQ ID NO:23. In certain aspects, the V_{H} CDR1 may comprise or consist of SEQ ID NO:29, the V_{H} CDR2 may comprise or consist of SEQ ID NO:30, and the V_{H} CDR3 may comprise or consist of SEQ ID NO:31. In certain aspects, the V_{H} CDR1 may comprise or consist of SEQ ID NO:37, V_{H} CDR2 may comprise or consist of SEQ ID NO:38, and the V_{H} CDR3 may comprise or consist of SEQ ID NO:39. In certain aspects, the V_{H} CDR1 may comprise or consist of SEQ ID NO:45, the V_{H} CDR2 may comprise or consist of SEQ ID NO:46, and the V_{H} CDR3 may comprise or consist of SEQ ID NO:47. In certain aspects, the V_{H} CDR1 may comprise or consist of SEQ ID NO:53, the V_{H} CDR2 may comprise or consist of SEQ ID NO:54, and the V_{H} CDR3 may comprise or consist of SEQ ID NO:55. In certain aspects, the V_{H} CDR1 may comprise or consist of SEQ ID NO:61, the V_{H} CDR2 may comprise or consist of SEQ ID NO:62, and the V_{H} CDR3 may comprise or consist of SEQ ID NO:63. In certain aspects, the V_{H} CDR1 may comprise or consist of SEQ ID NO:69, the V_{H} may comprise or consist of SEQ ID NO:70, and the V_{H} CDR3 may comprise or consist of SEQ ID NO:71. In certain aspects, the V_{H} CDR1 may comprise or consist of SEQ ID NO:77, the V_{H} CDR2 may comprise or consist of SEQ ID NO:78, and the V_{H} CDR3 may comprise or consist of SEQ ID NO:79. In certain aspects, the V_{H} CDR1 may comprise or consist of SEQ ID NO:85, the V_{H} CDR2 may comprise or consist of SEQ ID NO:86, and the V_{H} CDR3 may comprise or consist of SEQ ID NO:87. In certain aspects, the V_{H} CDR1 may comprise or consist of SEQ ID NO:93, the V_{H} CDR2 may comprise or consist of SEQ ID NO:94, and the V_{H} CDR3 may comprise or consist of SEQ ID NO:95. In certain aspects, the V_{H} CDR1 may comprise or consist of SEQ ID NO:101, the V_{H} CDR2 may comprise or consist of SEQ ID NO:102, and the V_{H} CDR3 may comprise or consist of SEQ ID NO:103. In certain aspects, the V_{H} CDR1 may comprise or consist of SEQ ID NO:109, the V_{H} CDR2 may comprise or consist of SEQ ID NO:110, and the V_{H} CDR3 may comprise or consist of SEQ ID NO:111. In certain aspects, the V_{H} CDR1 may comprise or consist of SEQ ID NO:117, the V_{H} CDR2 may comprise or consist of SEQ ID NO:118, and the V_{H} CDR3 may comprise or consist of SEQ ID NO:119. In certain aspects, the V_{H} CDR1 may comprise or consist of SEQ ID NO:125, the V_{H} CDR2 may comprise or consist of SEQ ID NO:126, and the V_{H} CDR3 may comprise or consist of SEQ ID NO:127. In certain aspects, the V_{H} CDR1 may comprise or consist of SEQ ID NO:133, the V_{H} CDR2 may comprise or consist of SEQ ID NO:134, and the V_{H} CDR3 may comprise or consist of SEQ ID NO:135. In certain aspects, the V_{H} CDR1 may comprise or consist of SEQ ID NO:141, the V_{H} CDR2 may comprise or consist of SEQ ID NO:142, and the V_{H} CDR3 may comprise or consist of SEQ ID NO:143. In certain aspects, the V_{H} CDR1 may comprise or consist of SEQ ID NO:149, the V_{H} CDR2 may comprise or consist of SEQ ID NO:150, and the V_{H} CDR3 may comprise or consist of SEQ ID NO:151. In certain aspects, the V_{H} CDR1 may comprise or consist of SEQ ID NO:157, the V_{H} CDR2 may comprise or consist of SEQ ID NO:158, and the V_{H} CDR3 may comprise or consist of SEQ ID NO:159. In certain aspects, the V_{H} CDR1 may comprise or consist of SEQ ID NO: 165, the V_{H} CDR2 may comprise or consist of SEQ ID NO:166, and the V_{H} CDR3 may comprise or consist of SEQ ID NO:167. In certain aspects, the V_{H} CDR1 may comprise or consist of SEQ ID NO:173, the V_{H} CDR2 may comprise or consist of SEQ ID NO:174, and the V_{H} CDR3 may comprise or consist of SEQ ID NO:175, In certain aspects, the V_{H} CDR1 may comprise or consist of SEQ ID NO:181, the V_{H} CDR2 may comprise or consist of SEQ ID NO:182, and the V_{H} CDR3 may comprise or consist of SEQ ID NO:183. In certain aspects, the V_{H} CDR1 may comprise or consist of SEQ ID NO:189, the V_{H} CDR2 may comprise or consist of SEQ ID NO:190, and the V_{H} CDR3 may comprise or consist of SEQ ID NO:191. In certain aspects, the V_{H} CDR1 may comprise or consist of SEQ ID NO:197, the V_{H} CDR2 may comprise or consist of SEQ ID NO:198, and the V_{H} CDR3 may comprise or consist of SEQ ID NO:199. In certain aspects, the V_{H} CDR1 may comprise or consist of SEQ ID NO:205, the V_{H} CDR2 may comprise or consist of SEQ ID NO:206, and the V_{H} CDR3 may comprise or consist of SEQ ID NO:207. In certain aspects, the V_{H} CDR1 may comprise or consist of SEQ ID NO:213, the V_{H} CDR2 may comprise or consist of SEQ ID NO:214, and the V_{H} CDR3 may comprise or consist of SEQ ID NO:215. In certain aspects, the V_{H} CDR1 may comprise or consist of SEQ ID NO:221, the V_{H} CDR2 may comprise or consist of SEQ ID NO:222, and the V_{H} CDR3 may comprise or consist of SEQ ID NO:223. In certain aspects, the V_{H} CDR1 may comprise or consist of SEQ ID NO:229, the V_{H} CDR2 may comprise or consist of SEQ ID NO:230, and the V_{H} CDR3 may comprise or consist of SEQ ID NO:231. In certain aspects, the V_{H} CDR1 may comprise or consist of SEQ ID NO:237, the V_{H} CDR2 may comprise or consist of SEQ ID NO:238, and the V_{H} CDR3 may comprise or consist of SEQ ID NO:239. In certain aspects, the V_{H} CDR1 may comprise or consist of SEQ ID NO:245, the V_{H} CDR2 may comprise or consist of SEQ ID NO:246, and the V_{H} CDR3 may comprise or consist of SEQ ID NO:247. In certain aspects, the V_{H} CDR1 may comprise or consist of SEQ ID NO:253, the V_{H} CDR2 may comprise or consist of SEQ ID NO:254, and the V_{H} CDR3 may comprise or consist of SEQ ID NO:255. In certain aspects, the V_{H} CDR1 may comprise or consist of SEQ ID NO:261, the V_{H} CDR2 may comprise or consist of SEQ ID NO:262, and the V_{H} CDR3 may comprise or consist of SEQ ID NO:263. In certain aspects, the V_{H} CDR1 may comprise or consist of SEQ ID NO:269, the V_{H} CDR2 may comprise or consist of SEQ ID NO:270, and the V_{H} CDR3 may comprise or consist of SEQ ID NO:271. In certain aspects, the V_{H} CDR1 may comprise or consist of SEQ ID NO:277, the V_{H} CDR2 may comprise or consist of SEQ ID NO:278, and the V_{H} CDR3 may comprise or consist of SEQ ID NO:279. In certain aspects, the V_{H} CDR1 may comprise or consist of SEQ ID NO:285, the V_{H} CDR2 may comprise or consist of SEQ ID NO:286, and the V_{H} CDR3 may comprise or consist of SEQ ID NO:287.

In certain aspects, the V_{L} CDR1 may comprise or consist of SEQ ID NO:1, the V_{L} CDR2 may comprise or consist of SEQ ID NO:2, the V_{L} CDR3 may comprise or consist of SEQ ID NO:3, the V_{H} CDR1 may comprise or consist of SEQ ID NO:5, the V_{H} CDR2 may comprise or consist of SEQ ID NO:6, and the V_{H} CDR3 may comprise or consist of SEQ ID NO:7. In certain aspects, the V_{L} CDR1 may comprise or consist of SEQ ID NO:9, the V_{L} CDR2 may comprise or consist of SEQ ID NO:10, the V_{L} CDR3 may comprise or consist of SEQ ID NO:11, the V_{H} CDR1 may comprise or consist of SEQ ID NO:13, the V_{H} CDR2 may comprise or consist of SEQ ID NO:14, and the V_{L} CDR3 may comprise or consist of SEQ ID NO:15. In certain aspects, the V_{L} CDR1 may comprise or consist of SEQ ID NO:17, the V_{L} CDR2 may comprise or consist of SEQ ID NO:18, the V_{L} CDR3 may comprise or consist of SEQ ID NO:19, the V_{H} CDR1 may comprise or consist of SEQ ID NO:21, the V_{H} CDR2 may comprise or consist of SEQ ID NO:22, and the V_{H} CDR3 may comprise or consist of SEQ ID NO:23. In certain aspects, the V_{L} CDR1 may comprise or consist of SEQ ID NO:25, the V_{L} CDR2 may comprise or consist of SEQ ID NO:26, the V_{L} CDR3 may comprise or consist of SEQ ID NO:27, the V_{H} CDR1 may comprise or consist of SEQ ID NO:29, the V_{H} CDR2 may comprise or consist of SEQ ID NO:30, and the V_{H} CDR3 may comprise or consist of SEQ ID NO:31. In certain aspects, the V_{L} CDR1 may comprise or consist of SEQ ID NO:33, the V_{L} CDR2 may comprise or consist of SEQ ID NO:34, and the V_{L} CDR3 may comprise or consist of SEQ ID NO:35, the V_{H} CDR1 may comprise or consist of SEQ ID NO:37, the V_{H} CDR2 may comprise or consist of SEQ ID NO:38, and the V_{H} CDR3 may comprise or consist of SEQ ID NO:39. In certain aspects, the V_{L} CDR1 may comprise or consist of SEQ ID NO:41, the V_{L} CDR2 may comprise or consist of SEQ ID NO:42, the V_{L} CDR3 may comprise or consist of SEQ ID NO:43, the V_{H} CDR1 may comprise or consist of SEQ ID NO:45, the V_{H} CDR2 may comprise or consist of SEQ ID NO:46, and the V_{H} CDR3 may comprise or consist of SEQ ID NO:47. In certain aspects, the V_{L} CDR1 may comprise or consist of SEQ ID NO:49, the V_{L} CDR2 may comprise or consist of SEQ ID NO:50, the V_{L} CDR3 may comprise or consist of SEQ ID NO:51, the V_{H} CDR1 may comprise or consist of SEQ ID NO:53, the V_{H} CDR2 may comprise or consist of SEQ ID NO:54, and the V_{H} CDR3 may comprise or consist of SEQ ID NO:55. In certain aspects, the V_{L} CDR1 may comprise or consist of SEQ ID NO:57, the V_{L} CDR2 may comprise or consist of SEQ ID NO:58, the V_{L} CDR3 may comprise or consist of SEQ ID NO:59, the V_{H} CDR1 may comprise or consist of SEQ ID NO:61, the V_{H} CDR2 may comprise or consist of SEQ ID NO:62, and the V_{H} CDR3 may comprise or consist of SEQ ID NO:63. In certain aspects, the V_{L} CDR1 may comprise or consist of SEQ ID NO:65, the V_{L} CDR2 may comprise or consist of SEQ ID NO:66, the V_{L} CDR3 may comprise or consist of SEQ ID NO:67, the V_{H} CDR1 may comprise or consist of SEQ ID NO:69, the V_{H} CDR2 may comprise or consist of SEQ ID NO:70, and the V_{H} CDR3 may comprise or consist of SEQ ID NO:71. In certain aspects, the V_{L} CDR1 may comprise or consist of SEQ ID NO:73, the V_{L} CDR2 may comprise or consist of SEQ ID NO:74, the V_{L} CDR3 may comprise or consist of SEQ ID NO:75, the V_{H} CDR1 may comprise or consist of SEQ ID NO:77, the V_{H} CDR2 may comprise or consist of SEQ ID NO:78, and the V_{H} CDR3 may comprise or consist of SEQ ID NO:79. In certain aspects, the V_{L} CDR1 may comprise or consist of SEQ ID NO:81, the V_{L} CDR2 may comprise or consist of SEQ ID NO:82, the V_{L} CDR3 may comprise or consist of SEQ ID NO:83, the V_{H} CDR1 may comprise or consist of SEQ ID NO:85, the V_{H} CDR2 may comprise or consist of SEQ ID NO:86, and the V_{H} CDR3 may comprise or consist of SEQ ID NO:87. In certain aspects, the V_{L} CDR1 may comprise or consist of SEQ ID NO:89, the V_{L} CDR2 may comprise or consist of SEQ ID NO:90, the V_{L} CDR3 may comprise or consist of SEQ ID NO:91, the V_{H} CDR1 may comprise or consist of SEQ ID NO:93, the V_{H} CDR2 may comprise or consist of SEQ ID NO:94, and the V_{H} CDR3 may comprise or consist of SEQ ID NO:95. In certain aspects, the V_{L} CDR1 may comprise or consist of SEQ ID NO:97, the V_{L} CDR2 may comprise or consist of SEQ ID NO:98, V_{L} CDR3 may comprise or consist of SEQ ID NO:99, the V_{H} CDR1 may comprise or consist of SEQ ID NO:101, the V_{H} CDR2 may comprise or consist of SEQ ID NO:102, and the V_{H} CDR3 may comprise or consist of SEQ ID NO:103. In certain aspects, the V_{L} CDR1 may comprise or consist of SEQ ID NO:105, the V_{L} CDR2 may comprise or consist of SEQ ID NO:106, the V_{L} CDR3 may comprise or consist of SEQ ID NO:107, the V_{H} CDR1 may comprise or consist of SEQ ID NO:109, the V_{H} CDR2 may comprise or consist of SEQ ID NO:110, and the V_{H} CDR3 may comprise or consist of SEQ ID NO:111. In certain aspects, the V_{L} CDR1 may comprise or consist of SEQ ID NO:113, the V_{L} CDR2 may comprise or consist of SEQ ID NO:114, the V_{L} CDR3 may comprise or consist of SEQ ID NO: 115, the V_{H} CDR1 may comprise or consist of SEQ ID NO:117, the V_{L} CDR2 may comprise or consist of SEQ ID NO:118, and the V_{H} CDR3 may comprise or consist of SEQ ID NO:119. In certain aspects, the V_{L} CDR1 may comprise or consist of SEQ ID NO:121, the V_{L} CDR2 may comprise or consist of SEQ ID NO:122, the V_{L} CDR3 may comprise or consist of SEQ ID NO: 123, the V_{H} CDR1 may comprise or consist of SEQ ID NO: 125, the V_{H} CDR2 may comprise or consist of SEQ ID NO:126, and the V_{H} CDR3 may comprise or consist of SEQ ID NO:127. In certain aspects, the V_{L} CDR1 may comprise or consist of SEQ ID NO:129, the V_{L} CDR2 may comprise or consist of SEQ ID NO:130, the V_{L} CDR3 may comprise or consist of SEQ ID NO:131, the V_{H} CDR1 may comprise or consist of SEQ ID NO:133, the V_{H} CDR2 may comprise or consist of SEQ ID NO:134, and the V_{H} CDR3 may comprise or consist of SEQ ID NO:135. In certain aspects, the V_{L} CDR1 may comprise or consist of SEQ ID NO:137, the V_{L} CDR2 may comprise or consist of SEQ ID NO:138, the V_{L} CDR3 may comprise or consist of SEQ ID NO:139, the V_{H} CDR1 may comprise or consist of SEQ ID NO:141, the V_{H} CDR2 may comprise or consist of SEQ ID NO:142, and the V_{H} CDR3 may comprise or consist of SEQ ID NO:143. In certain aspects, V_{L} CDR1 may comprise or consist of SEQ ID NO:145, the V_{L} CDR2 may comprise or consist of SEQ ID NO:146, the V_{L} CDR3 may comprise or consist of SEQ ID NO:147, the V_{H} CDR1 may comprise or consist of SEQ ID NO:149, the V_{H} CDR2 may comprise or consist of SEQ ID NO:150, and the V_{H} CDR3 may comprise or consist of SEQ ID NO:151. In certain aspects, the V_{L} CDR1 may comprise or consist of SEQ ID NO:153, the V_{L} CDR2 may comprise or consist of SEQ ID NO:154, the V_{L} CDR3 may comprise or consist of SEQ ID NO:155, the V_{H} CDR1 may comprise or consist of SEQ ID NO:157, the V_{H} CDR2 may comprise or consist of SEQ ID NO:158, and the V_{H} CDR3 may comprise or consist of SEQ ID NO:159. In certain aspects, the V_{L} CDR1 may comprise or consist of SEQ ID NO:161, the V_{L} CDR2 may comprise or consist of SEQ ID NO:162, the V_{L} CDR3 may comprise or consist of SEQ ID NO:163, the V_{H} CDR1 may comprise or consist of SEQ ID NO:165, the V_{H} CDR2 may comprise or consist of SEQ ID NO:166, and the V_{H} CDR3 may comprise or consist of SEQ ID NO:167. In certain aspects, the V_{L} CDR1 may comprise or consist of SEQ ID NO:169, the V_{L} CDR2 may comprise or consist of SEQ ID NO:170, the V_{L} CDR3 may comprise or consist of SEQ ID NO:171, the V_{H} CDR1 may comprise or consist of SEQ ID NO:173, the V_{H} CDR2 may comprise or consist of SEQ ID NO:174, and the V_{H} CDR3 may comprise or consist of SEQ ID NO:175. In certain aspects, the V_{L} CDR1 may comprise or consist of SEQ ID NO:177, the V_{L} CDR2 may comprise or consist of SEQ ID NO:178, the V_{L} CDR3 may comprise or consist of SEQ ID NO:179, the V_{H} CDR1 may comprise or consist of SEQ ID NO:181, the V_{L} CDR2 may comprise or consist of SEQ ID NO:182, and the V_{H} CDR3 may comprise or consist of SEQ ID NO:183. In certain aspects, the V_{L} CDR1 may comprise or consist of SEQ ID NO: 185, the V_{L} CDR2 may comprise or consist of SEQ ID NO:186, the V_{L} CDR3 may comprise or consist of SEQ ID NO:187, the V_{H} CDR1 may comprise or consist of SEQ ID NO:189, the V_{H} CDR2 may comprise or consist of SEQ ID NO:190, and the V_{H} CDR3 may comprise or consist of SEQ ID NO:191. In certain aspects, the V_{L} CDR1 may comprise or consist of SEQ ID NO:193, the V_{L} CDR2 may comprise or consist of SEQ ID NO:194, the V_{L} CDR3 may comprise or consist of SEQ ID NO:195, the V_{H} CDR1 may comprise or consist of SEQ ID NO:197, the V_{H} CDR2 may comprise or consist of SEQ ID NO:198, and the V_{H} CDR3 may comprise or consist of SEQ ID NO:199. In certain aspects, the V_{L} CDR1 may comprise or consist of SEQ ID NO:201, the V_{L} CDR2 may comprise or consist of SEQ ID NO:202, the V_{L} CDR3 may comprise or consist of SEQ ID NO:203, the V_{H} CDR1 may comprise or consist of SEQ ID NO:205, the V_{H} CDR2 may comprise or consist of SEQ ID NO:206, and the V_{H} CDR3 may comprise or consist of SEQ ID NO:207. In certain aspects, the V_{L} CDR1 may comprise or consist of SEQ ID NO:209, the V_{L} CDR2 may comprise or consist of SEQ ID NO:210, the V_{L} CDR3 may comprise or consist of SEQ ID NO:211, the V_{L} CDR1 may comprise or consist of SEQ ID NO:213, the V_{H} CDR2 may comprise or consist of SEQ ID NO:214, and the V_{H} CDR3 may comprise or consist of SEQ ID NO:215. In certain aspects, the V_{L} CDR1 may comprise or consist of SEQ ID NO:217, the V_{L} CDR2 may comprise or consist of SEQ ID NO:218, the V_{L} CDR3 may comprise or consist of SEQ ID NO:219, the V_{H} CDR1 may comprise or consist of SEQ ID NO:221, the V_{H} CDR2 may comprise or consist of SEQ ID NO:222, and the V_{H} CDR3 may comprise or consist of SEQ ID NO:223. In certain aspects, the V_{L} CDR1 may comprise or consist of SEQ ID NO:225, the V_{L} CDR2 may comprise or consist of SEQ ID NO:226, the V_{L} CDR3 may comprise or consist of SEQ ID NO:227, the V_{L} CDR1 may comprise or consist of SEQ ID NO:229, the V_{H} CDR2 may comprise or consist of SEQ ID NO:230, and the V_{H} CDR3 may comprise or consist of SEQ ID NO:231. In certain aspects, the V_{L} CDR1 may comprise or consist of SEQ ID NO:233, the V_{L} CDR2 may comprise or consist of SEQ ID NO:234, the V_{L} CDR3 may comprise or consist of SEQ ID NO:235, the V_{H} CDR1 may comprise or consist of SEQ ID NO:237, the V_{H} CDR2 may comprise or consist of SEQ ID NO:238, and the V_{H} CDR3 may comprise or consist of SEQ ID NO:239. In certain aspects, the V_{L} CDR1 may comprise or consist of SEQ ID NO:241, the V_{L} CDR2 may comprise or consist of SEQ ID NO:242, the V_{L} CDR3 may comprise or consist of SEQ ID NO:243, the V_{H} CDR1 may comprise or consist of SEQ ID NO:245, the V_{H} CDR2 may comprise or consist of SEQ ID NO:246, and the V_{H} CDR3 may comprise or consist of SEQ ID NO:247. In certain aspects, the V_{L} CDR1 may comprise or consist of SEQ ID NO:249, the V_{L} CDR2 may comprise or consist of SEQ ID NO:250, the V_{L} CDR3 may comprise or consist of SEQ ID NO:251, the V_{H} CDR1 may comprise or consist of SEQ ID NO:253, the V_{H} CDR2 may comprise or consist of SEQ ID NO:254, and the V_{H} CDR3 may comprise or consist of SEQ ID NO:255. In certain aspects, the V_{L} CDR1 may comprise or consist of SEQ ID NO:257, the V_{L} CDR2 may comprise or consist of SEQ ID NO:258, the V_{L} CDR3 may comprise or consist of SEQ ID NO:259, the V_{H} CDR1 may comprise or consist of SEQ ID NO:261, the V_{H} CDR2 may comprise or consist of SEQ ID NO:262, and the V_{H} CDR3 may comprise or consist of SEQ ID NO:263. In certain aspects, the V_{H} CDR1 may comprise or consist of SEQ ID NO:265, the V_{L} CDR2 may comprise or consist of SEQ ID NO:266, the V_{L} CDR3 may comprise or consist of SEQ ID NO:267, the V_{H} CDR1 may comprise or consist of SEQ ID NO:269, the V_{H} CDR2 may comprise or consist of SEQ ID NO:270, and the V_{H} CDR3 may comprise or consist of SEQ ID NO:271. In certain aspects, the V_{L} CDR1 may comprise or consist of SEQ ID NO:273, the V_{L} CDR2 may comprise or consist of SEQ ID NO:274, the V_{L} CDR3 may comprise or consist of SEQ ID NO:275, the V_{H} CDR1 may comprise or consist of SEQ ID NO:277, the V_{L} CDR2 may comprise or consist of SEQ ID NO:278, and the V_{H} CDR3 may comprise or consist of SEQ ID NO:279. In certain aspects, the V_{L} CDR1 may comprise or consist of SEQ ID NO:281, the V_{L} CDR2 may comprise or consist of SEQ ID NO:282, the V_{L} CDR3 may comprise or consist of SEQ ID NO:283, the V_{H} CDR1 may comprise or consist of SEQ ID NO:285, the V_{H} CDR2 may comprise or consist of SEQ ID NO:286, and the V_{H} CDR3 may comprise or consist of SEQ ID NO:287.

In one aspect, the isolated antibody may comprise a light chain comprising an amino acid sequence at least 80% identical, at least 85% identical, at least 90% identical, at least 95% identical, at last 97% identical, at least 99% identical, or 100% identical to SEQ ID NO:4, SEQ ID NO:12, SEQ ID NO:20, SEQ ID NO:28, SEQ ID NO:36, SEQ ID NO:44, SEQ ID NO:52, SEQ ID NO:60, SEQ ID NO:68, SEQ ID NO:76, SEQ ID NO:84, SEQ ID NO:92, SEQ ID NO:100, SEQ ID NO:108, SEQ ID NO:116, SEQ ID NO:124, SEQ ID NO:132, SEQ ID NO:140, SEQ ID NO:148, SEQ ID NO:156, SEQ ID NO:164, SEQ ID NO:172, SEQ ID NO:180, SEQ ID NO:188, SEQ ID NO:196, SEQ ID NO:204, SEQ ID NO:212, SEQ ID NO:220, SEQ ID NO:228, SEQ ID NO:236, SEQ ID NO:244, SEQ ID NO:252, SEQ ID NO:260, SEQ ID NO:268, SEQ ID NO:276, or SEQ ID NO:284.

In one aspect, the isolated antibody comprises a light chain selected from the group consisting of:
a light chain comprising an amino acid sequence at least 80% identical, at least 85% identical, at least 90% identical, at least 95% identical, at last 97% identical, or at least 99% identical to SEQ ID NO:4, wherein the light chain comprises V_{L} CDR1 comprising or consisting of SEQ ID NO:1, V_{L} CDR2 comprising or consisting of SEQ ID NO:2, and V_{L} CDR3 comprising or consisting of SEQ ID NO:3;
a light chain comprising an amino acid sequence at least 80% identical, at least 85% identical, at least 90% identical, at least 95% identical, at last 97% identical, or at least 99% identical to SEQ ID NO:12, wherein the light chain comprises V_{L} CDR1 comprising or consisting of SEQ ID NO:9, V_{L} CDR2 comprising or consisting of SEQ ID NO:10, and V_{L} CDR3 comprising or consisting of SEQ ID NO:11;
a light chain comprising an amino acid sequence at least 80% identical, at least 85% identical, at least 90% identical, at least 95% identical, at last 97% identical, or at least 99% identical to SEQ ID NO:20, wherein the light chain comprises V_{L} CDR1 comprising or consisting of SEQ ID NO:17, V_{L} CDR2 comprising or consisting of SEQ ID NO:18, and V_{L} CDR3 comprising or consisting of SEQ ID NO:19;
a light chain comprising an amino acid sequence at least 80% identical, at least 85% identical, at least 90% identical, at least 95% identical, at last 97% identical, or at least 99% identical to SEQ ID NO:28, wherein the light chain comprises V_{L} CDR1 comprising or consisting of SEQ ID NO:25, V_{L} CDR2 comprising or consisting of SEQ ID NO:26, and V_{L} CDR3 comprising or consisting of SEQ ID NO:27;
a light chain comprising an amino acid sequence at least 80% identical, at least 85% identical, at least 90% identical, at least 95% identical, at last 97% identical, or at least 99% identical to SEQ ID NO:36, wherein the light chain comprises V_{L} CDR1 comprising or consisting of SEQ ID NO:33, V_{L} CDR2 comprising or consisting of SEQ ID NO:34, and V_{L} CDR3 comprising or consisting of SEQ ID NO:35;
a light chain comprising an amino acid sequence at least 80% identical, at least 85% identical, at least 90% identical, at least 95% identical, at last 97% identical, or at least 99% identical to SEQ ID NO:44, wherein the light chain comprises V_{L} CDR1 comprising or consisting of SEQ ID NO:41, V_{L} CDR2 comprising or consisting of SEQ ID NO:42, and V_{L} CDR3 comprising or consisting of SEQ ID NO:43;
a light chain comprising an amino acid sequence at least 80% identical, at least 85% identical, at least 90% identical, at least 95% identical, at last 97% identical, or at least 99% identical to SEQ ID NO:52, wherein the light chain comprises V_{L} CDR1 comprising or consisting of SEQ ID NO:49, V_{L} CDR2 comprising or consisting of SEQ ID NO:50, and V_{L} CDR3 comprising or consisting of SEQ ID NO:51;
a light chain comprising an amino acid sequence at least 80% identical, at least 85% identical, at least 90% identical, at least 95% identical, at last 97% identical, or at least 99% identical to SEQ ID NO:60, wherein the light chain comprises V_{L} CDR1 comprising or consisting of SEQ ID NO:57, V_{L} CDR2 comprising or consisting of SEQ ID NO:58, and V_{L} CDR3 comprising or consisting of SEQ ID NO:59;
a light chain comprising an amino acid sequence at least 80% identical, at least 85% identical, at least 90% identical, at least 95% identical, at last 97% identical, or at least 99% identical to SEQ ID NO:68, wherein the light chain comprises V_{L} CDR1 comprising or consisting of SEQ ID NO:65, V_{L} CDR2 comprising or consisting of SEQ ID NO:66, and V_{L} CDR3 comprising or consisting of SEQ ID NO:67;
a light chain comprising an amino acid sequence at least 80% identical, at least 85% identical, at least 90% identical, at least 95% identical, at last 97% identical, or at least 99% identical to SEQ ID NO:76, wherein the light chain comprises V_{L} CDR1 comprising or consisting of SEQ ID NO:73, V_{L} CDR2 comprising or consisting of SEQ ID NO:74, and V_{L} CDR3 comprising or consisting of SEQ ID NO:75;
a light chain comprising an amino acid sequence at least 80% identical, at least 85% identical, at least 90% identical, at least 95% identical, at last 97% identical, or at least 99% identical to SEQ ID NO:84, wherein the light chain comprises V_{L} CDR1 comprising or consisting of SEQ ID NO:81, V_{L} CDR2 comprising or consisting of SEQ ID NO:82, and V_{L} CDR3 comprising or consisting of SEQ ID NO:83;
a light chain comprising an amino acid sequence at least 80% identical, at least 85% identical, at least 90% identical, at least 95% identical, at last 97% identical, or at least 99% identical to SEQ ID NO:92, wherein the light chain comprises V_{L} CDR1 comprising or consisting of SEQ ID NO:89, V_{L} CDR2 comprising or consisting of SEQ ID NO:90, and V_{L} CDR3 comprising or consisting of SEQ ID NO:91;
a light chain comprising an amino acid sequence at least 80% identical, at least 85% identical, at least 90% identical, at least 95% identical, at last 97% identical, or at least 99% identical to SEQ ID NO:100, wherein the light chain comprises V_{L} CDR1 comprising or consisting of SEQ ID NO:97, V_{L} CDR2 comprising or consisting of SEQ ID NO:98, and V_{L} CDR3 comprising or consisting of SEQ ID NO:99;
a light chain comprising an amino acid sequence at least 80% identical, at least 85% identical, at least 90% identical, at least 95% identical, at last 97% identical, or at least 99% identical to SEQ ID NO:108, wherein the light chain comprises V_{L} CDR1 comprising or consisting of SEQ ID NO:105, V_{L} CDR2 comprising or consisting of SEQ ID NO:106, and V_{L} CDR3 comprising or consisting of SEQ ID NO:107;
a light chain comprising an amino acid sequence at least 80% identical, at least 85% identical, at least 90% identical, at least 95% identical, at last 97% identical, or at least 99% identical to SEQ ID NO:116, wherein the light chain comprises V_{L} CDR1 comprising or consisting of SEQ ID NO:113, V_{L} CDR2 comprising or consisting of SEQ ID NO:114, and V_{L} CDR3 comprising or consisting of SEQ ID NO: 115;
a light chain comprising an amino acid sequence at least 80% identical, at least 85% identical, at least 90% identical, at least 95% identical, at last 97% identical, or at least 99% identical to SEQ ID NO:124, wherein the light chain comprises V_{L} CDR1 comprising or consisting of SEQ ID NO:121, V_{L} CDR2 comprising or consisting of SEQ ID NO:122, and V_{L} CDR3 comprising or consisting of SEQ ID NO:123;
a light chain comprising an amino acid sequence at least 80% identical, at least 85% identical, at least 90% identical, at least 95% identical, at last 97% identical, or at least 99% identical to SEQ ID NO:132, wherein the light chain comprises V_{L} CDR1 comprising or consisting of SEQ ID NO:129, V_{L} CDR2 comprising or consisting of SEQ ID NO:130, and V_{L} CDR3 comprising or consisting of SEQ ID NO:131;
a light chain comprising an amino acid sequence at least 80% identical, at least 85% identical, at least 90% identical, at least 95% identical, at last 97% identical, or at least 99% identical to SEQ ID NO:140, wherein the light chain comprises V_{L} CDR1 comprising or consisting of SEQ ID NO:137, V_{L} CDR2 comprising or consisting of SEQ ID NO:138, and V_{L} CDR3 comprising or consisting of SEQ ID NO:139;
a light chain comprising an amino acid sequence at least 80% identical, at least 85% identical, at least 90% identical, at least 95% identical, at last 97% identical, or at least 99% identical to SEQ ID NO:148, wherein the light chain comprises V_{L} CDR1 comprising or consisting of SEQ ID NO:145, V_{L} CDR2 comprising or consisting of SEQ ID NO:146, and V_{L} CDR3 comprising or consisting of SEQ ID NO:147;
a light chain comprising an amino acid sequence at least 80% identical, at least 85% identical, at least 90% identical, at least 95% identical, at last 97% identical, or at least 99% identical to SEQ ID NO:156, wherein the light chain comprises V_{L} CDR1 comprising or consisting of SEQ ID NO:153, V_{L} CDR2 comprising or consisting of SEQ ID NO: 154, and V_{L} CDR3 comprising or consisting of SEQ ID NO: 155;
a light chain comprising an amino acid sequence at least 80% identical, at least 85% identical, at least 90% identical, at least 95% identical, at last 97% identical, or at least 99% identical to SEQ ID NO:164, wherein the light chain comprises V_{L} CDR1 comprising or consisting of SEQ ID NO:161, V_{L} CDR2 comprising or consisting of SEQ ID NO: 162, and V_{L} CDR3 comprising or consisting of SEQ ID NO: 163;
a light chain comprising an amino acid sequence at least 80% identical, at least 85% identical, at least 90% identical, at least 95% identical, at last 97% identical, or at least 99% identical to SEQ ID NO:172, wherein the light chain comprises V_{L} CDR1 comprising or consisting of SEQ ID NO: 169, V_{L} CDR2 comprising or consisting of SEQ ID NO:170, and V_{L} CDR3 comprising or consisting of SEQ ID NO:171;
a light chain comprising an amino acid sequence at least 80% identical, at least 85% identical, at least 90% identical, at least 95% identical, at last 97% identical, or at least 99% identical to SEQ ID NO:180, wherein the light chain comprises V_{L} CDR1 comprising or consisting of SEQ ID NO: 177, V_{L} CDR2 comprising or consisting of SEQ ID NO:178, and V_{L} CDR3 comprising or consisting of SEQ ID NO:179;
a light chain comprising an amino acid sequence at least 80% identical, at least 85% identical, at least 90% identical, at least 95% identical, at last 97% identical, or at least 99% identical to SEQ ID NO:188, wherein the light chain comprises V_{L} CDR1 comprising or consisting of SEQ ID NO:185, V_{L} CDR2 comprising or consisting of SEQ ID NO:186, and V_{L} CDR3 comprising or consisting of SEQ ID NO:187;
a light chain comprising an amino acid sequence at least 80% identical, at least 85% identical, at least 90% identical, at least 95% identical, at last 97% identical, or at least 99% identical to SEQ ID NO:196, wherein the light chain comprises V_{L} CDR1 comprising or consisting of SEQ ID NO:193, V_{L} CDR2 comprising or consisting of SEQ ID NO:194, and V_{L} CDR3 comprising or consisting of SEQ ID NO:195;
a light chain comprising an amino acid sequence at least 80% identical, at least 85% identical, at least 90% identical, at least 95% identical, at last 97% identical, or at least 99% identical to SEQ ID NO:204, wherein the light chain comprises V_{L} CDR1 comprising or consisting of SEQ ID NO:201, V_{L} CDR2 comprising or consisting of SEQ ID NO:202, and V_{L} CDR3 comprising or consisting of SEQ ID NO:203;
a light chain comprising an amino acid sequence at least 80% identical, at least 85% identical, at least 90% identical, at least 95% identical, at last 97% identical, or at least 99% identical to SEQ ID NO:212, wherein the light chain comprises V_{L} CDR1 comprising or consisting of SEQ ID NO:209, V_{L} CDR2 comprising or consisting of SEQ ID NO:210, and V_{L} CDR3 comprising or consisting of SEQ ID NO:211;
a light chain comprising an amino acid sequence at least 80% identical, at least 85% identical, at least 90% identical, at least 95% identical, at last 97% identical, or at least 99% identical to SEQ ID NO:220, wherein the light chain comprises V_{L} CDR1 comprising or consisting of SEQ ID NO:217, V_{L} CDR2 comprising or consisting of SEQ ID NO:218, and V_{L} CDR3 comprising or consisting of SEQ ID NO:219;
a light chain comprising an amino acid sequence at least 80% identical, at least 85% identical, at least 90% identical, at least 95% identical, at last 97% identical, or at least 99% identical to SEQ ID NO:228, wherein the light chain comprises V_{L} CDR1 comprising or consisting of SEQ ID NO:225, V_{L} CDR2 comprising or consisting of SEQ ID NO:226, and V_{L} CDR3 comprising or consisting of SEQ ID NO:227;
a light chain comprising an amino acid sequence at least 80% identical, at least 85% identical, at least 90% identical, at least 95% identical, at last 97% identical, or at least 99% identical to SEQ ID NO:236, wherein the light chain comprises V_{L} CDR1 comprising or consisting of SEQ ID NO:233, V_{L} CDR2 comprising or consisting of SEQ ID NO:234, and V_{L} CDR3 comprising or consisting of SEQ ID NO:235;
a light chain comprising an amino acid sequence at least 80% identical, at least 85% identical, at least 90% identical, at least 95% identical, at last 97% identical, or at least 99% identical to SEQ ID NO:244, wherein the light chain comprises V_{L} CDR1 comprising or consisting of SEQ ID NO:241, V_{L} CDR2 comprising or consisting of SEQ ID NO:242, and V_{L} CDR3 comprising or consisting of SEQ ID NO:243;
a light chain comprising an amino acid sequence at least 80% identical, at least 85% identical, at least 90% identical, at least 95% identical, at last 97% identical, or at least 99% identical to SEQ ID NO:252, wherein the light chain comprises V_{L} CDR1 comprising or consisting of SEQ ID NO:249, V_{L} CDR2 comprising or consisting of SEQ ID NO:250, and V_{L} CDR3 comprising or consisting of SEQ ID NO:251;
a light chain comprising an amino acid sequence at least 80% identical, at least 85% identical, at least 90% identical, at least 95% identical, at last 97% identical, or at least 99% identical to SEQ ID NO:260, wherein the light chain comprises V_{L} CDR1 comprising or consisting of SEQ ID NO:257, V_{L} CDR2 comprising or consisting of SEQ ID NO:258, and V_{L} CDR3 comprising or consisting of SEQ ID NO:259;
a light chain comprising an amino acid sequence at least 80% identical, at least 85% identical, at least 90% identical, at least 95% identical, at last 97% identical, or at least 99% identical to SEQ ID NO:268, wherein the light chain comprises V_{L} CDR1 comprising or consisting of SEQ ID NO:265, V_{L} CDR2 comprising or consisting of SEQ ID NO:266, and V_{L} CDR3 comprising or consisting of SEQ ID NO:267;
a light chain comprising an amino acid sequence at least 80% identical, at least 85% identical, at least 90% identical, at least 95% identical, at last 97% identical, or at least 99% identical to SEQ ID NO:276, wherein the light chain comprises V_{L} CDR1 comprising or consisting of SEQ ID NO:273, V_{L} CDR2 comprising or consisting of SEQ ID NO:274, and V_{L} CDR3 comprising or consisting of SEQ ID NO:275; and,
a light chain comprising an amino acid sequence at least 80% identical, at least 85% identical, at least 90% identical, at least 95% identical, at last 97% identical, or at least 99% identical to SEQ ID NO:284, wherein the light chain comprises V_{L} CDR1 comprising or consisting of SEQ ID NO:281, V_{L} CDR2 comprising or consisting of SEQ ID NO:282, and V_{L} CDR3 comprising or consisting of SEQ ID NO:283.

In one aspect, the isolated antibody comprises a heavy chain comprising an amino acid sequence at least 80% identical, at least 85% identical, at least 90% identical, at least 95% identical, at last 97% identical, at least 99% identical, or 100% identical to SEQ ID NO:8, SEQ ID NO:16, SEQ ID NO:24, SEQ ID NO:32, SEQ ID NO:40, SEQ ID NO:48, SEQ ID NO:56, SEQ ID NO:64, SEQ ID NO:72, SEQ ID NO:80, SEQ ID NO:88, SEQ ID NO:96, SEQ ID NO:104, SEQ ID NO:112, SEQ ID NO:120, SEQ ID NO:128, SEQ ID NO:136, SEQ ID NO:144, SEQ ID NO:152, SEQ ID NO:160, SEQ ID NO:168, SEQ ID NO:176, SEQ ID NO:184, SEQ ID NO:192, SEQ ID NO:200, SEQ ID NO:208, SEQ ID NO:216, SEQ ID NO:224, SEQ ID NO:232, SEQ ID NO:240, SEQ ID NO:248, SEQ ID NO:256, SEQ ID NO:264, SEQ ID NO:272, SEQ ID NO:280, or SEQ ID NO:288.

In one aspect, the isolated antibody may comprise a heavy chain selected from the group consisting of:
a heavy chain comprising an amino acid sequence at least 80% identical, at least 85% identical, at least 90% identical, at least 95% identical, at last 97% identical, or at least 99% identical to SEQ ID NO:8, wherein the heavy chain comprises V_{H} CDR1 comprising or consisting of SEQ ID NO:5, V_{H} CDR2 comprising or consisting of SEQ ID NO:6, and V_{H} CDR3 comprising or consisting of SEQ ID NO:7;
a heavy chain comprising an amino acid sequence at least 80% identical, at least 85% identical, at least 90% identical, at least 95% identical, at last 97% identical, or at least 99% identical to SEQ ID NO:16, wherein the heavy chain comprises V_{H} CDR1 comprising or consisting of SEQ ID NO: 13, V_{H} CDR2 comprising or consisting of SEQ ID NO:14, and V_{H} CDR3 comprising or consisting of SEQ ID NO:15;
a heavy chain comprising an amino acid sequence at least 80% identical, at least 85% identical, at least 90% 213, at least 95% identical, at last 97% identical, or at least 99% identical to SEQ ID NO:24, wherein the heavy chain comprises V_{H} CDR1 comprising or consisting of SEQ ID NO:21, V_{H} CDR2 comprising or consisting of SEQ ID NO:22, and V_{H} CDR3 comprising or consisting of SEQ ID NO:23;
a heavy chain comprising an amino acid sequence at least 80% identical, at least 85% identical, at least 90% identical, at least 95% identical, at last 97% identical, or at least 99% identical to SEQ ID NO:32, wherein the heavy chain comprises V_{H} CDR1 comprising or consisting of SEQ ID NO:29, V_{H} CDR2 comprising or consisting of SEQ ID NO:30, and V_{H} CDR3 comprising or consisting of SEQ ID NO:31;
a heavy chain comprising an amino acid sequence at least 80% identical, at least 85% identical, at least 90% identical, at least 95% identical, at last 97% identical, or at least 99% identical to SEQ ID NO:40, wherein the heavy chain comprises V_{H} CDR1 comprising or consisting of SEQ ID NO:37, V_{H} CDR2 comprising or consisting of SEQ ID NO:38, and V_{H} CDR3 comprising or consisting of SEQ ID NO:39;
a heavy chain comprising an amino acid sequence at least 80% identical, at least 85% identical, at least 90% identical, at least 95% identical, at last 97% identical, or at least 99% identical to SEQ ID NO:48, wherein the heavy chain comprises V_{H} CDR1 comprising or consisting of SEQ ID NO:45, V_{H} CDR2 comprising or consisting of SEQ ID NO:46, and V_{H} CDR3 comprising or consisting of SEQ ID NO:47;
a heavy chain comprising an amino acid sequence at least 80% identical, at least 85% identical, at least 90% identical, at least 95% identical, at last 97% identical, or at least 99% identical to SEQ ID NO:56, wherein the heavy chain comprises V_{H} CDR1 comprising or consisting of SEQ ID NO:53, V_{H} CDR2 comprising or consisting of SEQ ID NO:54, and V_{H} CDR3 comprising or consisting of SEQ ID NO:55;
a heavy chain comprising an amino acid sequence at least 80% identical, at least 85% identical, at least 90% identical, at least 95% identical, at last 97% identical, or at least 99% identical to SEQ ID NO:64, wherein the heavy chain comprises V_{H} CDR1 comprising or consisting of SEQ ID NO:61, V_{H} CDR2 comprising or consisting of SEQ ID NO:62, and V_{H} CDR3 comprising or consisting of SEQ ID NO:63;
a heavy chain comprising an amino acid sequence at least 80% identical, at least 85% identical, at least 90% identical, at least 95% identical, at last 97% identical, or at least 99% identical to SEQ ID NO:72, wherein the heavy chain comprises V_{H} CDR1 comprising or consisting of SEQ ID NO:69, V_{H} CDR2 comprising or consisting of SEQ ID NO:70, and V_{H} CDR3 comprising or consisting of SEQ ID NO:71;
a heavy chain comprising an amino acid sequence at least 80% identical, at least 85% identical, at least 90% identical, at least 95% identical, at last 97% identical, or at least 99% identical to SEQ ID NO:80, wherein the heavy chain comprises V_{H} CDR1 comprising or consisting of SEQ ID NO:77, V_{H} CDR2 comprising or consisting of SEQ ID NO:78, and V_{H} CDR3 comprising or consisting of SEQ ID NO:79;
a heavy chain comprising an amino acid sequence at least 80% identical, at least 85% identical, at least 90% identical, at least 95% identical, at last 97% identical, or at least 99% identical to SEQ ID NO:88, wherein the heavy chain comprises V_{H} CDR1 comprising or consisting of SEQ ID NO:85, V_{H} CDR2 comprising or consisting of SEQ ID NO:86, and V_{H} CDR3 comprising or consisting of SEQ ID NO:87;
a heavy chain comprising an amino acid sequence at least 80% identical, at least 85% identical, at least 90% identical, at least 95% identical, at last 97% identical, or at least 99% identical to SEQ ID NO:96, wherein the heavy chain comprises V_{H} CDR1 comprising or consisting of SEQ ID NO:93, V_{H} CDR2 comprising or consisting of SEQ ID NO:94, and V_{H} CDR3 comprising or consisting of SEQ ID NO:95;
a heavy chain comprising an amino acid sequence at least 80% identical, at least 85% identical, at least 90% identical, at least 95% identical, at last 97% identical, or at least 99% identical to SEQ ID NO:104, wherein the heavy chain comprises V_{H} CDR1 comprising or consisting of SEQ ID NO:101, V_{H} CDR2 comprising or consisting of SEQ ID NO:102, and V_{H} CDR3 comprising or consisting of SEQ ID NO:103;
a heavy chain comprising an amino acid sequence at least 80% identical, at least 85% identical, at least 90% identical, at least 95% identical, at last 97% identical, or at least 99% identical to SEQ ID NO:112, wherein the heavy chain comprises V_{H} CDR1 comprising or consisting of SEQ ID NO:109, V_{H} CDR2 comprising or consisting of SEQ ID NO:110, and V_{H} CDR3 comprising or consisting of SEQ ID NO:111;
a heavy chain comprising an amino acid sequence at least 80% identical, at least 85% identical, at least 90% identical, at least 95% identical, at last 97% identical, or at least 99% identical to SEQ ID NO:120, wherein the heavy chain comprises V_{H} CDR1 comprising or consisting of SEQ ID NO:117, V_{H} CDR2 comprising or consisting of SEQ ID NO:118, and V_{H} CDR3 comprising or consisting of SEQ ID NO:119;
a heavy chain comprising an amino acid sequence at least 80% identical, at least 85% identical, at least 90% identical, at least 95% identical, at last 97% identical, or at least 99% identical to SEQ ID NO:128, wherein the heavy chain comprises V_{H} CDR1 comprising or consisting of SEQ ID NO:125, V_{H} CDR2 comprising or consisting of SEQ ID NO:126, and V_{H} CDR3 comprising or consisting of SEQ ID NO:127;
a heavy chain comprising an amino acid sequence at least 80% identical, at least 85% identical, at least 90% identical, at least 95% identical, at last 97% identical, or at least 99% identical to SEQ ID NO:136, wherein the heavy chain comprises V_{H} CDR1 comprising or consisting of SEQ ID NO:133, V_{H} CDR2 comprising or consisting of SEQ ID NO:134, and V_{H} CDR3 comprising or consisting of SEQ ID NO:135;
a heavy chain comprising an amino acid sequence at least 80% identical, at least 85% identical, at least 90% identical, at least 95% identical, at last 97% identical, or at least 99% identical to SEQ ID NO:144, wherein the heavy chain comprises V_{H} CDR1 comprising or consisting of SEQ ID NO:141, V_{H} CDR2 comprising or consisting of SEQ ID NO:142, and V_{H} CDR3 comprising or consisting of SEQ ID NO 143;
a heavy chain comprising an amino acid sequence at least 80% identical, at least 85% identical, at least 90% identical, at least 95% identical, at last 97% identical, or at least 99% identical to SEQ ID NO:152, wherein the heavy chain comprises V_{H} CDR1 comprising or consisting of SEQ ID NO:149, V_{H} CDR2 comprising or consisting of SEQ ID NO:150, and V_{H} CDR3 comprising or consisting of SEQ ID NO:151;
a heavy chain comprising an amino acid sequence at least 80% identical, at least 85% identical, at least 90% identical, at least 95% identical, at last 97% identical, or at least 99% identical to SEQ ID NO:160, wherein the heavy chain comprises V_{H} CDR1 comprising or consisting of SEQ ID NO:157, V_{H} CDR2 comprising or consisting of SEQ ID NO: 158, and V_{H} CDR3 comprising or consisting of SEQ ID NO:159;
a heavy chain comprising an amino acid sequence at least 80% identical, at least 85% identical, at least 90% identical, at least 95% identical, at last 97% identical, or at least 99% identical to SEQ ID NO:168, wherein the heavy chain comprises V_{H} CDR1 comprising or consisting of SEQ ID NO:165, V_{H} CDR2 comprising or consisting of SEQ ID NO: 166, and V_{H} CDR3 comprising or consisting of SEQ ID NO: 167;
a heavy chain comprising an amino acid sequence at least 80% identical, at least 85% identical, at least 90% identical, at least 95% identical, at last 97% identical, or at least 99% identical to SEQ ID NO:176, wherein the heavy chain comprises V_{H} CDR1 comprising or consisting of SEQ ID NO:173, V_{H} CDR2 comprising or consisting of SEQ ID NO:174, and V_{H} CDR3 comprising or consisting of SEQ ID NO:175;
a heavy chain comprising an amino acid sequence at least 80% identical, at least 85% identical, at least 90% identical, at least 95% identical, at last 97% identical, or at least 99% identical to SEQ ID NO:184, wherein the heavy chain comprises V_{H} CDR1 comprising or consisting of SEQ ID NO:181, V_{H} CDR2 comprising or consisting of SEQ ID NO: 182, and V_{H} CDR3 comprising or consisting of SEQ ID NO:183;
a heavy chain comprising an amino acid sequence at least 80% identical, at least 85% identical, at least 90% identical, at least 95% identical, at last 97% identical, or at least 99% identical to SEQ ID NO:192, wherein the heavy chain comprises V_{H} CDR1 comprising or consisting of SEQ ID NO:189, V_{H} CDR2 comprising or consisting of SEQ ID NO:190, and V_{H} CDR3 comprising or consisting of SEQ ID NO:191;
a heavy chain comprising an amino acid sequence at least 80% identical, at least 85% identical, at least 90% identical, at least 95% identical, at last 97% identical, or at least 99% identical to SEQ ID NO:200, wherein the heavy chain comprises V_{H} CDR1 comprising or consisting of SEQ ID NO:197, V_{H} CDR2 comprising or consisting of SEQ ID NO:198, and V_{H} CDR3 comprising or consisting of SEQ ID NO:199;
a heavy chain comprising an amino acid sequence at least 80% identical, at least 85% identical, at least 90% identical, at least 95% identical, at last 97% identical, or at least 99% identical to SEQ ID NO:208, wherein the heavy chain comprises V_{H} CDR1 comprising or consisting of SEQ ID NO:205, V_{H} CDR2 comprising or consisting of SEQ ID NO:206, and V_{H} CDR3 comprising or consisting of SEQ ID NO:207;
a heavy chain comprising an amino acid sequence at least 80% identical, at least 85% identical, at least 90% identical, at least 95% identical, at last 97% identical, or at least 99% identical to SEQ ID NO:216, wherein the heavy chain comprises V_{H} CDR1 comprising or consisting of SEQ ID NO:213, V_{H} CDR2 comprising or consisting of SEQ ID NO:214, and V_{H} CDR3 comprising or consisting of SEQ ID NO:215;
a heavy chain comprising an amino acid sequence at least 80% identical, at least 85% identical, at least 90% identical, at least 95% identical, at last 97% identical, or at least 99% identical to SEQ ID NO:224, wherein the heavy chain comprises V_{H} CDR1 comprising or consisting of SEQ ID NO:221, V_{H} CDR2 comprising or consisting of SEQ ID NO:222, and V_{H} CDR3 comprising or consisting of SEQ ID NO:223;
a heavy chain comprising an amino acid sequence at least 80% identical, at least 85% identical, at least 90% identical, at least 95% identical, at last 97% identical, or at least 99% identical to SEQ ID NO:232, wherein the heavy chain comprises V_{H} CDR1 comprising or consisting of SEQ ID NO:229, V_{H} CDR2 comprising or consisting of SEQ ID NO:230, and V_{H} CDR3 comprising or consisting of SEQ ID NO:231;
a heavy chain comprising an amino acid sequence at least 80% identical, at least 85% identical, at least 90% identical, at least 95% identical, at last 97% identical, or at least 99% identical to SEQ ID NO:240, wherein the heavy chain comprises V_{H} CDR1 comprising or consisting of SEQ ID NO:237, V_{H} CDR2 comprising or consisting of SEQ ID NO:238, and V_{H} CDR3 comprising or consisting of SEQ ID NO:239;
a heavy chain comprising an amino acid sequence at least 80% identical, at least 85% identical, at least 90% identical, at least 95% identical, at last 97% identical, or at least 99% identical to SEQ ID NO:248, wherein the heavy chain comprises V_{H} CDR1 comprising or consisting of SEQ ID NO:245, V_{H} CDR2 comprising or consisting of SEQ ID NO:246, and V_{H} CDR3 comprising or consisting of SEQ ID NO:247;
a heavy chain comprising an amino acid sequence at least 80% identical, at least 85% identical, at least 90% identical, at least 95% identical, at last 97% identical, or at least 99% identical to SEQ ID NO:256, wherein the heavy chain comprises V_{H} CDR1 comprising or consisting of SEQ ID NO:253, V_{H} CDR2 comprising or consisting of SEQ ID NO:254, and V_{H} CDR3 comprising or consisting of SEQ ID NO:255;
a heavy chain comprising an amino acid sequence at least 80% identical, at least 85% identical, at least 90% identical, at least 95% identical, at last 97% identical, or at least 99% identical to SEQ ID NO:264, wherein the heavy chain comprises V_{H} CDR1 comprising or consisting of SEQ ID NO:261, V_{H} CDR2 comprising or consisting of SEQ ID NO:262, and V_{H} CDR3 comprising or consisting of SEQ ID NO:263;
a heavy chain comprising an amino acid sequence at least 80% identical, at least 85% identical, at least 90% identical, at least 95% identical, at last 97% identical, or at least 99% identical to SEQ ID NO:272, wherein the heavy chain comprises V_{H} CDR1 comprising or consisting of SEQ ID NO:269, V_{H} CDR2 comprising or consisting of SEQ ID NO:270, and V_{H} CDR3 comprising or consisting of SEQ ID NO:271;
a heavy chain comprising an amino acid sequence at least 80% identical, at least 85% identical, at least 90% identical, at least 95% identical, at last 97% identical, or at least 99% identical to SEQ ID NO:280, wherein the heavy chain comprises V_{H} CDR1 comprising or consisting of SEQ ID NO:277, V_{H} CDR2 comprising or consisting of SEQ ID NO:278, and V_{H} CDR3 comprising or consisting of SEQ ID NO:279; and,
a heavy chain comprising an amino acid sequence at least 80% identical, at least 85% identical, at least 90% identical, at least 95% identical, at last 97% identical, or at least 99% identical to SEQ ID NO:288, wherein the heavy chain comprises V_{H} CDR1 comprising or consisting of SEQ ID NO:285, V_{H} CDR2 comprising or consisting of SEQ ID NO:286, and V_{H} CDR3 comprising or consisting of SEQ ID NO:287.

In one aspect, the isolated antibody comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:4, and the heavy chain comprises an amino acid sequence at least 80% identical, at least 85% identical, at least 90% identical, at least 95% identical, at least 97% identical, at least 99% identical, or 100% identical to SEQ ID NO:8. In certain aspects, the light chain may comprise a V_{L} CDR1 comprising or consisting of SEQ ID NO:1, a V_{L} CDR2 comprising or consisting of SEQ ID NO:2, and a V_{L} CDR3 comprising or consisting of SEQ ID NO:3. In certain aspects, the heavy chain may comprise a V_{H} CDR1 comprising or consisting of SEQ ID NO:5, a V_{H} CDR2 comprising or consisting of SEQ ID NO:6, and a V_{H} CDR3 comprising or consisting of SEQ ID NO:7. In certain aspects, the light chain may comprise SEQ ID NO:4 and the heavy chain may comprise SEQ ID NO:8.

In one aspect, the isolated antibody comprises a light chain and a heavy chain, wherein the light chain comprises amino acid sequence at least 80% identical, at least 85% identical, at least 90% identical, at least 95% identical, at last 97% identical, or at least 99% identical to SEQ ID NO:12, and the heavy chain comprises an amino acid sequence at least 80% identical, at least 85% identical, at least 90% identical, at least 95% identical, at last 97% identical, or at least 99% identical to SEQ ID NO: 16. In certain aspects, the light chain may comprise a V_{L} CDR1 comprising or consisting of SEQ ID NO:9, a V_{L} CDR2 comprising or consisting of SEQ ID NO:10, and a V_{L} CDR3 comprising or consisting of SEQ ID NO:11. In certain aspects, the heavy chain may comprise a V_{H} CDR1 comprising or consisting of SEQ ID NO:13, a V_{H} CDR2 comprising or consisting of SEQ ID NO:14, and a V_{H} CDR3 comprising or consisting of SEQ ID NO: 15. In certain aspects, the light chain may comprise SEQ ID NO:12 and the heavy chain may comprise SEQ ID NO:16.

In one aspect, the isolated antibody comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:20, and wherein the heavy chain comprises an amino acid sequence at least 80% identical, at least 85% identical, at least 90% identical, at least 95% identical, at last 97% identical, or at least 99% identical to SEQ ID NO:24. In certain aspects, the light chain may comprise a V_{L} CDR1 comprising or consisting of SEQ ID NO:17, a V_{L} CDR2 comprising or consisting of SEQ ID NO:18, and a V_{L} CDR3 comprising or consisting of SEQ ID NO:19. In certain aspects, the heavy chain may comprise a V_{H} CDR1 comprising or consisting of SEQ ID NO:21, a V_{H} CDR2 comprising or consisting of SEQ ID NO:22, and a V_{H} CDR3 comprising or consisting of SEQ ID NO:23. In certain aspects, the light chain may comprise SEQ ID NO:20 and the heavy chain may comprise SEQ ID NO:24.

In one aspect, the isolated antibody comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:28, and the heavy chain comprises an amino acid sequence at least 80% identical, at least 85% identical, at least 90% identical, at least 95% identical, at last 97% identical, or at least 99% identical to SEQ ID NO:32. In certain aspects, the light chain may comprise a V_{L} CDR1 comprising or consisting of SEQ ID NO:25, a V_{L} CDR2 comprising or consisting of SEQ ID NO:26, and a V_{L} CDR3 comprising or consisting of SEQ ID NO:27. In certain aspects, the heavy chain may comprise a V_{H} CDR1 comprising or consisting of SEQ ID NO:29, a V_{H} CDR2 comprising or consisting of SEQ ID NO:30, and a V_{H} CDR3 comprising or consisting of SEQ ID NO:31. In certain aspects, the light chain may comprise SEQ ID NO:28, and the heavy chain may comprise SEQ ID NO:32.

In one aspect, the isolated antibody comprises alight chain and a heavy chain, wherein the light chain comprises an amino acid sequence at least 80% identical, at least 85% identical, at least 90% identical, at least 95% identical, at last 97% identical, or at least 99% identical to SEQ ID NO:36, and the heavy chain comprises an amino acid sequence at least 80% identical, at least 85% identical, at least 90% identical, at least 95% identical, at last 97% identical, or at least 99% identical to SEQ ID NO:40. In certain aspects, the light chain may comprise a V_{L} CDR1 comprising or consisting of SEQ ID NO:33, a V_{L} CDR2 comprising or consisting of SEQ ID NO:34, and a V_{L} CDR3 comprising or consisting of SEQ ID NO:35. In certain aspects, the heavy chain may comprise a V_{H} CDR1 comprising or consisting of SEQ ID NO:37, a V_{H} CDR2 comprising or consisting of SEQ ID NO:38, and a V_{H} CDR3 comprising or consisting of SEQ ID NO:39. In certain aspects, the light chain may comprise SEQ ID NO:36, and the heavy chain may comprise SEQ ID NO:40.

In one aspect, the isolated antibody comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:44, and the heavy chain comprises an amino acid sequence at least 80% identical, at least 85% identical, at least 90% identical, at least 95% identical, at last 97% identical, or at least 99% identical to SEQ ID NO:48. In certain aspects, the light chain may comprise a V_{L} CDR1 comprising or consisting of SEQ ID NO:41, a V_{L} CDR2 comprising or consisting of SEQ ID NO:42, and a V_{L} CDR3 comprising or consisting of SEQ ID NO:43. In certain aspects, the heavy chain may comprise a V_{H} CDR1 comprising or consisting of SEQ ID NO:45, a V_{H} CDR2 comprising or consisting of SEQ ID NO:46, and a V_{H} CDR3 comprising or consisting of SEQ ID NO:47. In certain aspects, the light chain may comprise SEQ ID NO:44, and the heavy chain may comprise SEQ ID NO:48.

In one aspect, the isolated antibody comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:52, and the heavy chain comprises an amino acid sequence at least 80% identical, at least 85% identical, at least 90% identical, at least 95% identical, at last 97% identical, or at least 99% identical to SEQ ID NO:56. In certain aspects, the light chain may comprise a V_{L} CDR1 comprising or consisting of SEQ ID NO:49, a V_{L} CDR2 comprising or consisting of SEQ ID NO:50, and a V_{L} CDR3 comprising or consisting of SEQ ID NO:51. In certain aspects, the heavy chain may comprise a V_{H} CDR1 comprising or consisting of SEQ ID NO:53, a V_{H} CDR2 comprising or consisting of SEQ ID NO:54, and a V_{H} CDR3 comprising or consisting of SEQ ID NO:55. In certain aspects, the light chain may comprise SEQ ID NO:52 and the heavy chain may comprise SEQ ID NO:56.

In one aspect, the isolated antibody comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:60, and the heavy chain comprises an amino acid sequence at least 80% identical, at least 85% identical, at least 90% identical, at least 95% identical, at last 97% identical, or at least 99% identical to SEQ ID NO:64. In certain aspects, the light chain may comprise a V_{L} CDR1 comprising or consisting of SEQ ID NO:57, a V_{L} CDR2 comprising or consisting of SEQ ID NO:58, and a V_{L} CDR3 comprising or consisting of SEQ ID NO:59. In certain aspects, the heavy chain may comprise a V_{H} CDR1 comprising or consisting of SEQ ID NO:61, a V_{H} CDR2 comprising or consisting of SEQ ID NO:62, and a V_{H} CDR3 comprising or consisting of SEQ ID NO:63. In certain aspects, the light chain may comprise SEQ ID NO:60 and the heavy chain may comprise SEQ ID NO:64.

In one aspect, the isolated antibody comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:68, and the heavy chain comprises an amino acid sequence at least 80% identical, at least 85% identical, at least 90% identical, at least 95% identical, at last 97% identical, or at least 99% identical to SEQ ID NO:72. In certain aspects, the light chain may comprise a V_{L} CDR1 comprising or consisting of SEQ ID NO:65, a V_{L} CDR2 comprising or consisting of SEQ ID NO:66, and a V_{L} CDR3 comprising or consisting of SEQ ID NO:67. In certain aspects, the heavy chain may comprise a V_{H} CDR1 comprising or consisting of SEQ ID NO:69, a V_{H} CDR2 comprising or consisting of SEQ ID NO:70, and a V_{L} CDR3 comprising or consisting of SEQ ID NO:71. In certain aspects, the light chain may comprise SEQ ID NO:68 and the heavy chain may comprise SEQ ID NO:72.

In one aspect, the isolated antibody comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:76, and the heavy chain comprises an amino acid sequence at least 80% identical, at least 85% identical, at least 90% identical, at least 95% identical, at last 97% identical, or at least 99% identical to SEQ ID NO:80. In certain aspects, the light chain may comprise a V_{L} CDR1 comprising or consisting of SEQ ID NO:73, a V_{L} CDR2 comprising or consisting of SEQ ID NO:74, and a V_{L} CDR3 comprising or consisting of SEQ ID NO:75. In certain aspects, the heavy chain may comprise a V_{H} CDR1 comprising or consisting of SEQ ID NO:77, a V_{H} CDR2 comprising or consisting of SEQ ID NO:78, and a V_{H} CDR3 comprising or consisting of SEQ ID NO:79. In certain aspects, the light chain may comprise SEQ ID NO:76 and the heavy chain may comprise SEQ ID NO:80.

In one aspect, the isolated antibody comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:84, and the heavy chain comprises an amino acid sequence at least 80% identical, at least 85% identical, at least 90% identical, at least 95% identical, at last 97% identical, or at least 99% identical to SEQ ID NO:88. In certain aspects, the light chain may comprise a V_{L} CDR1 comprising or consisting of SEQ ID NO:81, a V_{L} CDR2 comprising or consisting of SEQ ID NO:82, and a V_{L} CDR3 comprising or consisting of SEQ ID NO:83. In certain aspects, the heavy chain may comprise a V_{H} CDR1 comprising or consisting of SEQ ID NO:85, a V_{H} CDR2 comprising or consisting of SEQ ID NO:86, and a V_{L} CDR3 comprising or consisting of SEQ ID NO:87. In certain aspects, the light chain may comprise SEQ ID NO:84 and the heavy chain may comprise SEQ ID NO:88.

In one aspect, the isolated antibody comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:92, and the heavy chain comprises an amino acid sequence at least 80% identical, at least 85% identical, at least 90% identical, at least 95% identical, at last 97% identical, or at least 99% identical to SEQ ID NO:96. In certain aspects, the light chain may comprise a V_{L} CDR1 comprising or consisting of SEQ ID NO:89, a V_{L} CDR2 comprising or consisting of SEQ ID NO:90, and a V_{L} CDR3 comprising or consisting of SEQ ID NO:91. In certain aspects, the heavy chain may comprise a V_{H} CDR1 comprising or consisting of SEQ ID NO:93, a V_{H} CDR2 comprising or consisting of SEQ ID NO:94, and a V_{H} CDR3 comprising or consisting of SEQ ID NO:95. In certain aspects, the light chain may comprise SEQ ID NO:92 and the heavy chain may comprise SEQ ID NO:96.

In one aspect, the isolated antibody comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:100, and the heavy chain comprises an amino acid sequence at least 80% identical, at least 85% identical, at least 90% identical, at least 95% identical, at last 97% identical, or at least 99% identical to SEQ ID NO: 104. In certain aspects, the light chain may comprise a V_{L} CDR1 comprising or consisting of SEQ ID NO:97, a V_{L} CDR2 comprising or consisting of SEQ ID NO:98, and a V_{L} CDR3 comprising or consisting of SEQ ID NO:99. In certain aspects, the heavy chain may comprise a V_{H} CDR1 comprising or consisting of SEQ ID NO:101, a V_{H} CDR2 comprising or consisting of SEQ ID NO:102, and a V_{H} CDR3 comprising or consisting of SEQ ID NO:103. In certain aspects, the light chain may comprise SEQ ID NO:100 and the heavy chain may comprise SEQ ID NO: 104.

In one aspect, the isolated antibody comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:108, and the heavy chain comprises an amino acid sequence at least 80% identical, at least 85% identical, at least 90% identical, at least 95% identical, at last 97% identical, or at least 99% identical to SEQ ID NO: 112. In certain aspects, the light chain may comprise a V_{L} CDR1 comprising or consisting of SEQ ID NO:105, a V_{L} CDR2 comprising or consisting of SEQ ID NO:106, and a V_{L} CDR3 comprising or consisting of SEQ ID NO:107. In certain aspects, the heavy chain may comprise a V_{H} CDR1 comprising or consisting of SEQ ID NO:109, a V_{H} CDR2 comprising or consisting of SEQ ID NO:110, and a V_{H} CDR3 comprising or consisting of SEQ ID NO:111. In certain aspects, the light chain may comprise SEQ ID NO:108 and the heavy chain may comprise SEQ ID NO: 112.

In one aspect, the isolated antibody comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:116, and the heavy chain comprises an amino acid sequence at least 80% identical, at least 85% identical, at least 90% identical, at least 95% identical, at last 97% identical, or at least 99% identical to SEQ ID NO: 120. In certain aspects the light chain may comprise a V_{L} CDR1 comprising or consisting of SEQ ID NO:113, a V_{L} CDR2 comprising or consisting of SEQ ID NO:114, and a V_{L} CDR3 comprising or consisting of SEQ ID NO: 115. In certain aspects, the heavy chain may comprise a V_{H} CDR1 comprising or consisting of SEQ ID NO:117, a V_{H} CDR2 comprising or consisting of SEQ ID NO:118, and a V_{H} CDR3 comprising or consisting of SEQ ID NO: 119. In certain aspects, the light chain may comprise SEQ ID NO: 116 and the heavy chain may comprise SEQ ID NO:120.

In one aspect, the isolated antibody comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:124, and the heavy chain comprise an amino acid sequence at least 80% identical, at least 85% identical, at least 90% identical, at least 95% identical, at last 97% identical, or at least 99% identical to SEQ ID NO: 128. In certain aspects, the light chain may comprise a V_{L} CDR1 comprising or consisting of SEQ ID NO:121, a V_{L} CDR2 comprising or consisting of SEQ ID NO:122, and a V_{L} CDR3 comprising or consisting of SEQ ID NO:123. In certain aspects, the heavy chain may comprise V_{H} CDR1 comprising or consisting of SEQ ID NO: 125, V_{H} CDR2 comprising or consisting of SEQ ID NO: 126, and V_{H} CDR3 comprising or consisting of SEQ ID NO:127. In certain aspects, the light chain may comprise SEQ ID NO: 124 and the heavy chain may comprise SEQ ID NO:128.

In one aspect, the isolated antibody comprises alight chain and a heavy chain, wherein the light chain comprises an amino acid sequence at least 80% identical, at least 85% identical, at least 90% identical, at least 95% identical, at last 97% identical, or at least 99% identical to SEQ ID NO:132, and the heavy chain comprises an amino acid sequence at least 80% identical, at least 85% identical, at least 90% identical, at least 95% identical, at last 97% identical, or at least 99% identical to SEQ ID NO: 136. In certain aspects the light chain may comprise a V_{L} CDR1 comprising or consisting of SEQ ID NO:129, a V_{L} CDR2 comprising or consisting of SEQ ID NO:130, and a V_{L} CDR3 comprising or consisting of SEQ ID NO:131. In certain aspects, the heavy chain may comprise a V_{H} CDR1 comprising or consisting of SEQ ID NO:133, a V_{H} CDR2 comprising or consisting of SEQ ID NO:134, and a V_{H} CDR3 comprising or consisting of SEQ ID NO:135. In certain aspects, the light chain may comprise SEQ ID NO: 132 and the heavy chain may comprise SEQ ID NO:136.

In one aspect, the isolated antibody comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:140, and the heavy chain comprises an amino acid sequence at least 80% identical, at least 85% identical, at least 90% identical, at least 95% identical, at last 97% identical, or at least 99% identical to SEQ ID NO:144. In certain aspects, the light chain may comprise a V_{L} CDR1 comprising or consisting of SEQ ID NO:137, a V_{L} CDR2 comprising or consisting of SEQ ID NO:138, and a V_{L} CDR3 comprising or consisting of SEQ ID NO: 139. In certain aspects, the heavy chain may comprise a V_{H} CDR1 comprising or consisting of SEQ ID NO:141, a V_{H} CDR2 comprising or consisting of SEQ ID NO:142, and a V_{H} CDR3 comprising or consisting of SEQ ID NO: 143. In certain aspects, the light chain may comprise SEQ ID NO: 140 and the heavy chain may comprise SEQ ID NO:144.

In one aspect, the isolated antibody comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:148, and the heavy chain comprises an amino acid sequence at least 80% identical, at least 85% identical, at least 90% identical, at least 95% identical, at last 97% identical, or at least 99% identical to SEQ ID NO: 152. In certain aspects, the light chain may comprise a V_{L} CDR1 comprising or consisting of SEQ ID NO: 145, a V_{L} CDR2 comprising or consisting of SEQ ID NO:146, and a V_{L} CDR3 comprising or consisting of SEQ ID NO:147. In certain aspects the heavy chain may comprise a V_{H} CDR1 comprising or consisting of SEQ ID NO:149, a V_{H} CDR2 comprising or consisting of SEQ ID NO:150, and a V_{H} CDR3 comprising or consisting of SEQ ID NO:151. In certain aspects, the light chain may comprise SEQ ID NO:148 and the heavy chain may comprise SEQ ID NO:152.

In one aspect, the isolated antibody comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:156, and the heavy chain comprises an amino acid sequence at least 80% identical, at least 85% identical, at least 90% identical, at least 95% identical, at last 97% identical, or at least 99% identical to SEQ ID NO: 160. In certain aspects, the light chain may comprise a V_{L} CDR1 comprising or consisting of SEQ ID NO:153, a V_{L} CDR2 comprising or consisting of SEQ ID NO:154, and a V_{L} CDR3 comprising or consisting of SEQ ID NO:155. In certain aspects, the heavy chain may comprise a V_{H} CDR1 comprising or consisting of SEQ ID NO:157, a V_{H} CDR2 comprising or consisting of SEQ ID NO:158, and a V_{H} CDR3 comprising or consisting of SEQ ID NO:159. In certain aspects, the light chain may comprise SEQ ID NO: 156 and the heavy chain may comprise SEQ ID NO: 160.

In one aspect, the isolated antibody comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:164, and the heavy chain comprises an amino acid sequence at least 80% identical, at least 85% identical, at least 90% identical, at least 95% identical, at last 97% identical, or at least 99% identical to SEQ ID NO: 168. In certain aspects, the light chain may comprise a V_{L} CDR1 comprising or consisting of SEQ ID NO:161, a V_{L} CDR2 comprising or consisting of SEQ ID NO:162, and a V_{L} CDR3 comprising or consisting of SEQ ID NO:163. In certain aspects, the heavy chain may comprise a V_{H} CDR1 comprising or consisting of SEQ ID NO:165, a V_{H} CDR2 comprising or consisting of SEQ ID NO:166, and a V_{H} CDR3 comprising or consisting of SEQ ID NO:167. In certain aspects, the light chain may comprise SEQ ID NO: 164 and the heavy chain may comprise SEQ ID NO:168.

In one aspect, the isolated antibody comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:172, the heavy chain comprises an amino acid sequence at least 80% identical, at least 85% identical, at least 90% identical, at least 95% identical, at last 97% identical, or at least 99% identical to SEQ ID NO:176. In certain aspects, the light chain may comprise a V_{L} CDR1 comprising or consisting of SEQ ID NO:169, a V_{L} CDR2 comprising or consisting of SEQ ID NO:170, and a V_{L} CDR3 comprising or consisting of SEQ ID NO:171. In certain aspects, the heavy chain may comprise a V_{H} CDR1 comprising or consisting of SEQ ID NO:173, a V_{H} CDR2 comprising or consisting of SEQ ID NO:174, and a V_{H} CDR3 comprising or consisting of SEQ ID NO: 175. In certain aspects, the light chain may comprise SEQ ID NO: 172 and the heavy chain may comprise SEQ ID NO: 176.

In one aspect, the isolated antibody comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:180, the heavy chain comprises an amino acid sequence at least 80% identical, at least 85% identical, at least 90% identical, at least 95% identical, at last 97% identical, or at least 99% identical to SEQ ID NO:184. In certain aspects, the light chain may comprise a V_{L} CDR1 comprising or consisting of SEQ ID NO:177, a V_{L} CDR2 comprising or consisting of SEQ ID NO:178, and a V_{L} CDR3 comprising or consisting of SEQ ID NO: 179. In certain aspects, the heavy chain may comprise a V_{H} CDR1 comprising or consisting of SEQ ID NO:181, a V_{H} CDR2 comprising or consisting of SEQ ID NO:182, and a V_{H} CDR3 comprising or consisting of SEQ ID NO:183. In certain aspects, the light chain may comprise SEQ ID NO: 180 and the heavy chain may comprise SEQ ID NO:184.

In one aspect, the isolated antibody comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:188, the heavy chain comprises an amino acid sequence at least 80% identical, at least 85% identical, at least 90% identical, at least 95% identical, at last 97% identical, or at least 99% identical to SEQ ID NO:192. In certain aspects, the light chain may comprise a V_{L} CDR1 comprising or consisting of SEQ ID NO:185, a V_{L} CDR2 comprising or consisting of SEQ ID NO: 186, and a V_{L} CDR3 comprising or consisting of SEQ ID NO:187. In certain aspects, the heavy chain may comprise a V_{H} CDR1 comprising or consisting of SEQ ID NO:189, a V_{H} CDR2 comprising or consisting of SEQ ID NO: 190, and a V_{H} CDR3 comprising or consisting of SEQ ID NO:191. In certain aspects, the light chain may comprise SEQ ID NO: 188 and the heavy chain may comprise SEQ ID NO:192.

In one aspect, the isolated antibody comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:196, the heavy chain comprises an amino acid sequence at least 80% identical, at least 85% identical, at least 90% identical, at least 95% identical, at last 97% identical, or at least 99% identical to SEQ ID NO:200. In certain aspects, the light chain may comprise a V_{L} CDR1 comprising or consisting of SEQ ID NO:193, a V_{L} CDR2 comprising or consisting of SEQ ID NO:194, and a V_{L} CDR3 comprising or consisting of SEQ ID NO: 195. In certain aspects, the heavy chain may comprise a V_{H} CDR1 comprising or consisting of SEQ ID NO:197, a V_{H} CDR2 comprising or consisting of SEQ ID NO:198, and a V_{H} CDR3 comprising or consisting of SEQ ID NO:199. In certain aspects, the light chain may comprise SEQ ID NO: 196 and the heavy chain may comprise SEQ ID NO:200.

In one aspect, the isolated antibody may comprise a light chain and a heavy chain, wherein the light chain may comprise an amino acid sequence at least 80% identical, at least 85% identical, at least 90% identical, at least 95% identical, at last 97% identical, or at least 99% identical to SEQ ID NO:204, and the heavy chain may comprise an amino acid sequence at least 80% identical, at least 85% identical, at least 90% identical, at least 95% identical, at last 97% identical, or at least 99% identical to SEQ ID NO:208. In certain aspects, the light chain may comprise a V_{L} CDR1 comprising or consisting of SEQ ID NO:201, a V_{L} CDR2 comprising or consisting of SEQ ID NO:202, and a V_{L} CDR3 comprising or consisting of SEQ ID NO:203. In certain aspects, the heavy chain may comprise a V_{H} CDR1 comprising or consisting of SEQ ID NO:205, a V_{H} CDR2 comprising or consisting of SEQ ID NO:206, and a V_{H} CDR3 comprising or consisting of SEQ ID NO:207. In certain aspects, the light chain may comprise SEQ ID NO:204 and the heavy chain may comprise SEQ ID NO:208.

In one aspect, the isolated antibody may comprise a light chain and a heavy chain, wherein the light chain may comprise an amino acid sequence at least 80% identical, at least 85% identical, at least 90% identical, at least 95% identical, at last 97% identical, or at least 99% identical to SEQ ID NO:212, and the heavy chain may comprise an amino acid sequence at least 80% identical, at least 85% identical, at least 90% identical, at least 95% identical, at last 97% identical, or at least 99% identical to SEQ ID NO:216. In certain aspects, the light chain may comprise a V_{L} CDR1 comprising or consisting of SEQ ID NO:209, a V_{L} CDR2 comprising or consisting of SEQ ID NO:210, and a V_{L} CDR3 comprising or consisting of SEQ ID NO:211. In certain aspects, the heavy chain may comprise a V_{H} CDR1 comprising or consisting of SEQ ID NO:213, a V_{H} CDR2 comprising or consisting of SEQ ID NO:214, and a V_{H} CDR3 comprising or consisting of SEQ ID NO:215. In certain aspects, the light chain may comprise SEQ ID NO:212 and the heavy chain may comprise SEQ ID NO:216.

In one aspect, the isolated antibody may comprise a light chain and a heavy chain, wherein the light chain may comprise an amino acid sequence at least 80% identical, at least 85% identical, at least 90% identical, at least 95% identical, at last 97% identical, or at least 99% identical to SEQ ID NO:220, and the heavy chain may comprise an amino acid sequence at least 80% identical, at least 85% identical, at least 90% identical, at least 95% identical, at last 97% identical, or at least 99% identical to SEQ ID NO:224. In certain aspects, the light chain may comprise a V_{L} CDR1 comprising or consisting of SEQ ID NO:217, a V_{L} CDR2 comprising or consisting of SEQ ID NO:218, and a V_{L} CDR3 comprising or consisting of SEQ ID NO:219. In certain aspects, the heavy chain may comprise a V_{H} CDR1 comprising or consisting of SEQ ID NO:221, a V_{H} CDR2 comprising or consisting of SEQ ID NO:222, and a V_{H} CDR3 comprising or consisting of SEQ ID NO:223. In certain aspects, the light chain may comprise SEQ ID NO:220 and the heavy chain may comprise SEQ ID NO:224.

In one aspect, the isolated antibody may comprise a light chain and a heavy chain, wherein the light chain may comprise an amino acid sequence at least 80% identical, at least 85% identical, at least 90% identical, at least 95% identical, at last 97% identical, or at least 99% identical to SEQ ID NO:228, and the heavy chain may comprise an amino acid sequence at least 80% identical, at least 85% identical, at least 90% identical, at least 95% identical, at last 97% identical, or at least 99% identical to SEQ ID NO:232. In certain aspects, the light chain may comprise a V_{L} CDR1 comprising or consisting of SEQ ID NO:225, a V_{L} CDR2 comprising or consisting of SEQ ID NO:226, and a V_{L} CDR3 comprising or consisting of SEQ ID NO:227. In certain aspects, the heavy chain may comprise a V_{H} CDR1 comprising or consisting of SEQ ID NO:229, a V_{H} CDR2 comprising or consisting of SEQ ID NO:230, and a V_{H} CDR3 comprising or consisting of SEQ ID NO:231. In certain aspects, the light chain may comprise SEQ ID NO:228 and the heavy chain may comprise SEQ ID NO:232.

In one aspect, the isolated antibody may comprise a light chain and a heavy chain, wherein the light chain may comprise an amino acid sequence at least 80% identical, at least 85% identical, at least 90% identical, at least 95% identical, at last 97% identical, or at least 99% identical to SEQ ID NO:236, and the heavy chain may comprise an amino acid sequence at least 80% identical, at least 85% identical, at least 90% identical, at least 95% identical, at last 97% identical, or at least 99% identical to SEQ ID NO:240. In certain aspects, the light chain may comprise a V_{L} CDR1 comprising or consisting of SEQ ID NO:233, a V_{L} CDR2 comprising or consisting of SEQ ID NO:234, and a V_{L} CDR3 comprising or consisting of SEQ ID NO:235. In certain aspects, the heavy chain may comprise a V_{H} CDR1 comprising or consisting of SEQ ID NO:237, a V_{H} CDR2 comprising or consisting of SEQ ID NO:238, and a V_{H} CDR3 comprising or consisting of SEQ ID NO:239. In certain aspects, the light chain may comprise SEQ ID NO:236 and the heavy chain may comprise SEQ ID NO:240.

In one aspect, the isolated antibody may comprise a light chain and a heavy chain, wherein the light chain may comprise an amino acid sequence at least 80% identical, at least 85% identical, at least 90% identical, at least 95% identical, at last 97% identical, or at least 99% identical to SEQ ID NO:244, and the heavy chain may comprise an amino acid sequence at least 80% identical, at least 85% identical, at least 90% identical, at least 95% identical, at last 97% identical, or at least 99% identical to SEQ ID NO:248. In certain aspects, the light chain may comprise a V_{L} CDR1 comprising or consisting of SEQ ID NO:241, a V_{L} CDR2 comprising or consisting of SEQ ID NO:242, and a V_{L} CDR3 comprising or consisting of SEQ ID NO:243. In certain aspects, the heavy chain may comprise a V_{H} CDR1 comprising or consisting of SEQ ID NO:245, a V_{H} CDR2 comprising or consisting of SEQ ID NO:246, and a V_{H} CDR3 comprising or consisting of SEQ ID NO:247. In certain aspects, the light chain may comprise SEQ ID NO:244; and the heavy chain may comprise SEQ ID NO:248.

In one aspect, the isolated antibody may comprise a light chain and a heavy chain, wherein the light chain may comprise an amino acid sequence at least 80% identical, at least 85% identical, at least 90% identical, at least 95% identical, at last 97% identical, or at least 99% identical to SEQ ID NO:252, and the heavy chain may comprise an amino acid sequence at least 80% identical, at least 85% identical, at least 90% identical, at least 95% identical, at last 97% identical, or at least 99% identical to SEQ ID NO:256. In certain aspects, the light chain may comprise a V_{L} CDR1 comprising or consisting of SEQ ID NO:249, a V_{L} CDR2 comprising or consisting of SEQ ID NO:250, and a V_{L} CDR3 comprising or consisting of SEQ ID NO:251. In certain aspects, the heavy chain may comprise a V_{H} CDR1 comprising or consisting of SEQ ID NO:253, a V_{H} CDR2 comprising or consisting of SEQ ID NO:254, and a V_{H} CDR3 comprising or consisting of SEQ ID NO:255. In certain aspects, the light chain may comprise SEQ ID NO:252 and the heavy chain may comprise SEQ ID NO:256.

In one aspect, the isolated antibody may comprise a light chain and a heavy chain, wherein the light chain may comprise an amino acid sequence at least 80% identical, at least 85% identical, at least 90% identical, at least 95% identical, at last 97% identical, or at least 99% identical to SEQ ID NO:260, and the heavy chain may comprise an amino acid sequence at least 80% identical, at least 85% identical, at least 90% identical, at least 95% identical, at last 97% identical, or at least 99% identical to SEQ ID NO:264. In certain aspects, the light chain may comprise a V_{L} CDR1 comprising or consisting of SEQ ID NO:257, a V_{L} CDR2 comprising or consisting of SEQ ID NO:258, and a V_{L} CDR3 comprising or consisting of SEQ ID NO:259. In certain aspects, the heavy chain may comprise a V_{H} CDR1 comprising or consisting of SEQ ID NO:261, a V_{H} CDR2 comprising or consisting of SEQ ID NO:262, and a V_{H} CDR3 comprising or consisting of SEQ ID NO:263. In certain aspects, the light chain may comprise SEQ ID NO:260 and the heavy chain may comprise SEQ ID NO:264.

In one aspect, the isolated antibody may comprise a light chain and a heavy chain, wherein the light chain may comprise an amino acid sequence at least 80% identical, at least 85% identical, at least 90% identical, at least 95% identical, at last 97% identical, or at least 99% identical to SEQ ID NO:268, and the heavy chain may comprise an amino acid sequence at least 80% identical, at least 85% identical, at least 90% identical, at least 95% identical, at last 97% identical, or at least 99% identical to SEQ ID NO:272. In certain aspects, the light chain may comprise a V_{L} CDR1 comprising or consisting of SEQ ID NO:265, a V_{L} CDR2 comprising or consisting of SEQ ID NO:266, and a V_{L} CDR3 comprising or consisting of SEQ ID NO:267. In certain aspects, the heavy chain may comprise a V_{H} CDR1 comprising or consisting of SEQ ID NO:269, a V_{H} CDR2 comprising or consisting of SEQ ID NO:270, and a V_{H} CDR3 comprising or consisting of SEQ ID NO:271. In certain aspects, the light chain may comprise SEQ ID NO:268 and the heavy chain may comprise SEQ ID NO:272.

In one aspect, the isolated antibody may comprise a light chain and a heavy chain, wherein the light chain may comprise an amino acid sequence at least 80% identical, at least 85% identical, at least 90% identical, at least 95% identical, at last 97% identical, or at least 99% identical to SEQ ID NO:276, and the heavy chain may comprise an amino acid sequence at least 80% identical, at least 85% identical, at least 90% identical, at least 95% identical, at last 97% identical, or at least 99% identical to SEQ ID NO:280. In certain aspects, the light chain may comprise a V_{L} CDR1 comprising or consisting of SEQ ID NO:273, a V_{L} CDR2 comprising or consisting of SEQ ID NO:274, and a V_{L} CDR3 comprising or consisting of SEQ ID NO:275. In certain aspects, the heavy chain may comprise a V_{H} CDR1 comprising or consisting of SEQ ID NO:277, a V_{H} CDR2 comprising or consisting of SEQ ID NO:278, and a V_{H} CDR3 comprising or consisting of SEQ ID NO:279. In certain aspects, the light chain may comprise SEQ ID NO:276 and the heavy chain may comprise SEQ ID NO:280.

In one aspect, the isolated antibody may comprise a light chain and a heavy chain, wherein the light chain may comprise an amino acid sequence at least 80% identical, at least 85% identical, at least 90% identical, at least 95% identical, at last 97% identical, or at least 99% identical to SEQ ID NO:284, and the heavy chain may comprise an amino acid sequence at least 80% identical, at least 85% identical, at least 90% identical, at least 95% identical, at last 97% identical, or at least 99% identical to SEQ ID NO:288. In certain aspects, the light chain may comprise a V_{L} CDR1 comprising or consisting of SEQ ID NO:281, a V_{L} CDR2 comprising or consisting of SEQ ID NO:282, and a V_{L} CDR3 comprising or consisting of SEQ ID NO:283. In certain aspects, the heavy chain may comprise a V_{H} CDR1 comprising or consisting of SEQ ID NO:285, a V_{H} CDR2 comprising or consisting of SEQ ID NO:286, and a V_{H} CDR3 comprising or consisting of SEQ ID NO:287. In certain aspects, the light chain may comprise SEQ ID NO:284 and the heavy chain may comprise SEQ ID NO:288.

In one aspect, the isolated antibody may comprise a light chain comprising an amino acid sequence at least 80% identical, at least 85% identical, at least 90% identical, at least 95% identical, at last 97% identical, at least 99% identical, or 100% identical to SEQ ID NO:290, SEQ ID NO:292, SEQ ID NO:294, SEQ ID NO:296, SEQ ID NO:298, SEQ ID NO:300, SEQ ID NO:302, SEQ ID NO:304, SEQ ID NO:306, SEQ ID NO:308, SEQ ID NO:310, SEQ ID NO:312, SEQ ID NO:314, SEQ ID NO:316, SEQ ID NO:318, SEQ ID NO:320, SEQ ID NO:322, SEQ ID NO:324, SEQ ID NO:326, SEQ ID NO:328, SEQ ID NO:330, SEQ ID NO:332, SEQ ID NO:334, SEQ ID NO:336, SEQ ID NO:338, SEQ ID NO:340, SEQ ID NO:342, SEQ ID NO:344, SEQ ID NO:346, SEQ ID NO:348, SEQ ID NO:350, SEQ ID NO:352, SEQ ID NO:354, SEQ ID NO:356, SEQ ID NO:358, or SEQ ID NO:360.

In one aspect, the isolated antibody may comprise a light chain selected from the group consisting of:
a light chain comprising an amino acid sequence at least 80% identical, at least 85% identical, at least 90% identical, at least 95% identical, at last 97% identical, or at least 99% identical to SEQ ID NO:290, wherein the light chain comprises V_{L} CDR1 comprising or consisting of SEQ ID NO:1, V_{L} CDR2 comprising or consisting of SEQ ID NO:2, and V_{L} CDR3 comprising or consisting of SEQ ID NO:3;
a light chain comprising an amino acid sequence at least 80% identical, at least 85% identical, at least 90% identical, at least 95% identical, at last 97% identical, or at least 99% identical to SEQ ID NO:292, wherein the light chain comprises V_{L} CDR1 comprising or consisting of SEQ ID NO:9, V_{L} CDR2 comprising or consisting of SEQ ID NO:10, and V_{L} CDR3 comprising or consisting of SEQ ID NO: 11;
a light chain comprising an amino acid sequence at least 80% identical, at least 85% identical, at least 90% identical, at least 95% identical, at last 97% identical, or at least 99% identical to SEQ ID NO:294, wherein the light chain comprises V_{L} CDR1 comprising or consisting of SEQ ID NO:17, V_{L} CDR2 comprising or consisting of SEQ ID NO:18, and V_{L} CDR3 comprising or consisting of SEQ ID NO:19;
a light chain comprising an amino acid sequence at least 80% identical, at least 85% identical, at least 90% identical, at least 95% identical, at last 97% identical, or at least 99% identical to SEQ ID NO:296, wherein the light chain comprises V_{L} CDR1 comprising or consisting of SEQ ID NO:25, V_{L} CDR2 comprising or consisting of SEQ ID NO:26, and V_{L} CDR3 comprising or consisting of SEQ ID NO:27;
a light chain comprising an amino acid sequence at least 80% identical, at least 85% identical, at least 90% identical, at least 95% identical, at last 97% identical, or at least 99% identical to SEQ ID NO:298, wherein the light chain comprises V_{L} CDR1 comprising or consisting of SEQ ID NO:33, V_{L} CDR2 comprising or consisting of SEQ ID NO:34, and V_{L} CDR3 comprising or consisting of SEQ ID NO:35;
a light chain comprising an amino acid sequence at least 80% identical, at least 85% identical, at least 90% identical, at least 95% identical, at last 97% identical, or at least 99% identical to SEQ ID NO:300, wherein the light chain comprises V_{L} CDR1 comprising or consisting of SEQ ID NO:41, V_{L} CDR2 comprising or consisting of SEQ ID NO:42, and V_{L} CDR3 comprising or consisting of SEQ ID NO:43;
a light chain comprising an amino acid sequence at least 80% identical, at least 85% identical, at least 90% identical, at least 95% identical, at last 97% identical, or at least 99% identical to SEQ ID NO:302, wherein the light chain comprises V_{L} CDR1 comprising or consisting of SEQ ID NO:49, V_{L} CDR2 comprising or consisting of SEQ ID NO:50, and V_{L} CDR3 comprising or consisting of SEQ ID NO:51;
a light chain comprising an amino acid sequence at least 80% identical, at least 85% identical, at least 90% identical, at least 95% identical, at last 97% identical, or at least 99% identical to SEQ ID NO:304, wherein the light chain comprises V_{L} CDR1 comprising or consisting of SEQ ID NO:57, V_{L} CDR2 comprising or consisting of SEQ ID NO:58, and V_{L} CDR3 comprising or consisting of SEQ ID NO:59;
a light chain comprising an amino acid sequence at least 80% identical, at least 85% identical, at least 90% identical, at least 95% identical, at last 97% identical, or at least 99% identical to SEQ ID NO:306, wherein the light chain comprises V_{L} CDR1 comprising or consisting of SEQ ID NO:65, V_{L} CDR2 comprising or consisting of SEQ ID NO:66, and V_{L} CDR3 comprising or consisting of SEQ ID NO:67;
a light chain comprising an amino acid sequence at least 80% identical, at least 85% identical, at least 90% identical, at least 95% identical, at last 97% identical, or at least 99% identical to SEQ ID NO:308, wherein the light chain comprises V_{L} CDR1 comprising or consisting of SEQ ID NO:73, V_{L} CDR2 comprising or consisting of SEQ ID NO:74, and V_{L} CDR3 comprising or consisting of SEQ ID NO:75;
a light chain comprising an amino acid sequence at least 80% identical, at least 85% identical, at least 90% identical, at least 95% identical, at last 97% identical, or at least 99% identical to SEQ ID NO:310, wherein the light chain comprises V_{L} CDR1 comprising or consisting of SEQ ID NO:81, V_{L} CDR2 comprising or consisting of SEQ ID NO:82, and V_{L} CDR3 comprising or consisting of SEQ ID NO:83;
a light chain comprising an amino acid sequence at least 80% identical, at least 85% identical, at least 90% identical, at least 95% identical, at last 97% identical, or at least 99% identical to SEQ ID NO:312, wherein the light chain comprises V_{L} CDR1 comprising or consisting of SEQ ID NO:89, V_{L} CDR2 comprising or consisting of SEQ ID NO:90, and V_{L} CDR3 comprising or consisting of SEQ ID NO:91;
a light chain comprising an amino acid sequence at least 80% identical, at least 85% identical, at least 90% identical, at least 95% identical, at last 97% identical, or at least 99% identical to SEQ ID NO:314, wherein the light chain comprises V_{L} CDR1 comprising or consisting of SEQ ID NO:97, V_{L} CDR2 comprising or consisting of SEQ ID NO:98, and V_{L} CDR3 comprising or consisting of SEQ ID NO:99;
a light chain comprising an amino acid sequence at least 80% identical, at least 85% identical, at least 90% identical, at least 95% identical, at last 97% identical, or at least 99% identical to SEQ ID NO:316, wherein the light chain comprises V_{L} CDR1 comprising or consisting of SEQ ID NO:105, V_{L} CDR2 comprising or consisting of SEQ ID NO:106, and V_{L} CDR3 comprising or consisting of SEQ ID NO:107;
a light chain comprising an amino acid sequence at least 80% identical, at least 85% identical, at least 90% identical, at least 95% identical, at last 97% identical, or at least 99% identical to SEQ ID NO:318, wherein the light chain comprises V_{L} CDR1 comprising or consisting of SEQ ID NO: 113, V_{L} CDR2 comprising or consisting of SEQ ID NO:114, and V_{L} CDR3 comprising or consisting of SEQ ID NO: 15;
a light chain comprising an amino acid sequence at least 80% identical, at least 85% identical, at least 90% identical, at least 95% identical, at last 97% identical, or at least 99% identical to SEQ ID NO:320, wherein the light chain comprises V_{L} CDR1 comprising or consisting of SEQ ID NO:121, V_{L} CDR2 comprising or consisting of SEQ ID NO:122, and V_{L} CDR3 comprising or consisting of SEQ ID NO:123;
a light chain comprising an amino acid sequence at least 80% identical, at least 85% identical, at least 90% identical, at least 95% identical, at last 97% identical, or at least 99% identical to SEQ ID NO:322, wherein the light chain comprises V_{L} CDR1 comprising or consisting of SEQ ID NO:129, V_{L} CDR2 comprising or consisting of SEQ ID NO:130, and V_{L} CDR3 comprising or consisting of SEQ ID NO:131;
a light chain comprising an amino acid sequence at least 80% identical, at least 85% identical, at least 90% identical, at least 95% identical, at last 97% identical, or at least 99% identical to SEQ ID NO:324, wherein the light chain comprises V_{L} CDR1 comprising or consisting of SEQ ID NO:137, V_{L} CDR2 comprising or consisting of SEQ ID NO:138, and V_{L} CDR3 comprising or consisting of SEQ ID NO:139;
a light chain comprising an amino acid sequence at least 80% identical, at least 85% identical, at least 90% identical, at least 95% identical, at last 97% identical, or at least 99% identical to SEQ ID NO:326, wherein the light chain comprises V_{L} CDR1 comprising or consisting of SEQ ID NO:145, V_{L} CDR2 comprising or consisting of SEQ ID NO:146, and V_{L} CDR3 comprising or consisting of SEQ ID NO:147;
a light chain comprising an amino acid sequence at least 80% identical, at least 85% identical, at least 90% identical, at least 95% identical, at last 97% identical, or at least 99% identical to SEQ ID NO:328, wherein the light chain comprises V_{L} CDR1 comprising or consisting of SEQ ID NO:153, V_{L} CDR2 comprising or consisting of SEQ ID NO:154, and V_{L} CDR3 comprising or consisting of SEQ ID NO:155;
a light chain comprising an amino acid sequence at least 80% identical, at least 85% identical, at least 90% identical, at least 95% identical, at last 97% identical, or at least 99% identical to SEQ ID NO:330, wherein the light chain comprises V_{L} CDR1 comprising or consisting of SEQ ID NO:161, V_{L} CDR2 comprising or consisting of SEQ ID NO:162, and V_{L} CDR3 comprising or consisting of SEQ ID NO:163;
a light chain comprising an amino acid sequence at least 80% identical, at least 85% identical, at least 90% identical, at least 95% identical, at last 97% identical, or at least 99% identical to SEQ ID NO:332, wherein the light chain comprises V_{L} CDR1 comprising or consisting of SEQ ID NO:169, V_{L} CDR2 comprising or consisting of SEQ ID NO:170, and V_{L} CDR3 comprising or consisting of SEQ ID NO:171;
a light chain comprising an amino acid sequence at least 80% identical, at least 85% identical, at least 90% identical, at least 95% identical, at last 97% identical, or at least 99% identical to SEQ ID NO:334, wherein the light chain comprises V_{L} CDR1 comprising or consisting of SEQ ID NO:177, V_{L} CDR2 comprising or consisting of SEQ ID NO: 178, and V_{L} CDR3 comprising or consisting of SEQ ID NO: 179;
a light chain comprising an amino acid sequence at least 80% identical, at least 85% identical, at least 90% identical, at least 95% identical, at last 97% identical, or at least 99% identical to SEQ ID NO:336, wherein the light chain comprises V_{L} CDR1 comprising or consisting of SEQ ID NO:185, V_{L} CDR2 comprising or consisting of SEQ ID NO: 186, and V_{L} CDR3 comprising or consisting of SEQ ID NO: 187;
a light chain comprising an amino acid sequence at least 80% identical, at least 85% identical, at least 90% identical, at least 95% identical, at last 97% identical, or at least 99% identical to SEQ ID NO:338, wherein the light chain comprises V_{L} CDR1 comprising or consisting of SEQ ID NO: 193, V_{L} CDR2 comprising or consisting of SEQ ID NO:194, and V_{L} CDR3 comprising or consisting of SEQ ID NO:195;
a light chain comprising an amino acid sequence at least 80% identical, at least 85% identical, at least 90% identical, at least 95% identical, at last 97% identical, or at least 99% identical to SEQ ID NO:340, wherein the light chain comprises V_{L} CDR1 comprising or consisting of SEQ ID NO:201, V_{L} CDR2 comprising or consisting of SEQ ID NO:202, and V_{L} CDR3 comprising or consisting of SEQ ID NO:203;
a light chain comprising an amino acid sequence at least 80% identical, at least 85% identical, at least 90% identical, at least 95% identical, at last 97% identical, or at least 99% identical to SEQ ID NO:342, wherein the light chain comprises V_{L} CDR1 comprising or consisting of SEQ ID NO:209, V_{L} CDR2 comprising or consisting of SEQ ID NO:210, and V_{L} CDR3 comprising or consisting of SEQ ID NO:211;
a light chain comprising an amino acid sequence at least 80% identical, at least 85% identical, at least 90% identical, at least 95% identical, at last 97% identical, or at least 99% identical to SEQ ID NO:344, wherein the light chain comprises V_{L} CDR1 comprising or consisting of SEQ ID NO:217, V_{L} CDR2 comprising or consisting of SEQ ID NO:218, and V_{L} CDR3 comprising or consisting of SEQ ID NO:219;
a light chain comprising an amino acid sequence at least 80% identical, at least 85% identical, at least 90% identical, at least 95% identical, at last 97% identical, or at least 99% identical to SEQ ID NO:346, wherein the light chain comprises V_{L} CDR1 comprising or consisting of SEQ ID NO:225, V_{L} CDR2 comprising or consisting of SEQ ID NO:226, and V_{L} CDR3 comprising or consisting of SEQ ID NO:227;
a light chain comprising an amino acid sequence at least 80% identical, at least 85% identical, at least 90% identical, at least 95% identical, at last 97% identical, or at least 99% identical to SEQ ID NO:348, wherein the light chain comprises V_{L} CDR1 comprising or consisting of SEQ ID NO:233, V_{L} CDR2 comprising or consisting of SEQ ID NO:234, and V_{L} CDR3 comprising or consisting of SEQ ID NO:235;
a light chain comprising an amino acid sequence at least 80% identical, at least 85% identical, at least 90% identical, at least 95% identical, at last 97% identical, or at least 99% identical to SEQ ID NO:350, wherein the light chain comprises V_{L} CDR1 comprising or consisting of SEQ ID NO:241, V_{L} CDR2 comprising or consisting of SEQ ID NO:242, and V_{L} CDR3 comprising or consisting of SEQ ID NO:243;
a light chain comprising an amino acid sequence at least 80% identical, at least 85% identical, at least 90% identical, at least 95% identical, at last 97% identical, or at least 99% identical to SEQ ID NO:352, wherein the light chain comprises V_{L} CDR1 comprising or consisting of SEQ ID NO:249, V_{L} CDR2 comprising or consisting of SEQ ID NO:250, and V_{L} CDR3 comprising or consisting of SEQ ID NO:251;
a light chain comprising an amino acid sequence at least 80% identical, at least 85% identical, at least 90% identical, at least 95% identical, at last 97% identical, or at least 99% identical to SEQ ID NO:354, wherein the light chain comprises V_{L} CDR1 comprising or consisting of SEQ ID NO:257, V_{L} CDR2 comprising or consisting of SEQ ID NO:258, and V_{L} CDR3 comprising or consisting of SEQ ID NO:259;
a light chain comprising an amino acid sequence at least 80% identical, at least 85% identical, at least 90% identical, at least 95% identical, at last 97% identical, or at least 99% identical to SEQ ID NO:356, wherein the light chain comprises V_{L} CDR1 comprising or consisting of SEQ ID NO:265, V_{L} CDR2 comprising or consisting of SEQ ID NO:266, and V_{L} CDR3 comprising or consisting of SEQ ID NO:267;
a light chain comprising an amino acid sequence at least 80% identical, at least 85% identical, at least 90% identical, at least 95% identical, at last 97% identical, or at least 99% identical to SEQ ID NO:358, wherein the light chain comprises V_{L} CDR1 comprising or consisting of SEQ ID NO:273, V_{L} CDR2 comprising or consisting of SEQ ID NO:274, and V_{L} CDR3 comprising or consisting of SEQ ID NO:275; and,
a light chain comprising an amino acid sequence at least 80% identical, at least 85% identical, at least 90% identical, at least 95% identical, at last 97% identical, or at least 99% identical to SEQ ID NO:360, wherein the light chain comprises V_{L} CDR1 comprising or consisting of SEQ ID NO:281, V_{L} CDR2 comprising or consisting of SEQ ID NO:282, and V_{L} CDR3 comprising or consisting of SEQ ID NO:283.

In one aspect, the isolated antibody may comprise a heavy chain comprising an amino acid sequence at least 80% identical, at least 85% identical, at least 90% identical, at least 95% identical, at last 97% identical, at least 99% identical, or 100% identical to SEQ ID NO:289, SEQ ID NO:291, SEQ ID NO:293, SEQ ID NO:295, SEQ ID NO:297, SEQ ID NO:299, SEQ ID NO:301, SEQ ID NO:303, SEQ ID NO:305, SEQ ID NO:307, SEQ ID NO:309, SEQ ID NO:311, SEQ ID NO:313, SEQ ID NO:315, SEQ ID NO:317, SEQ ID NO:319, SEQ ID NO:321, SEQ ID NO:323, SEQ ID NO:325, SEQ ID NO:327, SEQ ID NO:329, SEQ ID NO:331, SEQ ID NO:333, SEQ ID NO:335, SEQ ID NO:337, SEQ ID NO:339, SEQ ID NO:341, SEQ ID NO:343, SEQ ID NO:345, SEQ ID NO:347, SEQ ID NO:349, SEQ ID NO:351, SEQ ID NO:353, SEQ ID NO:355, SEQ ID NO:357, or SEQ ID NO:359.

In one aspect, the isolated antibody may comprise a heavy chain selected from the group consisting of:
a heavy chain comprising an amino acid sequence at least 80% identical, at least 85% identical, at least 90% identical, at least 95% identical, at last 97% identical, or at least 99% identical to SEQ ID NO:289, wherein the heavy chain comprises V_{H} CDR1 comprising or consisting of SEQ ID NO:5, V_{H} CDR2 comprising or consisting of SEQ ID NO:6, and V_{H} CDR3 comprising or consisting of SEQ ID NO:7;
a heavy chain comprising an amino acid sequence at least 80% identical, at least 85% identical, at least 90% identical, at least 95% identical, at last 97% identical, or at least 99% identical to SEQ ID NO:291, wherein the heavy chain comprises V_{H} CDR1 comprising or consisting of SEQ ID NO:13, V_{H} CDR2 comprising or consisting of SEQ ID NO:14, and V_{H} CDR3 comprising or consisting of SEQ ID NO:15;
a heavy chain comprising an amino acid sequence at least 80% identical, at least 85% identical, at least 90% 213, at least 95% identical, at last 97% identical, or at least 99% identical to SEQ ID NO:293, wherein the heavy chain comprises V_{H} CDR1 comprising or consisting of SEQ ID NO:21, V_{H} CDR2 comprising or consisting of SEQ ID NO:22, and V_{H} CDR3 comprising or consisting of SEQ ID NO:23;
a heavy chain comprising an amino acid sequence at least 80% identical, at least 85% identical, at least 90% identical, at least 95% identical, at last 97% identical, or at least 99% identical to SEQ ID NO:295, wherein the heavy chain comprises V_{H} CDR1 comprising or consisting of SEQ ID NO:29, V_{H} CDR2 comprising or consisting of SEQ ID NO:30, and V_{H} CDR3 comprising or consisting of SEQ ID NO:31;
a heavy chain comprising an amino acid sequence at least 80% identical, at least 85% identical, at least 90% identical, at least 95% identical, at last 97% identical, or at least 99% identical to SEQ ID NO:297, wherein the heavy chain comprises V_{H} CDR1 comprising or consisting of SEQ ID NO:37, V_{H} CDR2 comprising or consisting of SEQ ID NO:38, and V_{H} CDR3 comprising or consisting of SEQ ID NO:39;
a heavy chain comprising an amino acid sequence at least 80% identical, at least 85% identical, at least 90% identical, at least 95% identical, at last 97% identical, or at least 99% identical to SEQ ID NO:299, wherein the heavy chain comprises V_{H} CDR1 comprising or consisting of SEQ ID NO:45, V_{H} CDR2 comprising or consisting of SEQ ID NO:46, and V_{H} CDR3 comprising or consisting of SEQ ID NO:47;
a heavy chain comprising an amino acid sequence at least 80% identical, at least 85% identical, at least 90% identical, at least 95% identical, at last 97% identical, or at least 99% identical to SEQ ID NO:301, wherein the heavy chain comprises V_{H} CDR1 comprising or consisting of SEQ ID NO:53, V_{H} CDR2 comprising or consisting of SEQ ID NO:54, and V_{H} CDR3 comprising or consisting of SEQ ID NO:55;
a heavy chain comprising an amino acid sequence at least 80% identical, at least 85% identical, at least 90% identical, at least 95% identical, at last 97% identical, or at least 99% identical to SEQ ID NO:303, wherein the heavy chain comprises V_{H} CDR1 comprising or consisting of SEQ ID NO:61, V_{H} CDR2 comprising or consisting of SEQ ID NO:62, and V_{H} CDR3 comprising or consisting of SEQ ID NO:63;
a heavy chain comprising an amino acid sequence at least 80% identical, at least 85% identical, at least 90% identical, at least 95% identical, at last 97% identical, or at least 99% identical to SEQ ID NO:305, wherein the heavy chain comprises V_{H} CDR1 comprising or consisting of SEQ ID NO:69, V_{H} CDR2 comprising or consisting of SEQ ID NO:70, and V_{H} CDR3 comprising or consisting of SEQ ID NO:71;
a heavy chain comprising an amino acid sequence at least 80% identical, at least 85% identical, at least 90% identical, at least 95% identical, at last 97% identical, or at least 99% identical to SEQ ID NO:307, wherein the heavy chain comprises V_{H} CDR1 comprising or consisting of SEQ ID NO:77, V_{H} CDR2 comprising or consisting of SEQ ID NO:78, and V_{H} CDR3 comprising or consisting of SEQ ID NO:79;
a heavy chain comprising an amino acid sequence at least 80% identical, at least 85% identical, at least 90% identical, at least 95% identical, at last 97% identical, or at least 99% identical to SEQ ID NO:309, wherein the heavy chain comprises V_{H} CDR1 comprising or consisting of SEQ ID NO:85, V_{H} CDR2 comprising or consisting of SEQ ID NO:86, and V_{H} CDR3 comprising or consisting of SEQ ID NO:87;
a heavy chain comprising an amino acid sequence at least 80% identical, at least 85% identical, at least 90% identical, at least 95% identical, at last 97% identical, or at least 99% identical to SEQ ID NO:311, wherein the heavy chain comprises V_{H} CDR1 comprising or consisting of SEQ ID NO:93, V_{H} CDR2 comprising or consisting of SEQ ID NO:94, and V_{H} CDR3 comprising or consisting of SEQ ID NO:95;
a heavy chain comprising an amino acid sequence at least 80% identical, at least 85% identical, at least 90% identical, at least 95% identical, at last 97% identical, or at least 99% identical to SEQ ID NO:313, wherein the heavy chain comprises V_{H} CDR1 comprising or consisting of SEQ ID NO:101, V_{H} CDR2 comprising or consisting of SEQ ID NO:102, and V_{H} CDR3 comprising or consisting of SEQ ID NO:103;
a heavy chain comprising an amino acid sequence at least 80% identical, at least 85% identical, at least 90% identical, at least 95% identical, at last 97% identical, or at least 99% identical to SEQ ID NO:315, wherein the heavy chain comprises V_{H} CDR1 comprising or consisting of SEQ ID NO:109, V_{H} CDR2 comprising or consisting of SEQ ID NO:110, and V_{H} CDR3 comprising or consisting of SEQ ID NO:111;
a heavy chain comprising an amino acid sequence at least 80% identical, at least 85% identical, at least 90% identical, at least 95% identical, at last 97% identical, or at least 99% identical to SEQ ID NO:317, wherein the heavy chain comprises V_{H} CDR1 comprising or consisting of SEQ ID NO:117, V_{H} CDR2 comprising or consisting of SEQ ID NO: 118, and V_{H} CDR3 comprising or consisting of SEQ ID NO: 119;
a heavy chain comprising an amino acid sequence at least 80% identical, at least 85% identical, at least 90% identical, at least 95% identical, at last 97% identical, or at least 99% identical to SEQ ID NO:319, wherein the heavy chain comprises V_{H} CDR1 comprising or consisting of SEQ ID NO:125, V_{H} CDR2 comprising or consisting of SEQ ID NO:126, and V_{H} CDR3 comprising or consisting of SEQ ID NO:127;
a heavy chain comprising an amino acid sequence at least 80% identical, at least 85% identical, at least 90% identical, at least 95% identical, at last 97% identical, or at least 99% identical to SEQ ID NO:321, wherein the heavy chain comprises V_{H} CDR1 comprising or consisting of SEQ ID NO:133, V_{H} CDR2 comprising or consisting of SEQ ID NO:134, and V_{H} CDR3 comprising or consisting of SEQ ID NO:135;
a heavy chain comprising an amino acid sequence at least 80% identical, at least 85% identical, at least 90% identical, at least 95% identical, at last 97% identical, or at least 99% identical to SEQ ID NO:323, wherein the heavy chain comprises V_{H} CDR1 comprising or consisting of SEQ ID NO:141, V_{H} CDR2 comprising or consisting of SEQ ID NO: 142, and V_{H} CDR3 comprising or consisting of SEQ ID NO 143;
a heavy chain comprising an amino acid sequence at least 80% identical, at least 85% identical, at least 90% identical, at least 95% identical, at last 97% identical, or at least 99% identical to SEQ ID NO:325, wherein the heavy chain comprises V_{H} CDR1 comprising or consisting of SEQ ID NO:149, V_{H} CDR2 comprising or consisting of SEQ ID NO:150, and V_{H} CDR3 comprising or consisting of SEQ ID NO:151;
a heavy chain comprising an amino acid sequence at least 80% identical, at least 85% identical, at least 90% identical, at least 95% identical, at last 97% identical, or at least 99% identical to SEQ ID NO:327, wherein the heavy chain comprises V_{H} CDR1 comprising or consisting of SEQ ID NO:157, V_{H} CDR2 comprising or consisting of SEQ ID NO: 158, and V_{H} CDR3 comprising or consisting of SEQ ID NO:159;
a heavy chain comprising an amino acid sequence at least 80% identical, at least 85% identical, at least 90% identical, at least 95% identical, at last 97% identical, or at least 99% identical to SEQ ID NO:329, wherein the heavy chain comprises V_{H} CDR1 comprising or consisting of SEQ ID NO:165, V_{H} CDR2 comprising or consisting of SEQ ID NO:166, and V_{H} CDR3 comprising or consisting of SEQ ID NO:167;
a heavy chain comprising an amino acid sequence at least 80% identical, at least 85% identical, at least 90% identical, at least 95% identical, at last 97% identical, or at least 99% identical to SEQ ID NO:331, wherein the heavy chain comprises V_{H} CDR1 comprising or consisting of SEQ ID NO: 173, V_{H} CDR2 comprising or consisting of SEQ ID NO:174, and V_{H} CDR3 comprising or consisting of SEQ ID NO:175;
a heavy chain comprising an amino acid sequence at least 80% identical, at least 85% identical, at least 90% identical, at least 95% identical, at last 97% identical, or at least 99% identical to SEQ ID NO:333, wherein the heavy chain comprises V_{H} CDR1 comprising or consisting of SEQ ID NO:181, V_{H} CDR2 comprising or consisting of SEQ ID NO:182, and V_{H} CDR3 comprising or consisting of SEQ ID NO:183;
a heavy chain comprising an amino acid sequence at least 80% identical, at least 85% identical, at least 90% identical, at least 95% identical, at last 97% identical, or at least 99% identical to SEQ ID NO:335, wherein the heavy chain comprises V_{H} CDR1 comprising or consisting of SEQ ID NO:189, V_{H} CDR2 comprising or consisting of SEQ ID NO:190, and V_{H} CDR3 comprising or consisting of SEQ ID NO:191;
a heavy chain comprising an amino acid sequence at least 80% identical, at least 85% identical, at least 90% identical, at least 95% identical, at last 97% identical, or at least 99% identical to SEQ ID NO:337, wherein the heavy chain comprises V_{H} CDR1 comprising or consisting of SEQ ID NO:197, V_{H} CDR2 comprising or consisting of SEQ ID NO:198, and V_{H} CDR3 comprising or consisting of SEQ ID NO: 199; and,
a heavy chain comprising an amino acid sequence at least 80% identical, at least 85% identical, at least 90% identical, at least 95% identical, at last 97% identical, or at least 99% identical to SEQ ID NO:339, wherein the heavy chain comprises V_{H} CDR1 comprising or consisting of SEQ ID NO:205, V_{H} CDR2 comprising or consisting of SEQ ID NO:206, and V_{H} CDR3 comprising or consisting of SEQ ID NO:207;
a heavy chain comprising an amino acid sequence at least 80% identical, at least 85% identical, at least 90% identical, at least 95% identical, at last 97% identical, or at least 99% identical to SEQ ID NO:341, wherein the heavy chain comprises V_{H} CDR1 comprising or consisting of SEQ ID NO:213, V_{H} CDR2 comprising or consisting of SEQ ID NO:214, and V_{H} CDR3 comprising or consisting of SEQ ID NO:215;
a heavy chain comprising an amino acid sequence at least 80% identical, at least 85% identical, at least 90% identical, at least 95% identical, at last 97% identical, or at least 99% identical to SEQ ID NO:343, wherein the heavy chain comprises V_{H} CDR1 comprising or consisting of SEQ ID NO:221, V_{H} CDR2 comprising or consisting of SEQ ID NO:222, and V_{H} CDR3 comprising or consisting of SEQ ID NO:223;
a heavy chain comprising an amino acid sequence at least 80% identical, at least 85% identical, at least 90% identical, at least 95% identical, at last 97% identical, or at least 99% identical to SEQ ID NO:345, wherein the heavy chain comprises V_{H} CDR1 comprising or consisting of SEQ ID NO:229, V_{H} CDR2 comprising or consisting of SEQ ID NO:230, and V_{H} CDR3 comprising or consisting of SEQ ID NO:231;
a heavy chain comprising an amino acid sequence at least 80% identical, at least 85% identical, at least 90% identical, at least 95% identical, at last 97% identical, or at least 99% identical to SEQ ID NO:347, wherein the heavy chain comprises V_{H} CDR1 comprising or consisting of SEQ ID NO:237, V_{H} CDR2 comprising or consisting of SEQ ID NO:238, and V_{H} CDR3 comprising or consisting of SEQ ID NO:239;
a heavy chain comprising an amino acid sequence at least 80% identical, at least 85% identical, at least 90% identical, at least 95% identical, at last 97% identical, or at least 99% identical to SEQ ID NO:349, wherein the heavy chain comprises V_{H} CDR1 comprising or consisting of SEQ ID NO:245, V_{H} CDR2 comprising or consisting of SEQ ID NO:246 and V_{H} CDR3 comprising or consisting of SEQ ID NO:247;
a heavy chain comprising an amino acid sequence at least 80% identical, at least 85% identical, at least 90% identical, at least 95% identical, at last 97% identical, or at least 99% identical to SEQ ID NO:351, wherein the heavy chain comprises V_{H} CDR1 comprising or consisting of SEQ ID NO:253, V_{H} CDR2 comprising or consisting of SEQ ID NO:254, and V_{H} CDR3 comprising or consisting of SEQ ID NO:255;
a heavy chain comprising an amino acid sequence at least 80% identical, at least 85% identical, at least 90% identical, at least 95% identical, at last 97% identical, or at least 99% identical to SEQ ID NO:353, wherein the heavy chain comprises V_{H} CDR1 comprising or consisting of SEQ ID NO:261, V_{H} CDR2 comprising or consisting of SEQ ID NO:262, and V_{H} CDR3 comprising or consisting of SEQ ID NO:263;
a heavy chain comprising an amino acid sequence at least 80% identical, at least 85% identical, at least 90% identical, at least 95% identical, at last 97% identical, or at least 99% identical to SEQ ID NO:355, wherein the heavy chain comprises V_{H} CDR1 comprising or consisting of SEQ ID NO:269, V_{H} CDR2 comprising or consisting of SEQ ID NO:270, and V_{H} CDR3 comprising or consisting of SEQ ID NO:271;
a heavy chain comprising an amino acid sequence at least 80% identical, at least 85% identical, at least 90% identical, at least 95% identical, at last 97% identical, or at least 99% identical to SEQ ID NO:357, wherein the heavy chain comprises V_{H} CDR1 comprising or consisting of SEQ ID NO:277, V_{H} CDR2 comprising or consisting of SEQ ID NO:278, and V_{H} CDR3 comprising or consisting of SEQ ID NO:279; and,
a heavy chain comprising an amino acid sequence at least 80% identical, at least 85% identical, at least 90% identical, at least 95% identical, at last 97% identical, or at least 99% identical to SEQ ID NO:359, wherein the heavy chain comprises V_{H} CDR1 comprising or consisting of SEQ ID NO:285, V_{H} CDR2 comprising or consisting of SEQ ID NO:286, and V_{H} CDR3 comprising or consisting of SEQ ID NO:287.

### Define Each Isolated Antibody by Matched Light and Heavy Chain

In one aspect, the isolated antibody (e.g., SKW10) may comprise alight chain and a heavy chain, wherein the light chain comprises an amino acid sequence at least 80% identical, at least 85% identical, at least 90% identical, at least 95% identical, at last 97% identical, or at least 99% identical to SEQ ID NO:290, and the heavy chain comprises an amino acid sequence at least 80% identical, at least 85% identical, at least 90% identical, at least 95% identical, at least 97% identical, at least 99% identical, or 100% identical to SEQ ID NO:289. In certain aspects, the light chain may comprise a V_{L} CDR1 comprising or consisting of SEQ ID NO:1, a V_{L} CDR2 comprising or consisting of SEQ ID NO:2, and a V_{L} CDR3 comprising or consisting of SEQ ID NO:3. In certain aspects, the heavy chain may comprise a V_{H} CDR1 comprising or consisting of SEQ ID NO:5, a V_{H} CDR2 comprising or consisting of SEQ ID NO:6, and a V_{H} CDR3 comprising or consisting of SEQ ID NO:7. In certain aspects, the light chain may comprise SEQ ID NO:290 and the heavy chain may comprise SEQ ID NO:289.

In one aspect, the isolated antibody (e.g., SKW11) comprises a light chain and a heavy chain, wherein the light chain comprises amino acid sequence at least 80% identical, at least 85% identical, at least 90% identical, at least 95% identical, at last 97% identical, or at least 99% identical to SEQ ID NO:292, and the heavy chain comprises an amino acid sequence at least 80% identical, at least 85% identical, at least 90% identical, at least 95% identical, at last 97% identical, or at least 99% identical to SEQ ID NO:291. In certain aspects, the light chain may comprise a V_{L} CDR1 comprising or consisting of SEQ ID NO:9, a V_{L} CDR2 comprising or consisting of SEQ ID NO:10, and a V_{L} CDR3 comprising or consisting of SEQ ID NO:11. In certain aspects, the heavy chain may comprise a V_{H} CDR1 comprising or consisting of SEQ ID NO: 13, a V_{H} CDR2 comprising or consisting of SEQ ID NO: 14, and a V_{H} CDR3 comprising or consisting of SEQ ID NO:15. In certain aspects, the light chain may comprise SEQ ID NO:292 and the heavy chain may comprise SEQ ID NO:291.

In one aspect, the isolated antibody (e.g., SKW14) comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:294, and wherein the heavy chain comprises an amino acid sequence at least 80% identical, at least 85% identical, at least 90% identical, at least 95% identical, at last 97% identical, or at least 99% identical to SEQ ID NO:293. In certain aspects, the light chain may comprise a V_{L} CDR1 comprising or consisting of SEQ ID NO:17, a V_{L} CDR2 comprising or consisting of SEQ ID NO:18, and a V_{L} CDR3 comprising or consisting of SEQ ID NO:19. In certain aspects, the heavy chain may comprise a V_{H} CDR1 comprising or consisting of SEQ ID NO:21, a V_{H} CDR2 comprising or consisting of SEQ ID NO:22, and a V_{H} CDR3 comprising or consisting of SEQ ID NO:23. In certain aspects, the light chain may comprise SEQ ID NO:294 and the heavy chain may comprise SEQ ID NO:293.

In one aspect, the isolated antibody (SKW15) comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:296, and the heavy chain comprises an amino acid sequence at least 80% identical, at least 85% identical, at least 90% identical, at least 95% identical, at last 97% identical, or at least 99% identical to SEQ ID NO:295. In certain aspects, the light chain may comprise a V_{L} CDR1 comprising or consisting of SEQ ID NO:25, a V_{L} CDR2 comprising or consisting of SEQ ID NO:26, and a V_{L} CDR3 comprising or consisting of SEQ ID NO:27. In certain aspects, the heavy chain may comprise a V_{H} CDR1 comprising or consisting of SEQ ID NO:29, a V_{H} CDR2 comprising or consisting of SEQ ID NO:30, and a V_{H} CDR3 comprising or consisting of SEQ ID NO:31. In certain aspects, the light chain may comprise SEQ ID NO:296 and the heavy chain may comprise SEQ ID NO:295.

In one aspect, the isolated antibody (e.g., SKW16) comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:298, and the heavy chain comprises an amino acid sequence at least 80% identical, at least 85% identical, at least 90% identical, at least 95% identical, at last 97% identical, or at least 99% identical to SEQ ID NO:297. In certain aspects, the light chain may comprise a V_{L} CDR1 comprising or consisting of SEQ ID NO:33, a V_{L} CDR2 comprising or consisting of SEQ ID NO:34, and a V_{L} CDR3 comprising or consisting of SEQ ID NO:35. In certain aspects, the heavy chain may comprise a V_{L} CDR1 comprising or consisting of SEQ ID NO:37, a V_{H} CDR2 comprising or consisting of SEQ ID NO:38, and a V_{H} CDR3 comprising or consisting of SEQ ID NO:39. In certain aspects, the light chain may comprise SEQ ID NO:298 and the heavy chain may comprise SEQ ID NO:297.

In one aspect, the isolated antibody (e.g., SKW17) comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:300, and the heavy chain comprises an amino acid sequence at least 80% identical, at least 85% identical, at least 90% identical, at least 95% identical, at last 97% identical, or at least 99% identical to SEQ ID NO:299. In certain aspects, the light chain may comprise a V_{L} CDR1 comprising or consisting of SEQ ID NO:41, a V_{L} CDR2 comprising or consisting of SEQ ID NO:42, and a V_{L} CDR3 comprising or consisting of SEQ ID NO:43. In certain aspects, the heavy chain may comprise a V_{H} CDR1 comprising or consisting of SEQ ID NO:45, a V_{H} CDR2 comprising or consisting of SEQ ID NO:46, and a V_{H} CDR3 comprising or consisting of SEQ ID NO:47. In certain aspects, the light chain may comprise SEQ ID NO:300 and the heavy chain may comprise SEQ ID NO:299.

In one aspect, the isolated antibody (e.g., SKW18) comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:302, and the heavy chain comprises an amino acid sequence at least 80% identical, at least 85% identical, at least 90% identical, at least 95% identical, at last 97% identical, or at least 99% identical to SEQ ID NO:301. In certain aspects, the light chain may comprise a V_{L} CDR1 comprising or consisting of SEQ ID NO:49, a V_{L} CDR2 comprising or consisting of SEQ ID NO:50, and a V_{L} CDR3 comprising or consisting of SEQ ID NO:51. In certain aspects, the heavy chain may comprise a V_{H} CDR1 comprising or consisting of SEQ ID NO:53, a V_{H} CDR2 comprising or consisting of SEQ ID NO:54, and a V_{H} CDR3 comprising or consisting of SEQ ID NO:55. In certain aspects, the light chain may comprise SEQ ID NO:302 and the heavy chain may comprise SEQ ID NO:301.

In one aspect, the isolated antibody (e.g., SKW19) comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:304, and the heavy chain comprises an amino acid sequence at least 80% identical, at least 85% identical, at least 90% identical, at least 95% identical, at last 97% identical, or at least 99% identical to SEQ ID NO:303. In certain aspects, the light chain may comprise a V_{L} CDR1 comprising or consisting of SEQ ID NO:57, a V_{L} CDR2 comprising or consisting of SEQ ID NO:58, and a V_{L} CDR3 comprising or consisting of SEQ ID NO:59. In certain aspects, the heavy chain may comprise a V_{H} CDR1 comprising or consisting of SEQ ID NO:61, a V_{H} CDR2 comprising or consisting of SEQ ID NO:62, and a V_{H} CDR3 comprising or consisting of SEQ ID NO:63. In certain aspects, the light chain may comprise SEQ ID NO:304 and the heavy chain may comprise SEQ ID NO:303.

In one aspect, the isolated antibody (e.g., KW24) comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:306, and the heavy chain comprises an amino acid sequence at least 80% identical, at least 85% identical, at least 90% identical, at least 95% identical, at last 97% identical, or at least 99% identical to SEQ ID NO:305. In certain aspects, the light chain may comprise a V_{L} CDR1 comprising or consisting of SEQ ID NO:65, a V_{L} CDR2 comprising or consisting of SEQ ID NO:66, and a V_{L} CDR3 comprising or consisting of SEQ ID NO:67. In certain aspects, the heavy chain may comprise a V_{H} CDR1 comprising or consisting of SEQ ID NO:69, a V_{H} CDR2 comprising or consisting of SEQ ID NO:70, and a V_{H} CDR3 comprising or consisting of SEQ ID NO:71. In certain aspects, the light chain may comprise SEQ ID NO:306 and the heavy chain may comprise SEQ ID NO:305.

In one aspect, the isolated antibody (e.g., SKW29) comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:308, and the heavy chain comprises an amino acid sequence at least 80% identical, at least 85% identical, at least 90% identical, at least 95% identical, at last 97% identical, or at least 99% identical to SEQ ID NO:307. In certain aspects, the light chain may comprise a V_{L} CDR1 comprising or consisting of SEQ ID NO:73, a V_{L} CDR2 comprising or consisting of SEQ ID NO:74, and a V_{L} CDR3 comprising or consisting of SEQ ID NO:75. In certain aspects, the heavy chain may comprise a V_{H} CDR1 comprising or consisting of SEQ ID NO:77, a V_{H} CDR2 comprising or consisting of SEQ ID NO:78, and a V_{H} CDR3 comprising or consisting of SEQ ID NO:79. In certain aspects, the light chain may comprise SEQ ID NO:308 and the heavy chain may comprise SEQ ID NO:307.

In one aspect, the isolated antibody (e.g., SKW30) comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:310, and the heavy chain comprises an amino acid sequence at least 80% identical, at least 85% identical, at least 90% identical, at least 95% identical, at last 97% identical, or at least 99% identical to SEQ ID NO:309. In certain aspects, the light chain may comprise a V_{L} CDR1 comprising or consisting of SEQ ID NO:81, a V_{L} CDR2 comprising or consisting of SEQ ID NO:82, and a V_{L} CDR3 comprising or consisting of SEQ ID NO:83. In certain aspects, the heavy chain may comprise a V_{H} CDR1 comprising or consisting of SEQ ID NO:85, a V_{H} CDR2 comprising or consisting of SEQ ID NO:86, and a V_{H} CDR3 comprising or consisting of SEQ ID NO:87. In certain aspects, the light chain may comprise SEQ ID NO:310 and the heavy chain may comprise SEQ ID NO:309.

In one aspect, the isolated antibody (e.g., SKE25) comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:312, and the heavy chain comprises an amino acid sequence at least 80% identical, at least 85% identical, at least 90% identical, at least 95% identical, at last 97% identical, or at least 99% identical to SEQ ID NO:311. In certain aspects, the light chain may comprise a V_{L} CDR1 comprising or consisting of SEQ ID NO:89, a V_{L} CDR2 comprising or consisting of SEQ ID NO:90, and a V_{L} CDR3 comprising or consisting of SEQ ID NO:91. In certain aspects, the heavy chain may comprise a V_{H} CDR1 comprising or consisting of SEQ ID NO:93, a V_{H} CDR2 comprising or consisting of SEQ ID NO:94, and a V_{H} CDR3 comprising or consisting of SEQ ID NO:95. In certain aspects, the light chain may comprise SEQ ID NO:312 and the heavy chain may comprise SEQ ID NO:311.

In one aspect, the isolated antibody (e.g., SKE26) comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:314, and the heavy chain comprises an amino acid sequence at least 80% identical, at least 85% identical, at least 90% identical, at least 95% identical, at last 97% identical, or at least 99% identical to SEQ ID NO:313. In certain aspects, the light chain may comprise a V_{L} CDR1 comprising or consisting of SEQ ID NO:97, a V_{L} CDR2 comprising or consisting of SEQ ID NO:98, and a V_{L} CDR3 comprising or consisting of SEQ ID NO:99. In certain aspects, the heavy chain may comprise a V_{H} CDR1 comprising or consisting of SEQ ID NO:101, a V_{H} CDR2 comprising or consisting of SEQ ID NO:102, and a V_{H} CDR3 comprising or consisting of SEQ ID NO:103. In certain aspects, the light chain may comprise SEQ ID NO:314 and the heavy chain may comprise SEQ ID NO:313.

In one aspect, the isolated antibody (e.g., SKE27) comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:316, and the heavy chain comprises an amino acid sequence at least 80% identical, at least 85% identical, at least 90% identical, at least 95% identical, at last 97% identical, or at least 99% identical to SEQ ID NO:315. In certain aspects, the light chain may comprise a V_{L} CDR1 comprising or consisting of SEQ ID NO:105, a V_{L} CDR2 comprising or consisting of SEQ ID NO:106, and a V_{L} CDR3 comprising or consisting of SEQ ID NO:107. In certain aspects, the heavy chain may comprise a V_{H} CDR1 comprising or consisting of SEQ ID NO: 109, a V_{H} CDR2 comprising or consisting of SEQ ID NO: 110, and a V_{H} CDR3 comprising or consisting of SEQ ID NO:111. In certain aspects, the light chain may comprise SEQ ID NO:316 and the heavy chain may comprise SEQ ID NO:315.

In one aspect, the isolated antibody (e.g., SKE33) comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:318, and the heavy chain comprises an amino acid sequence at least 80% identical, at least 85% identical, at least 90% identical, at least 95% identical, at last 97% identical, or at least 99% identical to SEQ ID NO:317. In certain aspects the light chain may comprise a V_{L} CDR1 comprising or consisting of SEQ ID NO:113, a V_{L} CDR2 comprising or consisting of SEQ ID NO:114, and a V_{L} CDR3 comprising or consisting of SEQ ID NO:115. In certain aspects, the heavy chain may comprise a V_{L} CDR1 comprising or consisting of SEQ ID NO:117, a V_{H} CDR2 comprising or consisting of SEQ ID NO:118, and a V_{H} CDR3 comprising or consisting of SEQ ID NO:119. In certain aspects, the light chain may comprise SEQ ID NO:318 and the heavy chain may comprise SEQ ID NO:317.

In one aspect, the isolated antibody (e.g., SKV09) comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:320, and the heavy chain comprise an amino acid sequence at least 80% identical, at least 85% identical, at least 90% identical, at least 95% identical, at last 97% identical, or at least 99% identical to SEQ ID NO:319. In certain aspects, the light chain may comprise a V_{L} CDR1 comprising or consisting of SEQ ID NO:121, a V_{L} CDR2 comprising or consisting of SEQ ID NO:122, and a V_{L} CDR3 comprising or consisting of SEQ ID NO:123. In certain aspects, the heavy chain may comprise V_{H} CDR1 comprising or consisting of SEQ ID NO:125, V_{H} CDR2 comprising or consisting of SEQ ID NO: 126, and V_{H} CDR3 comprising or consisting of SEQ ID NO:127. In certain aspects, the light chain may comprise SEQ ID NO:320 and the heavy chain may comprise SEQ ID NO:319.

In one aspect, the isolated antibody (e.g., SKV11) comprises alight chain and a heavy chain, wherein the light chain comprises an amino acid sequence at least 80% identical, at least 85% identical, at least 90% identical, at least 95% identical, at last 97% identical, or at least 99% identical to SEQ ID NO:322, and the heavy chain comprises an amino acid sequence at least 80% identical, at least 85% identical, at least 90% identical, at least 95% identical, at last 97% identical, or at least 99% identical to SEQ ID NO:321. In certain aspects the light chain may comprise a V_{L} CDR1 comprising or consisting of SEQ ID NO:129, a V_{L} CDR2 comprising or consisting of SEQ ID NO:130, and a V_{L} CDR3 comprising or consisting of SEQ ID NO:131. In certain aspects, the heavy chain may comprise a V_{H} CDR1 comprising or consisting of SEQ ID NO:133, a V_{H} CDR2 comprising or consisting of SEQ ID NO:134, and a V_{H} CDR3 comprising or consisting of SEQ ID NO:135. In certain aspects, the light chain may comprise SEQ ID NO:322 and the heavy chain may comprise SEQ ID NO:321.

In one aspect, the isolated antibody (e.g., SKV16) comprises alight chain and a heavy chain, wherein the light chain comprises an amino acid sequence at least 80% identical, at least 85% identical, at least 90% identical, at least 95% identical, at last 97% identical, or at least 99% identical to SEQ ID NO:324, and the heavy chain comprises an amino acid sequence at least 80% identical, at least 85% identical, at least 90% identical, at least 95% identical, at last 97% identical, or at least 99% identical to SEQ ID NO:323. In certain aspects, the light chain may comprise a V_{L} CDR1 comprising or consisting of SEQ ID NO:137, a V_{L} CDR2 comprising or consisting of SEQ ID NO:138, and a V_{L} CDR3 comprising or consisting of SEQ ID NO:139. In certain aspects, the heavy chain may comprise a V_{H} CDR1 comprising or consisting of SEQ ID NO:141, a V_{H} CDR2 comprising or consisting of SEQ ID NO:142, and a V_{H} CDR3 comprising or consisting of SEQ ID NO 143. In certain aspects, the light chain may comprise SEQ ID NO:324 and the heavy chain may comprise SEQ ID NO:323.

In one aspect, the isolated antibody (e.g., SKV18) comprises alight chain and a heavy chain, wherein the light chain comprises an amino acid sequence at least 80% identical, at least 85% identical, at least 90% identical, at least 95% identical, at last 97% identical, or at least 99% identical to SEQ ID NO:326, and the heavy chain comprises an amino acid sequence at least 80% identical, at least 85% identical, at least 90% identical, at least 95% identical, at last 97% identical, or at least 99% identical to SEQ ID NO:325. In certain aspects, the light chain may comprise a V_{L} CDR1 comprising or consisting of SEQ ID NO:145, a V_{L} CDR2 comprising or consisting of SEQ ID NO:146, and a V_{L} CDR3 comprising or consisting of SEQ ID NO:147. In certain aspects the heavy chain may comprise a V_{H} CDR1 comprising or consisting of SEQ ID NO:149, a V_{H} CDR2 comprising or consisting of SEQ ID NO: 150, and a V_{H} CDR3 comprising or consisting of SEQ ID NO:151. In certain aspects, the light chain may comprise SEQ ID NO:326 and the heavy chain may comprise SEQ ID NO:325.

In one aspect, the isolated antibody (e.g., SKV20) comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:328, and the heavy chain comprises an amino acid sequence at least 80% identical, at least 85% identical, at least 90% identical, at least 95% identical, at last 97% identical, or at least 99% identical to SEQ ID NO:327. In certain aspects, the light chain may comprise a V_{L} CDR1 comprising or consisting of SEQ ID NO:153, a V_{L} CDR2 comprising or consisting of SEQ ID NO:154, and a V_{L} CDR3 comprising or consisting of SEQ ID NO:155. In certain aspects, the heavy chain may comprise a V_{H} CDR1 comprising or consisting of SEQ ID NO:157, a V_{H} CDR2 comprising or consisting of SEQ ID NO:158, and a V_{H} CDR3 comprising or consisting of SEQ ID NO:159. In certain aspects, the light chain may comprise SEQ ID NO:328 and the heavy chain may comprise SEQ ID NO:327.

In one aspect, the isolated antibody (e.g., SKT01) comprises alight chain and a heavy chain, wherein the light chain comprises an amino acid sequence at least 80% identical, at least 85% identical, at least 90% identical, at least 95% identical, at last 97% identical, or at least 99% identical to SEQ ID NO:330, and the heavy chain comprises an amino acid sequence at least 80% identical, at least 85% identical, at least 90% identical, at least 95% identical, at last 97% identical, or at least 99% identical to SEQ ID NO:329. In certain aspects, the light chain may comprise a V_{L} CDR1 comprising or consisting of SEQ ID NO:161, a V_{L} CDR2 comprising or consisting of SEQ ID NO:162, and a V_{L} CDR3 comprising or consisting of SEQ ID NO:163. In certain aspects, the heavy chain may comprise a V_{H} CDR1 comprising or consisting of SEQ ID NO:165, a V_{H} CDR2 comprising or consisting of SEQ ID NO:166, and a V_{H} CDR3 comprising or consisting of SEQ ID NO:167. In certain aspects, the light chain may comprise SEQ ID NO:330 and the heavy chain may comprise SEQ ID NO:329.

In one aspect, the isolated antibody (e.g., SKT02) comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:332, the heavy chain comprises an amino acid sequence at least 80% identical, at least 85% identical, at least 90% identical, at least 95% identical, at last 97% identical, or at least 99% identical to SEQ ID NO:331. In certain aspects, the light chain may comprise a V_{L} CDR1 comprising or consisting of SEQ ID NO:169, a V_{L} CDR2 comprising or consisting of SEQ ID NO:170, and a V_{L} CDR3 comprising or consisting of SEQ ID NO:171. In certain aspects, the heavy chain may comprise a V_{H} CDR1 comprising or consisting of SEQ ID NO:173, a V_{H} CDR2 comprising or consisting of SEQ ID NO:174, and a V_{H} CDR3 comprising or consisting of SEQ ID NO:175. In certain aspects, the light chain may comprise SEQ ID NO:332 and the heavy chain may comprise SEQ ID NO:331.

In one aspect, the isolated antibody (e.g., SKT05) comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:334, the heavy chain comprises an amino acid sequence at least 80% identical, at least 85% identical, at least 90% identical, at least 95% identical, at last 97% identical, or at least 99% identical to SEQ ID NO:333. In certain aspects, the light chain may comprise a V_{L} CDR1 comprising or consisting of SEQ ID NO:177, a V_{L} CDR2 comprising or consisting of SEQ ID NO:178, and a V_{L} CDR3 comprising or consisting of SEQ ID NO:179. In certain aspects, the heavy chain may comprise a V_{H} CDR1 comprising or consisting of SEQ ID NO:181, a V_{H} CDR2 comprising or consisting of SEQ ID NO:182, and a V_{H} CDR3 comprising or consisting of SEQ ID NO: 183. In certain aspects, the light chain may comprise SEQ ID NO:334 and the heavy chain may comprise SEQ ID NO:333.

In one aspect, the isolated antibody (e.g., SKT14) comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:336, the heavy chain comprises an amino acid sequence at least 80% identical, at least 85% identical, at least 90% identical, at least 95% identical, at last 97% identical, or at least 99% identical to SEQ ID NO:335. In certain aspects, the light chain may comprise a V_{L} CDR1 comprising or consisting of SEQ ID NO:185, a V_{L} CDR2 comprising or consisting of SEQ ID NO:186, and a V_{L} CDR3 comprising or consisting of SEQ ID NO:187. In certain aspects, the heavy chain may comprise a V_{H} CDR1 comprising or consisting of SEQ ID NO:189, a V_{H} CDR2 comprising or consisting of SEQ ID NO: 190, and a V_{H} CDR3 comprising or consisting of SEQ ID NO:191. In certain aspects, the light chain may comprise SEQ ID NO:336 and the heavy chain may comprise SEQ ID NO:335.

In one aspect, the isolated antibody (e.g., SKT19) comprises alight chain and a heavy chain, wherein the light chain comprises an amino acid sequence at least 80% identical, at least 85% identical, at least 90% identical, at least 95% identical, at last 97% identical, or at least 99% identical to SEQ ID NO:338, the heavy chain comprises an amino acid sequence at least 80% identical, at least 85% identical, at least 90% identical, at least 95% identical, at last 97% identical, or at least 99% identical to SEQ ID NO:337. In certain aspects, the light chain may comprise a V_{L} CDR1 comprising or consisting of SEQ ID NO:193, a V_{L} CDR2 comprising or consisting of SEQ ID NO:194, and a V_{L} CDR3 comprising or consisting of SEQ ID NO:195. In certain aspects, the heavy chain may comprise a V_{H} CDR1 comprising or consisting of SEQ ID NO: 197, a V_{H} CDR2 comprising or consisting of SEQ ID NO:198, and a V_{H} CDR3 comprising or consisting of SEQ ID NO:199. In certain aspects, the light chain may comprise SEQ ID NO:338 and the heavy chain may comprise SEQ ID NO:337.

In one aspect, the isolated antibody (e.g., SKT20) comprises alight chain and a heavy chain, wherein the light chain comprises an amino acid sequence at least 80% identical, at least 85% identical, at least 90% identical, at least 95% identical, at last 97% identical, or at least 99% identical to SEQ ID NO:340, the heavy chain comprises an amino acid sequence at least 80% identical, at least 85% identical, at least 90% identical, at least 95% identical, at last 97% identical, or at least 99% identical to SEQ ID NO:339. In certain aspects, the light chain may comprise a V_{L} CDR1 comprising or consisting of SEQ ID NO:201, a V_{L} CDR2 comprising or consisting of SEQ ID NO:202, and a V_{L} CDR3 comprising or consisting of SEQ ID NO:203. In certain aspects, the heavy chain may comprise a V_{H} CDR1 comprising or consisting of SEQ ID NO:205, a V_{H} CDR2 comprising or consisting of SEQ ID NO:206, and a V_{H} CDR3 comprising or consisting of SEQ ID NO:207. In certain aspects, the light chain may comprise SEQ ID NO:340 and the heavy chain may comprise SEQ ID NO:339.

In one aspect, the isolated antibody (e.g., SKT05.hu13) comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:342, the heavy chain comprises an amino acid sequence at least 80% identical, at least 85% identical, at least 90% identical, at least 95% identical, at last 97% identical, or at least 99% identical to SEQ ID NO:341. In certain aspects, the light chain may comprise a V_{L} CDR1 comprising or consisting of SEQ ID NO:209, a V_{L} CDR2 comprising or consisting of SEQ ID NO:210, and a V_{L} CDR3 comprising or consisting of SEQ ID NO:211. In certain aspects, the heavy chain may comprise a V_{H} CDR1 comprising or consisting of SEQ ID NO:213, a V_{H} CDR2 comprising or consisting of SEQ ID NO:214, and a V_{H} CDR3 comprising or consisting of SEQ ID NO:215. In certain aspects, the light chain may comprise SEQ ID NO:342 and the heavy chain may comprise SEQ ID NO:341.

In one aspect, the isolated antibody (e.g., SKT05.hu2l) comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:344, the heavy chain comprises an amino acid sequence at least 80% identical, at least 85% identical, at least 90% identical, at least 95% identical, at last 97% identical, or at least 99% identical to SEQ ID NO:343. In certain aspects, the light chain may comprise a V_{L} CDR1 comprising or consisting of SEQ ID NO:217, a V_{L} CDR2 comprising or consisting of SEQ ID NO:218, and a V_{L} CDR3 comprising or consisting of SEQ ID NO:219. In certain aspects, the heavy chain may comprise a V_{H} CDR1 comprising or consisting of SEQ ID NO:221, a V_{H} CDR2 comprising or consisting of SEQ ID NO:222, and a V_{H} CDR3 comprising or consisting of SEQ ID NO:223. In certain aspects, the light chain may comprise SEQ ID NO:344 and the heavy chain may comprise SEQ ID NO:343.

In one aspect, the isolated antibody (e.g., SKT05.hu25) comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:346, the heavy chain comprises an amino acid sequence at least 80% identical, at least 85% identical, at least 90% identical, at least 95% identical, at last 97% identical, or at least 99% identical to SEQ ID NO:345. In certain aspects, the light chain may comprise a V_{L} CDR1 comprising or consisting of SEQ ID NO:225, a V_{L} CDR2 comprising or consisting of SEQ ID NO:226, and a V_{L} CDR3 comprising or consisting of SEQ ID NO:227. In certain aspects, the heavy chain may comprise a V_{H} CDR1 comprising or consisting of SEQ ID NO:229, a V_{H} CDR2 comprising or consisting of SEQ ID NO:230, and a V_{H} CDR3 comprising or consisting of SEQ ID NO:231. In certain aspects, the light chain may comprise SEQ ID NO:346 and the heavy chain may comprise SEQ ID NO:345.

In one aspect, the isolated antibody (e.g., SKT05.hu29) comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:348, the heavy chain comprises an amino acid sequence at least 80% identical, at least 85% identical, at least 90% identical, at least 95% identical, at last 97% identical, or at least 99% identical to SEQ ID NO:347. In certain aspects, the light chain may comprise a V_{L} CDR1 comprising or consisting of SEQ ID NO:233, a V_{L} CDR2 comprising or consisting of SEQ ID NO:234, and a V_{L} CDR3 comprising or consisting of SEQ ID NO:235. In certain aspects, the heavy chain may comprise a V_{H} CDR1 comprising or consisting of SEQ ID NO:237, a V_{H} CDR2 comprising or consisting of SEQ ID NO:238, and a V_{H} CDR3 comprising or consisting of SEQ ID NO:239. In certain aspects, the light chain may comprise SEQ ID NO:348 and the heavy chain may comprise SEQ ID NO:347.

In one aspect, the isolated antibody (e.g., SKT05.hu37) comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:350, the heavy chain comprises an amino acid sequence at least 80% identical, at least 85% identical, at least 90% identical, at least 95% identical, at last 97% identical, or at least 99% identical to SEQ ID NO:349. In certain aspects, the light chain may comprise a V_{L} CDR1 comprising or consisting of SEQ ID NO:241, a V_{L} CDR2 comprising or consisting of SEQ ID NO:242, and a V_{L} CDR3 comprising or consisting of SEQ ID NO:243. In certain aspects, the heavy chain may comprise a V_{H} CDR1 comprising or consisting of SEQ ID NO:245, a V_{H} CDR2 comprising or consisting of SEQ ID NO:246, and a V_{H} CDR3 comprising or consisting of SEQ ID NO:247. In certain aspects, the light chain may comprise SEQ ID NO:350 and the heavy chain may comprise SEQ ID NO:349.

In one aspect, the isolated antibody (e.g., SKT05.hu41) comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:352, the heavy chain comprises an amino acid sequence at least 80% identical, at least 85% identical, at least 90% identical, at least 95% identical, at last 97% identical, or at least 99% identical to SEQ ID NO:351. In certain aspects, the light chain may comprise a V_{L} CDR1 comprising or consisting of SEQ ID NO:249, a V_{L} CDR2 comprising or consisting of SEQ ID NO:250, and a V_{L} CDR3 comprising or consisting of SEQ ID NO:251. In certain aspects, the heavy chain may comprise a V_{H} CDR1 comprising or consisting of SEQ ID NO:253, a V_{H} CDR2 comprising or consisting of SEQ ID NO:254, and a V_{H} CDR3 comprising or consisting of SEQ ID NO:255. In certain aspects, the light chain may comprise SEQ ID NO:352 and the heavy chain may comprise SEQ ID NO:351.

In one aspect, the isolated antibody (e.g., SKT05.hu47) comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:354, the heavy chain comprises an amino acid sequence at least 80% identical, at least 85% identical, at least 90% identical, at least 95% identical, at last 97% identical, or at least 99% identical to SEQ ID NO:353. In certain aspects, the light chain may comprise a V_{L} CDR1 comprising or consisting of SEQ ID NO:257, a V_{L} CDR2 comprising or consisting of SEQ ID NO:258, and a V_{L} CDR3 comprising or consisting of SEQ ID NO:259. In certain aspects, the heavy chain may comprise a V_{H} CDR1 comprising or consisting of SEQ ID NO:261, a V_{H} CDR2 comprising or consisting of SEQ ID NO:262, and a V_{H} CDR3 comprising or consisting of SEQ ID NO:263. In certain aspects, the light chain may comprise SEQ ID NO:354 and the heavy chain may comprise SEQ ID NO:353.

In one aspect, the isolated antibody (e.g., SKT05.hu52) comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:356, the heavy chain comprises an amino acid sequence at least 80% identical, at least 85% identical, at least 90% identical, at least 95% identical, at last 97% identical, or at least 99% identical to SEQ ID NO:355. In certain aspects, the light chain may comprise a V_{L} CDR1 comprising or consisting of SEQ ID NO:265, a V_{L} CDR2 comprising or consisting of SEQ ID NO:266, and a V_{L} CDR3 comprising or consisting of SEQ ID NO:267. In certain aspects, the heavy chain may comprise a V_{H} CDR1 comprising or consisting of SEQ ID NO:269, a V_{H} CDR2 comprising or consisting of SEQ ID NO:270, and a V_{H} CDR3 comprising or consisting of SEQ ID NO:271. In certain aspects, the light chain may comprise SEQ ID NO:356 and the heavy chain may comprise SEQ ID NO:355.

In one aspect, the isolated antibody (e.g., SKT05.hu56) comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:358, the heavy chain comprises an amino acid sequence at least 80% identical, at least 85% identical, at least 90% identical, at least 95% identical, at last 97% identical, or at least 99% identical to SEQ ID NO:357. In certain aspects, the light chain may comprise a V_{L} CDR1 comprising or consisting of SEQ ID NO:273, a V_{L} CDR2 comprising or consisting of SEQ ID NO:274, and a V_{L} CDR3 comprising or consisting of SEQ ID NO:275. In certain aspects, the heavy chain may comprise a V_{H} CDR1 comprising or consisting of SEQ ID NO:277, a V_{H} CDR2 comprising or consisting of SEQ ID NO:278, and a V_{H} CDR3 comprising or consisting of SEQ ID NO:279. In certain aspects, the light chain may comprise SEQ ID NO:358 and the heavy chain may comprise SEQ ID NO:357.

In one aspect, the isolated antibody (e.g., SKT05.hu60) comprises a light chain and a heavy chain, wherein the light chain comprises an amino acid sequence at least 80% identical, at least 85% identical, at least 90% identical, at least 95% identical, at last 97% identical, or at least 99% identical to SEQ ID NO:360, the heavy chain comprises an amino acid sequence at least 80% identical, at least 85% identical, at least 90% identical, at least 95% identical, at last 97% identical, or at least 99% identical to SEQ ID NO:359. In certain aspects, the light chain may comprise a V_{L} CDR1 comprising or consisting of SEQ ID NO:281, a V_{L} CDR2 comprising or consisting of SEQ ID NO:282, and a V_{L} CDR3 comprising or consisting of SEQ ID NO:283. In certain aspects, the heavy chain may comprise a V_{H} CDR1 comprising or consisting of SEQ ID NO:285, a V_{H} CDR2 comprising or consisting of SEQ ID NO:286, and a V_{H} CDR3 comprising or consisting of SEQ ID NO:287. In certain aspects, the light chain may comprise SEQ ID NO:360 and the heavy chain may comprise SEQ ID NO:359.

One aspect of the disclosure is a composition comprising an isolated antibody of the disclosure. In certain aspects, the composition may be a liquid composition. In certain aspects, the composition may comprise a solid composition. Compositions of the disclosure may comprise excipients, such as buffers, stabilizers, solubilizers, antioxidants, emulsifiers, and carriers. Examples of buffers include, but are not limited to, phosphate, citrate, succinate, acetate and other organic acids, histidine, arginine, Hanks' solution, Ringer's solution, or physiological saline buffer. Stabilizers help maintain the integrity of the composition and/or the structure of the antibody. They can also help reduce degradation of the antibody, thereby increasing the life of the antibody beyond the life of an antibody in the body. Examples of stabilizers include, but are not limited to, polyols, sugars, amino acids, amines, salts, cyclodextrins, and chelating agents, such as ethylenediaminetetraacetic acid (EDTA). Solubilizers may increase the solubility of ingredients, such as antibodies, in the composition. Examples of solubilizers include, but are not limited to, water-soluble organic solvents (polyethylene glycol 300, polyethylene glycol 400, ethanol, propylene glycol, glycerin. N-methyl-2-pyrrolidone, dimethylacetamide, and dimethylsulfoxide), non-ionic surfactants (Cremophor EL, Cremophor RH 40, Cremophor RH 60, d-alpha-tocopherol polyethylene glycol 1000 succinate, polysorbate 20, polysorbate 80, Solutol HS 15, sorbitan monooleate, poloxamer 407, Labrafil M-1944CS, Labrafil M-2125CS, Labrasol, Gellucire 44/14, Softigen 767, and mono- and di-fatty acid esters of PEG 300, 400, or 1750), water-insoluble lipids (castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil and palm seed oil), organic liquids/semi-solids (beeswax, d-alpha-tocopherol, oleic acid, medium-chain mono- and diglycerides), various cyclodextrins (alpha-cyclodextrin, beta-cyclodextrin, hydroxypropyl-beta-cyclodextrin, and sulfobutylether-beta-cyclodextrin), and phospholipids (hydrogenated soy phosphatidylcholine, disteamylphosphatidylglycerol, L-alpha-dimyristoylphosphatidylcholine, L-alpha-dimyristoylphosphatidylglycerol). Antioxidants protect ingredients in the composition from degradation and extend the life of the composition and particularly the antibody. Examples of antioxidants include, but are not limited to, ascorbic acid, tocopherols, cysteine, ascorbyl palmitate, monothioglycerol, sodium bisulfite, and butylated hydroxytoluene.

Compositions of the disclosure may be formulated in any suitable form, and, in large part, the form may be determined by the intended mode of administration. Compositions can be in the form of a solid, semi-solid or liquid. Examples of forms useful for administering isolated antibodies of the disclosure include, but are not limited to, liquid compositions, emulsions, suspensions, tablets, capsules, powders, troches, lozenges, suspensions, gels, pastes, slurries, soft-gel capsules, and syrups. Preferably the compositions are produced in a unit dosage form suitable for administration of a precise dose. Injectables may be prepared in conventional forms, either as liquid solutions or suspensions, solid forms suitable for solution or suspension in liquid prior to injection, or as emulsions.

Preparations for parenteral administration of an antibody include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

Compounds and compositions of the disclosure can be administered by any suitable route. Such routes of administration include, but are not limited to, injection, including intravenous, intraperitoneal, intramuscular, and subcutaneous injection, oral administration, transmucosal administration, transdermal administration, topical administration, nasal administration, ocular administration, or via suppository.

One aspect of the disclosure is a method of protecting an individual against infection with an alphavirus, comprising administering to the individual an isolated antibody of the disclosure, or a composition of the disclosure. The terms individual, subject, and patient are well-recognized in the art, and are herein used interchangeably to refer to any human or other animal susceptible to alphavirus infection. Examples include, but are not limited to, humans and other primates, including non-human primates such as chimpanzees and other apes and monkey species; farm animals such as cattle, sheep, pigs, seals, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like; and fish, such as salmon. The terms individual, subject, and patient by themselves, do not denote a particular age, sex, race, and the like. Thus, individuals of any age, whether male or female, are covered by the present disclosure and include, but are not limited to the elderly, adults, children, babies, infants, and toddlers. Likewise, the methods of the present invention can be applied to any race, including, for example, Caucasian (white), African-American (black), Native American, Native Hawaiian, Hispanic, Latino, Asian, and European.

In certain aspects, administration of the antibody, or composition, of the disclosure protects the individual against at least one species of alphavirus. In certain aspects, administration of the antibody, or composition, of the disclosure protects the individual against at least two species of alphavirus. In certain aspects, administration of the antibody, or composition, of the disclosure protects the individual against at least three or more species of alphavirus. In certain aspects, the at least one, at least two, or three or more species of alphavirus are selected from the group consisting of WEEV, EEEV, VEEV, CHKV, RRV, MAYV, ONNV, BFV, MV, NV, SFV, TV, UV, AV, BV and SV.

One aspect of the disclosure is a method of treating an individual for an alphavirus infection, comprising administering to the individual an isolated antibody of the disclosure, or a composition of the disclosure. In certain aspects, administration of the antibody, or composition, of the disclosure treats the individual for infection with at least one species of alphavirus. In certain aspects, administration of the antibody, or composition, of the disclosure treats the individual for infection with at least two species of alphavirus. In certain aspects, administration of the antibody, or composition, of the disclosure treats the individual for infection with at least three or more species of alphavirus. In certain aspects, the at least one, at least two, or three or more species of alphavirus are selected from the group consisting of WEEV, EEEV, VEEV, CHKV, RRV, MAYV, ONNV, BFV, MV, NV, SFV, TV, UV, AV, BV and SV.

In these aspects, the antibody or composition of the disclosure may be administered by any suitable means including, but not limited to, traditional syringes, needleless injection devices, pills. Solutions. Suitable routes of administration include, but are not limited to, parenteral delivery, such as intramuscular, intradermal, subcutaneous, intramedullary injections, as well as, intrathecal, direct intraventricular, intravenous, intraperitoneal, intranasal, or intraocular injections, just to name a few. For injection, the compounds of one embodiment of the invention may be formulated in aqueous solutions, preferably in physiologically compatible buffers such as Hanks' solution, Ringer's solution, or physiological saline buffer.

One aspect of the disclosure is a method of detecting at least one species of alphavirus in a sample, comprising contacting the sample with an isolated antibody, or a composition, of the disclosure under conditions suitable for forming an alphavirus/antibody complex, and detecting the alphavirus/antibody complex, if any, thereby detecting the at least one species of alphavirus. As used herein, the term “contacting” refers to the introduction of a sample putatively containing alphavirus to an antibody, or antibody-containing composition, of the disclosure, for example, by combining or mixing the sample with the antibody or composition. When an alphavirus is present in the sample, an alphavirus/antibody complex is then formed. Such complex formation refers to the ability of the antibody to selectively bind to the alphavirus to form a stable complex that can be detected. Detection may be qualitative, quantitative, or semi-quantitative. Binding may be measured using a variety of methods standard in the art including, but not limited to, enzyme immunoassays (e.g., ELISA), immunoprecipitations, immunoblot assays and other immunoassays. In certain s aspects, the isolated antibody may be labeled (e.g., fluorescently labeled). Such methods are known to those of skill in the art. In certain aspects, the method comprises detecting at least two species of alphavirus in a sample. In certain aspects, the method comprises detecting at least three species of alphavirus in a sample. In certain aspects, the at least one, at least two or at least three species of alphavirus are detected using a single antibody.

One aspect of the disclosure is a kit comprising an isolated antibody of the disclosure, or a composition of the disclosure. Such kit may also contain associated components, such as, but not limited to, buffers, labels, containers, inserts, tubing, vials, syringes and the like.

One aspect of the disclosure is an isolated antibody of the disclosure for use in preparation of a medicament for preventing infection of an individual by at least one species of alphavirus. In certain aspects, the isolated antibody may prevent infection of the individual by at least two species of alphavirus. In certain aspects, the isolated antibody may prevent infection of the individual by at least three species of alphavirus.

One aspect of the disclosure is an isolated antibody of the disclosure for use in preparation of a medicament for treating individual that has been infected by at least one species of alphavirus. In certain aspects, the isolated antibody may treat an individual infected by at least two species of alphavirus. In certain aspects, the isolated antibody may treat an individual infected by at least three species of alphavirus.

One aspect of the disclosure is a method of producing an antibody that recognizes at least three species of alphavirus, comprising administering a multivalent vaccine to an individual, wherein the multivalent vaccine comprises at least three species of alphavirus, or at least three species of virus-like particles (VLPs), each VLP being made from a unique species of alphavirus, wherein the multivalent vaccine is administered to the individual at three different time points. In certain aspects, the second administration of the multivalent vaccine is at least four weeks after the first administration of the multivalent vaccine. In certain aspects, the second administration of the multivalent vaccine is at least four weeks after the first administration of the multivalent vaccine and the third administration of the multivalent vaccine is at least 4 weeks, at least 8 weeks, at least 12 weeks, at least 16 weeks, or at least 17 weeks after the second administration of the multivalent vaccine. In certain aspects, the method comprises administering the multivalent vaccine at least one additional time following the third administration. In certain aspects, the multivalent vaccine comprises at least species of virus selected from the group consisting of WEEV, EEEV, VEEV, CHKV, RRV, MAYV, ONNV, BFV, MV, NV, SFV, TV, UV, AV, BV and SV. In certain aspects, the multivalent vaccine comprises at least species of virus selected from the group consisting of WEEV, EEEV, VEEV, CHKV, RRV, MAYV, ONNV. In certain aspects, multivalent vaccine comprises WEEV, EEEV, and VEEV. In certain aspects, the multivalent vaccine comprises at least species of VLPs, wherein each VLP is made from a unique species of alphavirus selected from the group consisting of WEEV, EEEV, VEEV, CHKV, RRV, MAYV, ONNV, BFV, MV, NV, SFV, TV, UV, AV, BV and SV. In certain aspects, the multivalent vaccine comprises at least species of VLPs, wherein each VLP is made from a unique species of alphavirus selected from the group consisting of WEEV, EEEV, VEEV, CHKV, RRV, MAYV, ONNV. In certain aspects, the multivalent vaccine comprises at least species of VLPs, wherein one VLP is made from WEEV proteins, one VLP is made from EEEV proteins, and one VLP is made from VEEV proteins. In certain aspects, the multivalent vaccine comprises an adjuvant.

This written description uses examples to disclose the disclosure, including the best mode, and to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

### Example 1. Trivalent VLP Immunization Elicits Single-Specific and Triple-Specific a-EEV mAbs in NHP

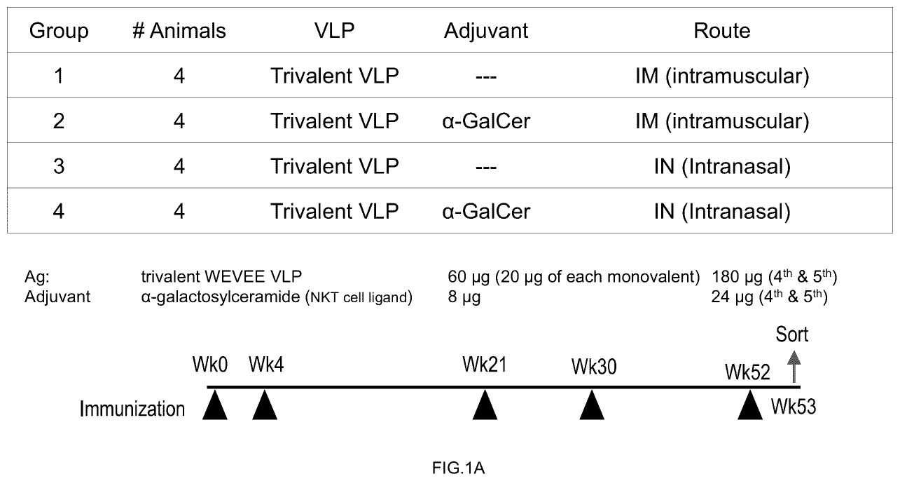

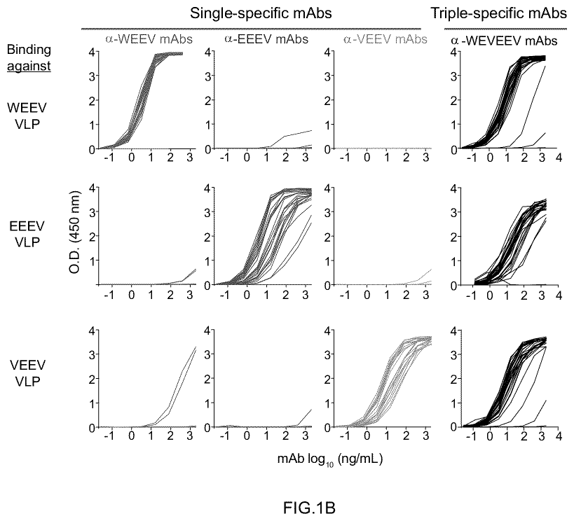

Previous work had shown that macaques immunized with a trivalent EEV VLP vaccine were protected from aerosol challenge with WEEV, EEEV, or VEEV (Ko et al., 2019). Passive transfer studies in mice suggested that this protection was due to humoral immunity. However, the mechanism of protection was not defined; i.e., protection could have been due to multiple single-specific mAbs vs. one or more triple-specific mAbs. A follow up study was conducted to try to improve immunogenicity and to define the mechanism of trivalent EEV VLP vaccine-mediated protection. Cynomolgus macaques (n=16) received a trivalent EEV vaccine composed of WEEV, EEEV, and VEEV VLPs. Immunizations were administered intramuscularly (IM) or intranasally (IN) five times over 52 weeks, with or without alpha-galactosylceramide (a-GalCer) as an adjuvant (FIG. 1A). Following each of the first four immunizations, systemic and mucosal humoral immune responses were evaluated in serum and bronchoalveolar lavage (BAL) fluid, respectively. After one month, IM immunization induced higher systemic binding IgG responses against all EEVs compared to IN immunization, while addition of a-GalCer adjuvant only generated increased systemic IgG responses in IN-vaccinated animals. Compared to systemic levels, IgG levels in BAL fluid were reduced and contamination with even small amounts of blood during sampling greatly affected the quantitation of bona fide mucosal antibody responses. Since IgA-dimers do not exist in blood, total secretory IgA dimer levels in BAL were quantitated to assess mucosal humoral immune responses. There was a trend that IN immunization with adjuvant generated greater IgA dimer response than IM immunization, but responses were variable.

One week after the 5^{th} immunization, PBMC were isolated and their ability to bind individual EEV VLPs, each labeled with distinct fluorescent lipophilic membrane staining dye, was evaluated. These labeled VLPs functioned well as probes for antigen-specific B cell sorting, and it was possible to identify B cells with surface IgG that putatively bound one or more of the VLPs. The animal with the greatest number of triple-specific B cells was selected for EEV-specific single B cell sorting; B cells were collected, and heavy and light chain immunoglobulins were sequenced and synthesized. In total, 109 a-EEV mAbs, comprising 70 lineages from a single NHP and having a range of single- and triple-specific binding were expressed.

The sorting strategy employed, in which B cells were sorted for selective binding activity, was confirmed by testing the 109 a-EEV mAbs for binding to each VLP by ELISA (FIG. 1B). For the a-WEEV mAbs, which were selected positively for binding the WEEV VLP and negatively for not binding EEEV and VEEV VLPs, all (30/30) bound the WEEV VLP, and two showed very low-level cross-reactivity for binding to VEEV VLP. Similarly, all a-VEEV mAbs (20/20) bound only the VEEV VLP with no cross-reactivity, and 27/32 a-EEEV mAbs bound only the EEEV VLP while 5/32 failed to bind any VLP. For putatively triple-specific mAbs (a-WEVEEV), 26/27 bound all three VLPs. Accordingly, the VLP probe-sorting strategy proved robust, and generated mAbs that displayed a high degree of binding specificity (94%) to the desired VLP(s).

### Example 2. Competition ELISAs Identify Distinct Binding Groups of Single-Specific and Triple-Specific a-EEV mAbs

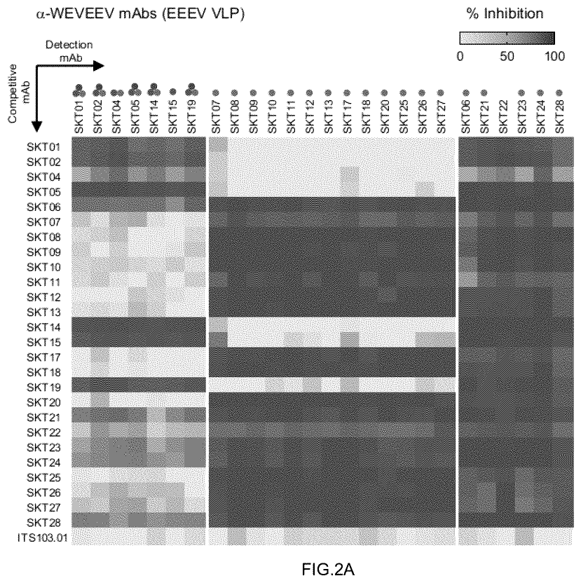

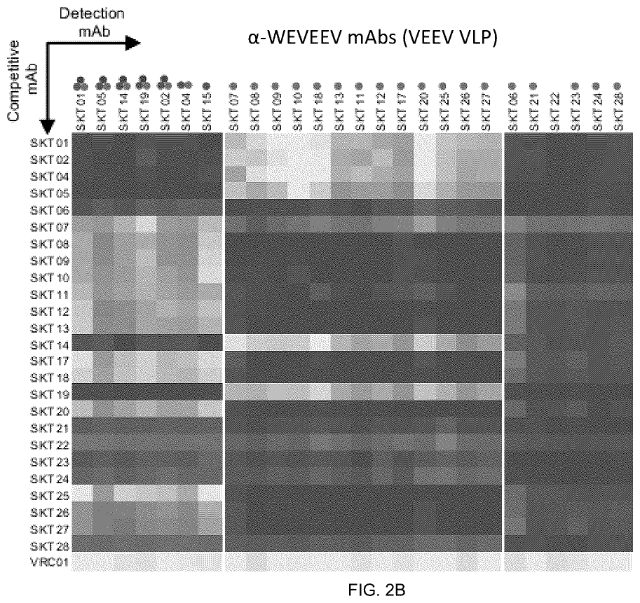

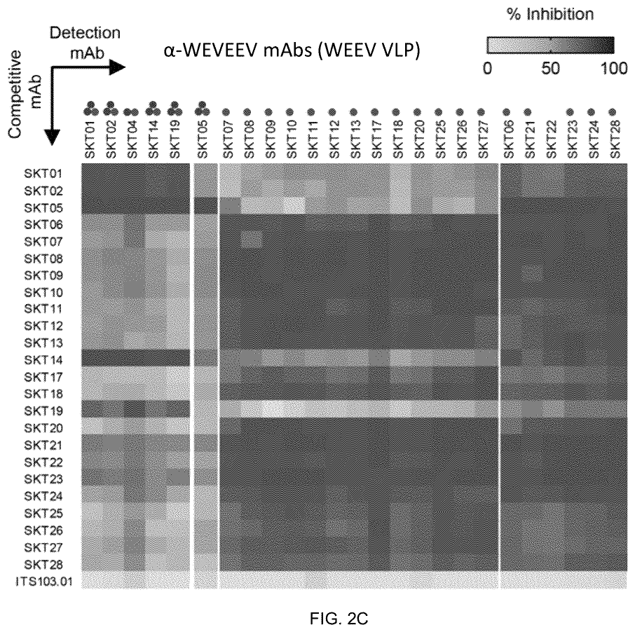

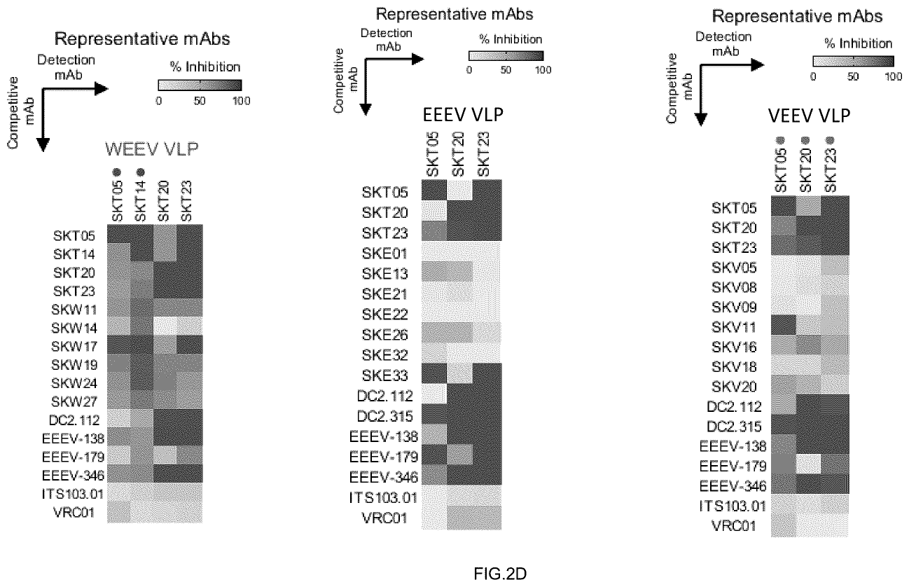

To estimate the number of major antigenic sites recognized by single-specific and triple-specific a-EEV mAbs, competitive-binding ELISAs were performed for each VLP (FIGS. 2A-2D). For single-specific mAbs, hierarchical clustering identified seven competition groups for a-EEEV mAbs, six for a-WEEV mAbs, and seven for a-VEEV. For triple-specific mAbs (a-WEVEEV), hierarchical clustering identified three competition groups for the EEEV VLP (FIG. 2A) and VEEV VLP (FIG. 2B) and four for the WEEV VLP (FIG. 2C). One mAb was chosen as a representative from each competition group of single-specific and triple-specific mAbs for an individual VLP. These representative single-specific mAbs, as well as previously published broadly reactive a-EEV mAbs (DC2.112, DC2.315, EEEV-138, EEEV-179, and EEEV-346), were tested as competitors against the representative triple-specific mAbs for each VLP (FIG. 2D) (Kim et al., 2021; Williamson et al., 2021). One group from each of the single-specific mAbs was found to overlap with at least one of the triple-specific mAbs. Thus, while the latter represent broadly reactive epitopes, there can still be strain-specific mAbs against those epitopes. Most of the previously published broadly reactive a-EEV mAbs fall into one of two triple-specific competition groups: those represented by SKT20 or SKT23. Only EEEV-179 appears to share a competition group with SKT05 when tested on the EEEV VLP and possibly the VEEV VLP; it is in its own competition group for the WEEV VLP. In total, using the disclosed mAbs allowed mapping of 21 binding regions on the surface of WEVEE VLPs.

### Example 3. Vaccine-Elicited a-EEV mAbs Bind, Neutralize, and Protect Against Encephalitic Alphavirus Challenge In Vivo

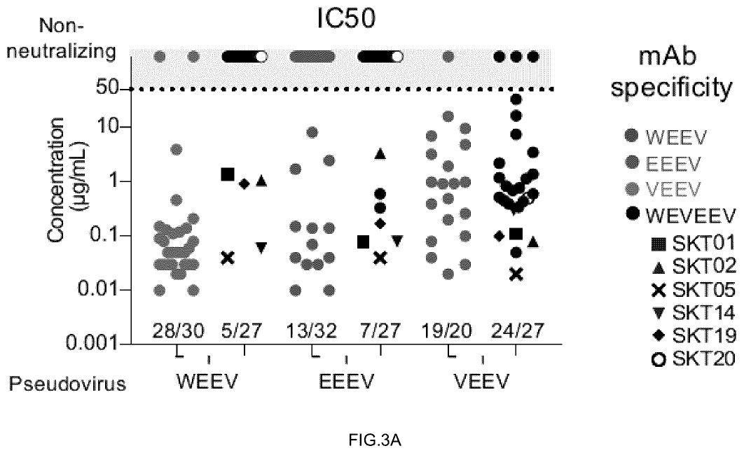

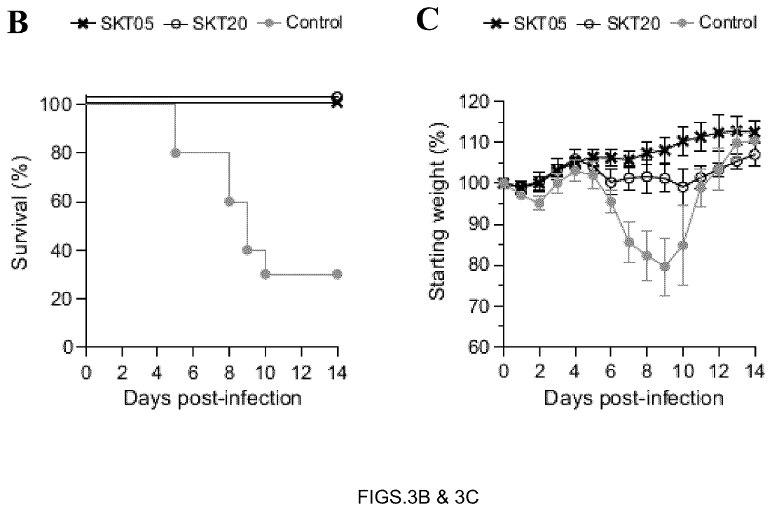

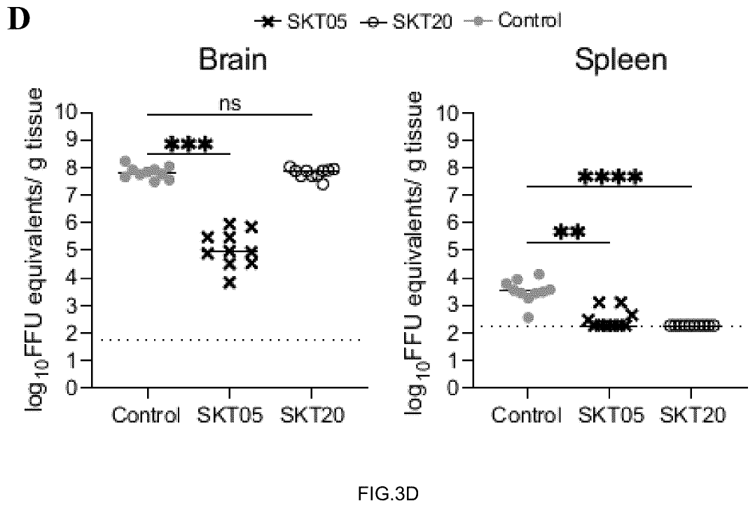

To evaluate the neutralization activity of single-specific and triple-specific a-EEV mAbs Env-pseudotyped lentiviral reporter viruses for WEEV, EEEV, and VEEV were used (FIG. 3A). For a-WEEV mAbs, 27 reached a half maximal inhibitory concentration (IC₅₀) of less than 1 μg/mL against the WEEV pseudovirus; of those, 13 were particularly potent with 80% inhibitory concentrations (IC₈₀) below 1 μg/mL. Neutralization of EEEV pseudovirus by a-EEEV mAbs was less prevalent, with 4 reaching IC₈₀ values below 1 μg/mL. Of note, three a-EEEV mAbs had exceptional potency with IC₈₀ values below 100 ng/mL. 19/20 a-VEEV mAbs neutralized VEEV pseudovirus, though to varying degrees of potency. Six a-VEEV mAbs neutralized with IC₈₀ values lower than 1 μg/mL. The triple-specific mAbs were also tested for neutralization of each EEV pseudovirus. Similar to the a-VEEV mAbs, nearly all (24/27) triple-specific mAbs neutralized VEEV pseudovirus with varying degrees of potency. While neutralization of WEEV and EEEV pseudovirus was less common, those that did neutralize at least one (i.e., SKT01, SKT02, SKT04, SKT05, SKT14, SKT15, and SKT19) did so with IC₅₀ titers that ranged from 40 ng/mL to 3.3 μg/mL. Of these, 5 mAbs (SKT01, SKT02, SKT05, SKT14, and SKT19) were identified that can neutralize all three EEV pseudoviruses. One triple-specific mAb (SKT05) exhibited exceptional potency against all three EEV pseudoviruses, with IC₈₀ values of 70 ng/mL (WEEV), 390 ng/mL (EEEV), and 60 ng/mL (VEEV).

Next, the neutralization activity of select single-specific and triple-specific mAbs against chimeric viruses expressing the nonstructural proteins of Sindbis virus (SINV) and the structural proteins of WEEV, EEEV, or VEEV was tested. Of the single-specific mAbs that were tested against their cognate SINV-EEV chimeric virus, 5/6 neutralized with IC50 values that ranged from 10 ng/mL to 1.4 mg/mL. In contrast, of the triple-specific mAbs that were tested only SKT20 weakly neutralized SINV-VEEV, while SKT05, SKT14, and SKT23 did not neutralize any chimeric virus. The neutralization activity of select single-specific and triple-specific mAbs against pathogenic WEEV, EEEV, and VEEV was also evaluated. Only one a-VEEV mAbs neutralized VEEV (strain TrD) with a PRNT50 value of 0.95 mg/mL, and while neutralization was not observed with any of the triple-specific mAbs tested, SKT05 did display reduced plaque sizes compared to virus-only and negative mAb controls that could reflect antiviral activity in this assay. All four a-WEEV mAbs neutralized WEEV (strain Fleming) with PRNT50 titers ranging from 60 ng/mL to 0.24 mg/mL; of the triple-specific mAbs tested, only SKT05 neutralized with a PRNT50 value of 0.23 mg/mL. All a-EEEV mAbs neutralized EEEV (strain FL93-939), one with a PRNT50 value of 0.95 mg/mL while another displayed exceptional potency with a PRNT50 value that was below 7.3 ng/mL, the lowest concentration tested. Of the triple-specific mAbs tested, 3/5 showed weak neutralization of pathogenic EEEV with PRNT50 values ranging from 7.4 mg/mL to 15.2 mg/mL. Of note, both SKT20 and SKT23 neutralized pathogenic EEEV despite not neutralizing EEEV pseudovirus.

In addition to testing neutralization activity, select single-specific and triple-specific mAbs were tested for their ability to bind pathogenic WEEV, EEEV, and VEEV. When tested against WEEV (strain CBA87), a-WEEV and a-WEVEEV mAbs were particularly potent with most endpoint titers <10 ng/mL. Endpoint titers for a-EEEV and a-WEVEEV mAbs binding to EEEV (strain FL93-939) were ~10 ng/mL. The largest variability in binding was observed using pathogenic VEEV (strain TrD); 4/5 a-VEEV mAbs bound with endpoint titers ranging from <10 ng/mL to 100 ng/mL, while a-WEVEEV mAbs ranged from <10 ng/mL to 1 mg/mL. Overall, these are exceptionally potent binding mAbs with a range of epitope and strain specificities.

Two triple-specific mAbs were selected from separate competition groups to determine whether protective effects could be conferred in vivo following encephalitic alphavirus challenge. SKT05 or SKT20 was administered to six-week-old C3H-HeN mice one day prior to intranasal inoculation with 10⁷ FFU of VEEV TC-83 (approximately 1 LD₅₀). The animals were followed for 14 days post-infection (dpi). SKT05 or SKT20 treatment resulted in 100% survival following lethal challenge (FIG. 3B). In agreement with the survival results, control mAb-treated mice exhibited substantial weight loss, as expected in this challenge model; weights of SKT20-treated mice remained relatively unchanged over the same time, while weights increased in SKT05-treated mice (FIG. 3C). An additional set of mice were sacrificed at 5 dpi and viral load was assessed in the brain and spleen. Mice administered SKT05, but not SKT20, had significantly reduced viral titers in the brain compared to a control mAb (FIG. 3D, left). In the spleen, both SKT05 and SKT20 administration prior to challenge resulted in significantly reduced viral titers compared to a control mAb (FIG. 3D, right). Thus, SKT05 and, to a lesser extent SKT20, provide protective benefits against encephalitic alphavirus challenge when administered prophylactically. These results also confirm the limited utility of in vitro assays as predictors of in vivo protection.

### Example 4. Broadly Neutralizing a-EEV mAbs are Rare

To better understand the impact of immunization timing on elicitation of broadly neutralizing a-EEV mAbs, next generation sequencing (NGS) was conducted on PBMC isolated at various timepoints after the first four immunizations of the animal from which broadly neutralizing antibodies were isolated. In total, 31 out of 70 mAb lineages were detected with heavy chain NGS. After the first two immunizations, only one new lineage of each specificity was detected. In contrast, the greatest number of new lineages appeared at week 23, two weeks after the third immunization; a similar trend was observed when the total number of transcripts from all lineages of each specificity, normalized to the total number of transcripts at each time point was assessed. The lineage representing three of the five broadly neutralizing triple-specific mAbs (SKT01, SKT02, SKT05) was first detected between week 23 and 32. Interestingly, at week 21 a lineage that was shared by one triple-specific mAb (SKT19) and one a-EEEV mAb (SKE12) was first detected, suggesting that ongoing somatic hypermutation might guide the immune response to greater breadth. One broadly neutralizing triple-specific lineage, consisting of SKT14, was not detected by heavy chain NGS prior to week 53.

Consistent with these data, there was no detection of triple-specific a-EEV mAbs capable of neutralizing all three EEV pseudoviruses from two subjects enrolled in a human phase 1 clinical trial evaluating the safety and immunogenicity of trivalent WEVEEV VLP immunization (Coates et al., 2022); these subjects received only two doses of VLPs. Two subjects were chosen for EEV-specific single B cell sorting based on neutralizing antibody titers against pathogenic WEEV, EEEV, and VEEV from sera collected two weeks after the final immunization. Both subjects were administered 60 μg of WEVEEV VLPs (20 μg of each monovalent VLP) twice over eight weeks. In total, only six triple-specific B cells were sorted from both participants and expressed the mAbs from these cells. Four of the human triple-specific mAbs bound all three EEV VLPs (hSKT1001, hSKT1002, hSKT1004, hSKT1006), while an additional one (hSKT1003) bound only the VEEV VLP; one a-WEVEEV mAb (hSKT1005) did not bind any VLP.

Triple-specific, human mAbs for EEV pseudovirus neutralization (Figure S3D, right) were also tested. Despite positive binding to all three VLPs, none neutralized WEEV or EEEV pseudovirus. Instead, these four mAbs, as well as the one that only bound the VEEV VLP, neutralized only VEEV pseudovirus. Given this observation, competitive-binding ELISAs were performed using each human triple-specific as competitors against the representative NHP a-WEVEEV mAbs. As predicted from the neutralization profiles, most of the human a-WEVEEV mAbs were in the SKT20 competition group that neutralized only VEEV pseudovirus despite binding all three EEV VLPs. However, when they were tested for binding to the surface of live cells infected with SINV-WEEV. SINV-EEEV, or SINV-VEEV, many bound at comparable levels as SKT05. Accordingly, these data along with the NHP NGS data suggest that three or more immunizations may be required to elicit high levels of SKT05-like broadly neutralizing a-EEV mAbs, though protective effects conferred by fewer immunizations may still be a possibility in the absence of broad neutralization.

### Example 5. Cryo-EM Structures of Neutralizing Antibodies with VLPs Reveal that Sequence Variation and Conformation Variability Inversely Correlate with Broad Recognition

To gain insight into the broad recognition and protective activity by SKT05 and SKT20, the cryo-EM structures of antibody Fab-VLP complexes were determined and compared with structures of ligand-free VLPs. While structures of ligand-free VEEV and EEEV VLPs have been determined at resolutions of 3.5 Å and 4.2 Å, respectively (Ma et al., 2021; Williamson et al., 2020), the ligand-free structure of WEEV VLP had not been defined. To gain insight into the ligand-free WEEV VLP structure as well as the surface diversity of WEVEEV, single-particle cryo-EM data was collected on a Titan Krios and the structure of ligand-free WEEV VLP determined at 3.4 Å resolution (,). Like other alphaviruses (Basore et al., 2019; Li et al., 2010), the WEEV VLP comprised a T=4 icosahedron, with 240 copies of each structural protein assembled into 80 trimers. The extracted E1E2 trimer showed moderate conformational variability, with an average backbone root-mean-square deviation (RMSD) between WEEV and VEEV of 2.1 Å and between WEEV and EEEV of 2.6 Å. Sequence variation was also calculated as a buried surface area (BSA)-weighted average of normalized Shannon entropy, based on WEEV, VEEV, EEEV, CHIKV, RRV, MAYV, and ONNV sequences. Overall, sequence variation tended to be greater with E2 and on the outer surface of the VLP—and to correlate with conformational variability, though the fusion peptide region was highly conserved, though structurally variable.

In terms of antibody Fab-VLP complexes, with dozens of identified antibodies, including 16 lineages of triple specific antibodies, not all of them could be characterized by cryo-EM. To create an appropriate matrix of information by which to understand broad binding, a suitable subset of antibodies was chosen for structural analysis. Two triple-specific antibodies, SKT05 and SKT20, were chosen and these were supplemented with single-specific antibodies, each from a different competition group and against a different target VLP.

With VEEV, complex structures were determined with the triple-specific mAb SKT20 as well as the VEEV-neutralizing single-specific mAbs SKV09 and SKV16. SKT20 recognized the conserved fusion peptide in domain II of the E1 protein. SKV09 bound E1 at the VLP pseudo-6-fold axis with a restricted stoichiometry of only 0.375 Fabs per trimer. By contrast, SKV16 and SKT20 both recognized the E1E2 trimer, with stoichiometries of three Fabs per trimer; SKV16 bound specifically to domain B of E2. With EEEV, the cryo-EM structure at 3.5 Å resolution for the antigen-binding fragment of the EEEV-neutralizing mAb SKE26, which bound primarily to the A domain of the E2 protein, was determined.

With WEEV, complex structure was determined with WEEV-neutralizing single-specific mAbs SKW11, SKW19 and SKW24, as well as the triple-specific mAb SKT05. The triple-specific mAb SKT05 showed almost lateral recognition, recognizing domain II of the E1 protein, proximal to the fusion peptide. Another notable feature of SKT05 was its restricted stoichiometry, with only a single Fab binding per E1E2 trimer. A novel refinement algorithm was developed, selecting and grouping only the appropriate, Fab-bound E1E2-protomers in the reconstruction density, which improved resolution from 5.7 Å to 4.2 Å. All three single-specific mAbs bound the E2 protein. SKW11 primarily bound the b-ribbon of the E2 protein with a ~45-degree approach angle. Both SKW19 and SKW24 primarily bound the B domain of the E2 protein, with SKW19 binding at a more equatorial approach angle compared to SKW24 that was almost lateral in its binding angle. All three of these recognized trimers with a stoichiometry of three Fabs per E1E2 trimer.

### Example 6. Assessment of Epitope Characteristics

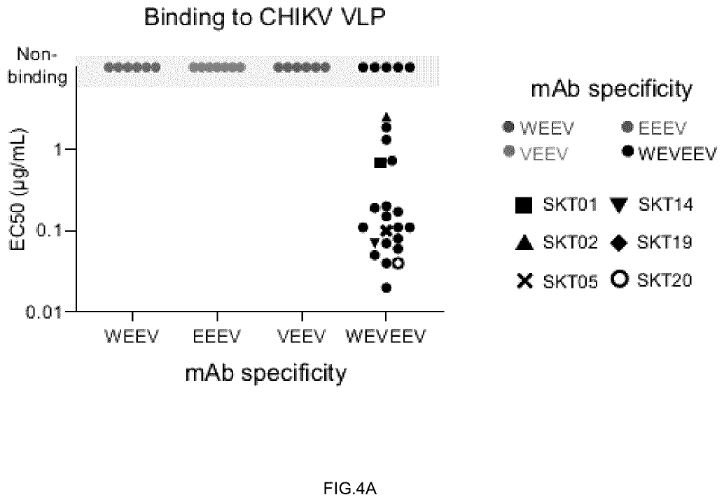

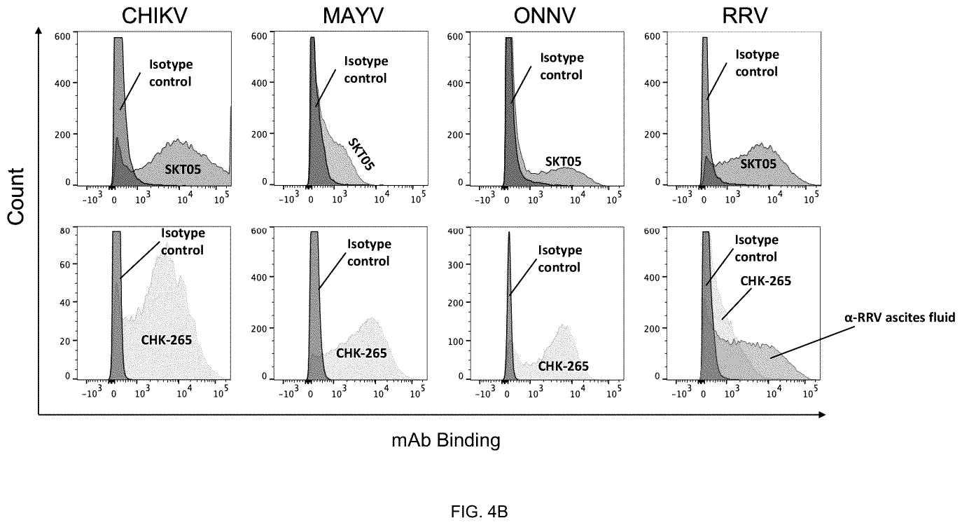

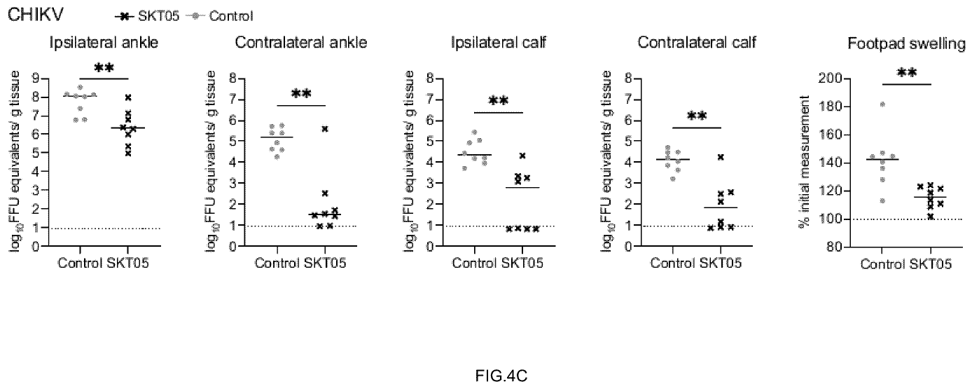

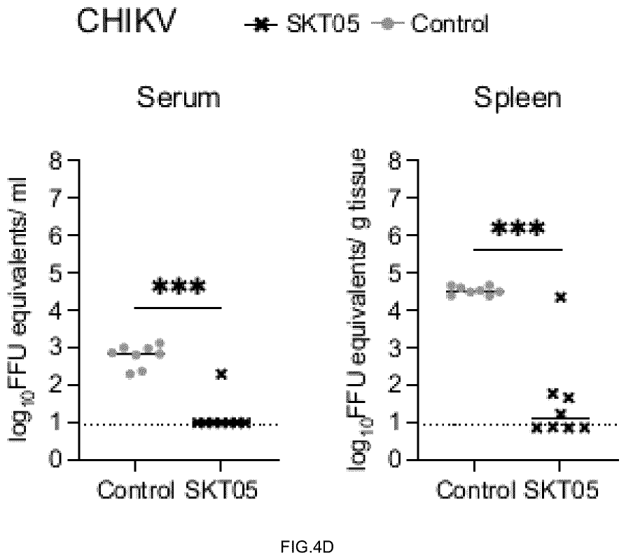

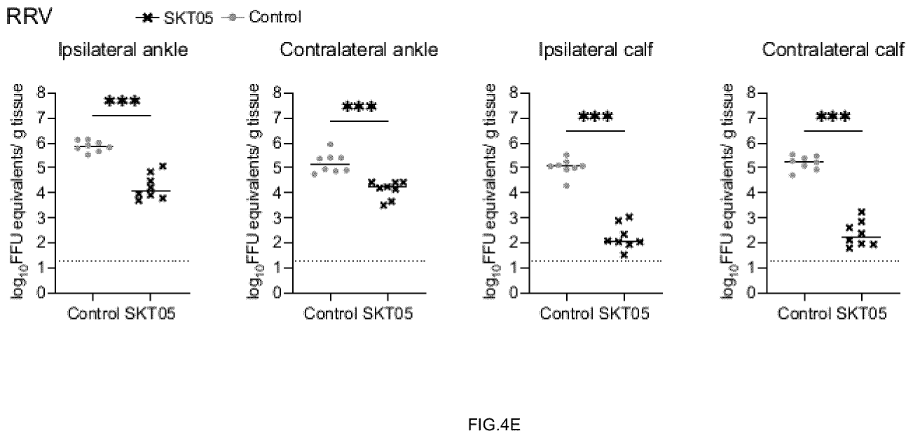

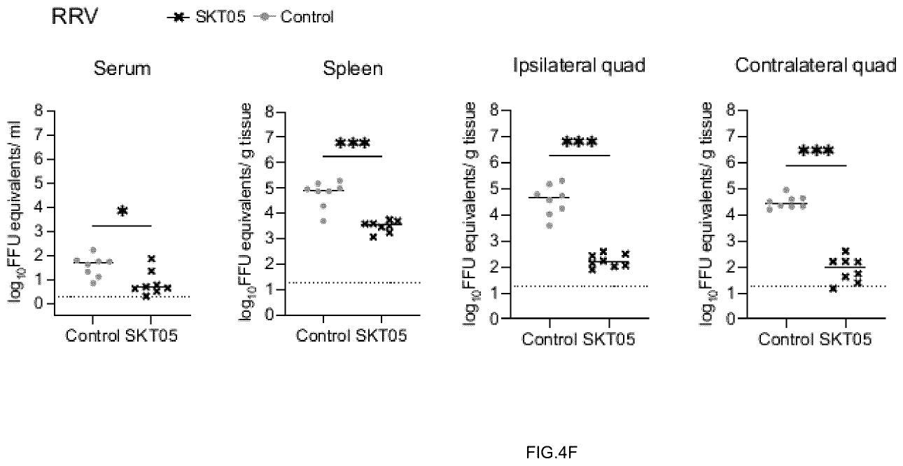

To provide insight into the relationship between recognized epitope and neutralization breadth, epitope characteristics were assessed (FIG. 4E; Table S4) and correlated with binding breadth. Recognition of E1 positively correlated with binding breadth, whereas recognition of E2 negatively correlated with binding breadth, although these correlations did not reach statistical significance. Other epitope characteristics such as depth, surface area, side chain recognition and main chain recognition showed less significant associations. However, two epitope characteristics showed significant correlation with binding breadth: sequence variation and conformational variability. Analysis of all a-EEV antibody-VLP structures in the PDB for structural and sequence variation revealed high correlation between these two epitope features (R=0.69, P=0.03). For this analysis, both the WEEV-SKT05 structure at 4.2 Å resolution, and the structure of VEEV-SKT05, at 3.5 Å resolution was included. Independent of the virus VLP used for structure determination, the epitopes for two triple-specific binders, SKT05 and SKT20, showed the lowest overall sequence and conformational variation. Overall, the collection of structures of neutralizing single-specific and triple-specific antibodies showed broad recognition to inversely correlate with two epitope features: sequence variation and conformational variability.

SKT05 recognizes different symmetry-related protomers in VEEV and WEEV VLPs.

In addition to differences in overall resolution, the structures of SKT05 with WEEV (4.2 Å) and with VEEV (3.5 Å) revealed SKT05 to recognize different symmetry-defined protomers in VEEV and WEEV VLPs. In particular, two SKT05 Fabs bound at the central two-fold axis of the VLP, where three potential binding orientations were possible. In both VEEV and WEEV, only one of the three potential binding orientations was used, and it was different for the two VLPs. In both cases, modeling indicated SKT05 to clash at the two orientations that were not used for each VLP.

Comparison of the recognition by SKT05 in VEEV and WEEV revealed changes in orientations of side chains, often related to differences in the sequences of the epitopes on VEEV and WEEV. Overall, the two structures were highly similar, with only 0.8 Å RMSD in epitope and 0.6 Å RMSD in paratope. The differences in SKT05 recognition of VEEV and WEEV protomers likely explains difference in competition between SKT05 and other antibodies, which differed depending on the VLP analyzed. For example, the triple-specific mAbs that neutralized all three pseudoviruses (SKT01, SKT02, SKT05, SKT14, and SKT19) all shared the same competition group for the EEEV and VEEV VLP (FIG. 2A and FIG. 2B), yet SKT05 was not blocked by these mAbs when competition was assessed with the WEEV VLP (FIG. 2C). Moreover, mAbs in the SKT23 competition group identified with the VEEV VLP blocked all other mAbs from binding, while this was not the case with the WEEV VLP. Of note, SKT20 clustered into the same competition group regardless of VLP. Collectively these data demonstrate that SKT05 recognizes symmetry-related protomers of VEEV and WEEV differently, and that this difference enables antibodies that bind to the same region on the E1E2 trimer to not compete in the context of the full VLP.

### Example 7. Mechanisms Enabling Broad Recognition by SKT05 and SKT20

Triple-specific mAbs fell into 3-4 competition groups: for EEEV and VEEV, those that competed with SKT05, those that competed with SKT20, and those that competed with both (FIG. 2D); for WEEV, SKT05 was in a competition group of its own compared to the other broadly neutralizing mAbs that shared its competition group for EEEV and VEEV. Thus, it was expected that all triple-specific mAbs would recognize regions defined by these two antibodies. Consistent with the competition data, SKT05 and SKT20 bound epitopes that were proximal, but not overlapping. SKT05 used complementarity-determining regions (CDRs) L1, L2 and H3 regions to bind an epitope immediately N terminal to the fusion peptide, whereas SKT20 utilized all CDRs except L2 to bind directly to the fusion peptide.

The SKT20 epitope was almost 100% conserved within EEVs, with sequence conservation of the epitope providing a clear mechanism for its broad recognition. The SKT05 epitope, in contrast, involved a mixture of conserved residues (mostly recognized by the light chain) and variable residue (mostly recognized by the heavy chain). The variable residues recognized by the heavy chain, however, involved backbone contacts not dependent on sequence conservation. Thus, SKT05 utilized a more complex mechanism of binding, with recognition of conserved residues intermixed with recognition of backbone for variable residues.

The conformational change induced in the VLPs by SKT05 and SKT20 was also examined. Some variation in the fusion loop was observed between EEEV, VEEV and WEEV (RMSD of 1.6 Å), but a much larger change in fusion loop conformation was induced by SKT20 (RMSD of 4.3 Å). By contrast, binding of SKT05 induced negligible backbone movements (RMSD of 0.5 Å).

Lastly, the SKT05 epitope was compared with broadly reactive DC2.112 and DC2.315 antibodies reported to also impact EEVs (Kim et al., 2021). The locations of residues identified by mutational analysis to impact binding of DC2.112 and DC2.315 were mapped; residues impacting DC2.112 and DC2.315 antibody recognition overlapped with the SKT20 epitope, but not with the SKT05 epitope. Thus, broad recognition by SKT05 and SKT20 appears to involve quite different mechanisms. For SKT05, broad recognition involved binding at a conformationally conserved but somewhat sequence variable epitope, utilizing backbone recognition. For SKT20, broad recognition involved binding at conformationally divergent but highly sequence conserved epitope.

### Example 8. SKT05 Recognizes and Protects In Vivo Against Arthritogenic Alphaviruses

Due to the unique structural characteristics revealed by cryo-EM, it was hypothesized that the triple-specific a-EEV mAbs might also bind additional alphaviruses. This possibility was first assessed by testing binding to CHIKV VLPs; representative single-specific mAbs and all triple-specific mAbs were tested for binding by ELISA. None of the single-specific mAbs bound CHIKV VLP. In contrast, 21 triple-specific mAbs bound CHIKV VLP with EC50s ranging from 20 ng/mL to 2.49 μg/mL (median 110 ng/mL) (FIG. 4A). Among the more potent triple-specific mAbs that bound CHIKV VLP, SKT05 had an EC50 value of 100 ng/mL. These results were substantiated by a docked structure of SKT05 Fab in complex with CHIKV VLP, which revealed compatible binding to those regions identified for WEEV and VEEV. Indeed, arthritogenic and encephalitic alphaviruses share greater sequence identity and similarity within the E1 protein than the E2 protein (Kim et al., 2021). However, SKT05 failed to neutralize CHIKV, MAYV, ONNV, and RRV (FIG. 4B). Thus, binding of select triple-specific mAbs to live cells infected with CHIKV, MAYV, ONNV, and RRV was tested. Notably, SKT05 bound CHIKV-, RRV-, and ONNV-infected cells, with binding to MAYV-infected cells also being observed but to a lesser extent. A similar trend was observed with the other broadly neutralizing triple-specific mAbs; SKT20 bound CHIKV- and MAYV-infected cells with limited binding to RRV- and ONNV-infected cells.

Given the cross-reactive binding to arthritogenic alphaviruses the ability of SKT05 to confer protective effects in vivo was determined. SKT05 was administered to four-week-old C57BL/6J mice one day prior to subcutaneous challenge with 10³ FFUs of CHIKV or RRV. Three days after challenge, mice were euthanized, and tissues were harvested to assess viral load. For CHIKV-infected mice, administration of SKT05 reduced footpad swelling and viral burden in all tissues assessed when compared to a control mAb (FIGS. 4C & 4D. Similarly, SKT05-treated mice also significantly reduced viral load in all tissues assessed following RRV challenge (FIGS. 4E &4F). Thus, when administered prophylactically, SKT05 protects not only against encephalitic alphavirus challenge, but also provides protective benefits against arthritogenic alphavirus challenge.

### Example 9. Humanized SKT05 Variants

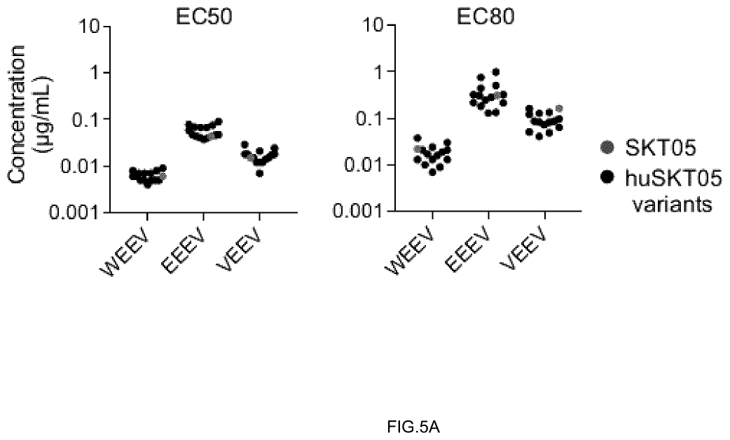

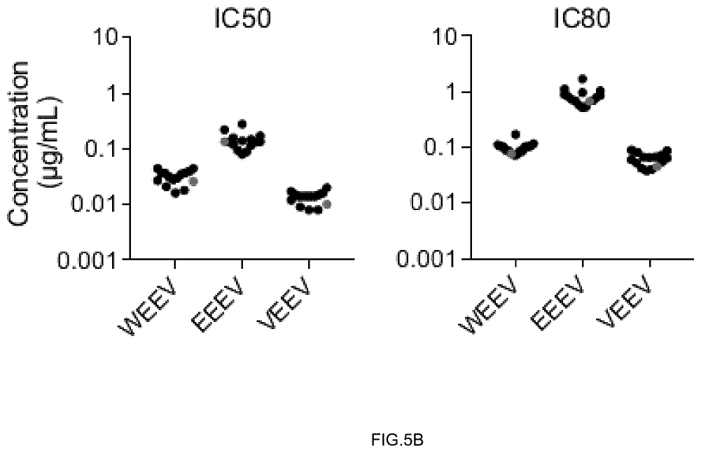

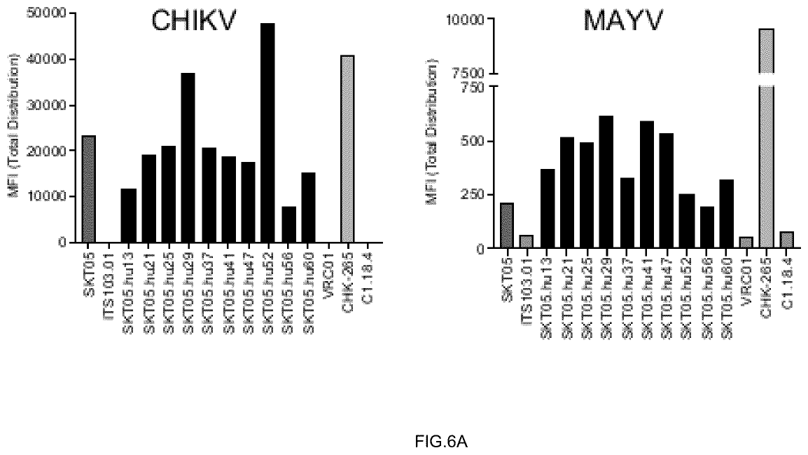

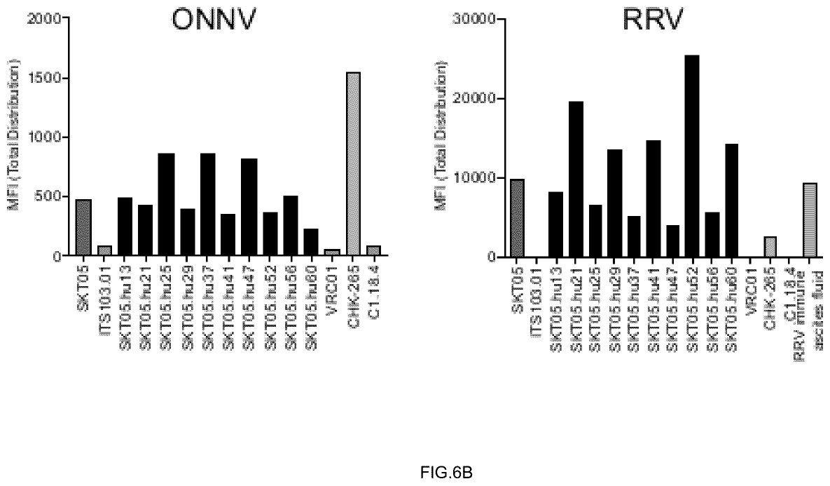

The close phylogenetic relationship between NHPs and humans provides a unique opportunity to modify NHP mAbs to resemble more closely those expressed by human germline genes with minimal mutations as potential clinically useful drugs. Accordingly, 6 amino acid framework positions of SKT05 were identified that differed from the closest human germline sequence and its alleles and created 64 SKT05 variants with different degrees of humanization (all combinations of the 6 mutations). Of these, 14 bound all three EEV VLPs with EC50 and EC80 values comparable to SKT05 (FIG. 5A). The same 14 SKT05 variants neutralized EEV pseudoviruses with IC50 and IC80 values comparable to SKT05 (FIG. 5B). 10 SKT05 variants that most closely resembled the human germline gene were selected to test for binding to live cells infected with CHIKV, MAYV, ONNV, and RRV. Similar to SKT05, the variants predominantly bound cells infected with CHIKV, RRV, and ONNV, while binding to MAYV appeared to be slightly improved (FIGS. 6A & 6B). Thus, this work identified several partially humanized SKT05 variants that retain binding and neutralization characteristics of SKT05 and may prove valuable in a clinical setting.

### Example 10. SKT05 Recognizes and Protects In Vivo Against Arthritogenic Alphaviruses

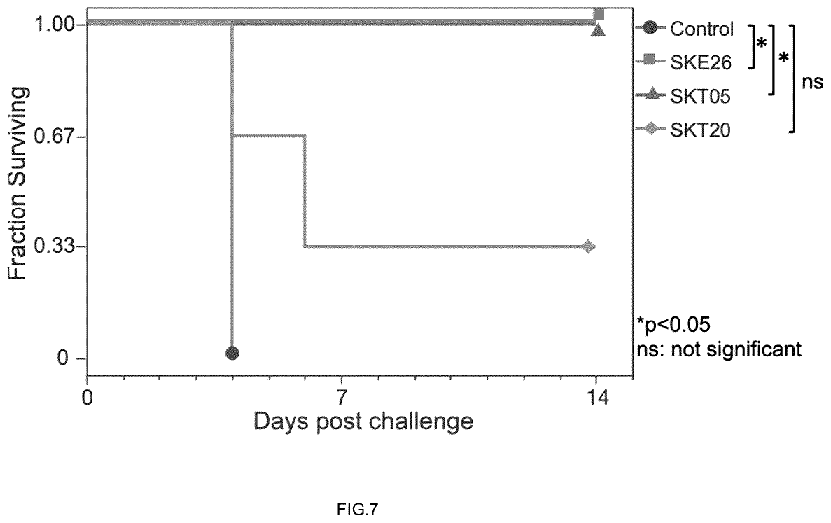

Given that protection against VEEV that was observed in mice, experiments were conducted to determine if protection could be achieved in a more lethal challenge model in NHP. This was tested using Eastern EEEV (strain FL93-939) and challenged via the aerosol route. Previously this challenge model has been shown to be lethal in naïve animals in less than 1 week. Two tri-specific antibodies (SKT05 and SKT20) were tested, one EEEV-specific mAb (SKE26), and an anti-SIV control antibody (ITS103.01). Each group consisted of 3 animals. Animals received 30 mg/kg of antibody 5 days prior to aerosol challenge with 10\hat{ }7 PFU (plaque forming units) EEEV and were monitored for 14 days. The results of this challenge are shown in FIG. 7.

### Discussion

This study describes the isolation and structural characterization of a-EEV mAbs with protective effects against both encephalitic and arthritogenic alphaviruses from trivalent VLP-immunized macaques. In total, 109 a-EEV mAbs were identified that (by experimental design) recognized either a single VLP, or all three VLPs, were identified. Competitive binding ELISAs revealed 6-7 single-specific competition groups for each virus and 3-4 triple-specific competition groups, and cryo-EM structural analysis of 6 single-specific mAbs and 2 triple-specific antibodies revealed that broad recognition inversely correlated with sequence and conformational variability. Two triple-specific mAbs, SKT05 and SKT20, bound epitopes proximal or including the highly conserved fusion peptide. Despite binding all three viruses at an epitope within the fusion peptide that is almost 100% conserved, SKT20 only neutralized VEEV. In contrast, SKT05 bound proximal to the fusion peptide and potently neutralized all three EEV pseudoviruses. While the SKT05 epitope contained a mixture of both conserved and variable residues, variable residues were recognized through backbone contacts not dependent on sequence conservation. Because of the high reliance of backbone interactions, it is less likely that a virus could select for mutations rendering it resistant to SKT5 neutralization.

Historically, the isolation of anti-alphavirus mAbs has relied heavily on sampling symptomatic individuals in regions following an outbreak. While elicitation of protective mAbs against individual alphaviruses has been reported following vaccination, the development of a vaccine or monoclonal antibody capable of providing protection against both classes of alphaviruses remains highly desirable. It had been previously demonstrated that vaccination with trivalent WEVEEV VLPs protects NHP from aerosol challenge with all three encephalitic alphaviruses, and that this protection was humoral (Ko et al., 2019). Using the same immunogens, the instant disclosure shows the elicitation of many a-EEV mAbs targeting a single, or all three, encephalitic alphaviruses. When administered prophylactically, two of these vaccine-elicited triple-specific antibodies, SKT05 and SKT20, provide protection against encephalitic alphavirus challenge. Furthermore, SKT05 also provides protection from arthritogenic alphavirus challenge. Notably, these immunogens have also been assessed in a phase 1 clinical trial and shown to be immunogenic and well tolerated (Coates et al., 2022). Four weeks after the second immunization, 76% of participants developed neutralizing antibody titers against all three encephalitic alphaviruses. Notably, the triple-specific mAbs isolated from two vaccinated subjects shared a competition group with SKT20. Given the large increase in the elicitation of triple-specific mAbs following a third (perhaps delayed) immunization in NHP, it is expected that superior clinical results will be achieved with a similar regimen.

SKT05 and SKT20 neutralized the VEEV pseudovirus and protected mice from weight loss and death during VEEV challenge. Interestingly, only SKT05 reduced viral burden in the brain, suggesting that SKT05 and SKT20 may have distinct mechanisms of protection. In this mouse model of VEEV infection, high viral load in the brain does not always correlate with lethality. Other factors such as increased blood brain barrier permeability and induction of cytokines, particularly IFN-γ, can lead to encephalitis and death (Hollidge et al., 2021; Julander et al., 2008). For example, SKT05 may be more efficient at reducing infection of the olfactory neuroepithelium through egress blockade or Fc-mediated effector functions and preventing central nervous system (CNS) infection, while SKT20 may be more primed to alter the inflammation that follows viral penetration of the blood brain barrier and replication in the CNS (Cain et al., 2017; Steele et al., 1998). However, the different neutralization and binding activity of SKT05 and SKT20 to VEEV may be associated with the in vivo protection and should be considered during future studies. Thus, while neutralization is likely a positive predictor of in vivo efficacy, the lack of in vitro neutralization does not predict lack of efficacy. It will be interesting to assess the in vivo efficacy of SKT05 both in NHP challenge models and clinically, as humanized variants retain recognition of both encephalitic and arthritogenic viruses.

A central finding of this work is the definition of the SKT05-binding site of vulnerability, which, unlike the site recognized by other pan-alphavirus mAbs, is accessible to antibody on the fully assembled VLP. The E1 binding site does contain some sequence variability, SKT05 employs a binding mode that surmounts this by recognizing backbone atoms of sequence variable residues. This mode of binding, which makes viral resistance more difficult to achieve, provides additional impetus to the clinical development of SKT05.

1. An isolated antibody, wherein the isolated antibody is selected from the group consisting of:
a. an isolated antibody in which the specificity determining residues of the isolated antibody interact with at least 20, optionally at least 22, optionally at least 24, optionally at least 26, optionally at least 28, optionally at least 30, optionally at least 31, optionally at least 32, or all, contact residues selected from the group consisting of G64 of the E1 protein of VEEV, S65 of the E1 protein of VEEV, Q66 of the E1 protein of VEEV, E67 of the E1 protein of VEEV, C68 of the E1 protein of VEEV, T69 of the E1 protein of VEEV, P70 of the E1 protein of VEEV, Y72 of the E1 protein of VEEV, D75 of the E1 protein of VEEV, E76 of the E1 protein of VEEV, Q77 of the E1 protein of VEEV, C78 of the E1 protein of VEEV, K79 of the E1 protein of VEEV, V80 of the E1 protein of VEEV, F81 of the E1 protein of VEEV, T82 of the E1 protein of VEEV, T98 of the E1 protein of VEEV, E99 of the E1 protein of VEEV, Y214 of the E1 protein of VEEV, A215 of the E1 protein of VEEV, N216 of the E1 protein of VEEV, N218 of the E1 protein of VEEV, L219 of the E1 protein of VEEV, V220 of the E1 protein of VEEV, R223 of the E1 protein of VEEV, S141 of the E1 protein of VEEV, V143 of the E1 protein of VEEV, D311 of the E1 protein of VEEV, K340 of the E1 protein of VEEV, T389 of the E1 protein of VEEV, H390 of the E1 protein of VEEV, Y393 of the E1 protein of VEEV;
b. an isolated antibody in which the specificity determining residues of the isolated antibody interact with at least 18, optionally at least 20, optionally at least 21, optionally at least 22, or all, contact residues selected from the group consisting of F87 of the E1 protein of VEEV, M88 of the E1 protein of VEEV, W89 of the E1 protein of VEEV, G90 of the E1 protein of VEEV, G91 of the E1 protein of VEEV, Y93 of the E1 protein of VEEV, F95 of the E1 protein of VEEV, C96 of the E1 protein of VEEV, D97 of the E1 protein of VEEV, E99 of the E1 protein of VEEV, A226 of the E1 protein of VEEV, G227 of the E1 protein of VEEV, H28 of the E2 protein of VEEV, L55 of the E2 protein of VEEV, G63 of the E2 protein of VEEV, R64 of the E2 protein of VEEV, D94 of the E2 protein of VEEV, G95 of the E2 protein of VEEV, H96 of the E2 protein of VEEV, H156 of the E2 protein of VEEV, D157 of the E2 protein of VEEV, A158 of the E2 protein of VEEV, and Q159 of the E2 protein of VEEV;
c. an isolated antibody in which the specificity determining residues of the isolated antibody interact with at least 20, optionally at least 22, optionally at least 24, optionally at least 26, optionally at least 28, optionally at least 30, optionally at least 32, optionally at least 34, optionally at least 36, optionally at least 37, or all, contact residues selected from the group consisting of Y89 of the E1 protein of EEEV, G90 of the E1 protein of EEEV, D3 of the E2 protein of EEEV, F6 of the E2 protein of EEEV, T7 of the E2 protein of EEEV, Y9 of the E2 protein of EEEV, K10 of the E2 protein of EEEV, L11 of the E2 protein of EEEV, R13 of the E2 protein of EEEV, T57 of the E2 protein of EEEV, D58 of the E2 protein of EEEV, G59 of the E2 protein of EEEV, M68 of the E2 protein of EEEV, N69 of the E2 protein of EEEV, G70 of the E2 protein of EEEV, K71 of the E2 protein of EEEV, T72 of the E2 protein of EEEV, Q73 of the E2 protein of EEEV, K74 of the E2 protein of EEEV, S75 of the E2 protein of EEEV5, V172, S176 of the E2 protein of EEEV, K186 of the E2 protein of EEEV, T188 of the E2 protein of EEEV, V189 of the E2 protein of EEEV, P190 of the E2 protein of EEEV, S191 of the E2 protein of EEEV, G192 of the E2 protein of EEEV, A193 of the E2 protein of EEEV, Q194 of the E2 protein of EEEV, T209 of the E2 protein of EEEV, S210 of the E2 protein of EEEV, S211 of the E2 protein of EEEV, L227 of the E2 protein of EEEV, I228 of the E2 protein of EEEV, D229 of the E2 protein of EEEV, N230 of the E2 protein of EEEV, K231 of the E2 protein of EEEV, and K232 of the E2 protein of EEEV;
d. an isolated antibody in which the specificity determining residues of the isolated antibody interact with at least 16, optionally at least 17, optionally at least 18, optionally at least 19, or all, contact residues selected from the group consisting of E348 of the E1 protein of VEEV, Q349 of the E1 protein of VEEV, K319 of the E1 protein of VEEV, S325 of the E1 protein of VEEV, T347 of the E1 protein of VEEV, E348 of the E1 protein of VEEV, Q349 of the E1 protein of VEEV, A298 of the E1 protein of VEEV, T317 of the E1 protein of VEEV, K319 of the E1 protein of VEEV, S323 of the E1 protein of VEEV, K324 of the E1 protein of VEEV, S325 of the E1 protein of VEEV, G326 of the E1 protein of VEEV, E345 of the E1 protein of VEEV, T347 of the E1 protein of VEEV, E348 of the E1 protein of VEEV, Q349 of the E1 protein of VEEV, G350 of the E1 protein of VEEV, S351 of the E1 protein of VEEV;
e. an isolated antibody in which the specificity determining residues of the isolated antibody interact with at least 16, optionally at least 17, optionally at least 18, optionally at least 19, or all, contact residues selected from the group consisting of S180 of the E2 protein of VEEV, L181 of the E2 protein of VEEV, S182 of the E2 protein of VEEV, G183 of the E2 protein of VEEV, S185 of the E2 protein of VEEV, T187 of the E2 protein of VEEV, T189 of the E2 protein of VEEV, P190 of the E2 protein of VEEV, P191 of the E2 protein of VEEV, D192 of the E2 protein of VEEV, G193 of the E2 protein of VEEV, E209 of the E2 protein of VEEV, T210 of the E2 protein of VEEV, 1211 of the E2 protein of VEEV, N212 of the E2 protein of VEEV, K213 of the E2 protein of VEEV, T214 of the E2 protein of VEEV, K215 of the E2 protein of VEEV, Q216 of the E2 protein of VEEV, S218 of the E2 protein of VEEV;
f. an isolated antibody in which the specificity determining residues of the isolated antibody interact with at least 20, optionally at least 22, optionally at least 24, optionally at least 26, or all, contact residues selected from the group consisting of C63 of the E1 protein of WEEV, S65 of the E1 protein of WEEV, L66 of the E1 protein of WEEV, E67 of the E1 protein of WEEV, C68 of the E1 protein of WEEV, K69 of the E1 protein of WEEV, A70 of the E1 protein of WEEV, S71 of the E1 protein of WEEV, D75 of the E1 protein of WEEV, Y76 of the E1 protein of WEEV, T77 of the E1 protein of WEEV, C78 of the E1 protein of WEEV, R79 of the E1 protein of WEEV, V80 of the E1 protein of WEEV, F81 of the E1 protein of WEEV, G82 of the E1 protein of WEEV, G83 of the E1 protein of WEEV, S98 of the E1 protein of WEEV, E99 of the E1 protein of WEEV, N100 of the E1 protein of WEEV, T101 of the E1 protein of WEEV, M195 of the E1 protein of WEEV, A215 of the E1 protein of WEEV, R216 of the E1 protein of WEEV, D218 of the E1 protein of WEEV, I219 of the E1 protein of WEEV, L221 of the E1 protein of WEEV, K223 of the E1 protein of WEEV;
g. an isolated antibody in which the specificity determining residues of the isolated antibody interact with at least 16, optionally at least 17, or all, contact residues selected from the group consisting of N57 of the E2 protein of WEEV, Q58 of the E2 protein of WEEV, A59 of the E2 protein of WEEV, T61 of the E2 protein of WEEV, R173 of the E2 protein of WEEV, G197 of the E2 protein of WEEV, K198 of the E2 protein of WEEV, K233 of the E2 protein of WEEV, D235 of the E2 protein of WEEV, Q236 of the E2 protein of WEEV, T237 of the E2 protein of WEEV, W239 of the E2 protein of WEEV, D251 of the E2 protein of WEEV, S253 of the E2 protein of WEEV, V254 of the E2 protein of WEEV, Q255 of the E2 protein of WEEV, G256 of the E2 protein of WEEV, K257 of the E2 protein of WEEV;
h. an isolated antibody in which the specificity determining residues of the isolated antibody interact with at least 20, or all, contact residues selected from the group consisting of K61 of the E1 protein of WEEV, C63 of the E1 protein of WEEV, G64 of the E1 protein of WEEV, S65 of the E1 protein of WEEV, F95 of the E1 protein of WEEV, N199 of the E2 protein of WEEV, T201 of the E2 protein of WEEV, Y202 of the E2 protein of WEEV, K205 of the E2 protein of WEEV, D208 of the E2 protein of WEEV, Y209 of the E2 protein of WEEV, S210 of the E2 protein of WEEV, T211 of the E2 protein of WEEV, G212 of the E2 protein of WEEV, I213 of the E2 protein of WEEV, V214 of the E2 protein of WEEV, S215 of the E2 protein of WEEV, T216 of the E2 protein of WEEV, T218 of the E2 protein of WEEV, K219 of the E2 protein of WEEV, M220 of the E2 protein of WEEV, N221 of the E2 protein of WEEV; and,
i. an isolated antibody in which the specificity determining residues of the isolated antibody interact with at least 20, optionally at least 22, optionally at least 24, optionally at least 26, optionally at least 28, optionally at least 30, or all, contact residues selected from the group consisting of Q59 of the E1 protein of WEEV, V60 of the E1 protein of WEEV, K61 of the E1 protein of WEEV, C62 of the E1 protein of WEEV, Q93 of the E1 protein of WEEV, C94 of the E1 protein of WEEV, F95 of the E1 protein of WEEV, R173 of the E2 protein of WEEV, P176 of the E2 protein of WEEV, A178 of the E2 protein of WEEV, T201 of the E2 protein of WEEV, Y202 of the E2 protein of WEEV, E203 of the E2 protein of WEEV, K205 of the E2 protein of WEEV, G207 of the E2 protein of WEEV, D208 of the E2 protein of WEEV, Y209 of the E2 protein of WEEV, S210 of the E2 protein of WEEV, T211 of the E2 protein of WEEV, G212 of the E2 protein of WEEV, I213 of the E2 protein of WEEV, N221 of the E2 protein of WEEV, 1230 of the E2 protein of WEEV, Y232 of the E2 protein of WEEV, S234 of the E2 protein of WEEV, Q236 of the E2 protein of WEEV, 1247 of the E2 protein of WEEV, R248 of the E2 protein of WEEV, H249 of the E2 protein of WEEV, T250 of the E2 protein of WEEV, D251 of the E2 protein of WEEV, S253 of the E2 protein of WEEV, Q255 of the E2 protein of WEEV.

2. The isolated antibody of claim 1, wherein the isolated antibody comprises a V_{L} CDR3 selected from the group consisting of: a V_{L} CDR3 comprising SEQ ID NO:179, a V_{L} CDR3 comprising SEQ ID NO:227, a V_{L} CDR3 comprising SEQ ID NO:235, a V_{L} CDR3 comprising SEQ ID NO:267, a V_{L} CDR3 comprising SEQ ID NO:3, a V_{L} CDR3 comprising SEQ ID NO:11, a V_{L} CDR3 comprising SEQ ID NO:19, a V_{L} CDR3 comprising SEQ ID NO:27, a V_{L} CDR3 comprising SEQ ID NO:35, a V_{L} CDR3 comprising SEQ ID NO:43, a V_{L} CDR3 comprising SEQ ID NO:51, a V_{L} CDR3 comprising SEQ ID NO:59, a V_{L} CDR3 comprising SEQ ID NO:67, a V_{L} CDR3 comprising SEQ ID NO:83, a V_{L} CDR3 comprising SEQ ID NO:91, a V_{L} CDR3 comprising SEQ ID NO:99, a V_{L} CDR3 comprising SEQ ID NO:107, a V_{L} CDR3 comprising SEQ ID NO:115, a V_{L} CDR3 comprising SEQ ID NO:123, a V_{L} CDR3 comprising SEQ ID NO:131, a V_{L} CDR3 comprising SEQ ID NO:139, a V_{L} CDR3 comprising SEQ ID NO: 147, a V_{L} CDR3 comprising SEQ ID NO:155, a V_{L} CDR3 comprising SEQ ID NO:163, a V_{L} CDR3 comprising SEQ ID NO:171, a V_{L} CDR3 comprising SEQ ID NO:187, a V_{L} CDR3 comprising SEQ ID NO:195, a V_{L} CDR3 comprising SEQ ID NO:203, a V_{L} CDR3 comprising SEQ ID NO:211, a V_{L} CDR3 comprising SEQ ID NO:219, a V_{L} CDR3 comprising SEQ ID NO:243, a V_{L} CDR3 comprising SEQ ID NO:251, a V_{L} CDR3 comprising SEQ ID NO:259, a V_{L} CDR3 comprising SEQ ID NO:275, and a V_{L} CDR3 comprising SEQ ID NO:273.

3. The isolated antibody of claim 1, wherein the isolated antibody comprises a V_{H} CDR3 selected from the group consisting of: a V_{H} CDR3 comprising SEQ ID NO:183, a V_{H} CDR3 comprising SEQ ID NO:231, a V_{H} CDR3 comprising SEQ ID NO:239, a V_{H} CDR3 comprising SEQ ID NO:271, a V_{H} CDR3 comprising SEQ ID NO:7, a V_{H} CDR3 comprising SEQ ID NO:15, a V_{H} CDR3 comprising SEQ ID NO:23, a V_{H} CDR3 comprising SEQ ID NO:31, a V_{H} CDR3 comprising SEQ ID NO:39, a V_{H} CDR3 comprising SEQ ID NO:47, a V_{H} CDR3 comprising SEQ ID NO:55, a V_{H} CDR3 comprising SEQ ID NO:63, a V_{H} CDR3 comprising SEQ ID NO:71, a V_{H} CDR3 comprising SEQ ID NO:79, a V_{H} CDR3 comprising SEQ ID NO:87, a V_{H} CDR3 comprising SEQ ID NO:95, a V_{H} CDR3 comprising SEQ ID NO:103, a V_{H} CDR3 comprising SEQ ID NO:111, a V_{H} CDR3 comprising SEQ ID NO:119, a V_{H} CDR3 comprising SEQ ID NO:127, a V_{H} CDR3 comprising SEQ ID NO:135, a V_{H} CDR3 comprising SEQ ID NO:143, a V_{H} CDR3 comprising SEQ ID NO:151, a V_{H} CDR3 comprising SEQ ID NO:159, a V_{H} CDR3 comprising SEQ ID NO:167, a V_{H} CDR3 comprising SEQ ID NO:175, a V_{H} CDR3 comprising SEQ ID NO:191, a V_{H} CDR3 comprising SEQ ID NO:199, a V_{H} CDR3 comprising SEQ ID NO:207, a V_{H} CDR3 comprising SEQ ID NO:215, a V_{H} CDR3 comprising SEQ ID NO:223, a V_{H} CDR3 comprising SEQ ID NO:247, a V_{H} CDR3 comprising SEQ ID NO:255, a V_{H} CDR3 comprising SEQ ID NO:263, a V_{H} CDR3 comprising SEQ ID NO:279, and a V_{H} CDR3 comprising SEQ ID NO:287.

4. The isolated antibody of claim 1, wherein the isolated antibody comprises:
i) a V_{L} CDR3 comprising or consisting of SEQ ID NO:179 and a V_{H} CDR3 comprising or consisting of SEQ ID NO:183;
ii) a V_{L} CDR3 comprising or consisting of SEQ ID NO:227; and a V_{H} CDR3 comprising or consisting of SEQ ID NO:231;
iii) a V_{L} CDR3 comprising or consisting of SEQ ID NO:235 and a V_{H} CDR3 comprising or consisting of SEQ ID NO:239;
iv) a V_{L} CDR3 comprising or consisting of SEQ ID NO:267 and a V_{H} CDR3 comprising or consisting of SEQ ID NO:271;
v) a V_{L} CDR3 comprising or consisting of SEQ ID NO: 3 and a V_{H} CDR3 comprising or consisting of SEQ ID NO:7;
vi) a V_{L} CDR3 comprising or consisting of SEQ ID NO:11 and a V_{H} CDR3 comprising or consisting of SEQ ID NO:15;
vii) a V_{L} CDR3 comprising or consisting of SEQ ID NO:19 and a V_{H} CDR3 comprising or consisting of SEQ ID NO:23;
viii) a V_{L} CDR3 comprising or consisting of SEQ ID NO:27 and a V_{H} CDR3 comprising or consisting of SEQ ID NO:31;
ix) a V_{L} CDR3 comprising or consisting of SEQ ID NO:35 and a V_{H} CDR3 comprising or consisting of SEQ ID NO:39;
x) a V_{L} CDR3 comprising or consisting of SEQ ID NO:43 and a V_{H} CDR3 comprising or consisting of SEQ ID NO:47;
xi) a V_{L} CDR3 comprising or consisting of SEQ ID NO:51 and a V_{H} CDR3 comprising or consisting of SEQ ID NO:55;
xii) a V_{L} CDR3 comprising or consisting of SEQ ID NO:59 and a V_{H} CDR3 comprising or consisting of SEQ ID NO:63;
xiii) a V_{L} CDR3 comprising or consisting of SEQ ID NO:67 and a V_{H} CDR3 comprising or consisting of SEQ ID NO:71;
xiv) a V_{L} CDR3 comprising or consisting of SEQ ID NO:75 and a V_{H} CDR3 comprising or consisting of SEQ ID NO:79;
xv) a V_{L} CDR3 comprising or consisting of SEQ ID NO:83 and a V_{H} CDR3 comprising or consisting of SEQ ID NO:87;
xvi) a V_{L} CDR3 comprising or consisting of SEQ ID NO:91 and a V_{H} CDR3 comprising or consisting of SEQ ID NO:95;
xvii) a V_{L} CDR3 comprising or consisting of SEQ ID NO:99 and a V_{H} CDR3 comprising or consisting of SEQ ID NO: 10;
xviii) a V_{L} CDR3 comprising or consisting of SEQ ID NO:107 and a V_{H} CDR3 comprising or consisting of SEQ ID NO:111;
xix) a V_{L} CDR3 comprising or consisting of SEQ ID NO:115 and a V_{H} CDR3 comprising or consisting of SEQ ID NO:119;
xx) a V_{L} CDR3 comprising or consisting of SEQ ID NO:123 and a V_{L} CDR3 comprising or consisting of SEQ ID NO:127;
xxi) a V_{L} CDR3 comprising or consisting of SEQ ID NO:131 and a V_{H} CDR3 comprising or consisting of SEQ ID NO:135;
xxii) a V_{L} CDR3 comprising or consisting of SEQ ID NO:139 and a V_{H} CDR3 comprising or consisting of SEQ ID NO:143;
xxiii) a V_{L} CDR3 comprising or consisting of SEQ ID NO:147 and a V_{H} CDR3 comprising or consisting of SEQ ID NO:151;
xxiv) a V_{L} CDR3 comprising or consisting of SEQ ID NO:155 and a V_{H} CDR3 comprising or consisting of SEQ ID NO:159;
xxv) a V_{L} CDR3 comprising or consisting of SEQ ID NO:163 and a V_{H} CDR3 comprising or consisting of SEQ ID NO:167;
xxvi) a V_{L} CDR3 comprising or consisting of SEQ ID NO:171 and a V_{H} CDR3 comprising or consisting of SEQ ID NO:175;
xxvii) a V_{L} CDR3 comprising or consisting of SEQ ID NO:187 and a V_{H} CDR3 comprising or consisting of SEQ ID NO:191;
xxviii) a V_{L} CDR3 comprising or consisting of SEQ ID NO:195 and a V_{H} CDR3 comprising or consisting of SEQ ID NO:199;
xxix) a V_{L} CDR3 comprising or consisting of SEQ ID NO:203 and a V_{H} CDR3 comprising or consisting of SEQ ID NO:207;
xxx) a V_{L} CDR3 comprising or consisting of SEQ ID NO 211; and a V_{H} CDR3 comprising or consisting of SEQ ID NO:215;
xxxi) a V_{L} CDR3 comprising or consisting of SEQ ID NO 219; and a V_{H} CDR3 comprising or consisting of SEQ ID NO:223;
xxxii) a V_{L} CDR3 comprising or consisting of SEQ ID NO:243 and a V_{H} CDR3 comprising or consisting of SEQ ID NO:247;
xxxiii) a V_{L} CDR3 comprising or consisting of SEQ ID NO:251 and a V_{H} CDR3 comprising or consisting of SEQ ID NO:255;
xxxiv) a V_{L} CDR3 comprising or consisting of SEQ ID NO 259; and a V_{H} CDR3 comprising or consisting of SEQ ID NO:263;
xxxv) a V_{L} CDR3 comprising or consisting of SEQ ID NO:275 and a V_{H} CDR3 comprising or consisting of SEQ ID NO:279; and,
xxxvi) a V_{L} CDR3 comprising or consisting of SEQ ID NO:283 and a V_{H} CDR3 comprising or consisting of SEQ ID NO:287.

5. The isolated antibody of claim 1, wherein the isolated antibody comprises:
a. a V_{L} CDR1 comprising or consisting of SEQ ID NO:177, a V_{L} CDR2 comprising or consisting of SEQ ID NO: 178, a V_{L} CDR3 comprising or consisting of SEQ ID NO:179, a V_{H} CDR1 comprising or consisting of SEQ ID NO:181, a V_{H} CDR2 comprising or consisting of SEQ ID NO:182, and a V_{H} CDR3 comprising or consisting of SEQ ID NO:183;
b. a V_{L} CDR1 comprising or consisting of SEQ ID NO:225, a V_{L} CDR2 comprising or consisting of SEQ ID NO:226, a V_{L} CDR3 comprising or consisting of SEQ ID NO:227, a V_{H} CDR1 comprising or consisting of SEQ ID NO:229, a V_{H} CDR2 comprising or consisting of SEQ ID NO:230, and a V_{H} CDR3 comprising or consisting of SEQ ID NO:231;
c. a V_{L} CDR1 comprising or consisting of SEQ ID NO:233, a V_{L} CDR2 comprising or consisting of SEQ ID NO:234, a V_{L} CDR3 comprising or consisting of SEQ ID NO:235, a V_{H} CDR1 comprising or consisting of SEQ ID NO:237, a V_{H} CDR2 comprising or consisting of SEQ ID NO:238, and a V_{H} CDR3 comprising or consisting of SEQ ID NO:239;
d. a V_{L} CDR1 comprising or consisting of SEQ ID NO:265, a V_{L} CDR2 comprising or consisting of SEQ ID NO:266, a V_{L} CDR3 comprising or consisting of SEQ ID NO:267, a V_{H} CDR1 comprising or consisting of SEQ ID NO:269, a V_{H} CDR2 comprising or consisting of SEQ ID NO:270, and a V_{H} CDR3 comprising or consisting of SEQ ID NO:271;
e. a V_{L} CDR1 comprising or consisting of SEQ ID NO:1, a V_{L} CDR2 comprising or consisting of SEQ ID NO:2, a V_{L} CDR3 comprising or consisting of SEQ ID NO:3, a V_{H} CDR1 comprising or consisting of SEQ ID NO:5, a V_{H} CDR2 comprising or consisting of SEQ ID NO:6, and a V_{H} CDR3 comprising or consisting of SEQ ID NO:7;
f. a V_{L} CDR1 comprising or consisting of SEQ ID NO:9, a V_{L} CDR2 comprising or consisting of SEQ ID NO:10, a V_{L} CDR3 comprising or consisting of SEQ ID NO:11, a V_{H} CDR1 comprising or consisting of SEQ ID NO:13, a V_{H} CDR2 comprising or consisting of SEQ ID NO:14, and a V_{H} CDR3 comprising or consisting of SEQ ID NO:1;
g. a V_{L} CDR1 comprising or consisting of SEQ ID NO: 17, a V_{L} CDR2 comprising or consisting of SEQ ID NO:18, a V_{L} CDR3 comprising or consisting of SEQ ID NO:19, a V_{H} CDR1 comprising or consisting of SEQ ID NO:21, a V_{H} CDR2 comprising or consisting of SEQ ID NO:22, and a V_{H} CDR3 comprising or consisting of SEQ ID NO:23;
h. a V_{L} CDR1 comprising or consisting of SEQ ID NO:25, a V_{H} CDR2 comprising or consisting of SEQ ID NO:26, a V_{L} CDR3 comprising or consisting of SEQ ID NO:27, a V_{H} CDR1 comprising or consisting of SEQ ID NO:29, a V_{H} CDR2 comprising or consisting of SEQ ID NO:30, and a V_{H} CDR3 comprising or consisting of SEQ ID NO:31;
i. a V_{L} CDR1 comprising or consisting of SEQ ID NO:33, a V_{L} CDR2 comprising or consisting of SEQ ID NO:34, a V_{L} CDR3 comprising or consisting of SEQ ID NO:35, a V_{H} CDR1 comprising or consisting of SEQ ID NO:37, a V_{H} CDR2 comprising or consisting of SEQ ID NO:38, and a V_{H} CDR3 comprising or consisting of SEQ ID NO:39;
j. a V_{L} CDR1 comprising or consisting of SEQ ID NO:41, a V_{L} CDR2 comprising or consisting of SEQ ID NO:42, a V_{L} CDR3 comprising or consisting of SEQ ID NO:43, a V_{L} CDR1 comprising or consisting of SEQ ID NO:45, a V_{H} CDR2 comprising or consisting of SEQ ID NO:46, and a V_{H} CDR3 comprising or consisting of SEQ ID NO:47;
k. a V_{L} CDR1 comprising or consisting of SEQ ID NO:49, a V_{L} CDR2 comprising or consisting of SEQ ID NO:50, a V_{L} CDR3 comprising or consisting of SEQ ID NO:51, a V_{H} CDR1 comprising or consisting of SEQ ID NO:53, a V_{H} CDR2 comprising or consisting of SEQ ID NO:54, and a V_{H} CDR3 comprising or consisting of SEQ ID NO:55;
l. a V_{L} CDR1 comprising or consisting of SEQ ID NO:57, a V_{L} CDR2 comprising or consisting of SEQ ID NO:58, a V_{L} CDR3 comprising or consisting of SEQ ID NO:59, a V_{H} CDR1 comprising or consisting of SEQ ID NO:61, a V_{H} CDR2 comprising or consisting of SEQ ID NO:62, and a V_{H} CDR3 comprising or consisting of SEQ ID NO:63;
m. A V_{L} CDR1 comprising or consisting of SEQ ID NO:65, a V_{L} CDR2 comprising or consisting of SEQ ID NO:66, a V_{L} CDR3 comprising or consisting of SEQ ID NO:67, a V_{H} CDR1 comprising or consisting of SEQ ID NO:69, a V_{H} CDR2 comprising or consisting of SEQ ID NO:70, and a V_{H} CDR3 comprising or consisting of SEQ ID NO:71;
n. a V_{L} CDR1 comprising or consisting of SEQ ID NO:73, a V_{L} CDR2 comprising or consisting of SEQ ID NO:74, a V_{L} CDR3 comprising or consisting of SEQ ID NO:75, a V_{H} CDR1 comprising or consisting of SEQ ID NO:77, a V_{H} CDR2 comprising or consisting of SEQ ID NO:78, and a V_{H} CDR3 comprising or consisting of SEQ ID NO:79;
o. a V_{L} CDR1 comprising or consisting of SEQ ID NO:81, a V_{L} CDR2 comprising or consisting of SEQ ID NO:82, a V_{L} CDR3 comprising or consisting of SEQ ID NO:83, a V_{H} CDR1 comprising or consisting of SEQ ID NO:85, a V_{H} CDR2 comprising or consisting of SEQ ID NO:86, and a V_{H} CDR3 comprising or consisting of SEQ ID NO:87;
p. a V_{L} CDR1 comprising or consisting of SEQ ID NO:89, a V_{L} CDR2 comprising or consisting of SEQ ID NO:90, a V_{L} CDR3 comprising or consisting of SEQ ID NO:91, a V_{H} CDR1 comprising or consisting of SEQ ID NO:93, a V_{H} CDR2 comprising or consisting of SEQ ID NO:94, and a V_{H} CDR3 comprising or consisting of SEQ ID NO:95;
q. a V_{L} CDR1 comprising or consisting of SEQ ID NO:97, a V_{L} CDR2 comprising or consisting of SEQ ID NO:98, a V_{L} CDR3 comprising or consisting of SEQ ID NO:99, a V_{H} CDR1 comprising or consisting of SEQ ID NO:101, a V_{H} CDR2 comprising or consisting of SEQ ID NO:102, and a V_{H} CDR3 comprising or consisting of SEQ ID NO:103;
r. a V_{L} CDR1 comprising or consisting of SEQ ID NO:105, a V_{L} CDR2 comprising or consisting of SEQ ID NO:106, a V_{L} CDR3 comprising or consisting of SEQ ID NO:107, a V_{H} CDR1 comprising or consisting of SEQ ID NO:109, a V_{H} CDR2 comprising or consisting of SEQ ID NO:110, and a V_{H} CDR3 comprising or consisting of SEQ ID NO:111;
s. a V_{L} CDR1 comprising or consisting of SEQ ID NO:113, a V_{L} CDR2 comprising or consisting of SEQ ID NO:114, a V_{L} CDR3 comprising or consisting of SEQ ID NO:115, a V_{H} CDR1 comprising or consisting of SEQ ID NO: 117, a V_{H} CDR2 comprising or consisting of SEQ ID NO:118, and a V_{H} CDR3 comprising or consisting of SEQ ID NO:119;
t. a V_{L} CDR1 comprising or consisting of SEQ ID NO:121, a V_{L} CDR2 comprising or consisting of SEQ ID NO:122, a V_{L} CDR3 comprising or consisting of SEQ ID NO:123, a V_{H} CDR1 comprising or consisting of SEQ ID NO:125, a V_{H} CDR2 comprising or consisting of SEQ ID NO:126, and a V_{H} CDR3 comprising or consisting of SEQ ID NO:127;
u. a V_{L} CDR1 comprising or consisting of SEQ ID NO:129, a V_{L} CDR2 comprising or consisting of SEQ ID NO:130, a V_{L} CDR3 comprising or consisting of SEQ ID NO:131, a V_{H} CDR1 comprising or consisting of SEQ ID NO:133, a V_{H} CDR2 comprising or consisting of SEQ ID NO:134, and a V_{H} CDR3 comprising or consisting of SEQ ID NO:135;
v. a V_{L} CDR1 comprising or consisting of SEQ ID NO:137, a V_{L} CDR2 comprising or consisting of SEQ ID NO:138, a V_{L} CDR3 comprising or consisting of SEQ ID NO:139, a V_{H} CDR1 comprising or consisting of SEQ ID NO:141, a V_{H} CDR2 comprising or consisting of SEQ ID NO:142, and a V_{H} CDR3 comprising or consisting of SEQ ID NO:143;
w. a V_{L} CDR1 comprising or consisting of SEQ ID NO:145, a V_{L} CDR2 comprising or consisting of SEQ ID NO: 146, a V_{L} CDR3 comprising or consisting of SEQ ID NO:147, a V_{H} CDR1 comprising or consisting of SEQ ID NO:149, a V_{H} CDR2 comprising or consisting of SEQ ID NO:150, and a V_{H} CDR3 comprising or consisting of SEQ ID NO:151;
x. a V_{L} CDR1 comprising or consisting of SEQ ID NO:153, a V_{L} CDR2 comprising or consisting of SEQ ID NO:154, a V_{L} CDR3 comprising or consisting of SEQ ID NO:155, a V_{H} CDR1 comprising or consisting of SEQ ID NO:157, a V_{H} CDR2 comprising or consisting of SEQ ID NO:158, and a V_{H} CDR3 comprising or consisting of SEQ ID NO:159;
y. a V_{L} CDR1 comprising or consisting of SEQ ID NO:161, a V_{L} CDR2 comprising or consisting of SEQ ID NO:162, a V_{L} CDR3 comprising or consisting of SEQ ID NO:163, a V_{H} CDR1 comprising or consisting of SEQ ID NO:165, a V_{H} CDR2 comprising or consisting of SEQ ID NO:166, and a V_{H} CDR3 comprising or consisting of SEQ ID NO:167;
z. a V_{L} CDR1 comprising or consisting of SEQ ID NO:169, a V_{L} CDR2 comprising or consisting of SEQ ID NO:170, a V_{L} CDR3 comprising or consisting of SEQ ID NO:171, a V_{H} CDR1 comprising or consisting of SEQ ID NO:173, a V_{H} CDR2 comprising or consisting of SEQ ID NO:174, and a V_{H} CDR3 comprising or consisting of SEQ ID NO:175;
aa. a V_{L} CDR1 comprising or consisting of SEQ ID NO:185, a V_{L} CDR2 comprising or consisting of SEQ ID NO:186, a V_{L} CDR3 comprising or consisting of SEQ ID NO:187, a V_{H} CDR1 comprising or consisting of SEQ ID NO:189, a V_{H} CDR2 comprising or consisting of SEQ ID NO:190, and a V_{H} CDR3 comprising or consisting of SEQ ID NO:19;
bb. a V_{L} CDR1 comprising or consisting of SEQ ID NO: 193, a V_{L} CDR2 comprising or consisting of SEQ ID NO:194, a V_{L} CDR3 comprising or consisting of SEQ ID NO:195, a V_{H} CDR1 comprising or consisting of SEQ ID NO:197, a V_{H} CDR2 comprising or consisting of SEQ ID NO:198, and a V_{H} CDR3 comprising or consisting of SEQ ID NO:199;
cc. a V_{L} CDR1 comprising or consisting of SEQ ID NO:201, a V_{L} CDR2 comprising or consisting of SEQ ID NO:202, a V_{L} CDR3 comprising or consisting of SEQ ID NO:203, a V_{H} CDR1 comprising or consisting of SEQ ID NO:205, a V_{H} CDR2 comprising or consisting of SEQ ID NO:206, and a V_{H} CDR3 comprising or consisting of SEQ ID NO:207;
dd. a V_{L} CDR1 comprising or consisting of SEQ ID NO:209, a V_{L} CDR2 comprising or consisting of SEQ ID NO:210, a V_{L} CDR3 comprising or consisting of SEQ ID NO:211, a V_{H} CDR1 comprising or consisting of SEQ ID NO:213, a V_{H} CDR2 comprising or consisting of SEQ ID NO:214, and a V_{H} CDR3 comprising or consisting of SEQ ID NO:215;
ee. a V_{L} CDR1 comprising or consisting of SEQ ID NO:217, a V_{L} CDR2 comprising or consisting of SEQ ID NO:218, a V_{L} CDR3 comprising or consisting of SEQ ID NO:219, a V_{H} CDR1 comprising or consisting of SEQ ID NO:221, a V_{H} CDR2 comprising or consisting of SEQ ID NO:222, and a V_{H} CDR3 comprising or consisting of SEQ ID NO:223;
ff. a V_{L} CDR1 comprising or consisting of SEQ ID NO:241, a V_{L} CDR2 comprising or consisting of SEQ ID NO:242, a V_{L} CDR3 comprising or consisting of SEQ ID NO:243, a V_{H} CDR1 comprising or consisting of SEQ ID NO:245, a V_{H} CDR2 comprising or consisting of SEQ ID NO:246, and a V_{H} CDR3 comprising or consisting of SEQ ID NO:247;
gg. a V_{L} CDR1 comprising or consisting of SEQ ID NO:249, a V_{L} CDR2 comprising or consisting of SEQ ID NO:250, a V_{L} CDR3 comprising or consisting of SEQ ID NO:251, a V_{H} CDR1 comprising or consisting of SEQ ID NO:253, a V_{H} CDR2 comprising or consisting of SEQ ID NO:254, and a V_{H} CDR3 comprising or consisting of SEQ ID NO:255;
hh. a V_{L} CDR1 comprising or consisting of SEQ ID NO:257, a V_{L} CDR2 comprising or consisting of SEQ ID NO:258, a V_{L} CDR3 comprising or consisting of SEQ ID NO:259, a V_{H} CDR1 comprising or consisting of SEQ ID NO:261, a V_{H} CDR2 comprising or consisting of SEQ ID NO:262, and a V_{H} CDR3 comprising or consisting of SEQ ID NO:263;
ii. a V_{L} CDR1 comprising or consisting of SEQ ID NO:273, a V_{L} CDR2 comprising or consisting of SEQ ID NO:274, a V_{L} CDR3 comprising or consisting of SEQ ID NO:275, a V_{H} CDR1 comprising or consisting of SEQ ID NO:277, a V_{H} CDR2 comprising or consisting of SEQ ID NO:278, and a V_{H} CDR3 comprising or consisting of SEQ ID NO:279; or,
jj. a V_{L} CDR1 comprising or consisting of SEQ ID NO:281, a V_{L} CDR2 comprising or consisting of SEQ ID NO:282, a V_{L} CDR3 comprising or consisting of SEQ ID NO:283, a V_{H} CDR1 comprising or consisting of SEQ ID NO:285, a V_{H} CDR2 comprising or consisting of SEQ ID NO:286, and a V_{H} CDR3 comprising or consisting of SEQ ID NO:287.

6. The isolated antibody of claim 1, wherein the isolated antibody comprises a light chain selected from the group consisting of:
a. a light chain comprising an amino acid sequence at least 80% identical, at least 85% identical, at least 90% identical, at least 95% identical, at last 97% identical, or at least 99% identical to SEQ ID NO:180, wherein the light chain comprises V_{L} CDR1 comprising or consisting of SEQ ID NO:177, V_{L} CDR2 comprising or consisting of SEQ ID NO:178, and V_{L} CDR3 comprising or consisting of SEQ ID NO:179;
b. a light chain comprising an amino acid sequence at least 80% identical, at least 85% identical, at least 90% identical, at least 95% identical, at last 97% identical, or at least 99% identical to SEQ ID NO:228, wherein the light chain comprises V_{L} CDR1 comprising or consisting of SEQ ID NO:225, V_{L} CDR2 comprising or consisting of SEQ ID NO:226, and V_{L} CDR3 comprising or consisting of SEQ ID NO:227;
c. a light chain comprising an amino acid sequence at least 80% identical, at least 85% identical, at least 90% identical, at least 95% identical, at last 97% identical, or at least 99% identical to SEQ ID NO:236, wherein the light chain comprises V_{L} CDR1 comprising or consisting of SEQ ID NO:233, V_{L} CDR2 comprising or consisting of SEQ ID NO:234, and V_{L} CDR3 comprising or consisting of SEQ ID NO:235;
d. a light chain comprising an amino acid sequence at least 80% identical, at least 85% identical, at least 90% identical, at least 95% identical, at last 97% identical, or at least 99% identical to SEQ ID NO:268, wherein the light chain comprises V_{L} CDR1 comprising or consisting of SEQ ID NO:265, V_{L} CDR2 comprising or consisting of SEQ ID NO:266, and V_{L} CDR3 comprising or consisting of SEQ ID NO:267;
e. a light chain comprising an amino acid sequence at least 80% identical, at least 85% identical, at least 90% identical, at least 95% identical, at least 97% identical, or at least 99% identical to SEQ ID NO:4, wherein the light chain comprises V_{L} CDR1 comprising or consisting of SEQ ID NO:1, V_{L} CDR2 comprising or consisting of SEQ ID NO:2, and V_{L} CDR3 comprising or consisting of SEQ ID NO:3;
f. a light chain comprising an amino acid sequence at least 80% identical, at least 85% identical, at least 90% identical, at least 95% identical, at least 97% identical, or at least 99% identical to SEQ ID NO:12, wherein the light chain comprises V_{L} CDR1 comprising or consisting of SEQ ID NO:9, V_{L} CDR2 comprising or consisting of SEQ ID NO:10, and V_{L} CDR3 comprising or consisting of SEQ ID NO:11;
g. a light chain comprising an amino acid sequence at least 80% identical, at least 85% identical, at least 90% identical, at least 95% identical, at last 97% identical, or at least 99% identical to SEQ ID NO:20, wherein the light chain comprises V_{L} CDR1 comprising or consisting of SEQ ID NO:17, V_{L} CDR2 comprising or consisting of SEQ ID NO:18, and V_{L} CDR3 comprising or consisting of SEQ ID NO:19;
h. a light chain comprising an amino acid sequence at least 80% identical, at least 85% identical, at least 90% identical, at least 95% identical, at last 97% identical, or at least 99% identical to SEQ ID NO:28, wherein the light chain comprises V_{L} CDR1 comprising or consisting of SEQ ID NO:25, V_{L} CDR2 comprising or consisting of SEQ ID NO:26, and V_{L} CDR3 comprising or consisting of SEQ ID NO:27;
i. a light chain comprising an amino acid sequence at least 80% identical, at least 85% identical, at least 90% identical, at least 95% identical, at last 97% identical, or at least 99% identical to SEQ ID NO:36, wherein the light chain comprises V_{L} CDR1 comprising or consisting of SEQ ID NO:33, V_{L} CDR2 comprising or consisting of SEQ ID NO:34, and V_{L} CDR3 comprising or consisting of SEQ ID NO:35;
j. a light chain comprising an amino acid sequence at least 80% identical, at least 85% identical, at least 90% identical, at least 95% identical, at last 97% identical, or at least 99% identical to SEQ ID NO:44, wherein the light chain comprises V_{L} CDR1 comprising or consisting of SEQ ID NO:41, V_{L} CDR2 comprising or consisting of SEQ ID NO:42, and V_{L} CDR3 comprising or consisting of SEQ ID NO:43;
k. a light chain comprising an amino acid sequence at least 80% identical, at least 85% identical, at least 90% identical, at least 95% identical, at last 97% identical, or at least 99% identical to SEQ ID NO:52, wherein the light chain comprises V_{L} CDR1 comprising or consisting of SEQ ID NO:49, V_{L} CDR2 comprising or consisting of SEQ ID NO:50, and V_{L} CDR3 comprising or consisting of SEQ ID NO:51;
l. a light chain comprising an amino acid sequence at least 80% identical, at least 85% identical, at least 90% identical, at least 95% identical, at last 97% identical, or at least 99% identical to SEQ ID NO:60, wherein the light chain comprises V_{L} CDR1 comprising or consisting of SEQ ID NO:57, V_{L} CDR2 comprising or consisting of SEQ ID NO:58, and V_{L} CDR3 comprising or consisting of SEQ ID NO:59;
m. a light chain comprising an amino acid sequence at least 80% identical, at least 85% identical, at least 90% identical, at least 95% identical, at last 97% identical, or at least 99% identical to SEQ ID NO:68, wherein the light chain comprises V_{L} CDR1 comprising or consisting of SEQ ID NO:65, V_{L} CDR2 comprising or consisting of SEQ ID NO:66, and V_{L} CDR3 comprising or consisting of SEQ ID NO:67;
n. a light chain comprising an amino acid sequence at least 80% identical, at least 85% identical, at least 90% identical, at least 95% identical, at last 97% identical, or at least 99% identical to SEQ ID NO:76, wherein the light chain comprises V_{L} CDR1 comprising or consisting of SEQ ID NO:73, V_{L} CDR2 comprising or consisting of SEQ ID NO:74, and V_{L} CDR3 comprising or consisting of SEQ ID NO:75;
o. a light chain comprising an amino acid sequence at least 80% identical, at least 85% identical, at least 90% identical, at least 95% identical, at last 97% identical, or at least 99% identical to SEQ ID NO:84, wherein the light chain comprises V_{L} CDR1 comprising or consisting of SEQ ID NO:81, V_{L} CDR2 comprising or consisting of SEQ ID NO:82, and V_{L} CDR3 comprising or consisting of SEQ ID NO:83;
p. a light chain comprising an amino acid sequence at least 80% identical, at least 85% identical, at least 90% identical, at least 95% identical, at last 97% identical, or at least 99% identical to SEQ ID NO:92, wherein the light chain comprises V_{L} CDR1 comprising or consisting of SEQ ID NO:89, V_{L} CDR2 comprising or consisting of SEQ ID NO:90, and V_{L} CDR3 comprising or consisting of SEQ ID NO:91;
q. a light chain comprising an amino acid sequence at least 80% identical, at least 85% identical, at least 90% identical, at least 95% identical, at last 97% identical, or at least 99% identical to SEQ ID NO:100, wherein the light chain comprises V_{L} CDR1 comprising or consisting of SEQ ID NO:97, V_{L} CDR2 comprising or consisting of SEQ ID NO:98, and V_{L} CDR3 comprising or consisting of SEQ ID NO:99;
r. a light chain comprising an amino acid sequence at least 80% identical, at least 85% identical, at least 90% identical, at least 95% identical, at last 97% identical, or at least 99% identical to SEQ ID NO:108, wherein the light chain comprises V_{L} CDR1 comprising or consisting of SEQ ID NO: 105, V_{L} CDR2 comprising or consisting of SEQ ID NO:106, and V_{L} CDR3 comprising or consisting of SEQ ID NO:107;
s. a light chain comprising an amino acid sequence at least 80% identical, at least 85% identical, at least 90% identical, at least 95% identical, at last 97% identical, or at least 99% identical to SEQ ID NO:116, wherein the light chain comprises V_{L} CDR1 comprising or consisting of SEQ ID NO:113, V_{L} CDR2 comprising or consisting of SEQ ID NO:114, and V_{L} CDR3 comprising or consisting of SEQ ID NO:115;
t. a light chain comprising an amino acid sequence at least 80% identical, at least 85% identical, at least 90% identical, at least 95% identical, at last 97% identical, or at least 99% identical to SEQ ID NO:124, wherein the light chain comprises V_{L} CDR1 comprising or consisting of SEQ ID NO:121, V_{L} CDR2 comprising or consisting of SEQ ID NO:122, and V_{L} CDR3 comprising or consisting of SEQ ID NO:123;
u. a light chain comprising an amino acid sequence at least 80% identical, at least 85% identical, at least 90% identical, at least 95% identical, at last 97% identical, or at least 99% identical to SEQ ID NO:132, wherein the light chain comprises V_{L} CDR1 comprising or consisting of SEQ ID NO:129, V_{L} CDR2 comprising or consisting of SEQ ID NO:130, and V_{L} CDR3 comprising or consisting of SEQ ID NO:131;
v. a light chain comprising an amino acid sequence at least 80% identical, at least 85% identical, at least 90% identical, at least 95% identical, at last 97% identical, or at least 99% identical to SEQ ID NO:140, wherein the light chain comprises V_{L} CDR1 comprising or consisting of SEQ ID NO:137, V_{L} CDR2 comprising or consisting of SEQ ID NO:138, and V_{L} CDR3 comprising or consisting of SEQ ID NO:139;
w. a light chain comprising an amino acid sequence at least 80% identical, at least 85% identical, at least 90% identical, at least 95% identical, at last 97% identical, or at least 99% identical to SEQ ID NO:148, wherein the light chain comprises V_{L} CDR1 comprising or consisting of SEQ ID NO: 145, V_{L} CDR2 comprising or consisting of SEQ ID NO:146, and V_{L} CDR3 comprising or consisting of SEQ ID NO:147;
x. a light chain comprising an amino acid sequence at least 80% identical, at least 85% identical, at least 90% identical, at least 95% identical, at last 97% identical, or at least 99% identical to SEQ ID NO:156, wherein the light chain comprises V_{L} CDR1 comprising or consisting of SEQ ID NO: 153, V_{L} CDR2 comprising or consisting of SEQ ID NO:154, and V_{L} CDR3 comprising or consisting of SEQ ID NO:155;
y. a light chain comprising an amino acid sequence at least 80% identical, at least 85% identical, at least 90% identical, at least 95% identical, at last 97% identical, or at least 99% identical to SEQ ID NO:164, wherein the light chain comprises V_{L} CDR1 comprising or consisting of SEQ ID NO:161, V_{L} CDR2 comprising or consisting of SEQ ID NO:162, and V_{L} CDR3 comprising or consisting of SEQ ID NO:163;
z. a light chain comprising an amino acid sequence at least 80% identical, at least 85% identical, at least 90% identical, at least 95% identical, at last 97% identical, or at least 99% identical to SEQ ID NO:172, wherein the light chain comprises V_{L} CDR1 comprising or consisting of SEQ ID NO:169, V_{L} CDR2 comprising or consisting of SEQ ID NO:170, and V_{L} CDR3 comprising or consisting of SEQ ID NO:171;
aa. a light chain comprising an amino acid sequence at least 80% identical, at least 85% identical, at least 90% identical, at least 95% identical, at last 97% identical, or at least 99% identical to SEQ ID NO:188, wherein the light chain comprises V_{L} CDR1 comprising or consisting of SEQ ID NO:185, V_{L} CDR2 comprising or consisting of SEQ ID NO:186, and V_{L} CDR3 comprising or consisting of SEQ ID NO:187;
bb. a light chain comprising an amino acid sequence at least 80% identical, at least 85% identical, at least 90% identical, at least 95% identical, at last 97% identical, or at least 99% identical to SEQ ID NO:196, wherein the light chain comprises V_{L} CDR1 comprising or consisting of SEQ ID NO:193, V_{L} CDR2 comprising or consisting of SEQ ID NO:194, and V_{L} CDR3 comprising or consisting of SEQ ID NO:195;
cc. a light chain comprising an amino acid sequence at least 80% identical, at least 85% identical, at least 90% identical, at least 95% identical, at last 97% identical, or at least 99% identical to SEQ ID NO:204, wherein the light chain comprises V_{L} CDR1 comprising or consisting of SEQ ID NO:201, V_{L} CDR2 comprising or consisting of SEQ ID NO:202, and V_{L} CDR3 comprising or consisting of SEQ ID NO:203;
dd. a light chain comprising an amino acid sequence at least 80% identical, at least 85% identical, at least 90% identical, at least 95% identical, at last 97% identical, or at least 99% identical to SEQ ID NO:212, wherein the light chain comprises V_{L} CDR1 comprising or consisting of SEQ ID NO:209, V_{L} CDR2 comprising or consisting of SEQ ID NO:210, and V_{L} CDR3 comprising or consisting of SEQ ID NO:21;
ee. a light chain comprising an amino acid sequence at least 80% identical, at least 85% identical, at least 90% identical, at least 95% identical, at last 97% identical, or at least 99% identical to SEQ ID NO:220, wherein the light chain comprises V_{L} CDR1 comprising or consisting of SEQ ID NO:217, V_{L} CDR2 comprising or consisting of SEQ ID NO:218, and V_{L} CDR3 comprising or consisting of SEQ ID NO:219;
ff. a light chain comprising an amino acid sequence at least 80% identical, at least 85% identical, at least 90% identical, at least 95% identical, at last 97% identical, or at least 99% identical to SEQ ID NO:244, wherein the light chain comprises V_{L} CDR1 comprising or consisting of SEQ ID NO:241, V_{L} CDR2 comprising or consisting of SEQ ID NO:242, and V_{L} CDR3 comprising or consisting of SEQ ID NO:243;
gg. a light chain comprising an amino acid sequence at least 80% identical, at least 85% identical, at least 90% identical, at least 95% identical, at last 97% identical, or at least 99% identical to SEQ ID NO:252, wherein the light chain comprises V_{L} CDR1 comprising or consisting of SEQ ID NO:249, V_{L} CDR2 comprising or consisting of SEQ ID NO:250, and V_{L} CDR3 comprising or consisting of SEQ ID NO:251;
hh. a light chain comprising an amino acid sequence at least 80% identical, at least 85% identical, at least 90% identical, at least 95% identical, at last 97% identical, or at least 99% identical to SEQ ID NO:260, wherein the light chain comprises V_{L} CDR1 comprising or consisting of SEQ ID NO:257, V_{L} CDR2 comprising or consisting of SEQ ID NO:258, and V_{L} CDR3 comprising or consisting of SEQ ID NO:259;
ii. a light chain comprising an amino acid sequence at least 80% identical, at least 85% identical, at least 90% identical, at least 95% identical, at last 97% identical, or at least 99% identical to SEQ ID NO:276, wherein the light chain comprises V_{L} CDR1 comprising or consisting of SEQ ID NO:273, V_{L} CDR2 comprising or consisting of SEQ ID NO:274, and V_{L} CDR3 comprising or consisting of SEQ ID NO:275;
jj. a light chain comprising an amino acid sequence at least 80% identical, at least 85% identical, at least 90% identical, at least 95% identical, at last 97% identical, or at least 99% identical to SEQ ID NO:284, wherein the light chain comprises V_{L} CDR1 comprising or consisting of SEQ ID NO:281, V_{L} CDR2 comprising or consisting of SEQ ID NO:282, and V_{L} CDR3 comprising or consisting of SEQ ID NO:283.

7. The isolated antibody of claim 1, wherein the isolated antibody comprises a heavy chain selected from the group consisting of:
a. a heavy chain comprising an amino acid sequence at least 80% identical, at least 85% identical, at least 90% identical, at least 95% identical, at last 97% identical, or at least 99% identical to SEQ ID NO:184, wherein the heavy chain comprises V_{H} CDR1 comprising or consisting of SEQ ID NO:181, V_{H} CDR2 comprising or consisting of SEQ ID NO:182, and V_{H} CDR3 comprising or consisting of SEQ ID NO:183;
b. a heavy chain comprising an amino acid sequence at least 80% identical, at least 85% identical, at least 90% identical, at least 95% identical, at last 97% identical, or at least 99% identical to SEQ ID NO:232, wherein the heavy chain comprises V_{L} CDR1 comprising or consisting of SEQ ID NO:229, V_{H} CDR2 comprising or consisting of SEQ ID NO:230, and V_{L} CDR3 comprising or consisting of SEQ ID NO:231;
c. a heavy chain comprising an amino acid sequence at least 80% identical, at least 85% identical, at least 90% identical, at least 95% identical, at last 97% identical, or at least 99% identical to SEQ ID NO:240, wherein the heavy chain comprises V_{H} CDR1 comprising or consisting of SEQ ID N0237, V_{H} CDR2 comprising or consisting of SEQ ID NO:238, and V_{H} CDR3 comprising or consisting of SEQ ID NO:239;
d. a heavy chain comprising an amino acid sequence at least 80% identical, at least 85% identical, at least 90% identical, at least 95% identical, at last 97% identical, or at least 99% identical to SEQ ID NO:272, wherein the heavy chain comprises V_{H} CDR1 comprising or consisting of SEQ ID NO:269, V_{H} CDR2 comprising or consisting of SEQ ID NO:270, and V_{H} CDR3 comprising or consisting of SEQ ID NO:271;
e. heavy chain comprising an amino acid sequence at least 80% identical, at least 85% identical, at least 90% identical, at least 95% identical, at last 97% identical, or at least 99% identical to SEQ ID NO:8, wherein the heavy chain comprises V_{H} CDR1 comprising or consisting of SEQ ID NO:5, V_{H} CDR2 comprising or consisting of SEQ ID NO:6, and V_{H} CDR3 comprising or consisting of SEQ ID NO:7;
f. a heavy chain comprising an amino acid sequence at least 80% identical, at least 85% identical, at least 90% identical, at least 95% identical, at last 97% identical, or at least 99% identical to SEQ ID NO:16, wherein the heavy chain comprises V_{H} CDR i comprising or consisting of SEQ ID NO:13, V_{H} CDR2 comprising or consisting of SEQ ID NO:14, and V_{H} CDR3 comprising or consisting of SEQ ID NO: 15;
g. a heavy chain comprising an amino acid sequence at least 80% identical, at least 85% identical, at least 90% 213, at least 95% identical, at last 97% identical, or at least 99% identical to SEQ ID NO:24, wherein the heavy chain comprises V_{H} CDR1 comprising or consisting of SEQ ID NO:21, V_{H} CDR2 comprising or consisting of SEQ ID NO:22, and V_{H} CDR3 comprising or consisting of SEQ ID NO:23;
h. a heavy chain comprising an amino acid sequence at least 80% identical, at least 85% identical, at least 90% identical, at least 95% identical, at last 97% identical, or at least 99% identical to SEQ ID NO:32, wherein the heavy chain comprises V_{H} CDR1 comprising or consisting of SEQ ID NO:29, V_{H} CDR2 comprising or consisting of SEQ ID NO:30, and V_{H} CDR3 comprising or consisting of SEQ ID NO:31;
i. a heavy chain comprising an amino acid sequence at least 80% identical, at least 85% identical, at least 90% identical, at least 95% identical, at last 97% identical, or at least 99% identical to SEQ ID NO:40, wherein the heavy chain comprises V_{H} CDR1 comprising or consisting of SEQ ID NO:37, V_{H} CDR2 comprising or consisting of SEQ ID NO:38, and V_{H} CDR3 comprising or consisting of SEQ ID NO:39;
j. a heavy chain comprising an amino acid sequence at least 80% identical, at least 85% identical, at least 90% identical, at least 95% identical, at last 97% identical, or at least 99% identical to SEQ ID NO:48, wherein the heavy chain comprises V_{H} CDR1 comprising or consisting of SEQ ID NO:45, V_{H} CDR2 comprising or consisting of SEQ ID NO:46, and V_{H} CDR3 comprising or consisting of SEQ ID NO:47;
k. a heavy chain comprising an amino acid sequence at least 80% identical, at least 85% identical, at least 90% identical, at least 95% identical, at last 97% identical, or at least 99% identical to SEQ ID NO:56, wherein the heavy chain comprises V_{H} CDR1 comprising or consisting of SEQ ID NO:53, V_{H} CDR2 comprising or consisting of SEQ ID NO:54, and V_{H} CDR3 comprising or consisting of SEQ ID NO:55;
l. a heavy chain comprising an amino acid sequence at least 80% identical, at least 85% identical, at least 90% identical, at least 95% identical, at last 97% identical, or at least 99% identical to SEQ ID NO:64, wherein the heavy chain comprises V_{H} CDR1 comprising or consisting of SEQ ID NO:61, V_{H} CDR2 comprising or consisting of SEQ ID NO:62, and V_{H} CDR3 comprising or consisting of SEQ ID NO:63;
m. a heavy chain comprising an amino acid sequence at least 80% identical, at least 85% identical, at least 90% identical, at least 95% identical, at last 97% identical, or at least 99% identical to SEQ ID NO:72, wherein the heavy chain comprises V_{H} CDR1 comprising or consisting of SEQ ID NO:69, V_{H} CDR2 comprising or consisting of SEQ ID NO:70, and V_{H} CDR3 comprising or consisting of SEQ ID NO:71;
n. a heavy chain comprising an amino acid sequence at least 80% identical, at least 85% identical, at least 90% identical, at least 95% identical, at last 97% identical, or at least 99% identical to SEQ ID NO:80, wherein the heavy chain comprises V_{H} CDR1 comprising or consisting of SEQ ID NO:77, V_{H} CDR2 comprising or consisting of SEQ ID NO:78, and V_{H} CDR3 comprising or consisting of SEQ ID NO:79;
o. a heavy chain comprising an amino acid sequence at least 80% identical, at least 85% identical, at least 90% identical, at least 95% identical, at last 97% identical, or at least 99% identical to SEQ ID NO:88, wherein the heavy chain comprises V_{H} CDR1 comprising or consisting of SEQ ID NO:85, V_{H} CDR2 comprising or consisting of SEQ ID NO:86, and V_{H} CDR3 comprising or consisting of SEQ ID NO:87;
p. a heavy chain comprising an amino acid sequence at least 80% identical, at least 85% identical, at least 90% identical, at least 95% identical, at last 97% identical, or at least 99% identical to SEQ ID NO:96, wherein the heavy chain comprises V_{H} CDR1 comprising or consisting of SEQ ID NO:93, V_{H} CDR2 comprising or consisting of SEQ ID NO:94, and V_{H} CDR3 comprising or consisting of SEQ ID NO:95;
q. a heavy chain comprising an amino acid sequence at least 80% identical, at least 85% identical, at least 90% identical, at least 95% identical, at last 97% identical, or at least 99% identical to SEQ ID NO:104, wherein the heavy chain comprises V_{H} CDR1 comprising or consisting of SEQ ID NO:101, V_{H} CDR2 comprising or consisting of SEQ ID NO:102, and V_{H} CDR3 comprising or consisting of SEQ ID NO:103;
r. a heavy chain comprising an amino acid sequence at least 80% identical, at least 85% identical, at least 90% identical, at least 95% identical, at last 97% identical, or at least 99% identical to SEQ ID NO:112, wherein the heavy chain comprises V_{H} CDR1 comprising or consisting of SEQ ID NO:109, V_{H} CDR2 comprising or consisting of SEQ ID NO:110, and V_{H} CDR3 comprising or consisting of SEQ ID NO:111;
s. a heavy chain comprising an amino acid sequence at least 80% identical, at least 85% identical, at least 90% identical, at least 95% identical, at last 97% identical, or at least 99% identical to SEQ ID NO:120, wherein the heavy chain comprises V_{H} CDR1 comprising or consisting of SEQ ID NO:117, V_{H} CDR2 comprising or consisting of SEQ ID NO: 118, and V_{H} CDR3 comprising or consisting of SEQ ID NO:119;
t. a heavy chain comprising an amino acid sequence at least 80% identical, at least 85% identical, at least 90% identical, at least 95% identical, at last 97% identical, or at least 99% identical to SEQ ID NO:128, wherein the heavy chain comprises V_{H} CDR1 comprising or consisting of SEQ ID NO:125, V_{H} CDR2 comprising or consisting of SEQ ID NO:126, and V_{L} CDR3 comprising or consisting of SEQ ID NO:127;
u. a heavy chain comprising an amino acid sequence at least 80% identical, at least 85% identical, at least 90% identical, at least 95% identical, at last 97% identical, or at least 99% identical to SEQ ID NO:136, wherein the heavy chain comprises V_{H} CDR1 comprising or consisting of SEQ ID NO:133, V_{H} CDR2 comprising or consisting of SEQ ID NO:134, and V_{H} CDR3 comprising or consisting of SEQ ID NO:135;
v. a heavy chain comprising an amino acid sequence at least 80% identical, at least 85% identical, at least 90% identical, at least 95% identical, at last 97% identical, or at least 99% identical to SEQ ID NO:144, wherein the heavy chain comprises V_{H} CDR1 comprising or consisting of SEQ ID NO:141, V_{H} CDR2 comprising or consisting of SEQ ID NO:142, and V_{H} CDR3 comprising or consisting of SEQ ID NO 143;
w. a heavy chain comprising an amino acid sequence at least 80% identical, at least 85% identical, at least 90% identical, at least 95% identical, at last 97% identical, or at least 99% identical to SEQ ID NO:152, wherein the heavy chain comprises V_{H} CDR1 comprising or consisting of SEQ ID NO:149, V_{H} CDR2 comprising or consisting of SEQ ID NO:150, and V_{H} CDR3 comprising or consisting of SEQ ID NO:151;
x. a heavy chain comprising an amino acid sequence at least 80% identical, at least 85% identical, at least 90% identical, at least 95% identical, at last 97% identical, or at least 99% identical to SEQ ID NO:160, wherein the heavy chain comprises V_{H} CDR1 comprising or consisting of SEQ ID NO:157, V_{H} CDR2 comprising or consisting of SEQ ID NO: 158, and V_{H} CDR3 comprising or consisting of SEQ ID NO:159;
y. a heavy chain comprising an amino acid sequence at least 80% identical, at least 85% identical, at least 90% identical, at least 95% identical, at last 97% identical, or at least 99% identical to SEQ ID NO:168, wherein the heavy chain comprises V_{H} CDR1 comprising or consisting of SEQ ID NO:165, V_{H} CDR2 comprising or consisting of SEQ ID NO: 166, and V_{H} CDR3 comprising or consisting of SEQ ID NO:167;
z. a heavy chain comprising an amino acid sequence at least 80% identical, at least 85% identical, at least 90% identical, at least 95% identical, at last 97% identical, or at least 99% identical to SEQ ID NO:176, wherein the heavy chain comprises V_{H} CDR1 comprising or consisting of SEQ ID NO:173, V_{H} CDR2 comprising or consisting of SEQ ID NO:174, and V_{H} CDR3 comprising or consisting of SEQ ID NO:175;
aa. heavy chain comprising an amino acid sequence at least 80% identical, at least 85% identical, at least 90% identical, at least 95% identical, at last 97% identical, or at least 99% identical to SEQ ID NO:192, wherein the heavy chain comprises V_{H} CDR1 comprising or consisting of SEQ ID NO: 189, V_{H} CDR2 comprising or consisting of SEQ ID NO:190, and V_{H} CDR3 comprising or consisting of SEQ ID NO:191;
bb. a heavy chain comprising an amino acid sequence at least 80% identical, at least 85% identical, at least 90% identical, at least 95% identical, at last 97% identical, or at least 99% identical to SEQ ID NO:200, wherein the heavy chain comprises V_{H} CDR1 comprising or consisting of SEQ ID NO:197, V_{H} CDR2 comprising or consisting of SEQ ID NO:198, and V_{H} CDR3 comprising or consisting of SEQ ID NO:199;
cc. a heavy chain comprising an amino acid sequence at least 80% identical, at least 85% identical, at least 90% identical, at least 95% identical, at last 97% identical, or at least 99% identical to SEQ ID NO:208, wherein the heavy chain comprises V_{H} CDR1 comprising or consisting of SEQ ID NO:205, V_{H} CDR2 comprising or consisting of SEQ ID NO:206, and V_{H} CDR3 comprising or consisting of SEQ ID NO:207;
dd. a heavy chain comprising an amino acid sequence at least 80% identical, at least 85% identical, at least 90% identical, at least 95% identical, at last 97% identical, or at least 99% identical to SEQ ID NO:216, wherein the heavy chain comprises V_{H} CDR1 comprising or consisting of SEQ ID NO:213, V_{H} CDR2 comprising or consisting of SEQ ID NO:214, and V_{H} CDR3 comprising or consisting of SEQ ID NO:215;
ee. a heavy chain comprising an amino acid sequence at least 80% identical, at least 85% identical, at least 90% identical, at least 95% identical, at last 97% identical, or at least 99% identical to SEQ ID NO:224, wherein the heavy chain comprises V_{H} CDR1 comprising or consisting of SEQ ID NO:221, V_{H} CDR2 comprising or consisting of SEQ ID NO:222, and V_{H} CDR3 comprising or consisting of SEQ ID NO:223;
ff. a heavy chain comprising an amino acid sequence at least 80% identical, at least 85% identical, at least 90% identical, at least 95% identical, at last 97% identical, or at least 99% identical to SEQ ID NO:248, wherein the heavy chain comprises V_{H} CDR1 comprising or consisting of SEQ ID NO:245, V_{H} CDR2 comprising or consisting of SEQ ID NO:246, and V_{H} CDR3 comprising or consisting of SEQ ID NO:247;
gg. a heavy chain comprising an amino acid sequence at least 80% identical, at least 85% identical, at least 90% identical, at least 95% identical, at last 97% identical, or at least 99% identical to SEQ ID NO:256, wherein the heavy chain comprises V_{H} CDR1 comprising or consisting of SEQ ID NO:253, V_{H} CDR2 comprising or consisting of SEQ ID NO:254, and V_{H} CDR3 comprising or consisting of SEQ ID NO:255;
hh. a heavy chain comprising an amino acid sequence at least 80% identical, at least 85% identical, at least 90% identical, at least 95% identical, at last 97% identical, or at least 99% identical to SEQ ID NO:264, wherein the heavy chain comprises V_{H} CDR1 comprising or consisting of SEQ ID NO:261, V_{H} CDR2 comprising or consisting of SEQ ID NO:262, and V_{H} CDR3 comprising or consisting of SEQ ID NO:263;
ii. a heavy chain comprising an amino acid sequence at least 80% identical, at least 85% identical, at least 90% identical, at least 95% identical, at last 97% identical, or at least 99% identical to SEQ ID NO:280, wherein the heavy chain comprises V_{H} CDR1 comprising or consisting of SEQ ID NO:277, V_{H} CDR2 comprising or consisting of SEQ ID NO:278, and V_{H} CDR3 comprising or consisting of SEQ ID NO:279; and,
jj. a heavy chain comprising an amino acid sequence at least 80% identical, at least 85% identical, at least 90% identical, at least 95% identical, at last 97% identical, or at least 99% identical to SEQ ID NO:288, wherein the heavy chain comprises V_{H} CDR1 comprising or consisting of SEQ ID NO:285, V_{H} CDR2 comprising or consisting of SEQ ID NO:286, and V_{H} CDR3 comprising or consisting of SEQ ID NO:287.

8. The isolated antibody of any one of claim 1, wherein the isolated antibody comprises a light chain selected from the group consisting of:
a. a light chain comprising an amino acid sequence at least 80% identical, at least 85% identical, at least 90% identical, at least 95% identical, at last 97% identical, or at least 99% identical to SEQ ID NO:334, wherein the light chain comprises V_{L} CDR1 comprising or consisting of SEQ ID NO: 177, V_{L} CDR2 comprising or consisting of SEQ ID NO:178, and V_{L} CDR3 comprising or consisting of SEQ ID NO:179;
b. a light chain comprising an amino acid sequence at least 80% identical, at least 85% identical, at least 90% identical, at least 95% identical, at last 97% identical, or at least 99% identical to SEQ ID NO:346, wherein the light chain comprises V_{L} CDR1 comprising or consisting of SEQ ID NO:225, V_{L} CDR2 comprising or consisting of SEQ ID NO:226, and V_{L} CDR3 comprising or consisting of SEQ ID NO:227;
c. a light chain comprising an amino acid sequence at least 80% identical, at least 85% identical, at least 90% identical, at least 95% identical, at last 97% identical, or at least 99% identical to SEQ ID NO:348, wherein the light chain comprises V_{L} CDR1 comprising or consisting of SEQ ID NO:233, V_{L} CDR2 comprising or consisting of SEQ ID NO:234, and V_{L} CDR3 comprising or consisting of SEQ ID NO:235;
d. a light chain comprising an amino acid sequence at least 80% identical, at least 85% identical, at least 90% identical, at least 95% identical, at last 97% identical, or at least 99% identical to SEQ ID NO:356, wherein the light chain comprises V_{L} CDR1 comprising or consisting of SEQ ID NO:265, V_{L} CDR2 comprising or consisting of SEQ ID NO:266, and V_{L} CDR3 comprising or consisting of SEQ ID NO:267;
e. a light chain comprising an amino acid sequence at least 80% identical, at least 85% identical, at least 90% identical, at least 95% identical, at last 97% identical, or at least 99% identical to SEQ ID NO:290, wherein the light chain comprises V_{L} CDR1 comprising or consisting of SEQ ID NO: 1, V_{L} CDR2 comprising or consisting of SEQ ID NO:2, and V_{L} CDR3 comprising or consisting of SEQ ID NO:3;
f. a light chain comprising an amino acid sequence at least 80% identical, at least 85% identical, at least 90% identical, at least 95% identical, at last 97% identical, or at least 99% identical to SEQ ID NO:292, wherein the light chain comprises V_{L} CDR1 comprising or consisting of SEQ ID NO:9, V_{L} CDR2 comprising or consisting of SEQ ID NO:10, and V_{L} CDR3 comprising or consisting of SEQ ID NO:11;
g. a light chain comprising an amino acid sequence at least 80% identical, at least 85% identical, at least 90% identical, at least 95% identical, at last 97% identical, or at least 99% identical to SEQ ID NO:294, wherein the light chain comprises V_{L} CDR1 comprising or consisting of SEQ ID NO:17, V_{L} CDR2 comprising or consisting of SEQ ID NO:18, and V_{L} CDR3 comprising or consisting of SEQ ID NO:19;
h. a light chain comprising an amino acid sequence at least 80% identical, at least 85% identical, at least 90% identical, at least 95% identical, at last 97% identical, or at least 99% identical to SEQ ID NO:296, wherein the light chain comprises V_{L} CDR1 comprising or consisting of SEQ ID NO:25, V_{L} CDR2 comprising or consisting of SEQ ID NO:26, and V_{L} CDR3 comprising or consisting of SEQ ID NO:27;
i. a light chain comprising an amino acid sequence at least 80% identical, at least 85% identical, at least 90% identical, at least 95% identical, at last 97% identical, or at least 99% identical to SEQ ID NO:298, wherein the light chain comprises V_{L} CDR1 comprising or consisting of SEQ ID NO:33, V_{L} CDR2 comprising or consisting of SEQ ID NO:34, and V_{L} CDR3 comprising or consisting of SEQ ID NO:35;
j. a light chain comprising an amino acid sequence at least 80% identical, at least 85% identical, at least 90% identical, at least 95% identical, at last 97% identical, or at least 99% identical to SEQ ID NO:300, wherein the light chain comprises V_{L} CDR1 comprising or consisting of SEQ ID NO:41, V_{L} CDR2 comprising or consisting of SEQ ID NO:42, and V_{L} CDR3 comprising or consisting of SEQ ID NO:43;
k. a light chain comprising an amino acid sequence at least 80% identical, at least 85% identical, at least 90% identical, at least 95% identical, at last 97% identical, or at least 99% identical to SEQ ID NO:302, wherein the light chain comprises V_{L} CDR1 comprising or consisting of SEQ ID NO:49, V_{L} CDR2 comprising or consisting of SEQ ID NO:50, and V_{L} CDR3 comprising or consisting of SEQ ID NO:51;
l. a light chain comprising an amino acid sequence at least 80% identical, at least 85% identical, at least 90% identical, at least 95% identical, at last 97% identical, or at least 99% identical to SEQ ID NO:304, wherein the light chain comprises V_{L} CDR1 comprising or consisting of SEQ ID NO:57, V_{L} CDR2 comprising or consisting of SEQ ID NO:58, and V_{L} CDR3 comprising or consisting of SEQ ID NO:59;
m. a light chain comprising an amino acid sequence at least 80% identical, at least 85% identical, at least 90% identical, at least 95% identical, at last 97% identical, or at least 99% identical to SEQ ID NO:306, wherein the light chain comprises V_{L} CDR1 comprising or consisting of SEQ ID NO:65, V_{L} CDR2 comprising or consisting of SEQ ID NO:66, and V_{L} CDR3 comprising or consisting of SEQ ID NO:67;
n. a light chain comprising an amino acid sequence at least 80% identical, at least 85% identical, at least 90% identical, at least 95% identical, at last 97% identical, or at least 99% identical to SEQ ID NO:308, wherein the light chain comprises V_{L} CDR1 comprising or consisting of SEQ ID NO:73, V_{L} CDR2 comprising or consisting of SEQ ID NO:74, and V_{L} CDR3 comprising or consisting of SEQ ID NO:75;
o. a light chain comprising an amino acid sequence at least 80% identical, at least 85% identical, at least 90% identical, at least 95% identical, at last 97% identical, or at least 99% identical to SEQ ID NO:310, wherein the light chain comprises V_{L} CDR1 comprising or consisting of SEQ ID NO:81, V_{L} CDR2 comprising or consisting of SEQ ID NO:82, and V_{L} CDR3 comprising or consisting of SEQ ID NO:83;
p. a light chain comprising an amino acid sequence at least 80% identical, at least 85% identical, at least 90% identical, at least 95% identical, at last 97% identical, or at least 99% identical to SEQ ID NO:312, wherein the light chain comprises V_{L} CDR1 comprising or consisting of SEQ ID NO:89, V_{L} CDR2 comprising or consisting of SEQ ID NO:90, and V_{L} CDR3 comprising or consisting of SEQ ID NO:91;
q. a light chain comprising an amino acid sequence at least 80% identical, at least 85% identical, at least 90% identical, at least 95% identical, at last 97% identical, or at least 99% identical to SEQ ID NO:314, wherein the light chain comprises V_{L} CDR1 comprising or consisting of SEQ ID NO:97, V_{L} CDR2 comprising or consisting of SEQ ID NO:98, and V_{L} CDR3 comprising or consisting of SEQ ID NO:99;
r. a light chain comprising an amino acid sequence at least 80% identical, at least 85% identical, at least 90% identical, at least 95% identical, at last 97% identical, or at least 99% identical to SEQ ID NO:316, wherein the light chain comprises V_{L} CDR1 comprising or consisting of SEQ ID NO:105, V_{L} CDR2 comprising or consisting of SEQ ID NO:106, and V_{L} CDR3 comprising or consisting of SEQ ID NO:107;
s. a light chain comprising an amino acid sequence at least 80% identical, at least 85% identical, at least 90% identical, at least 95% identical, at last 97% identical, or at least 99% identical to SEQ ID NO:318, wherein the light chain comprises V_{L} CDR1 comprising or consisting of SEQ ID NO:113, V_{L} CDR2 comprising or consisting of SEQ ID NO:114, and V_{L} CDR3 comprising or consisting of SEQ ID NO:115;
t. a light chain comprising an amino acid sequence at least 80% identical, at least 85% identical, at least 90% identical, at least 95% identical, at last 97% identical, or at least 99% identical to SEQ ID NO:320, wherein the light chain comprises V_{L} CDR1 comprising or consisting of SEQ ID NO:121, V_{L} CDR2 comprising or consisting of SEQ ID NO:122, and V_{L} CDR3 comprising or consisting of SEQ ID NO:123;
u. a light chain comprising an amino acid sequence at least 80% identical, at least 85% identical, at least 90% identical, at least 95% identical, at last 97% identical, or at least 99% identical to SEQ ID NO:322, wherein the light chain comprises V_{L} CDR1 comprising or consisting of SEQ ID NO:129, V_{L} CDR2 comprising or consisting of SEQ ID NO:130, and V_{L} CDR3 comprising or consisting of SEQ ID NO:131;
v. a light chain comprising an amino acid sequence at least 80% identical, at least 85% identical, at least 90% identical, at least 95% identical, at last 97% identical, or at least 99% identical to SEQ ID NO:324, wherein the light chain comprises V_{L} CDR1 comprising or consisting of SEQ ID NO: 137, V_{L} CDR2 comprising or consisting of SEQ ID NO:138, and V_{L} CDR3 comprising or consisting of SEQ ID NO:139;
w. a light chain comprising an amino acid sequence at least 80% identical, at least 85% identical, at least 90% identical, at least 95% identical, at last 97% identical, or at least 99% identical to SEQ ID NO:326, wherein the light chain comprises V_{L} CDR1 comprising or consisting of SEQ ID NO: 145, V_{L} CDR2 comprising or consisting of SEQ ID NO:146, and V_{L} CDR3 comprising or consisting of SEQ ID NO:147;
x. a light chain comprising an amino acid sequence at least 80% identical, at least 85% identical, at least 90% identical, at least 95% identical, at last 97% identical, or at least 99% identical to SEQ ID NO:328, wherein the light chain comprises V_{L} CDR1 comprising or consisting of SEQ ID NO:153, V_{L} CDR2 comprising or consisting of SEQ ID NO:154, and V_{L} CDR3 comprising or consisting of SEQ ID NO:155;
y. a light chain comprising an amino acid sequence at least 80% identical, at least 85% identical, at least 90% identical, at least 95% identical, at last 97% identical, or at least 99% identical to SEQ ID NO:330, wherein the light chain comprises V_{L} CDR1 comprising or consisting of SEQ ID NO:161, V_{L} CDR2 comprising or consisting of SEQ ID NO:162, and V_{L} CDR3 comprising or consisting of SEQ ID NO:163;
z. a light chain comprising an amino acid sequence at least 80% identical, at least 85% identical, at least 90% identical, at least 95% identical, at last 97% identical, or at least 99% identical to SEQ ID NO:332, wherein the light chain comprises V_{L} CDR1 comprising or consisting of SEQ ID NO:169, V_{L} CDR2 comprising or consisting of SEQ ID NO:170, and V_{L} CDR3 comprising or consisting of SEQ ID NO:171;
aa. a light chain comprising an amino acid sequence at least 80% identical, at least 85% identical, at least 90% identical, at least 95% identical, at last 97% identical, or at least 99% identical to SEQ ID NO:336, wherein the light chain comprises V_{L} CDR1 comprising or consisting of SEQ ID NO:185, V_{L} CDR2 comprising or consisting of SEQ ID NO:186, and V_{L} CDR3 comprising or consisting of SEQ ID NO:187;
bb. a light chain comprising an amino acid sequence at least 80% identical, at least 85% identical, at least 90% identical, at least 95% identical, at last 97% identical, or at least 99% identical to SEQ ID NO:338, wherein the light chain comprises V_{L} CDR1 comprising or consisting of SEQ ID NO: 193, V_{L} CDR2 comprising or consisting of SEQ ID NO:194, and V_{L} CDR3 comprising or consisting of SEQ ID NO:195;
cc. a light chain comprising an amino acid sequence at least 80% identical, at least 85% identical, at least 90% identical, at least 95% identical, at last 97% identical, or at least 99% identical to SEQ ID NO:340, wherein the light chain comprises V_{L} CDR1 comprising or consisting of SEQ ID NO:201, V_{L} CDR2 comprising or consisting of SEQ ID NO:202, and V_{L} CDR3 comprising or consisting of SEQ ID NO:203;
dd. a light chain comprising an amino acid sequence at least 80% identical, at least 85% identical, at least 90% identical, at least 95% identical, at last 97% identical, or at least 99% identical to SEQ ID NO:342, wherein the light chain comprises V_{L} CDR1 comprising or consisting of SEQ ID NO:209, V_{L} CDR2 comprising or consisting of SEQ ID NO:210, and V_{L} CDR3 comprising or consisting of SEQ ID NO:211;
ee. a light chain comprising an amino acid sequence at least 80% identical, at least 85% identical, at least 90% identical, at least 95% identical, at last 97% identical, or at least 99% identical to SEQ ID NO:344, wherein the light chain comprises V_{L} CDR1 comprising or consisting of SEQ ID NO:217, V_{L} CDR2 comprising or consisting of SEQ ID NO:218, and V_{L} CDR3 comprising or consisting of SEQ ID NO:219;
ff. a light chain comprising an amino acid sequence at least 80% identical, at least 85% identical, at least 90% identical, at least 95% identical, at last 97% identical, or at least 99% identical to SEQ ID NO:350, wherein the light chain comprises V_{L} CDR1 comprising or consisting of SEQ ID NO:241, V_{L} CDR2 comprising or consisting of SEQ ID NO:242, and V_{L} CDR3 comprising or consisting of SEQ ID NO:243;
gg. a light chain comprising an amino acid sequence at least 80% identical, at least 85% identical, at least 90% identical, at least 95% identical, at last 97% identical, or at least 99% identical to SEQ ID NO:352, wherein the light chain comprises V_{L} CDR1 comprising or consisting of SEQ ID NO:249, V_{L} CDR2 comprising or consisting of SEQ ID NO:250, and V_{L} CDR3 comprising or consisting of SEQ ID NO:251;
hh. a light chain comprising an amino acid sequence at least 80% identical, at least 85% identical, at least 90% identical, at least 95% identical, at last 97% identical, or at least 99% identical to SEQ ID NO:354, wherein the light chain comprises V_{L} CDR1 comprising or consisting of SEQ ID NO:257, V_{L} CDR2 comprising or consisting of SEQ ID NO:258, and V_{L} CDR3 comprising or consisting of SEQ ID NO:259;
ii. a light chain comprising an amino acid sequence at least 80% identical, at least 85% identical, at least 90% identical, at least 95% identical, at last 97% identical, or at least 99% identical to SEQ ID NO:358, wherein the light chain comprises V_{L} CDR1 comprising or consisting of SEQ ID NO:273, V_{L} CDR2 comprising or consisting of SEQ ID NO:274, and V_{L} CDR3 comprising or consisting of SEQ ID NO:275; and,
jj. a light chain comprising an amino acid sequence at least 80% identical, at least 85% identical, at least 90% identical, at least 95% identical, at last 97% identical, or at least 99% identical to SEQ ID NO:360, wherein the light chain comprises V_{L} CDR1 comprising or consisting of SEQ ID NO:281, V_{L} CDR2 comprising or consisting of SEQ ID NO:282, and V_{L} CDR3 comprising or consisting of SEQ ID NO:283.

9. The isolated antibody of claim 1, wherein the isolated antibody comprises a heavy chain selected from the group consisting of:
a. heavy chain comprising an amino acid sequence at least 80% identical, at least 85% identical, at least 90% identical, at least 95% identical, at last 97% identical, or at least 99% identical to SEQ ID NO:333, wherein the heavy chain comprises V_{H} CDR1 comprising or consisting of SEQ ID NO:181, V_{H} CDR2 comprising or consisting of SEQ ID NO:182, and V_{H} CDR3 comprising or consisting of SEQ ID NO:183;
b. a heavy chain comprising an amino acid sequence at least 80% identical, at least 85% identical, at least 90% identical, at least 95% identical, at last 97% identical, or at least 99% identical to SEQ ID NO:345, wherein the heavy chain comprises V_{H} CDR1 comprising or consisting of SEQ ID NO:229, V_{H} CDR2 comprising or consisting of SEQ ID NO:230, and V_{H} CDR3 comprising or consisting of SEQ ID NO:231;
c. a heavy chain comprising an amino acid sequence at least 80% identical, at least 85% identical, at least 90% identical, at least 95% identical, at last 97% identical, or at least 99% identical to SEQ ID NO:347, wherein the heavy chain comprises V_{H} CDR1 comprising or consisting of SEQ ID NO:237, V_{H} CDR2 comprising or consisting of SEQ ID NO:238, and V_{H} CDR3 comprising or consisting of SEQ ID NO:2239;
d. a heavy chain comprising an amino acid sequence at least 80% identical, at least 85% identical, at least 90% identical, at least 95% identical, at last 97% identical, or at least 99% identical to SEQ ID NO:355, wherein the heavy chain comprises V_{H} CDR1 comprising or consisting of SEQ ID NO:261, V_{H} CDR2 comprising or consisting of SEQ ID NO:262, and V_{H} CDR3 comprising or consisting of SEQ ID NO:263;
e. a heavy chain comprising an amino acid sequence at least 80% identical, at least 85% identical, at least 90% identical, at least 95% identical, at last 97% identical, or at least 99% identical to SEQ ID NO:289, wherein the heavy chain comprises V_{H} CDR1 comprising or consisting of SEQ ID NO:5, V_{H} CDR2 comprising or consisting of SEQ ID NO:6, and V_{H} CDR3 comprising or consisting of SEQ ID NO:7;
f. a heavy chain comprising an amino acid sequence at least 80% identical, at least 85% identical, at least 90% identical, at least 95% identical, at last 97% identical, or at least 99% identical to SEQ ID NO:291, wherein the heavy chain comprises V_{H} CDR1 comprising or consisting of SEQ ID NO:13, V_{H} CDR2 comprising or consisting of SEQ ID NO:14, and V_{H} CDR3 comprising or consisting of SEQ ID NO:15;
g. a heavy chain comprising an amino acid sequence at least 80% identical, at least 85% identical, at least 90% 213, at least 95% identical, at last 97% identical, or at least 99% identical to SEQ ID NO:293, wherein the heavy chain comprises V_{H} CDR1 comprising or consisting of SEQ ID NO:21, V_{H} CDR2 comprising or consisting of SEQ ID NO:22, and V_{H} CDR3 comprising or consisting of SEQ ID NO:23;
h. a heavy chain comprising an amino acid sequence at least 80% identical, at least 85% identical, at least 90% identical, at least 95% identical, at last 97% identical, or at least 99% identical to SEQ ID NO:295, wherein the heavy chain comprises V_{H} CDR1 comprising or consisting of SEQ ID NO:29, V_{H} CDR2 comprising or consisting of SEQ ID NO:30, and V_{H} CDR3 comprising or consisting of SEQ ID NO:31;
i. a heavy chain comprising an amino acid sequence at least 80% identical, at least 85% identical, at least 90% identical, at least 95% identical, at last 97% identical, or at least 99% identical to SEQ ID NO:297, wherein the heavy chain comprises V_{H} CDR1 comprising or consisting of SEQ ID NO:37, V_{H} CDR2 comprising or consisting of SEQ ID NO:38, and V_{H} CDR3 comprising or consisting of SEQ ID NO:39;
j. a heavy chain comprising an amino acid sequence at least 80% identical, at least 85% identical, at least 90% identical, at least 95% identical, at last 97% identical, or at least 99% identical to SEQ ID NO:299, wherein the heavy chain comprises V_{H} CDR1 comprising or consisting of SEQ ID NO:45, V_{H} CDR2 comprising or consisting of SEQ ID NO:46, and V_{H} CDR3 comprising or consisting of SEQ ID NO:47;
k. a heavy chain comprising an amino acid sequence at least 80% identical, at least 85% identical, at least 90% identical, at least 95% identical, at last 97% identical, or at least 99% identical to SEQ ID NO:301, wherein the heavy chain comprises V_{H} CDR1 comprising or consisting of SEQ ID NO:53, V_{H} CDR2 comprising or consisting of SEQ ID NO:54, and V_{H} CDR3 comprising or consisting of SEQ ID NO:55;
l. a heavy chain comprising an amino acid sequence at least 80% identical, at least 85% identical, at least 90% identical, at least 95% identical, at last 97% identical, or at least 99% identical to SEQ ID NO:303, wherein the heavy chain comprises V_{H} CDR1 comprising or consisting of SEQ ID NO:61, V_{H} CDR2 comprising or consisting of SEQ ID NO:62, and V_{H} CDR3 comprising or consisting of SEQ ID NO:63;
m. a heavy chain comprising an amino acid sequence at least 80% identical, at least 85% identical, at least 90% identical, at least 95% identical, at last 97% identical, or at least 99% identical to SEQ ID NO:305, wherein the heavy chain comprises V_{H} CDR1 comprising or consisting of SEQ ID NO:69, V_{H} CDR2 comprising or consisting of SEQ ID NO:70, and V_{H} CDR3 comprising or consisting of SEQ ID NO:71;
n. a heavy chain comprising an amino acid sequence at least 80% identical, at least 85% identical, at least 90% identical, at least 95% identical, at last 97% identical, or at least 99% identical to SEQ ID NO:307, wherein the heavy chain comprises V_{H} CDR1 comprising or consisting of SEQ ID NO:77, V_{H} CDR2 comprising or consisting of SEQ ID NO:78, and V_{H} CDR3 comprising or consisting of SEQ ID NO:79;
o. a heavy chain comprising an amino acid sequence at least 80% identical, at least 85% identical, at least 90% identical, at least 95% identical, at last 97% identical, or at least 99% identical to SEQ ID NO:309, wherein the heavy chain comprises V_{H} CDR1 comprising or consisting of SEQ ID NO:85, V_{H} CDR2 comprising or consisting of SEQ ID NO:86, and V_{H} CDR3 comprising or consisting of SEQ ID NO:87;
p. a heavy chain comprising an amino acid sequence at least 80% identical, at least 85% identical, at least 90% identical, at least 95% identical, at last 97% identical, or at least 99% identical to SEQ ID NO:311, wherein the heavy chain comprises V_{H} CDR1 comprising or consisting of SEQ ID NO:93, V_{H} CDR2 comprising or consisting of SEQ ID NO:94, and V_{H} CDR3 comprising or consisting of SEQ ID NO:95;
q. a heavy chain comprising an amino acid sequence at least 80% identical, at least 85% identical, at least 90% identical, at least 95% identical, at last 97% identical, or at least 99% identical to SEQ ID NO:313, wherein the heavy chain comprises V_{H} CDR1 comprising or consisting of SEQ ID NO:101, V_{H} CDR2 comprising or consisting of SEQ ID NO:102, and V_{H} CDR3 comprising or consisting of SEQ ID NO:103;
r. a heavy chain comprising an amino acid sequence at least 80% identical, at least 85% identical, at least 90% identical, at least 95% identical, at last 97% identical, or at least 99% identical to SEQ ID NO:315, wherein the heavy chain comprises V_{H} CDR1 comprising or consisting of SEQ ID NO:109, V_{H} CDR2 comprising or consisting of SEQ ID NO:110, and V_{H} CDR3 comprising or consisting of SEQ ID NO:111;
s. a heavy chain comprising an amino acid sequence at least 80% identical, at least 85% identical, at least 90% identical, at least 95% identical, at last 97% identical, or at least 99% identical to SEQ ID NO:317, wherein the heavy chain comprises V_{H} CDR1 comprising or consisting of SEQ ID NO:117, V_{H} CDR2 comprising or consisting of SEQ ID NO:118, and V_{H} CDR3 comprising or consisting of SEQ ID NO:119;
t. a heavy chain comprising an amino acid sequence at least 80% identical, at least 85% identical, at least 90% identical, at least 95% identical, at last 97% identical, or at least 99% identical to SEQ ID NO:319, wherein the heavy chain comprises V_{H} CDR1 comprising or consisting of SEQ ID NO:125, V_{H} CDR2 comprising or consisting of SEQ ID NO:126, and V_{H} CDR3 comprising or consisting of SEQ ID NO:127;
u. a heavy chain comprising an amino acid sequence at least 80% identical, at least 85% identical, at least 90% identical, at least 95% identical, at last 97% identical, or at least 99% identical to SEQ ID NO:321, wherein the heavy chain comprises V_{H} CDR1 comprising or consisting of SEQ ID NO:133, V_{H} CDR2 comprising or consisting of SEQ ID NO:134, and V_{H} CDR3 comprising or consisting of SEQ ID NO:135;
v. a heavy chain comprising an amino acid sequence at least 80% identical, at least 85% identical, at least 90% identical, at least 95% identical, at last 97% identical, or at least 99% identical to SEQ ID NO:323, wherein the heavy chain comprises V_{H} CDR1 comprising or consisting of SEQ ID NO:141, V_{H} CDR2 comprising or consisting of SEQ ID NO:142, and V_{H} CDR3 comprising or consisting of SEQ ID NO 143;
w. a heavy chain comprising an amino acid sequence at least 80% identical, at least 85% identical, at least 90% identical, at least 95% identical, at last 97% identical, or at least 99% identical to SEQ ID NO:325, wherein the heavy chain comprises V_{H} CDR1 comprising or consisting of SEQ ID NO:149, V_{H} CDR2 comprising or consisting of SEQ ID NO: 150, and V_{H} CDR3 comprising or consisting of SEQ ID NO:151;
x. a heavy chain comprising an amino acid sequence at least 80% identical, at least 85% identical, at least 90% identical, at least 95% identical, at last 97% identical, or at least 99% identical to SEQ ID NO:327, wherein the heavy chain comprises V_{H} CDR1 comprising or consisting of SEQ ID NO:157, V_{H} CDR2 comprising or consisting of SEQ ID NO: 158, and V_{H} CDR3 comprising or consisting of SEQ ID NO:159;
y. a heavy chain comprising an amino acid sequence at least 80% identical, at least 85% identical, at least 90% identical, at least 95% identical, at last 97% identical, or at least 99% identical to SEQ ID NO:329, wherein the heavy chain comprises V_{H} CDR1 comprising or consisting of SEQ ID NO:165, V_{H} CDR2 comprising or consisting of SEQ ID NO:166, and V_{H} CDR3 comprising or consisting of SEQ ID NO:167;
z. a heavy chain comprising an amino acid sequence at least 80% identical, at least 85% identical, at least 90% identical, at least 95% identical, at last 97% identical, or at least 99% identical to SEQ ID NO:331, wherein the heavy chain comprises V_{H} CDR1 comprising or consisting of SEQ ID NO:173, V_{H} CDR2 comprising or consisting of SEQ ID NO:174, and V_{H} CDR3 comprising or consisting of SEQ ID NO:175;
aa. a heavy chain comprising an amino acid sequence at least 80% identical, at least 85% identical, at least 90% identical, at least 95% identical, at last 97% identical, or at least 99% identical to SEQ ID NO:335, wherein the heavy chain comprises V_{H} CDR1 comprising or consisting of SEQ ID NO:189, V_{H} CDR2 comprising or consisting of SEQ ID NO:190, and V_{H} CDR3 comprising or consisting of SEQ ID NO:191;
bb. a heavy chain comprising an amino acid sequence at least 80% identical, at least 85% identical, at least 90% identical, at least 95% identical, at last 97% identical, or at least 99% identical to SEQ ID NO:337, wherein the heavy chain comprises V_{H} CDR1 comprising or consisting of SEQ ID NO:197, V_{H} CDR2 comprising or consisting of SEQ ID NO:198, and V_{H} CDR3 comprising or consisting of SEQ ID NO:199;
cc. a heavy chain comprising an amino acid sequence at least 80% identical, at least 85% identical, at least 90% identical, at least 95% identical, at last 97% identical, or at least 99% identical to SEQ ID NO:339, wherein the heavy chain comprises V_{H} CDR1 comprising or consisting of SEQ ID NO:205, V_{H} CDR2 comprising or consisting of SEQ ID NO:206, and V_{H} CDR3 comprising or consisting of SEQ ID NO:207;
dd. a heavy chain comprising an amino acid sequence at least 80% identical, at least 85% identical, at least 90% identical, at least 95% identical, at last 97% identical, or at least 99% identical to SEQ ID NO:341, wherein the heavy chain comprises V_{H} CDR1 comprising or consisting of SEQ ID NO:213, V_{H} CDR2 comprising or consisting of SEQ ID NO:214, and V_{H} CDR3 comprising or consisting of SEQ ID NO:215;
ee. a heavy chain comprising an amino acid sequence at least 80% identical, at least 85% identical, at least 90% identical, at least 95% identical, at last 97% identical, or at least 99% identical to SEQ ID NO:343, wherein the heavy chain comprises V_{H} CDR1 comprising or consisting of SEQ ID NO:221, V_{H} CDR2 comprising or consisting of SEQ ID NO:222, and V_{H} CDR3 comprising or consisting of SEQ ID NO:223;
ff. a heavy chain comprising an amino acid sequence at least 80% identical, at least 85% identical, at least 90% identical, at least 95% identical, at last 97% identical, or at least 99% identical to SEQ ID NO:349, wherein the heavy chain comprises V_{H} CDR1 comprising or consisting of SEQ ID NO:245, V_{H} CDR2 comprising or consisting of SEQ ID NO:246, and V_{H} CDR3 comprising or consisting of SEQ ID NO:247;
gg. a heavy chain comprising an amino acid sequence at least 80% identical, at least 85% identical, at least 90% identical, at least 95% identical, at last 97% identical, or at least 99% identical to SEQ ID NO:351, wherein the heavy chain comprises V_{H} CDR1 comprising or consisting of SEQ ID NO:253, V_{H} CDR2 comprising or consisting of SEQ ID NO:254, and V_{H} CDR3 comprising or consisting of SEQ ID NO:255;
hh. a heavy chain comprising an amino acid sequence at least 80% identical, at least 85% identical, at least 90% identical, at least 95% identical, at last 97% identical, or at least 99% identical to SEQ ID NO:353, wherein the heavy chain comprises V_{H} CDR1 comprising or consisting of SEQ ID NO:261, V_{H} CDR2 comprising or consisting of SEQ ID NO:262, and V_{H} CDR3 comprising or consisting of SEQ ID NO:263;
ii. a heavy chain comprising an amino acid sequence at least 80% identical, at least 85% identical, at least 90% identical, at least 95% identical, at last 97% identical, or at least 99% identical to SEQ ID NO:357, wherein the heavy chain comprises V_{H} CDR1 comprising or consisting of SEQ ID NO:269, V_{H} CDR2 comprising or consisting of SEQ ID NO:270, and V_{H} CDR3 comprising or consisting of SEQ ID NO:271; and
jj. a heavy chain comprising an amino acid sequence at least 80% identical, at least 85% identical, at least 90% identical, at least 95% identical, at last 97% identical, or at least 99% identical to SEQ ID NO:359, wherein the heavy chain comprises V_{H} CDR1 comprising or consisting of SEQ ID NO:285, V_{H} CDR2 comprising or consisting of SEQ ID NO:286, and V_{H} CDR3 comprising or consisting of SEQ ID NO:287.

10. The isolated antibody of claim 1, wherein the isolated antibody is selected from the group consisting of:
a. an isolated antibody comprising:
a heavy chain comprising an amino acid sequence at least 80% identical, at least 85% identical, at least 90% identical, at least 95% identical, at last 97% identical, or at least 99% identical to SEQ ID NO:333, wherein the heavy chain comprises V_{H} CDR1 comprising or consisting of SEQ ID NO:181, V_{H} CDR2 comprising or consisting of SEQ ID NO: 182, and V_{H} CDR3 comprising or consisting of SEQ ID NO:183; and,
a light chain comprising an amino acid sequence at least 80% identical, at least 85% identical, at least 90% identical, at least 95% identical, at last 97% identical, or at least 99% identical to SEQ ID NO:334, wherein the light chain comprises V_{L} CDR1 comprising or consisting of SEQ ID NO: 177, V_{L} CDR2 comprising or consisting of SEQ ID NO:178, and V_{L} CDR3 comprising or consisting of SEQ ID NO:179;
b. an isolated antibody comprising:
a heavy chain comprising an amino acid sequence at least 80% identical, at least 85% identical, at least 90% identical, at least 95% identical, at last 97% identical, or at least 99% identical to SEQ ID NO:345, wherein the heavy chain comprises V_{H} CDR1 comprising or consisting of SEQ ID NO:229, V_{H} CDR2 comprising or consisting of SEQ ID NO:230, and V_{H} CDR3 comprising or consisting of SEQ ID NO:231; and,
a light chain comprising an amino acid sequence at least 80% identical, at least 85% identical, at least 90% identical, at least 95% identical, at last 97% identical, or at least 99% identical to SEQ ID NO:346, wherein the light chain comprises V_{L} CDR1 comprising or consisting of SEQ ID NO:225, V_{L} CDR2 comprising or consisting of SEQ ID NO:226, and V_{L} CDR3 comprising or consisting of SEQ ID NO:227;
c. an isolated antibody comprising:
a heavy chain comprising an amino acid sequence at least 80% identical, at least 85% identical, at least 90% identical, at least 95% identical, at last 97% identical, or at least 99% identical to SEQ ID NO:347, wherein the heavy chain comprises V_{H} CDR1 comprising or consisting of SEQ ID NO:237, V_{H} CDR2 comprising or consisting of SEQ ID NO:238, and V_{H} CDR3 comprising or consisting of SEQ ID NO:2239, and,
a light chain comprising an amino acid sequence at least 80% identical, at least 85% identical, at least 90% identical, at least 95% identical, at last 97% identical, or at least 99% identical to SEQ ID NO:348, wherein the light chain comprises V_{L} CDR1 comprising or consisting of SEQ ID NO:233, V_{L} CDR2 comprising or consisting of SEQ ID NO:234, and V_{L} CDR3 comprising or consisting of SEQ ID NO:235;
d. an isolated antibody comprising:
a heavy chain comprising an amino acid sequence at least 80% identical, at least 85% identical, at least 90% identical, at least 95% identical, at last 97% identical, or at least 99% identical to SEQ ID NO:355, wherein the heavy chain comprises V_{H} CDR1 comprising or consisting of SEQ ID NO:261, V_{H} CDR2 comprising or consisting of SEQ ID NO:262, and V_{H} CDR3 comprising or consisting of SEQ ID NO:263, and,
a light chain comprising an amino acid sequence at least 80% identical, at least 85% identical, at least 90% identical, at least 95% identical, at last 97% identical, or at least 99% identical to SEQ ID NO:356, wherein the light chain comprises V_{L} CDR1 comprising or consisting of SEQ ID NO:265, V_{L} CDR2 comprising or consisting of SEQ ID NO:266, and V_{L} CDR3 comprising or consisting of SEQ ID NO:267;
e. an isolated antibody comprising:
a heavy chain comprising an amino acid sequence at least 80% identical, at least 85% identical, at least 90% identical, at least 95% identical, at last 97% identical, or at least 99% identical to SEQ ID NO:289, wherein the heavy chain comprises V_{H} CDR1 comprising or consisting of SEQ ID NO:5, V_{H} CDR2 comprising or consisting of SEQ ID NO:6, and V_{H} CDR3 comprising or consisting of SEQ ID NO:7, and,
a light chain comprising an amino acid sequence at least 80% identical, at least 85% identical, at least 90% identical, at least 95% identical, at last 97% identical, or at least 99% identical to SEQ ID NO:290, wherein the light chain comprises V_{L} CDR1 comprising or consisting of SEQ ID NO:1, V_{L} CDR2 comprising or consisting of SEQ ID NO:2, and V_{L} CDR3 comprising or consisting of SEQ ID NO:3;
f. an isolated antibody comprising:
a heavy chain comprising an amino acid sequence at least 80% identical, at least 85% identical, at least 90% identical, at least 95% identical, at last 97% identical, or at least 99% identical to SEQ ID NO:291, wherein the heavy chain comprises V_{H} CDR1 comprising or consisting of SEQ ID NO:13, V_{H} CDR2 comprising or consisting of SEQ ID NO: 14, and V_{H} CDR3 comprising or consisting of SEQ ID NO:15; and,
a light chain comprising an amino acid sequence at least 80% identical, at least 85% identical, at least 90% identical, at least 95% identical, at last 97% identical, or at least 99% identical to SEQ ID NO:292, wherein the light chain comprises V_{L} CDR1 comprising or consisting of SEQ ID NO:9, V_{L} CDR2 comprising or consisting of SEQ ID NO:10, and V_{L} CDR3 comprising or consisting of SEQ ID NO:11;
g. an isolated antibody comprising:
a heavy chain comprising an amino acid sequence at least 80% identical, at least 85% identical, at least 90% 213, at least 95% identical, at last 97% identical, or at least 99% identical to SEQ ID NO:293, wherein the heavy chain comprises V_{H} CDR1 comprising or consisting of SEQ ID NO:21, V_{H} CDR2 comprising or consisting of SEQ ID NO:22, and V_{H} CDR3 comprising or consisting of SEQ ID NO:23; and,
a light chain comprising an amino acid sequence at least 80% identical, at least 85% identical, at least 90% identical, at least 95% identical, at last 97% identical, or at least 99% identical to SEQ ID NO:294, wherein the light chain comprises V_{L} CDR1 comprising or consisting of SEQ ID NO:17, V_{L} CDR2 comprising or consisting of SEQ ID NO:18, and V_{L} CDR3 comprising or consisting of SEQ ID NO:19;
h. an isolated antibody comprising:
a heavy chain comprising an amino acid sequence at least 80% identical, at least 85% identical, at least 90% identical, at least 95% identical, at last 97% identical, or at least 99% identical to SEQ ID NO:295, wherein the heavy chain comprises V_{H} CDR1 comprising or consisting of SEQ ID NO:29, V_{H} CDR2 comprising or consisting of SEQ ID NO:30, and V_{H} CDR3 comprising or consisting of SEQ ID NO:31; and,
a light chain comprising an amino acid sequence at least 80% identical, at least 85% identical, at least 90% identical, at least 95% identical, at last 97% identical, or at least 99% identical to SEQ ID NO:296, wherein the light chain comprises V_{L} CDR1 comprising or consisting of SEQ ID NO:25, V_{L} CDR2 comprising or consisting of SEQ ID NO:26, and V_{L} CDR3 comprising or consisting of SEQ ID NO:27;
i. an isolated antibody comprising:
a heavy chain comprising an amino acid sequence at least 80% identical, at least 85% identical, at least 90% identical, at least 95% identical, at last 97% identical, or at least 99% identical to SEQ ID NO:297, wherein the heavy chain comprises V_{H} CDR1 comprising or consisting of SEQ ID NO:37, V_{H} CDR2 comprising or consisting of SEQ ID NO:38, and V_{H} CDR3 comprising or consisting of SEQ ID NO:39; and,
a light chain comprising an amino acid sequence at least 80% identical, at least 85% identical, at least 90% identical, at least 95% identical, at last 97% identical, or at least 99% identical to SEQ ID NO:298, wherein the light chain comprises V_{L} CDR1 comprising or consisting of SEQ ID NO:33, V_{L} CDR2 comprising or consisting of SEQ ID NO:34, and V_{L} CDR3 comprising or consisting of SEQ ID NO:35;
j. an isolated antibody comprising:
a heavy chain comprising an amino acid sequence at least 80% identical, at least 85% identical, at least 90% identical, at least 95% identical, at last 97% identical, or at least 99% identical to SEQ ID NO:299, wherein the heavy chain comprises V_{H} CDR1 comprising or consisting of SEQ ID NO:45, V_{H} CDR2 comprising or consisting of SEQ ID NO:46, and V_{H} CDR3 comprising or consisting of SEQ ID NO:47; and,
a light chain comprising an amino acid sequence at least 80% identical, at least 85% identical, at least 90% identical, at least 95% identical, at last 97% identical, or at least 99% identical to SEQ ID NO:300, wherein the light chain comprises V_{L} CDR1 comprising or consisting of SEQ ID NO:41, V_{L} CDR2 comprising or consisting of SEQ ID NO:42, and V_{L} CDR3 comprising or consisting of SEQ ID NO:43;
k. an isolated antibody comprising:
a heavy chain comprising an amino acid sequence at least 80% identical, at least 85% identical, at least 90% identical, at least 95% identical, at last 97% identical, or at least 99% identical to SEQ ID NO:301, wherein the heavy chain comprises V_{H} CDR1 comprising or consisting of SEQ ID NO:53, V_{H} CDR2 comprising or consisting of SEQ ID NO:54, and V_{H} CDR3 comprising or consisting of SEQ ID NO:55; and,
a light chain comprising an amino acid sequence at least 80% identical, at least 85% identical, at least 90% identical, at least 95% identical, at last 97% identical, or at least 99% identical to SEQ ID NO:302, wherein the light chain comprises V_{L} CDR1 comprising or consisting of SEQ ID NO:49, V_{L} CDR2 comprising or consisting of SEQ ID NO:50, and V_{L} CDR3 comprising or consisting of SEQ ID NO:51;
l. an isolated antibody comprising:
a heavy chain comprising an amino acid sequence at least 80% identical, at least 85% identical, at least 90% identical, at least 95% identical, at last 97% identical, or at least 99% identical to SEQ ID NO:303, wherein the heavy chain comprises V_{H} CDR1 comprising or consisting of SEQ ID NO:61, V_{H} CDR2 comprising or consisting of SEQ ID NO:62, and V_{H} CDR3 comprising or consisting of SEQ ID NO:63; and,
a light chain comprising an amino acid sequence at least 80% identical, at least 85% identical, at least 90% identical, at least 95% identical, at last 97% identical, or at least 99% identical to SEQ ID NO:304, wherein the light chain comprises V_{L} CDR1 comprising or consisting of SEQ ID NO:57, V_{L} CDR2 comprising or consisting of SEQ ID NO:58, and V_{L} CDR3 comprising or consisting of SEQ ID NO:59;
m. an isolated antibody comprising:
a heavy chain comprising an amino acid sequence at least 80% identical, at least 85% identical, at least 90% identical, at least 95% identical, at last 97% identical, or at least 99% identical to SEQ ID NO:305, wherein the heavy chain comprises V_{H} CDR1 comprising or consisting of SEQ ID NO:69, V_{H} CDR2 comprising or consisting of SEQ ID NO:70, and V_{L} CDR3 comprising or consisting of SEQ ID NO:71; and,
a light chain comprising an amino acid sequence at least 80% identical, at least 85% identical, at least 90% identical, at least 95% identical, at last 97% identical, or at least 99% identical to SEQ ID NO:306, wherein the light chain comprises V_{L} CDR1 comprising or consisting of SEQ ID NO:65, V_{L} CDR2 comprising or consisting of SEQ ID NO:66, and V_{L} CDR3 comprising or consisting of SEQ ID NO:67;
n. an isolated antibody comprising:
a heavy chain comprising an amino acid sequence at least 80% identical, at least 85% identical, at least 90% identical, at least 95% identical, at last 97% identical, or at least 99% identical to SEQ ID NO:307, wherein the heavy chain comprises V_{H} CDR1 comprising or consisting of SEQ ID NO:77, V_{H} CDR2 comprising or consisting of SEQ ID NO:78, and V_{H} CDR3 comprising or consisting of SEQ ID NO:79; and,
a light chain comprising an amino acid sequence at least 80% identical, at least 85% identical, at least 90% identical, at least 95% identical, at last 97% identical, or at least 99% identical to SEQ ID NO:308, wherein the light chain comprises V_{L} CDR1 comprising or consisting of SEQ ID NO:73, V_{L} CDR2 comprising or consisting of SEQ ID NO:74, and V_{L} CDR3 comprising or consisting of SEQ ID NO:75;
o. an isolated antibody comprising:
a heavy chain comprising an amino acid sequence at least 80% identical, at least 85% identical, at least 90% identical, at least 95% identical, at last 97% identical, or at least 99% identical to SEQ ID NO:309, wherein the heavy chain comprises V_{H} CDR1 comprising or consisting of SEQ ID NO:85, V_{H} CDR2 comprising or consisting of SEQ ID NO:86, and V_{H} CDR3 comprising or consisting of SEQ ID NO:87; and,
a light chain comprising an amino acid sequence at least 80% identical, at least 85% identical, at least 90% identical, at least 95% identical, at last 97% identical, or at least 99% identical to SEQ ID NO:310, wherein the light chain comprises V_{L} CDR1 comprising or consisting of SEQ ID NO:81, V_{L} CDR2 comprising or consisting of SEQ ID NO:82, and V_{L} CDR3 comprising or consisting of SEQ ID NO:83;
p. an isolated antibody comprising:
a heavy chain comprising an amino acid sequence at least 80% identical, at least 85% identical, at least 90% identical, at least 95% identical, at last 97% identical, or at least 99% identical to SEQ ID NO:311, wherein the heavy chain comprises V_{H} CDR1 comprising or consisting of SEQ ID NO:93, V_{H} CDR2 comprising or consisting of SEQ ID NO:94, and V_{H} CDR3 comprising or consisting of SEQ ID NO:95; and,
a light chain comprising an amino acid sequence at least 80% identical, at least 85% identical, at least 90% identical, at least 95% identical, at last 97% identical, or at least 99% identical to SEQ ID NO:312, wherein the light chain comprises V_{L} CDR1 comprising or consisting of SEQ ID NO:89, V_{L} CDR2 comprising or consisting of SEQ ID NO:90, and V_{L} CDR3 comprising or consisting of SEQ ID NO:91;
q. an isolated antibody comprising:
a heavy chain comprising an amino acid sequence at least 80% identical, at least 85% identical, at least 90% identical, at least 95% identical, at last 97% identical, or at least 99% identical to SEQ ID NO:313, wherein the heavy chain comprises V_{H} CDR1 comprising or consisting of SEQ ID NO:101, V_{H} CDR2 comprising or consisting of SEQ ID NO: 102, and V_{H} CDR3 comprising or consisting of SEQ ID NO:103; and,
a light chain comprising an amino acid sequence at least 80% identical, at least 85% identical, at least 90% identical, at least 95% identical, at last 97% identical, or at least 99% identical to SEQ ID NO:314, wherein the light chain comprises V_{L} CDR1 comprising or consisting of SEQ ID NO:97, V_{L} CDR2 comprising or consisting of SEQ ID NO:98, and V_{L} CDR3 comprising or consisting of SEQ ID NO:99;
r. an isolated antibody comprising:
a heavy chain comprising an amino acid sequence at least 80% identical, at least 85% identical, at least 90% identical, at least 95% identical, at last 97% identical, or at least 99% identical to SEQ ID NO:315, wherein the heavy chain comprises V_{H} CDR1 comprising or consisting of SEQ ID NO:109, V_{H} CDR2 comprising or consisting of SEQ ID NO:110, and V_{H} CDR3 comprising or consisting of SEQ ID NO:111; and,
a light chain comprising an amino acid sequence at least 80% identical, at least 85% identical, at least 90% identical, at least 95% identical, at last 97% identical, or at least 99% identical to SEQ ID NO:316, wherein the light chain comprises V_{L} CDR1 comprising or consisting of SEQ ID NO:105, V_{L} CDR2 comprising or consisting of SEQ ID NO:106, and V_{L} CDR3 comprising or consisting of SEQ ID NO: 107;
s. an isolated antibody comprising:
a heavy chain comprising an amino acid sequence at least 80% identical, at least 85% identical, at least 90% identical, at least 95% identical, at last 97% identical, or at least 99% identical to SEQ ID NO:317, wherein the heavy chain comprises V_{H} CDR1 comprising or consisting of SEQ ID NO: 117, V_{H} CDR2 comprising or consisting of SEQ ID NO:118, and V_{H} CDR3 comprising or consisting of SEQ ID NO:119; and,
a light chain comprising an amino acid sequence at least 80% identical, at least 85% identical, at least 90% identical, at least 95% identical, at last 97% identical, or at least 99% identical to SEQ ID NO:318, wherein the light chain comprises V_{L} CDR1 comprising or consisting of SEQ ID NO:113, V_{L} CDR2 comprising or consisting of SEQ ID NO: 114, and V_{L} CDR3 comprising or consisting of SEQ ID NO: 115;
t. an isolated antibody comprising:
a heavy chain comprising an amino acid sequence at least 80% identical, at least 85% identical, at least 90% identical, at least 95% identical, at last 97% identical, or at least 99% identical to SEQ ID NO:319, wherein the heavy chain comprises V_{H} CDR1 comprising or consisting of SEQ ID NO:125, V_{H} CDR2 comprising or consisting of SEQ ID NO:126, and V_{H} CDR3 comprising or consisting of SEQ ID NO:127; and,
a light chain comprising an amino acid sequence at least 80% identical, at least 85% identical, at least 90% identical, at least 95% identical, at last 97% identical, or at least 99% identical to SEQ ID NO:320, wherein the light chain comprises V_{L} CDR1 comprising or consisting of SEQ ID NO:121, V_{L} CDR2 comprising or consisting of SEQ ID NO:122, and V_{L} CDR3 comprising or consisting of SEQ ID NO:123;
u. an isolated antibody comprising:
a heavy chain comprising an amino acid sequence at least 80% identical, at least 85% identical, at least 90% identical, at least 95% identical, at last 97% identical, or at least 99% identical to SEQ ID NO:321, wherein the heavy chain comprises V_{H} CDR1 comprising or consisting of SEQ ID NO: 133, V_{H} CDR2 comprising or consisting of SEQ ID NO: 134, and V_{H} CDR3 comprising or consisting of SEQ ID NO:135; and,
a light chain comprising an amino acid sequence at least 80% identical, at least 85% identical, at least 90% identical, at least 95% identical, at last 97% identical, or at least 99% identical to SEQ ID NO:322, wherein the light chain comprises V_{L} CDR1 comprising or consisting of SEQ ID NO: 129, V_{L} CDR2 comprising or consisting of SEQ ID NO:130, and V_{L} CDR3 comprising or consisting of SEQ ID NO:131;
v. an isolated antibody comprising:
a heavy chain comprising an amino acid sequence at least 80% identical, at least 85% identical, at least 90% identical, at least 95% identical, at last 97% identical, or at least 99% identical to SEQ ID NO:323, wherein the heavy chain comprises V_{H} CDR1 comprising or consisting of SEQ ID NO:141, V_{H} CDR2 comprising or consisting of SEQ ID NO: 142, and V_{H} CDR3 comprising or consisting of SEQ ID NO 143; and,
a light chain comprising an amino acid sequence at least 80% identical, at least 85% identical, at least 90% identical, at least 95% identical, at last 97% identical, or at least 99% identical to SEQ ID NO:324, wherein the light chain comprises V_{L} CDR1 comprising or consisting of SEQ ID NO:137, V_{L} CDR2 comprising or consisting of SEQ ID NO:138, and V_{L} CDR3 comprising or consisting of SEQ ID NO:139;
w. an isolated antibody comprising:
a heavy chain comprising an amino acid sequence at least 80% identical, at least 85% identical, at least 90% identical, at least 95% identical, at last 97% identical, or at least 99% identical to SEQ ID NO:325, wherein the heavy chain comprises V_{H} CDR1 comprising or consisting of SEQ ID NO:149, V_{H} CDR2 comprising or consisting of SEQ ID NO:150, and V_{H} CDR3 comprising or consisting of SEQ ID NO:151; and,
a light chain comprising an amino acid sequence at least 80% identical, at least 85% identical, at least 90% identical, at least 95% identical, at last 97% identical, or at least 99% identical to SEQ ID NO:326, wherein the light chain comprises V_{L} CDR1 comprising or consisting of SEQ ID NO:145, V_{L} CDR2 comprising or consisting of SEQ ID NO:146, and V_{L} CDR3 comprising or consisting of SEQ ID NO: 147;
x. an isolated antibody comprising:
a heavy chain comprising an amino acid sequence at least 80% identical, at least 85% identical, at least 90% identical, at least 95% identical, at last 97% identical, or at least 99% identical to SEQ ID NO:327, wherein the heavy chain comprises V_{H} CDR1 comprising or consisting of SEQ ID NO:157, V_{H} CDR2 comprising or consisting of SEQ ID NO:158, and V_{H} CDR3 comprising or consisting of SEQ ID NO:159; and,
a light chain comprising an amino acid sequence at least 80% identical, at least 85% identical, at least 90% identical, at least 95% identical, at last 97% identical, or at least 99% identical to SEQ ID NO:328, wherein the light chain comprises V_{L} CDR1 comprising or consisting of SEQ ID NO:153, V_{L} CDR2 comprising or consisting of SEQ ID NO:154, and V_{L} CDR3 comprising or consisting of SEQ ID NO: 155;
y. an isolated antibody comprising:
a heavy chain comprising an amino acid sequence at least 80% identical, at least 85% identical, at least 90% identical, at least 95% identical, at last 97% identical, or at least 99% identical to SEQ ID NO:329, wherein the heavy chain comprises V_{H} CDR1 comprising or consisting of SEQ ID NO:165, V_{H} CDR2 comprising or consisting of SEQ ID NO:166, and V_{H} CDR3 comprising or consisting of SEQ ID NO:167; and,
a light chain comprising an amino acid sequence at least 80% identical, at least 85% identical, at least 90% identical, at least 95% identical, at last 97% identical, or at least 99% identical to SEQ ID NO:330, wherein the light chain comprises V_{L} CDR1 comprising or consisting of SEQ ID NO:161, V_{L} CDR2 comprising or consisting of SEQ ID NO:162, and V_{L} CDR3 comprising or consisting of SEQ ID NO:163;
z. an isolated antibody comprising:
a heavy chain comprising an amino acid sequence at least 80% identical, at least 85% identical, at least 90% identical, at least 95% identical, at last 97% identical, or at least 99% identical to SEQ ID NO:331, wherein the heavy chain comprises V_{H} CDR1 comprising or consisting of SEQ ID NO:173, V_{H} CDR2 comprising or consisting of SEQ ID NO: 174, and V_{H} CDR3 comprising or consisting of SEQ ID NO:175; and,
a light chain comprising an amino acid sequence at least 80% identical, at least 85% identical, at least 90% identical, at least 95% identical, at last 97% identical, or at least 99% identical to SEQ ID NO:332, wherein the light chain comprises V_{L} CDR1 comprising or consisting of SEQ ID NO: 169, V_{L} CDR2 comprising or consisting of SEQ ID NO:170, and V_{L} CDR3 comprising or consisting of SEQ ID NO:171;
aa. an isolated antibody comprising:
a heavy chain comprising an amino acid sequence at least 80% identical, at least 85% identical, at least 90% identical, at least 95% identical, at last 97% identical, or at least 99% identical to SEQ ID NO:335, wherein the heavy chain comprises V_{H} CDR1 comprising or consisting of SEQ ID NO:189, V_{H} CDR2 comprising or consisting of SEQ ID NO: 190, and V_{H} CDR3 comprising or consisting of SEQ ID NO:191; and,
a light chain comprising an amino acid sequence at least 80% identical, at least 85% identical, at least 90% identical, at least 95% identical, at last 97% identical, or at least 99% identical to SEQ ID NO:336, wherein the light chain comprises V_{L} CDR1 comprising or consisting of SEQ ID NO:185, V_{L} CDR2 comprising or consisting of SEQ ID NO:186, and V_{L} CDR3 comprising or consisting of SEQ ID NO:187;
bb. an isolated antibody comprising:
a heavy chain comprising an amino acid sequence at least 80% identical, at least 85% identical, at least 90% identical, at least 95% identical, at last 97% identical, or at least 99% identical to SEQ ID NO:337, wherein the heavy chain comprises V_{H} CDR1 comprising or consisting of SEQ ID NO:197, V_{H} CDR2 comprising or consisting of SEQ ID NO:198, and V_{H} CDR3 comprising or consisting of SEQ ID NO:199; and,
a light chain comprising an amino acid sequence at least 80% identical, at least 85% identical, at least 90% identical, at least 95% identical, at last 97% identical, or at least 99% identical to SEQ ID NO:338, wherein the light chain comprises V_{L} CDR1 comprising or consisting of SEQ ID NO:193, V_{L} CDR2 comprising or consisting of SEQ ID NO:194, and V_{L} CDR3 comprising or consisting of SEQ ID NO: 195;
cc. an isolated antibody comprising:
a heavy chain comprising an amino acid sequence at least 80% identical, at least 85% identical, at least 90% identical, at least 95% identical, at last 97% identical, or at least 99% identical to SEQ ID NO:339, wherein the heavy chain comprises V_{H} CDR1 comprising or consisting of SEQ ID NO:205, V_{H} CDR2 comprising or consisting of SEQ ID NO:206, and V_{H} CDR3 comprising or consisting of SEQ ID NO:207; and,
a light chain comprising an amino acid sequence at least 80% identical, at least 85% identical, at least 90% identical, at least 95% identical, at last 97% identical, or at least 99% identical to SEQ ID NO:340, wherein the light chain comprises V_{L} CDR1 comprising or consisting of SEQ ID NO:201, V_{L} CDR2 comprising or consisting of SEQ ID NO:202, and V_{L} CDR3 comprising or consisting of SEQ ID NO:203;
dd. an isolated antibody comprising:
a heavy chain comprising an amino acid sequence at least 80% identical, at least 85% identical, at least 90% identical, at least 95% identical, at last 97% identical, or at least 99% identical to SEQ ID NO:341, wherein the heavy chain comprises V_{H} CDR1 comprising or consisting of SEQ ID NO:213, V_{H} CDR2 comprising or consisting of SEQ ID NO:214, and V_{H} CDR3 comprising or consisting of SEQ ID NO:215; and,
a light chain comprising an amino acid sequence at least 80% identical, at least 85% identical, at least 90% identical, at least 95% identical, at last 97% identical, or at least 99% identical to SEQ ID NO:342, wherein the light chain comprises V_{L} CDR1 comprising or consisting of SEQ ID NO:209, V_{L} CDR2 comprising or consisting of SEQ ID NO:210, and V_{L} CDR3 comprising or consisting of SEQ ID NO:211;
ee. an isolated antibody comprising:
a heavy chain comprising an amino acid sequence at least 80% identical, at least 85% identical, at least 90% identical, at least 95% identical, at last 97% identical, or at least 99% identical to SEQ ID NO:343, wherein the heavy chain comprises V_{H} CDR1 comprising or consisting of SEQ ID NO:221, V_{H} CDR2 comprising or consisting of SEQ ID NO:222, and V_{H} CDR3 comprising or consisting of SEQ ID NO:223; and,
a light chain comprising an amino acid sequence at least 80% identical, at least 85% identical, at least 90% identical, at least 95% identical, at last 97% identical, or at least 99% identical to SEQ ID NO:344, wherein the light chain comprises V_{L} CDR1 comprising or consisting of SEQ ID NO:217, V_{L} CDR2 comprising or consisting of SEQ ID NO:218, and V_{L} CDR3 comprising or consisting of SEQ ID NO:219;
ff. an isolated antibody comprising:
a heavy chain comprising an amino acid sequence at least 80% identical, at least 85% identical, at least 90% identical, at least 95% identical, at last 97% identical, or at least 99% identical to SEQ ID NO:349, wherein the heavy chain comprises V_{H} CDR1 comprising or consisting of SEQ ID NO:245, V_{H} CDR2 comprising or consisting of SEQ ID NO:246, and V_{H} CDR3 comprising or consisting of SEQ ID NO:247; and,
a light chain comprising an amino acid sequence at least 80% identical, at least 85% identical, at least 90% identical, at least 95% identical, at last 97% identical, or at least 99% identical to SEQ ID NO:350, wherein the light chain comprises V_{L} CDR1 comprising or consisting of SEQ ID NO:241, V_{L} CDR2 comprising or consisting of SEQ ID NO:242, and V_{L} CDR3 comprising or consisting of SEQ ID NO:243;
gg. an isolated antibody comprising:
a heavy chain comprising an amino acid sequence at least 80% identical, at least 85% identical, at least 90% identical, at least 95% identical, at last 97% identical, or at least 99% identical to SEQ ID NO:351, wherein the heavy chain comprises V_{H} CDR1 comprising or consisting of SEQ ID NO:253, V_{H} CDR2 comprising or consisting of SEQ ID NO:254, and V_{H} CDR3 comprising or consisting of SEQ ID NO:255; and,
a light chain comprising an amino acid sequence at least 80% identical, at least 85% identical, at least 90% identical, at least 95% identical, at last 97% identical, or at least 99% identical to SEQ ID NO:352, wherein the light chain comprises V_{L} CDR1 comprising or consisting of SEQ ID NO:249, V_{L} CDR2 comprising or consisting of SEQ ID NO:250, and V_{L} CDR3 comprising or consisting of SEQ ID NO:251;
hh. an isolated antibody comprising:
a heavy chain comprising an amino acid sequence at least 80% identical, at least 85% identical, at least 90% identical, at least 95% identical, at last 97% identical, or at least 99% identical to SEQ ID NO:353, wherein the heavy chain comprises V_{H} CDR1 comprising or consisting of SEQ ID NO:261, V_{H} CDR2 comprising or consisting of SEQ ID NO:262, and V_{H} CDR3 comprising or consisting of SEQ ID NO:263; and,
a light chain comprising an amino acid sequence at least 80% identical, at least 85% identical, at least 90% identical, at least 95% identical, at last 97% identical, or at least 99% identical to SEQ ID NO:354, wherein the light chain comprises V_{L} CDR1 comprising or consisting of SEQ ID NO:257, V_{L} CDR2 comprising or consisting of SEQ ID NO:258, and V_{L} CDR3 comprising or consisting of SEQ ID NO:259;
ii. an isolated antibody comprising:
a heavy chain comprising an amino acid sequence at least 80% identical, at least 85% identical, at least 90% identical, at least 95% identical, at last 97% identical, or at least 99% identical to SEQ ID NO:357, wherein the heavy chain comprises V_{H} CDR1 comprising or consisting of SEQ ID NO:269, V_{H} CDR2 comprising or consisting of SEQ ID NO:270, and V_{H} CDR3 comprising or consisting of SEQ ID NO:271; and,
a light chain comprising an amino acid sequence at least 80% identical, at least 85% identical, at least 90% identical, at least 95% identical, at last 97% identical, or at least 99% identical to SEQ ID NO:358, wherein the light chain comprises V_{L} CDR1 comprising or consisting of SEQ ID NO:273, V_{L} CDR2 comprising or consisting of SEQ ID NO:274, and V_{L} CDR3 comprising or consisting of SEQ ID NO:275; and,
jj. an isolated antibody comprising:
a heavy chain comprising an amino acid sequence at least 80% identical, at least 85% identical, at least 90% identical, at least 95% identical, at last 97% identical, or at least 99% identical to SEQ ID NO:359, wherein the heavy chain comprises V_{H} CDR1 comprising or consisting of SEQ ID NO:285, V i CDR2 comprising or consisting of SEQ ID NO:286, and V_{H} CDR3 comprising or consisting of SEQ ID NO:287; and,
a light chain comprising an amino acid sequence at least 80% identical, at least 85% identical, at least 90% identical, at least 95% identical, at last 97% identical, or at least 99% identical to SEQ ID NO:360, wherein the light chain comprises V_{L} CDR1 comprising or consisting of SEQ ID NO:281, V_{L} CDR2 comprising or consisting of SEQ ID NO:282, and V_{L} CDR3 comprising or consisting of SEQ ID NO:283.

11. A method of preventing infection of a cell by an alphavirus, comprising administering to the individual the isolated antibody of claim 1.

12. A method of protecting an individual against infection by an alphavirus, comprising contacting the cell with the isolated antibody of claim 1.

13. A method of treating an individual for an alphavirus infection, comprising administering to the individual the isolated antibody of claim 1.

14. The method of claim 1, wherein the alphavirus is EEEV, WEEV, VEEV, CHKV, or Ross River Virus.


SEQUENCE LISTING (360 sequences)

SEQ ID NO: 1 - protein, 6 aa, Macaca fascicularis
QNVMTL

SEQ ID NO: 2 - skipped

SEQ ID NO: 3 - protein, 11 aa, Macaca fascicularis [sequence omitted]

SEQ ID NO: 4 - protein, 107 aa, Macaca fascicularis
DIQMTQSPSSLSASVGDTVTITCRASQNVMTLLAWYQHKPGKAPKLLIYQASSLHIGVPSRFSGSGSGTDFTLTISSLQSEDFATYSCQQYDSRPWTFGQGTKVDIK

SEQ ID NO: 5 - protein, 8 aa, Macaca fascicularis
GASITSNY

SEQ ID NO: 6 - protein, 8 aa, Macaca fascicularis
VYGDAGST

SEQ ID NO: 7 - protein, 15 aa, Macaca fascicularis
CASRSSVADNWFDVW

SEQ ID NO: 8 - protein, 120 aa, Macaca fascicularis
QVQLRESGPGLVKPSETLSLTCAVSGASITSNYWTWIRQPPGKGLEWIGRVYGDAGSTDYNPSLKSRVTISMDMSKNHFSLDLTSVTAADTALYYCASRSSVADNWFDVWGPGVLVTVSS

SEQ ID NO: 9 - protein, 6 aa, Macaca fascicularis
QSISSL

SEQ ID NO: 10 - skipped

SEQ ID NO: 11 - protein, 11 aa, Macaca fascicularis
CQQHDSRPWTF

SEQ ID NO: 12 - protein, 107 aa, Macaca fascicularis
DIQMTQSPSSLSASVGDTVTITCRATQSISSLLAWYQYKPGKAPKLLIYQASSLHIGVPSRFSGSGSGTDFTLTISSLQSEDFATYYCQQHDSRPWTFGQGTKVDIK

SEQ ID NO: 13 - protein, 8 aa, Macaca fascicularis
GDSISNNY

SEQ ID NO: 14 - protein, 8 aa, Macaca fascicularis
IYGDAGST

SEQ ID NO: 15 - protein, 15 aa, Macaca fascicularis
CASRTTVADNWFDVW

SEQ ID NO: 16 - protein, 120 aa, Macaca fascicularis
QVQLQESGPGLVKPSETLSLTCAVSGDSISNNYWTWIRHFPGKGLEWIGRIYGDAGSTDYNPSLKSRVTISMDLSKNQFSLDLTSVTVADTALYYCASRTTVADNWFDVWGPGVLVTVSS

SEQ ID NO: 17 - protein, 9 aa, Macaca fascicularis
SSDIGVYNY

SEQ ID NO: 18 - skipped

SEQ ID NO: 19 - protein, 14 aa, Macaca fascicularis
CSSYSGIDTDTYIF

SEQ ID NO: 20 - protein, 112 aa, Macaca fascicularis
QSVLTQPPSVSGSPGQSVTISCTGTSSDIGVYNYVSWYQQHPGKAPKLMVSEVSIRPSGVSDRFSGSKSGNTASLTISGLQAEDEADYYCSSYSGIDTDTYIFGTGTRLTVL

SEQ ID NO: 21 - protein, 9 aa, Macaca fascicularis
SGSISGYSY

SEQ ID NO: 22 - protein, 8 aa, Macaca fascicularis
IYGSGGSS

SEQ ID NO: 23 - protein, 14 aa, Macaca fascicularis
CALAKYSGSPGDYW

SEQ ID NO: 24 - protein, 120 aa, Macaca fascicularis
QVQLQESGPGLVKPSETLSLTCAVSSGSISGYSYWSWIRQPPGKGLEWIGNIYGSGGSSYLNPSLKSRVTLSLDTSKNHFSLRVTSVTAADTAVYYCALAKYSGSPGDYWGQGVLATVSS

SEQ ID NO: 25 - protein, 9 aa, Macaca fascicularis
SSDIGVYNY

SEQ ID NO: 26 - skipped

SEQ ID NO: 27 - protein, 14 aa, Macaca fascicularis
CSSYSGSETDTYIF

SEQ ID NO: 28 - protein, 111 aa, Macaca fascicularis
QFVLTQPPSVSGSPGQSVTISCTGTSSDIGVYNYVSWYQQYPGKAPKLMVSEVSLRPSGVSHRFSGSKSGNTASLTISGLQAEDEADYYCSSYSGSETDTYIFGTGTRLTV

SEQ ID NO: 29 - protein, 9 aa, Macaca fascicularis
SGSISGFSY

SEQ ID NO: 30 - protein, 8 aa, Macaca fascicularis
IYGSGGSS

SEQ ID NO: 31 - protein, 14 aa, Macaca fascicularis
CALAKYSGSPGDYW

SEQ ID NO: 32 - protein, 120 aa, Macaca fascicularis
QVQLQESGPGLVKPSETLSLTCAVSSGSISGFSYWSWIRQPPGRGLEWIGNIYGSGGSSYVNPSLKSRVTLSLDTSKNQFSLRVTSVTAADTAVYYCALAKYSGSPGDYWGQGVLATVSS

SEQ ID NO: 33 - protein, 12 aa, Macaca fascicularis
QSLLYNTNNKEY

SEQ ID NO: 34 - skipped

SEQ ID NO: 35 - protein, 11 aa, Macaca fascicularis [sequence omitted]

SEQ ID NO: 36 - protein, 113 aa, Macaca fascicularis
DVVMTQSPESLAVSLGERVTISCKSSQSLLYNTNNKEYLAWYQQKPGQAPKLLFYWASTRESGVPNRFSGSGSGTDFTLTISGLQAEDVAVYYCQQYYSSPRTFGQGTKVEIK

SEQ ID NO: 37 - protein, 10 aa, Macaca fascicularis
GVSISTKYHY

SEQ ID NO: 38 - protein, 7 aa, Macaca fascicularis
ISYTGGT

SEQ ID NO: 39 - protein, 11 aa, Macaca fascicularis [sequence omitted]

SEQ ID NO: 40 - protein, 116 aa, Macaca fascicularis
QVQLQESGPGLVKPSETLSLTCVVSGVSISTKYHYWSWIRQAPGKGLEWIGYISYTGGTSYNPSLQSRVTISRDTSKNHFSLSFNSVTPADTALYFCASDENKPYWGQGVLVTVSS

SEQ ID NO: 41 - protein, 12 aa, Macaca fascicularis
QSLLYNSNNKNY

SEQ ID NO: 42 - skipped

SEQ ID NO: 43 - protein, 11 aa, Macaca fascicularis [sequence omitted]

SEQ ID NO: 44 - protein, 113 aa, Macaca fascicularis
DIVMTQSPDSVAVSLGERVTINCKSGQSLLYNSNNKNYLAWYQQKPGQAPNLLIYWASTRESGVPNRFSGSGSGTNFTLTISGLQAEDVAVYYCQQYYSSPRTFGQGTKVDIK

SEQ ID NO: 45 - protein, 10 aa, Macaca fascicularis
GGSLTTNYYY

SEQ ID NO: 46 - protein, 7 aa, Macaca fascicularis
ISYTGST

SEQ ID NO: 47 - protein, 10 aa, Macaca fascicularis
CVGDENKIHW

SEQ ID NO: 48 - protein, 116 aa, Macaca fascicularis
QVQLRESGPGLVKPSETLSLTCAVSGGSLTTNYYYWSWIRQAPGKGLEWIGYISYTGSTNYNPSLQSRVTISRDTSKNQFSLNLSSMKVADTAVYFCVGDENKIHWGPGVLVIVSS

SEQ ID NO: 49 - protein, 12 aa, Macaca fascicularis
QSLLYSSNNKNY

SEQ ID NO: 50 - skipped

SEQ ID NO: 51 - protein, 10 aa, Macaca fascicularis [sequence omitted]

SEQ ID NO: 52 - protein, 112 aa, Macaca fascicularis
DIVMTQSPDSLAVSLGERVTINCKSSQSLLYSSNNKNYLAWYQQKPGQAPRLLIYWASTRESGVPNRFSGSGSGTDFTLTINGLQAEDVAVYYCQQYYNSRTFGQGTKVEIK

SEQ ID NO: 53 - protein, 10 aa, Macaca fascicularis
GGSISSSYSY

SEQ ID NO: 54 - protein, 7 aa, Macaca fascicularis
ISYTGST

SEQ ID NO: 55 - protein, 10 aa, Macaca fascicularis
CATDRNTLAW

SEQ ID NO: 56 - protein, 116 aa, Macaca fascicularis
QVQLQESGPGLVKPSETLSLTCAVSGGSISSSYSYWSWIRQAPGKGLEWIGYISYTGSTNYNPSLKSRVTISRDTSKNQFSLELTSMTTADTAVYYCATDRNTLAWGPGVLVTVSS

SEQ ID NO: 57 - protein, 11 aa, Macaca fascicularis
QSLVHSNGNTS

SEQ ID NO: 58 - skipped

SEQ ID NO: 59 - protein, 11 aa, Macaca fascicularis
CGQGAHLPPAF

SEQ ID NO: 60 - protein, 112 aa, Macaca fascicularis
DVVMTQSPLSLPVTPGQPASISCRSSQSLVHSNGNTSLSWYQQKAGHPPRLLIYQVSNRYSGVPDRFSGTGAGTDFTLTISRVETEDVGVYYCGQGAHLPPAFGQGTKVEIK

SEQ ID NO: 61 - protein, 8 aa, Macaca fascicularis
GFTFSSYA

SEQ ID NO: 62 - protein, 7 aa, Macaca fascicularis
ISSHYST

SEQ ID NO: 63 - protein, 26 aa, Macaca fascicularis
CAKVGRSGYCTASGCNALLFDWFDVW

SEQ ID NO: 64 - protein, 130 aa, Macaca fascicularis
EVQLVESGGGLAKPGGSLRLSCAASGFTFSSYAMHWVRQAPGKGLEWVSVISSHYSTYYADSVKGRFTISRDNSKNTVSLQVNSLRAEDTAVYYCAKVGRSGYCTASGCNALLFDWFDVWGPGVLVTVSS

SEQ ID NO: 65 - protein, 6 aa, Macaca fascicularis
NIGSEL

SEQ ID NO: 66 - skipped

SEQ ID NO: 67 - protein, 14 aa, Macaca fascicularis
CQLWDISSDHNYIF

SEQ ID NO: 68 - protein, 109 aa, Macaca fascicularis
SYELTQPPSVSASPGQTARITCGGINIGSELVHWYQQKPPQAPVLVIYANGERPSGIPERFSGSNSGNTATLTISGVEAGDEADYYCQLWDISSDHNYIFGDGTRLTVL

SEQ ID NO: 69 - protein, 8 aa, Macaca fascicularis
GFTFSDYR

SEQ ID NO: 70 - protein, 8 aa, Macaca fascicularis
IKPSGTNT

SEQ ID NO: 71 - protein, 18 aa, Macaca fascicularis
CARVVTESRPPAAWFDVW

SEQ ID NO: 72 - protein, 124 aa, Macaca fascicularis
EVQLVESGGGLVQPGGSLRLSCVASGFTFSDYRMAWVRQATGKGLEWVSTISQPSGTNTYYLDPVKGRFTVSRDNAKNTLYLQMHSLRAEDTAVYYCARVVTESRPPAAWFDVWGPGVLVTVSS

SEQ ID NO: 73 - protein, 12 aa, Macaca fascicularis
QSLFDSGDVTTY

SEQ ID NO: 74 - skipped

SEQ ID NO: 75 - protein, 10 aa, Macaca fascicularis
MQPLEYPRTF

SEQ ID NO: 76 - protein, 113 aa, Macaca fascicularis
DIVMTQTPPSLPVTLGEPASISCRSSQSLFDSGDVTTYLDWYLQKPGQSPQLLIFEVSKRASGVPERFSGSGSDTDFTLQISRVEAEDVGVYYCMQPLEYPRTFGQGTRVEIK

SEQ ID NO: 77 - protein, 8 aa, Macaca fascicularis
GFTFSSYG

SEQ ID NO: 78 - protein, 7 aa, Macaca fascicularis
ITSGGNT

SEQ ID NO: 79 - protein, 23 aa, Macaca fascicularis
CAKEKYSSYSAYGRGYPNSLDVW

SEQ ID NO: 80 - protein, 128 aa, Macaca fascicularis
EVQLVESGGGLGKPGGSLRLSCAASGFTFSSYGMHWVRQAPGKGLEWASSITSSGGNTDYADSVKGRFTISRDNSKNTLSLQMNSLRPEDSATYYCAKEKYSSYSAYGRGYPNSLDVWGRGVLVTVSS

SEQ ID NO: 81 - protein, 12 aa, Macaca fascicularis
QSLFDSGDVTTY

SEQ ID NO: 82 - skipped

SEQ ID NO: 83 - protein, 11 aa, Macaca fascicularis
CMQPLEYPRTF

SEQ ID NO: 84 - protein, 113 aa, Macaca fascicularis
DVVMTQTPLSLPVTLGEPASISCRSSQSLFDSGDVTTYLDWYLQKPGQSPQLLIFEVSKRASGVPERFSGSGSDTDFTLQISRVEAEDVGVYYCMQPLEYPRTFGQGTKVEIK

SEQ ID NO: 85 - protein, 8 aa, Macaca fascicularis
GFTFSSYG

SEQ ID NO: 86 - protein, 7 aa, Macaca fascicularis
ITSGGNT

SEQ ID NO: 87 - protein, 23 aa, Macaca fascicularis
CAKEKYSSYSAYGRGYPNSLDVW

SEQ ID NO: 88 - protein, 128 aa, Macaca fascicularis
EVQLVESGGGLGKPGGSLRLSCAASGFTFSSYGMHWVRQAPGKGLEWASSITSSGGNTDYADSVKGRFTISRDNSKNTLYLQMNSLRPEDSGTYYCAKEKYSSYSAYGRGYPNSLDVWGRGVLVTVSS

SEQ ID NO: 89 - protein, 11 aa, Macaca fascicularis
QSLLDSNGNTY

SEQ ID NO: 90 - skipped

SEQ ID NO: 91 - protein, 11 aa, Macaca fascicularis
CMQVLQVPLTF

SEQ ID NO: 92 - protein, 112 aa, Macaca fascicularis
DVVMTQTPLSLPVTPGEPTSISCRSSQSLLDSNGNTYLHWYLQKPGQSPQLLIYGGSNRPSGVPDRFSGSGSGTDFTLKISNVEAGDVGTYYCMQVLQVPLTFGGGTKVDIK

SEQ ID NO: 93 - protein, 8 aa, Macaca fascicularis
ANSFTGSW

SEQ ID NO: 94 - protein, 8 aa, Macaca fascicularis
IYGGDSDL

SEQ ID NO: 95 - protein, 17 aa, Macaca fascicularis
CARSPWSGYYIGWFDVW

SEQ ID NO: 96 - protein, 122 aa, Macaca fascicularis
VVQLVQSGAEVKRPGESLRISCKTSANSFTGSWINWVRQVPGKGLEWMGSIYGGDSDLRYNPSFRGHVTFTADKSISTAYLQWTSLQASDTATYYCARSPWSGYYIGWFDVWGPGVLVTVSS

SEQ ID NO: 97 - protein, 11 aa, Macaca fascicularis
QSLLDSNGNTY

SEQ ID NO: 98 - skipped

SEQ ID NO: 99 - protein, 11 aa, Macaca fascicularis
CMQVIQVPLTF

SEQ ID NO: 100 - protein, 112 aa, Macaca fascicularis
DVVMTQTPLSLPVTPGEPASISCRSSQSLLDSNGNTYLHWYLQKPGQSPQLLIYGGSNRASGVPDRFSGSGSGTDFTLTINNVEAGDVGTYYCMQVIQVPLTFGGGTKVDIK

SEQ ID NO: 101 - protein, 8 aa, Macaca fascicularis
GYRFTDIW

SEQ ID NO: 102 - protein, 8 aa, Macaca fascicularis
IYLDDSDI

SEQ ID NO: 103 - protein, 17 aa, Macaca fuscata
CARSVWSGYYIGWFDVW

SEQ ID NO: 104 - protein, 122 aa, Macaca fascicularis
VVQLVQSGAEVKRPGESLRISCKTSGYRFTDIWINWVRQRPGKGLEWMGSIYLDDSDIRFNPSFQGHVTISADKSTSTTYLQWSSLKASDTATYFCARSVWSGYYIGWFDVWGPGVLVTVSS

SEQ ID NO: 105 - protein, 11 aa, Macaca fascicularis
QSLLDSNGNTY

SEQ ID NO: 106 - skipped

SEQ ID NO: 107 - protein, 11 aa, Macaca fascicularis
CMQVIQVPLTF

SEQ ID NO: 108 - protein, 112 aa, Macaca fascicularis
DVVMTQTPLSLPVTPGESASISCRSSQSLLDSNGNTYLHWYLQKPGQSPQLLIYGGSNRASGVPDRFSGSGSGTDFTLKISNVEAGDVGSYYCMQVIQVPLTFGGGTKVEIK

SEQ ID NO: 109 - protein, 8 aa, Macaca fascicularis
GYSFTDIW

SEQ ID NO: 110 - protein, 8 aa, Macaca fascicularis
LYLDDSDI

SEQ ID NO: 111 - protein, 17 aa, Macaca fascicularis
CARSVWSGYYIGWFDVW

SEQ ID NO: 112 - protein, 122 aa, Macaca fascicularis
VVQLVQSGAGLKRPGESLRISCKTSGYSFTDIWISWVRQMPGKGLEWMGSLYLDDSDIRFNPSFQGHVTMSADKSTSTAYLQWSSLRASDTATYYCARSVWSGYYIGWFDVWGPGVLVAVSS

SEQ ID NO: 113 - protein, 6 aa, Macaca fascicularis
QDISSF

SEQ ID NO: 114 - skipped

SEQ ID NO: 115 - protein, 11 aa, Macaca fascicularis
CQQGHSIPYSF

SEQ ID NO: 116 - protein, 107 aa, Macaca fascicularis
DIQMSQSPSSLSASVGDRVSITCRASQDISSFLNWYQQKPGKAPNLLIHYGEFLAGGVPSRFSGSGSGTDYTLTISSLQPEDLGTYYCQQGHSIPYSFGQGTKVEIK

SEQ ID NO: 117 - protein, 8 aa, Macaca fascicularis
GFTFSDYE

SEQ ID NO: 118 - protein, 8 aa, Macaca fascicularis
VGQPSDST

SEQ ID NO: 119 - protein, 21 aa, Macaca fascicularis
CARLFRGYCSSTFCFKWFDVW

SEQ ID NO: 120 - protein, 127 aa, Macaca fascicularis
EVQLVESGGGLAQPGGSLRLSCVASGFTFSDYEMAWVRQATGKGLEWVSSVGQPSDSQTYYLDPVKGRFTISRDNAKNTLYLQMNSLGAEDTAVYYCARLFRGYCSSTFCFKWFDVWGPGVLVTVSS

SEQ ID NO: 121 - protein, 6 aa, Macaca fascicularis
QSISSW

SEQ ID NO: 122 - skipped

SEQ ID NO: 123 - protein, 11 aa, Macaca fascicularis
CQQYISRPWTF

SEQ ID NO: 124 - protein, 107 aa, Macaca fascicularis
DIQMTQSPSSLSASVGDTVTITCRASQSISSWLAWYQQKPGKAPNLLIYKASSLQTGVPSRFSGSGSETHFTLTISSLQSEDFATYYCQQYISRPWTFGQGTKVEIK

SEQ ID NO: 125 - protein, 8 aa, Macaca fascicularis
GGSISGYY

SEQ ID NO: 126 - protein, 8 aa, Macaca fascicularis
MYDSTGDT

SEQ ID NO: 127 - protein, 15 aa, Macaca fascicularis
CASGSGDWFGYFYRW

SEQ ID NO: 128 - protein, 120 aa, Macaca fascicularis
QVQLQESGPGLVKPSETLSLTCTVSGGSISGYYWNWIRQSPGKGLEWIGNMYDSTGDTNYNPSLKSRVTLSIDTSKNQFSMKLNSVTAADTAVYFCASGSGDWFGYFYRWGQGVLVTVSS

SEQ ID NO: 129 - protein, 6 aa, Macaca fascicularis
HNINSF

SEQ ID NO: 130 - skipped

SEQ ID NO: 131 - protein, 11 aa, Macaca fascicularis
CLQHSRHPFSF

SEQ ID NO: 132 - protein, 107 aa, Macaca fascicularis
DIQMTQSPSALSASVGDRVTITCRASHNINSFLAWYQQKPGQVPKLLIYAASSLENGVPSRFSGSGSGTEFTLTIRGLQPEDFATYFCLQHSRHPFSFGPGTKVEIK

SEQ ID NO: 133 - protein, 10 aa, Macaca fascicularis
GGSISGGDYY

SEQ ID NO: 134 - protein, 8 aa, Macaca fascicularis
IHGNWGNT

SEQ ID NO: 135 - protein, 13 aa, Macaca fascicularis
CVREAYGTYFYFW

SEQ ID NO: 136 - protein, 120 aa, Macaca fascicularis
IVQLQESGPGLVKPSETLSLTCAVSGGSISGGDYYWTWIRQSPGKGLEWIGYIHGNWGNTLYNPSLESRITISKDTSNNQFSLSLRSVTAADTAVYYCVREAYGTYFYFWGQGVLVTVSS

SEQ ID NO: 137 - protein, 6 aa, Macaca fascicularis
NIGSKS

SEQ ID NO: 138 - skipped

SEQ ID NO: 139 - protein, 12 aa, Macaca fascicularis [sequence omitted]

SEQ ID NO: 140 - protein, 107 aa, Macaca fascicularis
PMRLTQPRSVSVSPGQTARITCGGDNIGSKSVHWYQQKPPQAPVLVIYDDRARPSGIPERFSGSKSGNTATLAISGVEAGDEADYYCQVWDSSSDVLFGGGTRLTTV

SEQ ID NO: 141 - protein, 8 aa, Macaca fascicularis
GFRFSDFH

SEQ ID NO: 142 - protein, 8 aa, Macaca fascicularis
ISHGGVTT

SEQ ID NO: 143 - protein, 18 aa, Macaca fascicularis
CARDSPYWGDYSHSLDVW

SEQ ID NO: 144 - protein, 123 aa, Macaca fascicularis
EVQLVESGGGLAKPGGSLRLSCAAAGFRFSDFHMHWVRQAPGKGLEWVSRISHGGVTTRFADSVKGRFTISRENAKNTLYLQMDNLRAEDTAVYYCARDSPYWGDYSHSLDVWGRGVLVTVSS

SEQ ID NO: 145 - protein, 6 aa, Macaca fascicularis
QDINKY

SEQ ID NO: 146 - skipped

SEQ ID NO: 147 - protein, 11 aa, Macaca fascicularis
CQQHNISPYNF

SEQ ID NO: 148 - protein, 107 aa, Macaca fascicularis
DIQMTQSPSSLTASVGDRVTITCRASQDINKYLSWYQQKPGKAPKSLIYLASSLERGVPTRFSGSGSGTHYILTISSLQPEDIGTYYCQQHNISPYNFGQGTKIQIK

SEQ ID NO: 149 - protein, 8 aa, Macaca fascicularis
GFPFSDYD

SEQ ID NO: 150 - protein, 10 aa, Macaca fascicularis
IKSKPGGGTP

SEQ ID NO: 151 - protein, 7 aa, Macaca fascicularis
CTTSVVW

SEQ ID NO: 152 - protein, 114 aa, Macaca fascicularis
EVQLVESGGGLVQPGGSLRLSCVASGFPFSDYDMNWVRQAPGKGLEWIGFIKSKPGGGTPYYAASVKGRFTISRDDSKNTLYLQMNSLKIEDTAVFYCTTSVVWGQGALVTVSS

SEQ ID NO: 153 - protein, 8 aa, Macaca fascicularis
TSNIGDYY

SEQ ID NO: 154 - skipped

SEQ ID NO: 155 - protein, 13 aa, Macaca fascicularis
CGTWDNSLTSVLF

SEQ ID NO: 156 - protein, 110 aa, Macaca fascicularis
PFVLTQPPSVSGDPGQKVTISCSGSTSNIGDYYVHWYQQFPGTAPKLLIFENNRRPSGVSDRFSGSKSGSSASLTISGLQPGDEADYYCGTWDNSLTSVLFGGGTRLTTV

SEQ ID NO: 157 - protein, 10 aa, Macaca fascicularis
GFSISSTGIG

SEQ ID NO: 158 - protein, 7 aa, Macaca fascicularis
IYWNDNT

SEQ ID NO: 159 - protein, 14 aa, Macaca fascicularis
CAFLVVLTKWFDVW

SEQ ID NO: 160 - protein, 120 aa, Macaca fascicularis
QVTLKESGPALVKPTQTLTLTCTFSGFSISSTGIGVGWIRQPPGKALEWLANIYWNDNTDYNTSLKNRLTISKDTSKNQVVLTMTNMDPVDTATYHCAFLVVLTKWFDVWGPGVLVTVSS

SEQ ID NO: 161 - protein, 6 aa, Macaca fascicularis
QAISSS

SEQ ID NO: 162 - skipped

SEQ ID NO: 163 - protein, 11 aa, Macaca fascicularis
CLQYNSAPFTF

SEQ ID NO: 164 - protein, 107 aa, Macaca fascicularis
DIQMTQSPSSLSASAGDRVTITCRASQAISSSLAWYQQKPGKAPKRLIFDASSLQGGVPSRFSGSGSGTDFTLTISSLQPEDFATYFCLQYNSAPFTFGPGTKLDIM

SEQ ID NO: 165 - protein, 8 aa, Macaca fascicularis
GFTFSDSW

SEQ ID NO: 166 - protein, 10 aa, Macaca fascicularis
IKRRADGETA

SEQ ID NO: 167 - protein, 21 aa, Macaca fascicularis
CTTDDRTYCSRGVCYAAFYHW

SEQ ID NO: 168 - protein, 128 aa, Macaca fascicularis
EVQLVESGAGLVQPGGSLRLSCAASGFTFSDSWMSWVRQSPGKGLEWVARIKRRADGETADYAPSVKGRFIVSRDDSNNTLYLQMNSLKTEDTAVYFCTTDDRTYCSRGVCYAAFYHWGQGVLVTVSS

SEQ ID NO: 169 - protein, 6 aa, Macaca fascicularis
QDISSY

SEQ ID NO: 170 - skipped

SEQ ID NO: 171 - protein, 11 aa, Macaca fascicularis
CLQYNSAPFTF

SEQ ID NO: 172 - protein, 107 aa, Macaca fascicularis
DIQMTQSPSSLSASAGDRVSITCRASQDISSYLAWYQQKPGKPPKRLIFDASSLQGGVPSRFSGSGSGTDFTLTISSLQPEDFATYFCLQYNSAPFTFGPGTQLDIK

SEQ ID NO: 173 - protein, 8 aa, Macaca fascicularis
GFTFSNSW

SEQ ID NO: 174 - protein, 10 aa, Macaca fascicularis
IKRKADYETP

SEQ ID NO: 175 - protein, 21 aa, Macaca fascicularis
CTTDDRTSCSRGVCYAAFYSW

SEQ ID NO: 176 - protein, 128 aa, Macaca fascicularis
EVQLVESGAAVVQPGGSLRLSCAASGFTFSNSWMSWVRQSPGKGPEWVARIKRKADYETPDYAASVKGRFTISRDDSKNTLYLQMNSLKSEDTAVYFCTTDDRTSCSRGVCYAAFYSWGQGVLVTVSS

SEQ ID NO: 177 - protein, 6 aa, Macaca fascicularis
QAISFY

SEQ ID NO: 178 - skipped

SEQ ID NO: 179 - protein, 11 aa, Macaca fascicularis
CLQYDSPPFTF

SEQ ID NO: 180 - protein, 107 aa, Macaca fascicularis
DIQMTQSPSSLSASAGDRVTLTCRASQAISFYLAWYQQKPGKAPKRLIYDASELQGGVPSRFSGSGSGTDFTLSINSLQPEDSATYFCLQYDSPPFTFGPGTKVEIK

SEQ ID NO: 181 - protein, 8 aa, Macaca fascicularis
GFTFSDSW

SEQ ID NO: 182 - protein, 10 aa, Macaca fascicularis
IKGKPDGETA

SEQ ID NO: 183 - protein, 21 aa, Macaca fascicularis
CTRDDRTSCRRGVCYAAFHSW

SEQ ID NO: 184 - protein, 128 aa, Macaca fascicularis
EVQLVESGAGLVQPGGSLRLSCAASGFTFSDSWMSWVRQSPGKGLEWVGRIKGKPDGETAAYAASVKGRFSISRDDSKNTLYLQMNSLKTEDTAVYYCTRDDRTSCRRGVCYAAFHSWGQGVLVTVSS

SEQ ID NO: 185 - protein, 6 aa, Macaca fascicularis
NIGTEV

SEQ ID NO: 186 - skipped

SEQ ID NO: 187 - protein, 13 aa, Macaca fascicularis
CQVWDISSDHGLF

SEQ ID NO: 188 - protein, 108 aa, Macaca fascicularis
SYELTQPPSVSVSPGQTARITCGGDNIGTEVVHWYQQKPPQAPVLVIYADSERPSGIPERFSGSNSGNTATLTISGVEAGDEADYYCQVWDISSDHGLFGGGTRLTVL

SEQ ID NO: 189 - protein, 8 aa, Macaca fascicularis
GFTFSNYW

SEQ ID NO: 190 - protein, 10 aa, Macaca fascicularis
IKQKDDGEAA

SEQ ID NO: 191 - protein, 21 aa, Macaca fascicularis
CVRGWRVYCRNSGCSSAFDVW

SEQ ID NO: 192 - protein, 128 aa, Macaca fascicularis
EVQLVESGGGLVQPGGSLRLSCVASGFTFSNYWMSWVRQAPGKGLEWVAFIKQKDDGEAAAFAESVKGRFTISRDDSKNTLYLQMNSLKTEDTAAYYCVRGWRVYCRNSGCSSAFDVWGPGVLVTVSS

SEQ ID NO: 193 - protein, 6 aa, Macaca fascicularis
QSVSDF

SEQ ID NO: 194 - skipped

SEQ ID NO: 195 - protein, 10 aa, Macaca fascicularis
CFQYSSGWTF

SEQ ID NO: 196 - protein, 106 aa, Macaca fascicularis
EIVMTQSPATLSLSPGERVTLSCRASQSVSDFLAWYHQKPGQAPRLLIHSVSSRATGIPDRFSGSGSGTEFTLTISSLEPEDVGVYHCFQYSSGWTFGQGTKVEIK

SEQ ID NO: 197 - protein, 8 aa, Macaca fascicularis
GGSFGPYW

SEQ ID NO: 198 - protein, 7 aa, Macaca fascicularis
ISSDWGN

SEQ ID NO: 199 - protein, 21 aa, Macaca fascicularis
CARRRSEYCNNTYCTGAFDVW

SEQ ID NO: 200 - protein, 125 aa, Macaca fascicularis
QVQLQESGPGLVKPSETLSLTCTVSGGSFGPYWWGWIRQPPGKGLEWIGHISSDWGNYLHPSLVNRVTLSVDTTKNQFSLKLRSVVTADTAVYYCARRRSEYCNNTYCTGAFDVWGQGLRVTVSV

SEQ ID NO: 201 - protein, 11 aa, Macaca fascicularis
QSLLHSNGNTY

SEQ ID NO: 202 - skipped

SEQ ID NO: 203 - protein, 11 aa, Macaca fascicularis
CVQAIAFPWTF

SEQ ID NO: 204 - protein, 112 aa, Macaca fascicularis
DVVMTQTPLSLPITPGEPASISCRSSQSLLHSNGNTYLHWYLQKPGQSPQLLIYGGSNRASGVPDRFSGSGSGTDFTLKISKVEAEDVGVYYCVQAIAFPWTFGQGTKVEIK

SEQ ID NO: 205 - protein, 10 aa, Macaca fascicularis
SGSINDDSYY

SEQ ID NO: 206 - protein, 8 aa, Macaca fascicularis
IHGGTGKS

SEQ ID NO: 207 - protein, 19 aa, Macaca fascicularis
CARSHFCSNTFCYGWFDVW

SEQ ID NO: 208 - protein, 126 aa, Macaca fascicularis
QVQVQESGPGLVKPSETLSLTCAVSSGSINDDSYYWTWIRQSPGKGLEWLGFIHGGTGKSFYNPSLESRVTISKDTSRNQFSLTLSSVSAADTAVYYCARSHFCSNTFCYGWFDVWGPGIRVTVSS

SEQ ID NO: 209 - protein, 6 aa, Macaca fascicularis
QAISFY

SEQ ID NO: 210 - skipped

SEQ ID NO: 211 - protein, 11 aa, Macaca fascicularis
CLQYDSPPFTF

SEQ ID NO: 212 - protein, 107 aa, synthetic construct
DIQMTQSPSSLSASVGDRVTLTCRASQAISFYLAWFQQKPGKAPKRLIYDASELQGGVPSRFSGSGSGTDFTLSINSLQPEDSATYFCLQYDSPPFTFGPGTKVEIK

SEQ ID NO: 213 - protein, 8 aa, Macaca fascicularis
GFTFSDSW

SEQ ID NO: 214 - protein, 10 aa, Macaca fascicularis
IKGKPDGETA

SEQ ID NO: 215 - protein, 21 aa, Macaca fascicularis
CTRDDRTSCRRGVCYAAFHSW

SEQ ID NO: 216 - protein, 128 aa, Macaca fascicularis; identical to SEQ ID NO: 184

SEQ ID NO: 217 - protein, 6 aa, Macaca fascicularis
QAISFY

SEQ ID NO: 218 - skipped

SEQ ID NO: 219 - protein, 11 aa, Macaca fascicularis
CLQYDSPPFTF

SEQ ID NO: 220 - protein, 107 aa, synthetic construct
DIQMTQSPSSLSASVGDRVTLTCRASQAISFYLAWYQQKPGKAPKRLIYDASELQGGVPSRFSGSGSGTDFTLTINSLQPEDSATYFCLQYDSPPFTFGPGTKVEIK

SEQ ID NO: 221 - protein, 8 aa, Macaca fascicularis
GFTFSDSW

SEQ ID NO: 222 - protein, 10 aa, Macaca fascicularis
IKGKPDGETA

SEQ ID NO: 223 - protein, 21 aa, Macaca fascicularis
CTRDDRTSCRRGVCYAAFHSW

SEQ ID NO: 224 - protein, 128 aa, Macaca fascicularis; identical to SEQ ID NO: 184

SEQ ID NO: 225 - protein, 6 aa, Macaca fascicularis
QAISFY

SEQ ID NO: 226 - skipped

SEQ ID NO: 227 - protein, 11 aa, Macaca fascicularis
CLQYDSPPFTF

SEQ ID NO: 228 - protein, 107 aa, synthetic construct
DIQMTQSPSSLSASAGDRVTLTCRASQAISFYLAWFQQKPGKAPKRLIYDASELQGGVPSRFSGSGSGTDFTLTINSLQPEDSATYFCLQYDSPPFTFGPGTKVEIK

SEQ ID NO: 229 - protein, 8 aa, Macaca fascicularis
GFTFSDSW

SEQ ID NO: 230 - protein, 10 aa, Macaca fascicularis
IKGKPDGETA

SEQ ID NO: 231 - protein, 21 aa, Macaca fascicularis
CTRDDRTSCRRGVCYAAFHSW

SEQ ID NO: 232 - protein, 128 aa, Macaca fascicularis; identical to SEQ ID NO: 184

SEQ ID NO: 233 - protein, 6 aa, Macaca fascicularis
QAISFY

SEQ ID NO: 234 - skipped

SEQ ID NO: 235 - protein, 11 aa, Macaca fascicularis
CLQYDSPPFTF

SEQ ID NO: 236 - protein, 107 aa, synthetic construct
DIQMTQSPSSLSASVGDRVTLTCRASQAISFYLAWFQQKPGKAPKRLIYDASELQGGVPSRFSGSGSGTDFTLTINSLQPEDSATYFCLQYDSPPFTFGPGTKVEIK

SEQ ID NO: 237 - protein, 8 aa, Macaca fascicularis
GFTFSDSW

SEQ ID NO: 238 - protein, 10 aa, Macaca fascicularis
IKGKPDGETA

SEQ ID NO: 239 - protein, 21 aa, Macaca fascicularis
CTRDDRTSCRRGVCYAAFHSW

SEQ ID NO: 240 - protein, 128 aa, Macaca fascicularis; identical to SEQ ID NO: 184

SEQ ID NO: 241 - protein, 6 aa, Macaca fascicularis
QAISFY

SEQ ID NO: 242 - skipped

SEQ ID NO: 243 - protein, 11 aa, Macaca fascicularis
CLQYDSPPFTF

SEQ ID NO: 244 - protein, 107 aa, synthetic construct
DIQMTQSPSSLSASVGDRVTLTCRASQAISFYLAWYQQKPGKAPKRLIYDASELQGGVPSRFSGSGSGTDFTLSINSLQPEDSATYFCLQYDSPPFTFGPGTKVDIK

SEQ ID NO: 245 - protein, 8 aa, Macaca fascicularis
GFTFSDSW

SEQ ID NO: 246 - protein, 10 aa, Macaca fascicularis
IKGKPDGETA

SEQ ID NO: 247 - protein, 21 aa, Macaca fascicularis
CTRDDRTSCRRGVCYAAFHSW

SEQ ID NO: 248 - protein, 128 aa, Macaca fascicularis; identical to SEQ ID NO: 184

SEQ ID NO: 249 - protein, 6 aa, Macaca fascicularis
QAISFY

SEQ ID NO: 250 - skipped

SEQ ID NO: 251 - protein, 11 aa, Macaca fascicularis
CLQYDSPPFTF

SEQ ID NO: 252 - protein, 107 aa, synthetic construct
DIQMTQSPSSLSASAGDRVTLTCRASQAISFYLAWFQQKPGKAPKRLIYDASELQGGVPSRFSGSGSGTDFTLSINSLQPEDSATYFCLQYDSPPFTFGPGTKVDIK

SEQ ID NO: 253 - protein, 8 aa, Macaca fascicularis
GFTFSDSW

SEQ ID NO: 254 - protein, 10 aa, Macaca fascicularis
IKGKPDGETA

SEQ ID NO: 255 - protein, 21 aa, Macaca fascicularis
CTRDDRTSCRRGVCYAAFHSW

SEQ ID NO: 256 - protein, 128 aa, Macaca fascicularis; identical to SEQ ID NO: 184

SEQ ID NO: 257 - protein, 6 aa, Macaca fascicularis
QAISFY

SEQ ID NO: 258 - skipped

SEQ ID NO: 259 - protein, 11 aa, Macaca fascicularis
CLQYDSPPFTF

SEQ ID NO: 260 - protein, 107 aa, synthetic construct
DIQMTQSPSSLSASVGDRVTLTCRASQAISFYLAWFQQKPGKAPKRLIYDASELQGGVPSRFSGSGSGTDFTLSINSLQPEDSATYFCLQYDSPPFTFGPGTKVDIK

SEQ ID NO: 261 - protein, 8 aa, Macaca fascicularis
GFTFSDSW

SEQ ID NO: 262 - protein, 10 aa, Macaca fascicularis
IKGKPDGETA

SEQ ID NO: 263 - protein, 21 aa, Macaca fascicularis
CTRDDRTSCRRGVCYAAFHSW

SEQ ID NO: 264 - protein, 128 aa, Macaca fascicularis
EVQLVESGGGLVQPGGSLRLSCAASGFTFSDSWMSWVRQSPGKGLEWVGRIKGKPDGETAAYAASVKGRFSISRDDSKNTLYLQMNSLKTEDTAVYYCTRDDRTSCRRGVCYAAFHSWGQGTLVTVSS

SEQ ID NO: 265 - protein, 6 aa, Macaca fascicularis
QAISFY

SEQ ID NO: 266 - skipped

SEQ ID NO: 267 - protein, 11 aa, Macaca fascicularis
CLQYDSPPFTF

SEQ ID NO: 268 - protein, 107 aa, synthetic construct
DIQMTQSPSSLSASVGDRVTLTCRASQAISFYLAWYQQKPGKAPKRLIYDASELQGGVPSRFSGSGSGTDFTLTINSLQPEDSATYFCLQYDSPPFTFGPGTKVDIK

SEQ ID NO: 269 - protein, 8 aa, Macaca fascicularis
GFTFSDSW

SEQ ID NO: 270 - protein, 10 aa, Macaca fascicularis
IKGKPDGETA

SEQ ID NO: 271 - protein, 21 aa, Macaca fascicularis
CTRDDRTSCRRGVCYAAFHSW

SEQ ID NO: 272 - protein, 128 aa, Macaca fascicularis; identical to SEQ ID NO: 184

SEQ ID NO: 273 - protein, 6 aa, Macaca fascicularis
QAISFY

SEQ ID NO: 274 - skipped

SEQ ID NO: 275 - protein, 11 aa, Macaca fascicularis
CLQYDSPPFTF

SEQ ID NO: 276 - protein, 107 aa, synthetic construct
DIQMTQSPSSLSASAGDRVTLTCRASQAISFYLAWFQQKPGKAPKRLIYDASELQGGVPSRFSGSGSGTDFTLTINSLQPEDSATYFCLQYDSPPFTFGPGTKVDIK

SEQ ID NO: 277 - protein, 8 aa, Macaca fascicularis
GFTFSDSW

SEQ ID NO: 278 - protein, 10 aa, Macaca fascicularis
IKGKPDGETA

SEQ ID NO: 279 - protein, 21 aa, Macaca fascicularis
CTRDDRTSCRRGVCYAAFHSW

SEQ ID NO: 280 - protein, 128 aa, Macaca fascicularis; identical to SEQ ID NO: 184

SEQ ID NO: 281 - protein, 6 aa, Macaca fascicularis
QAISFY

SEQ ID NO: 282 - skipped

SEQ ID NO: 283 - protein, 11 aa, Macaca fascicularis
CLQYDSPPFTF

SEQ ID NO: 284 - protein, 107 aa, synthetic construct
DIQMTQSPSSLSASVGDRVTLTCRASQAISFYLAWFQQKPGKAPKRLIYDASELQGGVPSRFSGSGSGTDFTLTINSLQPEDSATYFCLQYDSPPFTFGPGTKVDIK

SEQ ID NO: 285 - protein, 8 aa, Macaca fascicularis
GFTFSDSW

SEQ ID NO: 286 - protein, 10 aa, Macaca fascicularis
IKGKPDGETA

SEQ ID NO: 287 - protein, 21 aa, Macaca fascicularis
CTRDDRTSCRRGVCYAAFHSW

SEQ ID NO: 288 - protein, 128 aa, Macaca fascicularis; identical to SEQ ID NO: 184

SEQ ID NO: 289 - protein, 453 aa, Macaca fascicularis
QVQLRESGPGLVKPSETLSLTCAVSGASITSNYWTWIRQPPGKGLEWIGRVYGDAGSTDYNPSLKSRVTISMDMSKNHFSLDLTSVTAADTALYYCASRSSVADNWFDVWGPGVLVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 290 - protein, 214 aa, Macaca fascicularis
DIQMTQSPSSLSASVGDTVTITCRASQNVMTLLAWYQHKPGKAPKLLIYQASSLHIGVPSRFSGSGSGTDFTLTISSLQSEDFATYSCQQYDSRPWTFGQGTKVDIKRTVAAPSVFIFPPSEDQVKSGTVSVVCLLNNFYPREASVKWKVDGALKTGNSQESVTEQDSKDNTYSLSSTLTLSSTEYQSHKVYACEVTHQGLSSPVTKSFNRGEC

SEQ ID NO: 291 - protein, 453 aa, Macaca fascicularis
QVQLQESGPGLVKPSETLSLTCAVSGDSISNNYWTWIRHFPGKGLEWIGRIYGDAGSTDYNPSLKSRVTISMDLSKNQFSLDLTSVTVADTALYYCASRTTVADNWFDVWGPGVLVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 292 - protein, 214 aa, Macaca fascicularis
DIQMTQSPSSLSASVGDTVTITCRATQSISSLLAWYQYKPGKAPKLLIYQASSLHIGVPSRFSGSGSGTDFTLTISSLQSEDFATYYCQQHDSRPWTFGQGTKVDIKRTVAAPSVFIFPPSEDQVKSGTVSVVCLLNNFYPREASVKWKVDGALKTGNSQESVTEQDSKDNTYSLSSTLTLSSTEYQSHKVYACEVTHQGLSSPVTKSFNRGEC

SEQ ID NO: 293 - protein, 453 aa, Macaca fascicularis
QVQLQESGPGLVKPSETLSLTCAVSSGSISGYSYWSWIRQPPGKGLEWIGNIYGSGGSSYLNPSLKSRVTLSLDTSKNHFSLRVTSVTAADTAVYYCALAKYSGSPGDYWGQGVLATVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 294 - protein, 218 aa, Macaca fascicularis
QSVLTQPPSVSGSPGQSVTISCTGTSSDIGVYNYVSWYQQHPGKAPKLMVSEVSIRPSGVSDRFSGSKSGNTASLTISGLQAEDEADYYCSSYSGIDTDTYIFGTGTRLTVLGQPKAAPSVTLFPPSSEELQANKATLVCLISDFYPGAVEVAWKADGSAVNAGVETTKPSKQSNNKYAASSYLSLTSDQWKSHKSYSCQVTHEGSTVEKTVAPAECS

SEQ ID NO: 295 - protein, 453 aa, Macaca fascicularis
QVQLQESGPGLVKPSETLSLTCAVSSGSISGFSYWSWIRQPPGRGLEWIGNIYGSGGSSYVNPSLKSRVTLSLDTSKNQFSLRVTSVTAADTAVYYCALAKYSGSPGDYWGQGVLATVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 296 - protein, 217 aa, Macaca fascicularis
QFVLTQPPSVSGSPGQSVTISCTGTSSDIGVYNYVSWYQQYPGKAPKLMVSEVSLRPSGVSHRFSGSKSGNTASLTISGLQAEDEADYYCSSYSGSETDTYIFGTGTRLTVGQPKAAPSVTLFPPSSEELQANKATLVCLISDFYPGAVEVAWKADGSAVNAGVETTKPSKQSNNKYAASSYLSLTSDQWKSHKSYSCQVTHEGSTVEKTVAPAECS

SEQ ID NO: 297 - protein, 449 aa, Macaca fascicularis
QVQLQESGPGLVKPSETLSLTCVVSGVSISTKYHYWSWIRQAPGKGLEWIGYISYTGGTSYNPSLQSRVTISRDTSKNHFSLSFNSVTPADTALYFCASDENKPYWGQGVLVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 298 - protein, 220 aa, Macaca fascicularis
DVVMTQSPESLAVSLGERVTISCKSSQSLLYNTNNKEYLAWYQQKPGQAPKLLFYWASTRESGVPNRFSGSGSGTDFTLTISGLQAEDVAVYYCQQYYSSPRTFGQGTKVEIKRTVAAPSVFIFPPSEDQVKSGTVSVVCLLNNFYPREASVKWKVDGALKTGNSQESVTEQDSKDNTYSLSSTLTLSSTEYQSHKVYACEVTHQGLSSPVTKSFNRGEC

SEQ ID NO: 299 - protein, 449 aa, Macaca fascicularis
QVQLRESGPGLVKPSETLSLTCAVSGGSLTTNYYYWSWIRQAPGKGLEWIGYISYTGSTNYNPSLQSRVTISRDTSKNQFSLNLSSMKVADTAVYFCVGDENKIHWGPGVLVIVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 300 - protein, 220 aa, Macaca fascicularis
DIVMTQSPDSVAVSLGERVTINCKSGQSLLYNSNNKNYLAWYQQKPGQAPNLLIYWASTRESGVPNRFSGSGSGTNFTLTISGLQAEDVAVYYCQQYYSSPRTFGQGTKVDIKRTVAAPSVFIFPPSEDQVKSGTVSVVCLLNNFYPREASVKWKVDGALKTGNSQESVTEQDSKDNTYSLSSTLTLSSTEYQSHKVYACEVTHQGLSSPVTKSFNRGEC

SEQ ID NO: 301 - protein, 449 aa, Macaca fascicularis
QVQLQESGPGLVKPSETLSLTCAVSGGSISSSYSYWSWIRQAPGKGLEWIGYISYTGSTNYNPSLKSRVTISRDTSKNQFSLELTSMTTADTAVYYCATDRNTLAWGPGVLVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 302 - protein, 219 aa, Macaca fascicularis
DIVMTQSPDSLAVSLGERVTINCKSSQSLLYSSNNKNYLAWYQQKPGQAPRLLIYWASTRESGVPNRFSGSGSGTDFTLTINGLQAEDVAVYYCQQYYNSRTFGQGTKVEIKRTVAAPSVFIFPPSEDQVKSGTVSVVCLLNNFYPREASVKWKVDGALKTGNSQESVTEQDSKDNTYSLSSTLTLSSTEYQSHKVYACEVTHQGLSSPVTKSFNRGEC

SEQ ID NO: 303 - protein, 463 aa, Macaca fascicularis
EVQLVESGGGLAKPGGSLRLSCAASGFTFSSYAMHWVRQAPGKGLEWVSVISSHYSTYYADSVKGRFTISRDNSKNTVSLQVNSLRAEDTAVYYCAKVGRSGYCTASGCNALLFDWFDVWGPGVLVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 304 - protein, 219 aa, Macaca fascicularis
DVVMTQSPLSLPVTPGQPASISCRSSQSLVHSNGNTSLSWYQQKAGHPPRLLIYQVSNRYSGVPDRFSGTGAGTDFTLTISRVETEDVGVYYCGQGAHLPPAFGQGTKVEIKRTVAAPSVFIFPPSEDQVKSGTVSVVCLLNNFYPREASVKWKVDGALKTGNSQESVTEQDSKDNTYSLSSTLTLSSTEYQSHKVYACEVTHQGLSSPVTKSFNRGEC

SEQ ID NO: 305 - protein, 457 aa, Macaca fascicularis
EVQLVESGGGLVQPGGSLRLSCVASGFTFSDYRMAWVRQATGKGLEWVSTISQPSGTNTYYLDPVKGRFTVSRDNAKNTLYLQMHSLRAEDTAVYYCARVVTESRPPAAWFDVWGPGVLVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 306 - protein, 215 aa, Macaca fascicularis
SYELTQPPSVSASPGQTARITCGGINIGSELVHWYQQKPPQAPVLVIYANGERPSGIPERFSGSNSGNTATLTISGVEAGDEADYYCQLWDISSDHNYIFGDGTRLTVLGQPKAAPSVTLFPPSSEELQANKATLVCLISDFYPGAVEVAWKADGSAVNAGVETTKPSKQSNNKYAASSYLSLTSDQWKSHKSYSCQVTHEGSTVEKTVAPAECS

SEQ ID NO: 307 - protein, 461 aa, Macaca fascicularis
EVQLVESGGGLGKPGGSLRLSCAASGFTFSSYGMHWVRQAPGKGLEWASSITSSGGNTDYADSVKGRFTISRDNSKNTLSLQMNSLRPEDSATYYCAKEKYSSYSAYGRGYPNSLDVWGRGVLVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 308 - protein, 220 aa, Macaca fascicularis
DIVMTQTPPSLPVTLGEPASISCRSSQSLFDSGDVTTYLDWYLQKPGQSPQLLIFEVSKRASGVPERFSGSGSDTDFTLQISRVEAEDVGVYYCMQPLEYPRTFGQGTRVEIKRTVAAPSVFIFPPSEDQVKSGTVSVVCLLNNFYPREASVKWKVDGALKTGNSQESVTEQDSKDNTYSLSSTLTLSSTEYQSHKVYACEVTHQGLSSPVTKSFNRGEC

SEQ ID NO: 309 - protein, 461 aa, Macaca fascicularis
EVQLVESGGGLGKPGGSLRLSCAASGFTFSSYGMHWVRQAPGKGLEWASSITSSGGNTDYADSVKGRFTISRDNSKNTLYLQMNSLRPEDSGTYYCAKEKYSSYSAYGRGYPNSLDVWGRGVLVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 310 - protein, 220 aa, Macaca fascicularis
DVVMTQTPLSLPVTLGEPASISCRSSQSLFDSGDVTTYLDWYLQKPGQSPQLLIFEVSKRASGVPERFSGSGSDTDFTLQISRVEAEDVGVYYCMQPLEYPRTFGQGTKVEIKRTVAAPSVFIFPPSEDQVKSGTVSVVCLLNNFYPREASVKWKVDGALKTGNSQESVTEQDSKDNTYSLSSTLTLSSTEYQSHKVYACEVTHQGLSSPVTKSFNRGEC

SEQ ID NO: 311 - protein, 455 aa, Macaca fascicularis
VVQLVQSGAEVKRPGESLRISCKTSANSFTGSWINWVRQVPGKGLEWMGSIYGGDSDLRYNPSFRGHVTFTADKSISTAYLQWTSLQASDTATYYCARSPWSGYYIGWFDVWGPGVLVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 312 - protein, 219 aa, Macaca fascicularis
DVVMTQTPLSLPVTPGEPTSISCRSSQSLLDSNGNTYLHWYLQKPGQSPQLLIYGGSNRPSGVPDRFSGSGSGTDFTLKISNVEAGDVGTYYCMQVLQVPLTFGGGTKVDIKRTVAAPSVFIFPPSEDQVKSGTVSVVCLLNNFYPREASVKWKVDGALKTGNSQESVTEQDSKDNTYSLSSTLTLSSTEYQSHKVYACEVTHQGLSSPVTKSFNRGEC

SEQ ID NO: 313 - protein, 455 aa, Macaca fascicularis
VVQLVQSGAEVKRPGESLRISCKTSGYRFTDIWINWVRQRPGKGLEWMGSIYLDDSDIRFNPSFQGHVTISADKSTSTTYLQWSSLKASDTATYFCARSVWSGYYIGWFDVWGPGVLVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 314 - protein, 219 aa, Macaca fascicularis
DVVMTQTPLSLPVTPGEPASISCRSSQSLLDSNGNTYLHWYLQKPGQSPQLLIYGGSNRASGVPDRFSGSGSGTDFTLTINNVEAGDVGTYYCMQVIQVPLTFGGGTKVDIKRTVAAPSVFIFPPSEDQVKSGTVSVVCLLNNFYPREASVKWKVDGALKTGNSQESVTEQDSKDNTYSLSSTLTLSSTEYQSHKVYACEVTHQGLSSPVTKSFNRGEC

SEQ ID NO: 315 - protein, 455 aa, Macaca fascicularis
VVQLVQSGAGLKRPGESLRISCKTSGYSFTDIWISWVRQMPGKGLEWMGSLYLDDSDIRFNPSFQGHVTMSADKSTSTAYLQWSSLRASDTATYYCARSVWSGYYIGWFDVWGPGVLVAVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 316 - protein, 219 aa, Macaca fascicularis
DVVMTQTPLSLPVTPGESASISCRSSQSLLDSNGNTYLHWYLQKPGQSPQLLIYGGSNRASGVPDRFSGSGSGTDFTLKISNVEAGDVGSYYCMQVIQVPLTFGGGTKVEIKRTVAAPSVFIFPPSEDQVKSGTVSVVCLLNNFYPREASVKWKVDGALKTGNSQESVTEQDSKDNTYSLSSTLTLSSTEYQSHKVYACEVTHQGLSSPVTKSFNRGEC

SEQ ID NO: 317 - protein, 460 aa, Macaca fascicularis
EVQLVESGGGLAQPGGSLRLSCVASGFTFSDYEMAWVRQATGKGLEWVSSVGQPSDSQTYYLDPVKGRFTISRDNAKNTLYLQMNSLGAEDTAVYYCARLFRGYCSSTFCFKWFDVWGPGVLVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 318 - protein, 214 aa, Macaca fascicularis
DIQMSQSPSSLSASVGDRVSITCRASQDISSFLNWYQQKPGKAPNLLIHYGEFLAGGVPSRFSGSGSGTDYTLTISSLQPEDLGTYYCQQGHSIPYSFGQGTKVEIKRTVAAPSVFIFPPSEDQVKSGTVSVVCLLNNFYPREASVKWKVDGALKTGNSQESVTEQDSKDNTYSLSSTLTLSSTEYQSHKVYACEVTHQGLSSPVTKSFNRGEC

SEQ ID NO: 319 - protein, 453 aa, Macaca fascicularis
QVQLQESGPGLVKPSETLSLTCTVSGGSISGYYWNWIRQSPGKGLEWIGNMYDSTGDTNYNPSLKSRVTLSIDTSKNQFSMKLNSVTAADTAVYFCASGSGDWFGYFYRWGQGVLVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 320 - protein, 214 aa, Macaca fascicularis
DIQMTQSPSSLSASVGDTVTITCRASQSISSWLAWYQQKPGKAPNLLIYKASSLQTGVPSRFSGSGSETHFTLTISSLQSEDFATYYCQQYISRPWTFGQGTKVEIKRTVAAPSVFIFPPSEDQVKSGTVSVVCLLNNFYPREASVKWKVDGALKTGNSQESVTEQDSKDNTYSLSSTLTLSSTEYQSHKVYACEVTHQGLSSPVTKSFNRGEC

SEQ ID NO: 321 - protein, 453 aa, Macaca fascicularis
IVQLQESGPGLVKPSETLSLTCAVSGGSISGGDYYWTWIRQSPGKGLEWIGYIHGNWGNTLYNPSLESRITISKDTSNNQFSLSLRSVTAADTAVYYCVREAYGTYFYFWGQGVLVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 322 - protein, 214 aa, Macaca fascicularis
DIQMTQSPSALSASVGDRVTITCRASHNINSFLAWYQQKPGQVPKLLIYAASSLENGVPSRFSGSGSGTEFTLTIRGLQPEDFATYFCLQHSRHPFSFGPGTKVEIKRTVAAPSVFIFPPSEDQVKSGTVSVVCLLNNFYPREASVKWKVDGALKTGNSQESVTEQDSKDNTYSLSSTLTLSSTEYQSHKVYACEVTHQGLSSPVTKSFNRGEC

SEQ ID NO: 323 - protein, 456 aa, Macaca fascicularis
EVQLVESGGGLAKPGGSLRLSCAAAGFRFSDFHMHWVRQAPGKGLEWVSRISHGGVTTRFADSVKGRFTISRENAKNTLYLQMDNLRAEDTAVYYCARDSPYWGDYSHSLDVWGRGVLVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 324 - protein, 213 aa, Macaca fascicularis
PMRLTQPRSVSVSPGQTARITCGGDNIGSKSVHWYQQKPPQAPVLVIYDDRARPSGIPERFSGSKSGNTATLAISGVEAGDEADYYCQVWDSSSDVLFGGGTRLTTVGQPKAAPSVTLFPPSSEELQANKATLVCLISDFYPGAVEVAWKADGSAVNAGVETTKPSKQSNNKYAASSYLSLTSDQWKSHKSYSCQVTHEGSTVEKTVAPAECS

SEQ ID NO: 325 - protein, 447 aa, Macaca fascicularis
EVQLVESGGGLVQPGGSLRLSCVASGFPFSDYDMNWVRQAPGKGLEWIGFIKSKPGGGTPYYAASVKGRFTISRDDSKNTLYLQMNSLKIEDTAVFYCTTSVVWGQGALVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 326 - protein, 214 aa, Macaca fascicularis
DIQMTQSPSSLTASVGDRVTITCRASQDINKYLSWYQQKPGKAPKSLIYLASSLERGVPTRFSGSGSGTHYILTISSLQPEDIGTYYCQQHNISPYNFGQGTKIQIKRTVAAPSVFIFPPSEDQVKSGTVSVVCLLNNFYPREASVKWKVDGALKTGNSQESVTEQDSKDNTYSLSSTLTLSSTEYQSHKVYACEVTHQGLSSPVTKSFNRGEC

SEQ ID NO: 327 - protein, 453 aa, Macaca fascicularis
QVTLKESGPALVKPTQTLTLTCTFSGFSISSTGIGVGWIRQPPGKALEWLANIYWNDNTDYNTSLKNRLTISKDTSKNQVVLTMTNMDPVDTATYHCAFLVVLTKWFDVWGPGVLVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 328 - protein, 216 aa, Macaca fascicularis
PFVLTQPPSVSGDPGQKVTISCSGSTSNIGDYYVHWYQQFPGTAPKLLIFENNRRPSGVSDRFSGSKSGSSASLTISGLQPGDEADYYCGTWDNSLTSVLFGGGTRLTTVGQPKAAPSVTLFPPSSEELQANKATLVCLISDFYPGAVEVAWKADGSAVNAGVETTKPSKQSNNKYAASSYLSLTSDQWKSHKSYSCQVTHEGSTVEKTVAPAECS

SEQ ID NO: 329 - protein, 461 aa, Macaca fascicularis
EVQLVESGAGLVQPGGSLRLSCAASGFTFSDSWMSWVRQSPGKGLEWVARIKRRADGETADYAPSVKGRFIVSRDDSNNTLYLQMNSLKTEDTAVYFCTTDDRTYCSRGVCYAAFYHWGQGVLVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 330 - protein, 214 aa, Macaca fascicularis
DIQMTQSPSSLSASAGDRVTITCRASQAISSSLAWYQQKPGKAPKRLIFDASSLQGGVPSRFSGSGSGTDFTLTISSLQPEDFATYFCLQYNSAPFTFGPGTKLDIMRTVAAPSVFIFPPSEDQVKSGTVSVVCLLNNFYPREASVKWKVDGALKTGNSQESVTEQDSKDNTYSLSSTLTLSSTEYQSHKVYACEVTHQGLSSPVTKSFNRGEC

SEQ ID NO: 331 - protein, 461 aa, Macaca fascicularis
EVQLVESGAAVVQPGGSLRLSCAASGFTFSNSWMSWVRQSPGKGPEWVARIKRKADYETPDYAASVKGRFTISRDDSKNTLYLQMNSLKSEDTAVYFCTTDDRTSCSRGVCYAAFYSWGQGVLVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 332 - protein, 214 aa, Macaca fascicularis
DIQMTQSPSSLSASAGDRVSITCRASQDISSYLAWYQQKPGKPPKRLIFDASSLQGGVPSRFSGSGSGTDFTLTISSLQPEDFATYFCLQYNSAPFTFGPGTQLDIKRTVAAPSVFIFPPSEDQVKSGTVSVVCLLNNFYPREASVKWKVDGALKTGNSQESVTEQDSKDNTYSLSSTLTLSSTEYQSHKVYACEVTHQGLSSPVTKSFNRGEC

SEQ ID NO: 333 - protein, 461 aa, Macaca fascicularis
EVQLVESGAGLVQPGGSLRLSCAASGFTFSDSWMSWVRQSPGKGLEWVGRIKGKPDGETAAYAASVKGRFSISRDDSKNTLYLQMNSLKTEDTAVYYCTRDDRTSCRRGVCYAAFHSWGQGVLVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 334 - protein, 214 aa, Macaca fascicularis
DIQMTQSPSSLSASAGDRVTLTCRASQAISFYLAWYQQKPGKAPKRLIYDASELQGGVPSRFSGSGSGTDFTLSINSLQPEDSATYFCLQYDSPPFTFGPGTKVEIKRTVAAPSVFIFPPSEDQVKSGTVSVVCLLNNFYPREASVKWKVDGALKTGNSQESVTEQDSKDNTYSLSSTLTLSSTEYQSHKVYACEVTHQGLSSPVTKSFNRGEC

SEQ ID NO: 335 - protein, 461 aa, Macaca fascicularis
EVQLVESGGGLVQPGGSLRLSCVASGFTFSNYWMSWVRQAPGKGLEWVAFIKQKDDGEAAAFAESVKGRFTISRDDSKNTLYLQMNSLKTEDTAAYYCVRGWRVYCRNSGCSSAFDVWGPGVLVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 336 - protein, 214 aa, Macaca fascicularis
SYELTQPPSVSVSPGQTARITCGGDNIGTEVVHWYQQKPPQAPVLVIYADSERPSGIPERFSGSNSGNTATLTISGVEAGDEADYYCQVWDISSDHGLFGGGTRLTVLGQPKAAPSVTLFPPSSEELQANKATLVCLISDFYPGAVEVAWKADGSAVNAGVETTKPSKQSNNKYAASSYLSLTSDQWKSHKSYSCQVTHEGSTVEKTVAPAECS

SEQ ID NO: 337 - protein, 458 aa, Macaca fascicularis
QVQLQESGPGLVKPSETLSLTCTVSGGSFGPYWWGWIRQPPGKGLEWIGHISSDWGNYLHPSLVNRVTLSVDTTKNQFSLKLRSVVTADTAVYYCARRRSEYCNNTYCTGAFDVWGQGLRVTVSVASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 338 - protein, 213 aa, Macaca fascicularis
EIVMTQSPATLSLSPGERVTLSCRASQSVSDFLAWYHQKPGQAPRLLIHSVSSRATGIPDRFSGSGSGTEFTLTISSLEPEDVGVYHCFQYSSGWTFGQGTKVEIKRTVAAPSVFIFPPSEDQVKSGTVSVVCLLNNFYPREASVKWKVDGALKTGNSQESVTEQDSKDNTYSLSSTLTLSSTEYQSHKVYACEVTHQGLSSPVTKSFNRGEC

SEQ ID NO: 339 - protein, 459 aa, Macaca fascicularis
QVQVQESGPGLVKPSETLSLTCAVSSGSINDDSYYWTWIRQSPGKGLEWLGFIHGGTGKSFYNPSLESRVTISKDTSRNQFSLTLSSVSAADTAVYYCARSHFCSNTFCYGWFDVWGPGIRVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 340 - protein, 219 aa, Macaca fascicularis
DVVMTQTPLSLPITPGEPASISCRSSQSLLHSNGNTYLHWYLQKPGQSPQLLIYGGSNRASGVPDRFSGSGSGTDFTLKISKVEAEDVGVYYCVQAIAFPWTFGQGTKVEIKRTVAAPSVFIFPPSEDQVKSGTVSVVCLLNNFYPREASVKWKVDGALKTGNSQESVTEQDSKDNTYSLSSTLTLSSTEYQSHKVYACEVTHQGLSSPVTKSFNRGEC

SEQ ID NO: 341 - protein, 458 aa, Macaca fascicularis
EVQLVESGAGLVQPGGSLRLSCAASGFTFSDSWMSWVRQSPGKGLEWVGRIKGKPDGETAAYAASVKGRFSISRDDSKNTLYLQMNSLKTEDTAVYYCTRDDRTSCRRGVCYAAFHSWGQGVLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 342 - protein, 214 aa, synthetic construct
DIQMTQSPSSLSASVGDRVTLTCRASQAISFYLAWFQQKPGKAPKRLIYDASELQGGVPSRFSGSGSGTDFTLSINSLQPEDSATYFCLQYDSPPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 343 - protein, 458 aa, Macaca fascicularis; identical to SEQ ID NO: 341

SEQ ID NO: 344 - protein, 214 aa, synthetic construct
DIQMTQSPSSLSASVGDRVTLTCRASQAISFYLAWYQQKPGKAPKRLIYDASELQGGVPSRFSGSGSGTDFTLTINSLQPEDSATYFCLQYDSPPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 345 - protein, 458 aa, Macaca fascicularis; identical to SEQ ID NO: 341

SEQ ID NO: 346 - protein, 214 aa, synthetic construct
DIQMTQSPSSLSASAGDRVTLTCRASQAISFYLAWFQQKPGKAPKRLIYDASELQGGVPSRFSGSGSGTDFTLTINSLQPEDSATYFCLQYDSPPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 347 - protein, 458 aa, Macaca fascicularis; identical to SEQ ID NO: 341

SEQ ID NO: 348 - protein, 214 aa, synthetic construct
DIQMTQSPSSLSASVGDRVTLTCRASQAISFYLAWFQQKPGKAPKRLIYDASELQGGVPSRFSGSGSGTDFTLTINSLQPEDSATYFCLQYDSPPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 349 - protein, 458 aa, Macaca fascicularis; identical to SEQ ID NO: 341

SEQ ID NO: 350 - protein, 214 aa, synthetic construct
DIQMTQSPSSLSASVGDRVTLTCRASQAISFYLAWYQQKPGKAPKRLIYDASELQGGVPSRFSGSGSGTDFTLSINSLQPEDSATYFCLQYDSPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 351 - protein, 458 aa, Macaca fascicularis; identical to SEQ ID NO: 341

SEQ ID NO: 352 - protein, 214 aa, synthetic construct
DIQMTQSPSSLSASAGDRVTLTCRASQAISFYLAWFQQKPGKAPKRLIYDASELQGGVPSRFSGSGSGTDFTLSINSLQPEDSATYFCLQYDSPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 353 - protein, 458 aa, Macaca fascicularis
EVQLVESGGGLVQPGGSLRLSCAASGFTFSDSWMSWVRQSPGKGLEWVGRIKGKPDGETAAYAASVKGRFSISRDDSKNTLYLQMNSLKTEDTAVYYCTRDDRTSCRRGVCYAAFHSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 354 - protein, 214 aa, synthetic construct
DIQMTQSPSSLSASVGDRVTLTCRASQAISFYLAWFQQKPGKAPKRLIYDASELQGGVPSRFSGSGSGTDFTLSINSLQPEDSATYFCLQYDSPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 355 - protein, 458 aa, Macaca fascicularis; identical to SEQ ID NO: 341

SEQ ID NO: 356 - protein, 214 aa, synthetic construct
DIQMTQSPSSLSASVGDRVTLTCRASQAISFYLAWYQQKPGKAPKRLIYDASELQGGVPSRFSGSGSGTDFTLTINSLQPEDSATYFCLQYDSPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 357 - protein, 458 aa, Macaca fascicularis; identical to SEQ ID NO: 341

SEQ ID NO: 358 - protein, 214 aa, synthetic construct
DIQMTQSPSSLSASAGDRVTLTCRASQAISFYLAWFQQKPGKAPKRLIYDASELQGGVPSRFSGSGSGTDFTLTINSLQPEDSATYFCLQYDSPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 359 - protein, 458 aa, Macaca fascicularis; identical to SEQ ID NO: 341

SEQ ID NO: 360 - protein, 214 aa, synthetic construct
DIQMTQSPSSLSASVGDRVTLTCRASQAISFYLAWFQQKPGKAPKRLIYDASELQGGVPSRFSGSGSGTDFTLTINSLQPEDSATYFCLQYDSPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC